(19) United States
(12) Patent Application Publication
(10) Pub. No.: US 2018/0110223 A1
(43) Pub. Date: Apr. 26, 2018

(54) PHENOXY- OR PYRIDIN-2-YLOXY-AMINOTRIAZINE COMPOUNDS AND THEIR USE AS HERBICIDES

(71) Applicant: BASF SE, Ludwigshafen am Rhein (DE)

(72) Inventors: Florian VOGT, Mannheim (DE); Frederick CALO, Duesseldorf (DE); Julia MAJOR, Freinsheim (DE); Thomas SEITZ, Viernheim (DE); Doreen SCHACHTSCHABEL, Mannheim (DE); Trevor William NEWTON, Neustadt (DE); Kristin HANZLIK, Bobenheim am Berg (DE); Johannes HUTZLER, Waldsee (DE); Klaus KREUZ, Denzlingen (DE); Stefan TRESCH, Kirchheim (DE)

(21) Appl. No.: 15/568,531

(22) PCT Filed: Apr. 14, 2016
(86) PCT No.: PCT/EP2016/058206

§ 371 Date: Oct. 23, 2017

(30) Foreign Application Priority Data
Apr. 24, 2015 (EP) 15164950.6

(51) Int. Cl. A01N 43/66; A01N 43/68; C07D 401/12; C07D 251/16; C07D 409/04; C07D 405/04; C07D 413/04; A01N 43/76

(52) U.S. Cl. CPC A01N 43/66; A01N 43/68; C07D 401/12; A01N 43/76; C07D 409/04; C07D 405/04; C07D 413/04; C07D 251/16

(57) ABSTRACT

The present invention relates to phenoxy- or pyridyloxy-aminotriazine compounds and to their use as herbicides. The present invention also relates to agrochemical compositions for crop protection and to a method for controlling unwanted vegetation.

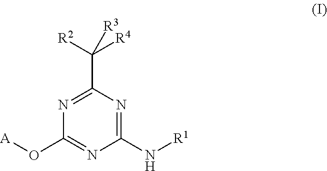

wherein
A is a radical of formula (A.1)

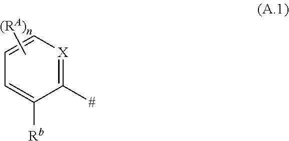

and
X is CR^{A4} or N;
R¹ is inter alia H, OH, S(O)₂NH₂, CN, C₁-C₆-alkyl, C₂-C₆-alkenyl, C₂-C₆-alkynyl, (C₃-C₆-cycloalkyl)-C₁-C₄-alkyl, C₁-C₆-alkoxy, (C₁-C₆-alkoxy)-C₁-C₆-alkyl, etc.;
R² is inter alia H, halogen, OH, CN, C₁-C₆-alkyl, C₂-C₆-alkenyl, C₃-C₆-alkynyl, (C₁-C₆-alkoxy)-C₁-C₆-alkyl, C₃-C₆-cycloalkyl, C₃-C₆-cycloalkenyl, (C₃-C₆-cycloalkyl)-C₁-C₄-alkyl, C₁-C₆-alkoxy, C₂-C₆-alkenyloxy, C₂-C₆-alkynyloxy, C₃-C₆-cycloalkoxy or (C₃-C₆-cycloalkyl)-C₁-C₄-alkoxy, etc.;
R³ is inter alia selected from the group consisting of H, halogen, CN, C₁-C₆-alkyl, C₁-C₆-haloalkyl, C₁-C₆-alkoxy and C₁-C₆-haloalkoxy;
R⁴ is inter alia selected from the group consisting of H, halogen, CN, C₁-C₆-alkyl, C₂-C₆-alkenyl, C₃-C₆-alkynyl, C₃-C₆-cycloalkyl, etc.;
R³ and R⁴ together with the carbon atom to which they are attached form a moiety selected from the group consisting of carbonyl, C₂-C₆-alkenyl, C₃-C₆-cycloalkyl, C₃-C₆-cycloalkenyl and three- to six-membered heterocyclyl, etc.
   including their agriculturally acceptable salts.

The present invention relates to phenoxy- or pyridyloxy-aminotriazine compounds and to their use as herbicides. The present invention also relates to agrochemical compositions for crop protection and to a method for controlling unwanted vegetation.

U.S. Pat. No. 3,816,419 describes 4-haloalkyl or 4-haloalkenyl-2,4-aminotriazines and their use as herbicides. Similar compounds are known from U.S. Pat. No. 3,932,167.

DE 197 44 711 describes herbicidally active 2,4-diamino-1,3,5-triazine compounds, which carry a group A-Z in the 6-position, where A is alkylene and Z is a carbocylic or heterocyclic radical.

DE 198 30 902 describes amino-chloro-triazine compounds and their use as herbicides.

EP 0545 149 describes 6-trifluoromethyl-1,3,5-triazine compounds and their use as intermediates for crop protecting agents.

However, the herbicidal properties of the known triazine type compounds are not always entirely satisfactory.

Earlier filed WO 2014/064094 describes 2-phenylamino-6-aminotriazine compounds having herbicide activity.

Earlier filed WO 2015/007711 describes 2-(hetaryl)amino-6-aminotriazine compounds having herbicide activity.

EP 0 373 472 discloses the following compounds 4-(2-fluorophenoxy)-6-methyl-1,3,5-triazin-2-amine, 4-(2-chlorophenoxy)-6-methyl-1,3,5-triazin-2-amine, 2-[(4-amino-6-methyl-1,3,5-triazin-2-yl)oxy]benzonitrile, 4-methyl-6-(2,4,6-trichlorophenoxy)-1,3,5-triazin-2-amine, 4-[2-chloro-4-(trifluoromethyl)phenoxy]-6-methyl-1,3,5-triazin-2-amine, 4-(2-fluorophenoxy)-6-(trifluoromethyl)-1,3,5-triazin-2-amine, 4-(2-chlorophenoxy)-6-(trifluoromethyl)-1,3,5-triazin-2-amine, 2-[(4-amino-6-(trifluoromethyl)-1,3,5-triazin-2-yl)oxy]benzonitrile, 4-(2,4,6-trichlorophenoxy)-6-(trifluoromethyl)-1,3,5-triazin-2-amine, 4-[2-chloro-4-(trifluoromethyl)phenoxy]-6-(trifluoromethyl)-1,3,5-triazin-2-amine. These compounds are used as precursor for the preparation of substituted sulfonylureas.

GB 943 637 discloses 4-(2,6-dichlorophenoxy)-6-(trifluoromethyl)-1,3,5-triazin-2-amine which is used as an intermediate for the preparation of polymers.

WO 97/35580 discloses 4-(2-bromo-4-isopropenyl-6-methoxy-phenoxy)-N-[1-(methoxymethyl)propyl]-6-methyl-1,3,5-triazin-2-amine and 4-(2-bromo-4-isopropyl-6-methoxyphenoxy)-N-[1-(methoxymethyl)propyl]-6-methyl-1,3,5-triazin-2-amine. These compounds are used in pharmaceutical compositions for the treatment of several diseases such as anxiety, depression, Alzheimer's disease etc.

The following compounds 4-[2-bromo-4-methoxy-6-(1-methylethenyl)phenoxy]-N-[1-(methoxymethyl)propyl]-6-methyl-1,3,5-triazin-2-amine, 4-[2-bromo-4-methoxy-6-(1-methylethyl)phenoxy]-N-[1-(methoxymethyl)propyl]-6-methyl-1,3,5-triazin-2-amine, 1-[3-bromo-5-methoxy-4-[[4-[[1-(methoxymethyl)propyl]amino]-6-methyl-1,3,5-triazin-2-yl]oxy]phenyl]-ethanone are known from chemical abstracts CAS-RN: 1349525-90-1, CAS-RN: 1349522-79-7 and CAS-RN: 1348221-62-4, respectively.

It is an object of the present invention to provide compounds having improved herbicidal action, in particular good herbicide activity at low application rates. Moreover, the herbicides should be sufficiently compatible with crop plants for commercial utilization.

These and further objects are achieved by aminotriazine compounds of formula (I), defined below, and by their agriculturally suitable salts:

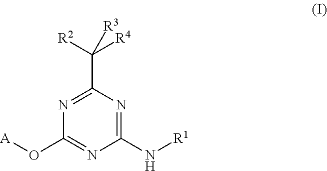

In formula (I) the variables A, R¹, R², R³ and R⁴ are as defined herein:
A is a radical of formula (A.1)

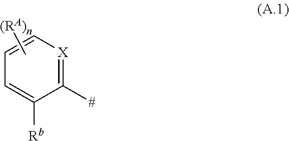

wherein # indicates the point of attachment to the oxygen
   X is CR^{A4} or N;
   R^{A} is selected from the group consisting of halogen, OH, CN, amino, NO₂, C₁-C₆-alkyl, C₂-C₆-alkenyl, C₂-C₆-alkynyl, C₁-C₆-alkoxy, C₂-C₆-alkenyloxy, C₂-C₆-alkynyloxy, (C₁-C₆-alkoxy)-C₁-C₆-alkyl, (C₁-C₆-alkoxy)-C₁-C₆-alkoxy, (C₁-C₆-alkoxy)-C₂-C₆-alkenyl, (C₁-C₆-alkoxy)-C₂-C₆-alkynyl, C₁-C₆-alkylthio, (C₁-C₆-alkyl)sulfinyl, (C₁-C₆-alkyl)sulfonyl, (C₁-C₆-alkyl)amino, di(C₁-C₆-alkyl)amino, (C₁-C₆-alkyl)-carbonyl, (C₁-C₆-alkoxy)-carbonyl, (C₁-C₆-alkyl)-carbonyloxy, C₃-C₆-cycloalkyl, C₃-C₆-cycloalkoxy, (C₃-C₆-cycloalkyl)-C₁-C₄-alkyl, (C₃-C₆-cycloalkyl)-C₁-C₄-alkoxy, where the aliphatic and cycloaliphatic parts of the 22 aforementioned radicals are unsubstituted, partly or completely halogenated and where the cycloaliphatic parts of the last 4 mentioned radicals may carry 1, 2, 3, 4, 5 or 6 methyl groups, it being possible that two radicals R^{A} which are bound to adjacent ring atoms of phenyl or 6-membered hetaryl may form a fused 5- or 6-membered carbocyclic or heterocyclic ring, which itself is substituted or unsubstituted by 1, 2, 3, 4 or 5 identical or different substituents R^{B} selected from the group consisting of halogen, OH, CN, amino, NO₂, C₁-C₆-alkyl, C₂-C₆-alkenyl, C₂-C₆-alkynyl, C₁-C₆-alkoxy, C₂-C₆-alkenyloxy, C₂-C₆-alkynyloxy, (C₁-C₆-alkoxy)-C₁-C₆-alkyl, (C₁-C₆-alkoxy)-C₁-C₆-alkoxy, (C₁-C₆-alkoxy)-C₂-C₆-alkenyl, (C₁-C₆-alkoxy)-C₂-C₆-alkynyl, C₁-C₆-alkylthio, (C₁-C₆-alkyl)sulfinyl, (C₁-C₆-alkyl)sulfonyl, (C₁-C₆-alkyl)amino, di(C₁-C₆-alkyl)amino, (C₁-C₆-alkyl)-carbonyl, (C₁-C₆-alkoxy)-carbonyl, (C₁-C₆-alkyl)-carbonyloxy, C₃-C₆-cycloalkyl, C₃-C₆-cycloalkoxy, (C₃-C₆-cycloalkyl)-C₁-C₄-alkyl, (C₃-C₆-cycloalkyl)-C₁-C₄-alkoxy, where the aliphatic and cycloaliphatic parts of the 22 aforementioned radicals are unsubstituted, partly or completely halogenated and where the cycloaliphatic parts of the last 4 mentioned radicals may carry 1, 2, 3, 4, 5 or 6 methyl groups, it being possible that two radicals R^{B} which are bound at the same carbon atom may together be ═O or ═NR^{b};
   R^{A4} is hydrogen or has one of the meanings given for R^{A};
   n is 0, 1, 2 or 3;
   R^{b} is selected from the group consisting of halogen and CN;
R¹ is selected from the group consisting of H, OH, S(O)₂NH₂, CN, C₁-C₆-alkyl, C₂-C₆-alkenyl, C₂-C₆-alkynyl, (C₃-C₆-cycloalkyl)-C₁-C₄-alkyl, C₁-C₆-alkoxy, (C₁-C₆-alkoxy)-C₁-C₆-alkyl, (C₁-C₆-alkyl)carbonyl, (C₁-C₆-alkoxy)carbonyl, (C₁-C₆-alkyl)sulfonyl, (C₁-C₆-alkylamino)carbonyl, di(C₁-C₆-alkyl)aminocarbonyl, (C₁-C₆-alkylamino)sulfonyl, di(C₁-C₆-alkyl)aminosulfonyl and (C₁-C₆-alkoxy)sulfonyl, where the aliphatic and cycloaliphatic parts of the 14 aforementioned radicals are unsubstituted, partly or completely halogenated,
   phenyl, phenylsulfonyl, phenylaminosulfonyl, phenyl-C₁-C₆ alkyl, phenoxy, phenylcarbonyl and phenoxycarbonyl,
   wherein phenyl in the last 7 mentioned radicals is unsubstituted or substituted by 1, 2, 3, 4 or 5 identical or different substituents selected from the group consisting of halogen, CN, NO₂, C₁-C₆-alkyl, C₁-C₆-haloalkyl, C₁-C₆-alkoxy and C₁-C₆-haloalkoxy;
R² is H, halogen, OH, CN, C₁-C₆-alkyl, C₂-C₆-alkenyl, C₃-C₆-alkynyl, (C₁-C₆-alkoxy)-C₁-C₆-alkyl, C₃-C₆-cycloalkyl, C₃-C₆-cycloalkenyl, (C₃-C₆-cycloalkyl)-C₁-C₄-alkyl, C₁-C₆-alkoxy, C₂-C₆-alkenyloxy, C₂-C₆-alkynyloxy, C₃-C₆-cycloalkoxy or (C₃-C₆-cycloalkyl)C₁-C₄-alkoxy, where the aliphatic and cycloaliphatic parts of the 12 aforementioned radicals are unsubstituted, partly or completely halogenated,
   phenyl, phenyl-C₁-C₆-alkyl,
   wherein phenyl in the last 2 mentioned radicals are unsubstituted or substituted by 1, 2, 3, 4 or 5 identical or different substituents selected from the group consisting of halogen, CN, NO₂, C₁-C₆-alkyl, C₁-C₆-haloalkyl, C₁-C₆-alkoxy and C₁-C₆-haloalkoxy,
R³ is selected from the group consisting of H, halogen, CN, C₁-C₆-alkyl, C₁-C₆-haloalkyl, C₁-C₆-alkoxy and C₁-C₆-haloalkoxy;
R⁴ is selected from the group consisting of H, halogen, CN, C₁-C₆-alkyl, C₂-C₆-alkenyl, C₃-C₆-alkynyl, C₃-C₆-cycloalkyl, (C₃-C₆-cycloalkyl)-C₁-C₄-alkyl, C₃-C₆-cycloalkenyl and C₁-C₆-alkoxy-C₁-C₆-alkyl, where the aliphatic and cycloaliphatic parts of the 7 aforementioned radicals are unsubstituted, partly or completely halogenated; or
R³ and R⁴ together with the carbon atom to which they are attached form a moiety selected from the group consisting of carbonyl, C₂-C₆-alkenyl, C₃-C₆-cycloalkyl, C₃-C₆-cycloalkenyl and three- to six-membered saturated or partially unsaturated heterocyclyl,
   wherein the C₃-C₆-cycloalkyl, C₃-C₆-cycloalkenyl, or three- to six-membered heterocyclyl is unsubstituted or substituted by one to six substituents selected from halogen, CN, C₁-C₆-alkyl and C₁-C₆-alkoxy; or

R² and R³ and R⁴ together with the carbon atom to which they are attached may form a moiety selected from a 5- or 6-membered aromatic heterocyclyl, which is unsubstituted or substituted by one to six substituents selected from halogen, CN, C₁-C₆-alkyl and C₁-C₆-alkoxy.

According to a first aspect, the present invention also relates to the use of aminotriazine compound of formula (I) as defined above or of its agriculturally acceptable salts as herbicides, i.e. for controlling unwanted and/or harmful vegetation or plants.

According to a second aspect, the present invention relates to the use of aminotriazine compound of formula (I) as defined above or of its agriculturally acceptable salts for the desiccation/defoliation of plants.

According to a third aspect, the present invention also relates to agrochemical compositions comprising at least one aminotriazine compound of formula (I) and at least one auxiliary customary for formulating crop protection agents.

The present invention furthermore provides a method for controlling unwanted plants. The method includes allowing a herbicidally effective amount of at least one aminotriazine compound of the formula (I) as defined above or its agriculturally acceptable salts to act on the unwanted plants or vegetation, their seeds and/or their habitat. Application can be done before, during and/or after, preferably during and/or after, the emergence of the unwanted plants.

Moreover, the invention relates to processes for preparing aminotriazine compound of formula (I) as defined above or its agriculturally acceptable salts and to intermediates. In yet a further aspect, the present invention relates to compounds of the general formula (I) and their agriculturally acceptable salts, except for
4-(2-fluorophenoxy)-6-methyl-1,3,5-triazin-2-amine,
4-(2-chlorophenoxy)-6-methyl-1,3,5-triazin-2-amine,
2-[(4-amino-6-methyl-1,3,5-triazin-2-yl)oxy]benzonitrile,
4-methyl-6-(2,4,6-trichlorophenoxy)-1,3,5-triazin-2-amine,
4-[2-chloro-4-(trifluoromethyl)phenoxy]-6-methyl-1,3,5-triazin-2-amine,
4-(2-fluorophenoxy)-6-(trifluoromethyl)-1,3,5-triazin-2-amine,
4-(2-chlorophenoxy)-6-(trifluoromethyl)-1,3,5-triazin-2-amine,
2-[(4-amino-6-(trifluoromethyl)-1,3,5-triazin-2-yl)oxy]benzonitrile,
4-(2,4,6-trichlorophenoxy)-6-(trifluoromethyl)-1,3,5-triazin-2-amine,
4-[2-chloro-4-(trifluoromethyl)phenoxy]-6-(trifluoromethyl)-1,3,5-triazin-2-amine,
4-(2,6-dichlorophenoxy)-6-(trifluoromethyl)-1,3,5-triazin-2-amine,
4-(2-bromo-4-isopropenyl-6-methoxy-phenoxy)-N-[1-(methoxymethyl)propyl]-6-methyl-1,3,5-triazin-2-amine,
4-(2-bromo-4-isopropyl-6-methoxy-phenoxy)-N-[1-(methoxymethyl)propyl]-6-methyl-1,3,5-triazin-2-amine,
4-[2-bromo-4-methoxy-6-(1-methylethenyl)phenoxy]-N-[1-(methoxymethyl)propyl]-6-methyl-1,3,5-triazin-2-amine,
4-[2-bromo-4-methoxy-6-(1-methylethyl)phenoxy]-N-[1-(methoxymethyl)propyl]-6-methyl-1,3,5-triazin-2-amine and
1-[3-bromo-5-methoxy-4-[[4-[[1-(methoxymethyl)propyl]amino]-6-methyl-1,3,5-triazin-2-yl]oxy]phenyl]-ethanone.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

As used herein, the terms “controlling” and “combating” are synonyms.

As used herein, the terms “undesirable vegetation”, “unwanted vegetation”, unwanted plants” and “harmful plants” are synonyms.

In the context of substituents, the term “one or more substitutents” means that the number of substituents is e.g. from 1 to 10, in particular 1, 2, 3, 4, 5, 6, 7 or 8.

If the aminotriazine compounds of formula (I) as described herein is capable of forming geometrical isomers, for example E/Z isomers, the invention relates to both the pure isomers and mixtures thereof. Likewise, the invention relates to the use of the pure isomers and to the use of their mixtures and to compositions containing the pure isomers or mixtures thereof.

If the aminotriazine compounds of formula (I) as described herein have one or more centres of chirality and, as a consequence, are present as enantiomers or diastereomers, the invention relates to both the pure enantiomers or diastereomers, and mixtures thereof. Likewise, the invention relates to the use of the pure enantiomers or diasteromers and to the use of the mixtures thereof and to compositions containing the pure enantiomers or diastereomers or mixtures thereof.

If the aminotriazine compounds of formula (I) as described herein have ionizable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four hydrogen atoms are replaced by C₁-C₄-alkyl, hydroxy-C₁-C₄-alkyl, (C₁-C₄-alkoxy)-C₁-C₄-alkyl, hydroxy-(C₁-C₄-alkoxy)-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethyl-ammonium (olamine salt), 2-(2-hydroxyeth-1-oxy)eth-1-ylammonium (diglycolamine salt), di(2-hydroxyeth-1-yl)-ammonium (diolamine salt), tris(2-hydroxyethyl)ammonium (trolamine salt), tris(2-hydroxypropyl)ammonium, benzyltrimethylammonium, benzyltriethylammonium, N,N,N-trimethylethanolammonium (choline salt), furthermore phosphonium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

The organic moieties mentioned in the definition of the variables, e.g. A, A.1, R^{A}, R^{A4}, R^{b}, R¹, R², R³, R⁴, X are—like the term halogen—collective terms for individual enumerations of the individual group members. The term halogen denotes in each case fluorine, chlorine, bromine or iodine. All hydrocarbon chains, i.e. all alkyl, haloalkyl, alkenyl, alkynyl, alkoxy, alkylthio, alkylsulfinyl, alkylsulfonyl, (alkyl)amino, di(alkyl)amino, alkoxyalkyl, (alky)carbonyl, (alkoxy)carbonyl chains can be straight-chain or branched, the prefix Cₙ-Cₘ denoting in each case the possible number of carbon atoms in the group. The same applies to composed radicals, such as cycloalkylalkyl and phenylalkyl.

Examples of such meanings are:

C₁-C₄-alkyl: for example CH₃, C₂H₅, n-propyl, CH(CH₃)₂, n-butyl, CH(CH₃)—C₂H₅, CH₂—CH(CH₃)₂ and C(CH₃)₃;

C₁-C₆-alkyl and also the C₁-C₆-alkyl moieties of (C₁-C₆-alkyl)carbonyl, C₁-C₆-alkyoxy-C₁-C₆-alkyl, C₃-C₆-cycloalkyl-C₁-C₆-alky, phenyl-C₁-C₆-alkyl, C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;

C₂-C₆-alkenyl: a linear or branched ethylenically unsaturated hydrocarbon group having 2 to 6 carbon atoms and a C═C-double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

C₂-C₆-alkynyl: linear or branched unsaturated hydrocarbon group having 2 to 6 carbon atoms and containing at least one C—C-triple bond, such as ethynyl, propynyl, 1-butynyl, 2-butynyl, and the like;

C₁-C₄-haloalkyl: a C₁-C₄-alkyl radical as mentioned above which is partially or fully substituted by fluorine, chlorine, bromine and/or iodine, for example, chloro-methyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 1,1,2,2,-tetrafluoroethyl and 1-trifluoromethyl-1,2,2,2-tetrafluoroethyl;

C₁-C₆-haloalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;

C₃-C₆-cycloalkyl: monocyclic saturated hydrocarbons having 3 to 6 ring members, such as cyclopropyl, cyclobutyl, cyclopentyl and cyclohexyl;

C₁-C₄-alkoxy: for example methoxy, ethoxy, propoxy, 1-methylethoxy butoxy, 1-methylpropoxy, 2-methylpropoxy and 1,1-dimethylethoxy;

C₁-C₆-alkoxy and also the C₁-C₆-alkoxy moieties of (C₁-C₆-alkoxy)carbonyl, (C₁-C₆-alkoxy)-C₁-C₆-alkyl: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

C₁-C₄-haloalkoxy: a C₁-C₄-alkoxy radical as mentioned above which is partially or fully substituted by fluorine, chlorine, bromine and/or iodine, for example, chloro-methoxy, dichloromethoxy, trichloromethoxy, fluoromethoxy, difluoromethoxy, trifluoromethoxy, chlorofluoromethoxy, dichlorofluoromethoxy, chlorodifluoromethoxy2-fluoroethoxy, 2-chloroethoxy, 2-bromoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 4-fluorobutoxy, nonafluorobutoxy, 1,1,2,2,-tetrafluoroethoxy and 1-trifluoromethyl-1,2,2,2-tetrafluoroethoxy;

C₁-C₆-haloalkoxy: C₁-C₄-alkoxy as mentioned above: C₁-C₄-haloalkoxy as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and dodecafluorohexyl;

C₂-C₆-alkenyloxy: is a C₂-C₆-alkenyl group, as defined above, attached via an oxygen atom, for example ethenyloxy, propen-1-yloxy, allyloxy (propen-2-yl-oxy), buten-1-oxy, buten-2-oxy, buten-3-oxy, and the like;

C₂-C₆-haloalkenyloxy: is a C₂-C₆-haloalkenyl group, as defined above, attached via an oxygen atom;

C₂-C₆-alkynyloxy: is a C₂-C₆-alkynyl group, as defined above, attached via an oxygen atom, for example ethynyloxy, propyn-1-yloxy, propargyloxy (propyn-2-yl-oxy), butyn-1-oxy, butyn-2-oxy, butyn-3-oxy, and the like;

C₂-C₆-haloalkynyloxy: is a C₂-C₆-haloalkynyl group, as defined above, attached via an oxygen atom;

C₁-C₄-alkoxy-C₁-C₄-alkyl: refers to a straight-chain or branched alkyl group having 1 to 4 carbon atoms, as defined above, where one hydrogen atom is replaced by a C₁-C₄-alkoxy group, as defined above;

C₁-C₆-alkoxy-C₁-C₄-alkyl: refers to a straight-chain or branched alkyl group having 1 to 4 carbon atoms, as defined above, where one hydrogen atom is replaced by a C₁-C₆-alkoxy group, as defined above, for example methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like;

C₁-C₄-alkoxy-methyl: refers to methyl in which one hydrogen atom is replaced by a C₁-C₄-alkoxy group, as defined above;

C₁-C₆-alkoxy-methyl: refers to methyl in which one hydrogen atom is replaced by a C₁-C₆-alkoxy group, as defined above, for example methoxymethyl, ethoxymethyl, propoxymethyl, isopropoxymethyl, n-butoxymethyl, sec-butoxymethyl, isobutoxymethyl, tert-butoxymethyl, pentyloxymethyl, hexyloxymethyl and the like;

C₁-C₆-haloalkoxy-C₁-C₄-alkyl: is a straight-chain or branched alkyl group having from 1 to 4 carbon atoms, wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by halogen atoms;

C₁-C₄-haloalkoxy-C₁-C₄-alkyl is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by halogen atoms, for example difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoro-methoxy-methyl (CH₃OCF₂), 1,1-difluoro-2-methoxyethyl, 2,2-difluoro-2-methoxyethyl and the like;

(C₁-C₆-alkoxy)-C₁-C₆-alkoxy: is a C₁-C₆-alkoxy group, in particular C₁-C₄-alkoxy as defined above, such as methoxy or ethoxy, wherein 1 hydrogen atom is replaced by C₁-C₆-alkoxy as defined above, examples including methoxymethoxy, ethoxymethoxy, n-propoxymethoxy, butoxymethoxy, 2-methoxyethoxy, 2-ethoxyethoxy, 2-(n-propoxy)ethoxy and 2-butoxyethoxy;

(C₁-C₆-alkoxy)-C₂-C₆-alkenyl: is a C₁-C₆-alkoxy group, in particular C₁-C₄-alkoxy as defined above, such as methoxy or ethoxy, wherein 1 hydrogen atom is replaced by C₂-C₆-alkenyl as defined above;

(C₁-C₆-alkoxy)-C₂-C₆-alkynyl: is a C₁-C₆-alkoxy group, in particular C₁-C₄-alkoxy as defined above, such as methoxy or ethoxy, wherein 1 hydrogen atom is replaced by C₂-C₆-alkynyl as defined above;

alkylcarbonyl: is a C₁-C₆-alkyl (“C₁-C₆-alkylcarbonyl”), preferably a C₁-C₄-alkyl (“C₁-C₄-alkylcarbonyl”) group, as defined above, attached via a carbonyl [C(═O)] group, for example acetyl (methylcarbonyl), propionyl (ethylcarbonyl), propylcarbonyl, isopropylcarbonyl, n-butylcarbonyl and the like;

haloalkylcarbonyl: is a C₁-C₆-haloalkyl (“C₁-C₆-haloalkylcarbonyl”), preferably a C₁-C₄-haloalkyl (“C₁-C₄-haloalkylcarbonyl”) group, as defined above, attached via a carbonyl [C(═O)] group, for example trifluoromethylcarbonyl, 2,2,2-trifluoroethylcarbonyl and the like;

alkoxycarbonyl: is a C₁-C₆-alkoxy (“C₁-C₆-alkoxycarbonyl”), preferably a C₁-C₄-alkoxy (“C₁-C₄-alkoxycarbonyl”) group, as defined above, attached via a carbonyl [C(═O)] group, for example methoxycarbonyl, ethoxycarbonyl, propoxycarbonyl, isopropoxycarbonyl, n-butoxycarbonyl and the like;

haloalkoxycarbonyl: is a C₁-C₆-haloalkoxy (“C₁-C₆-haloalkoxycarbonyl”), preferably a C₁-C₄-haloalkoxy (“C₁-C₄-haloalkoxycarbonyl”) group, as defined above, attached via a carbonyl [C(═O)] group, for example trifluoromethoxycarbonyl, 2,2,2-trifluoroethoxycarbonyl and the like;

C₁-C₄-alkylthio: for example methylthio, ethylthio, propylthio, 1-methylethylthio, butylthio, 1-methylpropylthio, 2-methylpropylthio and 1,1-dimethylethylthio;

C₁-C₆-alkylthio: C₁-C₄-alkylthio as mentioned above, and also, for example, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

C₁-C₆-alkylsulfinyl (C₁-C₆-alkyl-S(═O)—): z.B. methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentyl-sulfinyl, 1,1-dimethylbutylsulfinyl, 1,2-dimethylbutylsulfinyl, 1,3-dimethylbutyl-sulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl;

C₁-C₆-alkylsulfonyl (C₁-C₆-alkyl-S(O)₂—): for example methylsulfonyl, ethylsulfonyl, propylsulfonyl, 1-methylethylsulfonyl, butylsulfonyl, 1-methylpropylsulfonyl, 2-methyl-propylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethyl propylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;

(C₁-C₄-alkyl)amino: for example methylamino, ethylamino, propylamino, 1-methylethylamino, butylamino, 1-methylpropylamino, 2-methylpropylamino or 1,1-dimethylethylamino;

(C₁-C₆-alkyl)amino: (C₁-C₄-alkylamino) as mentioned above, and also, for example, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutyl-amino 3,3-dimethylbutylamino, 1-ethylbutylamino, 2-ethylbutylamino, 1,1,2-trimethylpropylamino, 1,2,2-trimethyl-propylamino, 1-ethyl-1-methylpropylamino or 1-ethyl-2-methylpropylamino;

di(C₁-C₄-alkyl)amino: for example N,N-dimethylamino, N,N-diethylamino, N,N-di(1-methylethyl)amino, N,N-dipropylamino, N,N-dibutylamino, N,N-di(1-methylpropyl)amino, N,N-di(2-methylpropyl)amino, N,N-di(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino;

di(C₁-C₆-alkyl)amino: di(C₁-C₄-alkyl)amino as mentioned above, and also, for example, N-methyl-N-pentylamino, N-methyl-N-(1-methylbutyl)amino, N-methyl-N-(2-methylbutyl)amino, N-methyl-N-(3-methylbutyl)amino, N-methyl-N-(2,2-dimethylpropyl)amino, N-methyl-N-(1-ethylpropyl)amino, N-methyl-N-hexylamino, N-methyl-N-(1,1-dimethylpropyl)amino, N-methyl-N-(1,2-dimethylpropyl)amino, N-methyl-N-(1-methylpentyl)amino, N-methyl-N-(2-methylpentyl)amino, N-methyl-N-(3-methylpentyl)amino, N-methyl-N-(4-methylpentyl)amino, N-methyl-N-(1,1-dimethylbutyl)amino, N-methyl-N-(1,2-dimethylbutyl)amino, N-methyl-N-(1,3-dimethylbutyl)amino, N-methyl-N-(2,2-dimethylbutyl)amino, N-methyl-N-(2,3-dimethylbutyl)amino, N-methyl-N-(3,3-dimethylbutyl)amino, N-methyl-N-(1-ethylbutyl)amino, N-methyl-N-(2-ethylbutyl)amino, N-methyl-N-(1,1,2-trimethylpropyl)amino, N-methyl-N-(1,2,2-trimethylpropyl)amino, N-methyl-N-(1-ethyl-1-methylpropyl)amino, N-methyl-N-(1-ethyl-2-methylpropyl)amino, N-ethyl-N-pentylamino, N-ethyl-N-(1-methylbutyl)amino, N-ethyl-N-(2-methylbutyl)amino, N-ethyl-N-(3-methylbutyl)amino, N-ethyl-N-(2,2-dimethylpropyl)amino, N-ethyl-N-(1-ethylpropyl)amino, N-ethyl-N-hexylamino, N-ethyl-N-(1,1-dimethylpropyl)amino, N-ethyl-N-(1,2-dimethylpropyl)amino, N-ethyl-N-(1-methylpentyl)amino, N-ethyl-N-(2-methylpentyl)amino, N-ethyl-N-(3-methylpentyl)amino, N-ethyl-N-(4-methylpentyl)amino, N-ethyl-N-(1,1-dimethylbutyl)amino, N-ethyl-N-(1,2-dimethylbutyl)amino, N-ethyl-N-(1,3-dimethylbutyl)amino, N-ethyl-N-(2,2-dimethylbutyl)amino, N-ethyl-N-(2,3-dimethylbutyl)amino, N-ethyl-N-(3,3-dimethylbutyl)amino, N-ethyl-N-(1-ethylbutyl)amino, N-ethyl-N-(2-ethylbutyl)amino, N-ethyl-N-(1,1,2-trimethylpropyl)amino, N-ethyl-N-(1,2,2-trimethylpropyl)amino, N-ethyl-N-(1-ethyl-1-methylpropyl)amino, N-ethyl-N-(1-ethyl-2-methylpropyl)amino, N-propyl-N-pentylamino, N-butyl-N-pentylamino, N,N-dipentylamino, N-propyl-N-hexylamino, N-butyl-N-hexylamino, N-pentyl-N-hexylamino or N,N-dihexylamino;

C₃-C₆-cycloalkyl: a cycloaliphatic radical having 3 to 6 carbon atoms, such as cyclopropyl, cyclobutyl, cyclopentyl and cyclohexyl;

C₃-C₆-cycloalkyl-C₁-C₆-alkyl: C₁-C₆-alkyl, in particular C₁-C₄-alkyl as defined above, such as methyl or ethyl, wherein 1 hydrogen atom is replaced by C₃-C₆-cycloalkyl as defined above, examples including cyclopropylmethyl (CH₂-cyclopropyl), cyclobutylmethyl, cyclopentylmethyl, cycloexylmethyl, 1-cyclopropylethyl (CH(CH₃)-cyclopropyl), 1-cyclobutylethyl, 1-cyclopentylethyl, 1-cycloexylethyl, 1-cyclopropylethyl (CH(CH₃)-cyclopropyl), 1-cyclobutylethyl, 1-cyclopentylethyl or 1-cycloexylethyl;

C₃-C₆-cycloalkoxy: is a C₃-C₆-cycloalkyl group, as defined above, which is attached via an oxygen atom, C₃-C₈-cycloalkoxy: is a C₃-C₈-cycloalkyl group, as defined above, which is attached via an oxygen atom, for example C₃-C₆-cycloalkoxy comprise cyclopropoxy, cyclobutoxy, cyclopentoxy and cyclohexoxy, for example C₃-C₈-cycloalkoxy comprise, in addition to those mentioned for C₃-C₆-cycloalkoxy, cycloheptoxy and cyclooctyloxy;

C₃-C₆-halocycloalkoxy: is a C₃-C₆-halocycloalkyl group, as defined above, which is attached via an oxygen atom, C₃-C₈-halocycloalkoxy: is a C₃-C₈-halocycloalkyl group, as defined above, which is attached via an oxygen atom;

C₃-C₈-Cycloalkyl-C₁-C₄-alkoxy: is C₁-C₄-alkoxy, as defined above wherein one of the hydrogen atoms is replaced by a C₃-C₈-cycloalkyl group;

C₃-C₈-Halocycloalkyl-C₁-C₄-alkoxy: is C₁-C₄-alkoxy, as defined above wherein one of the hydrogen atoms is replaced by a C₃-C₈-cycloalkyl group and wherein at least one, e.g. 1, 2, 3, 4 or all of the remaining hydrogen atoms (either in the cycloalkyl moiety or in the alkoxy moiety or in both) are replaced by halogen atoms;

aminocarbonyl: is a group —C(O)—NH₂;

C₁-C₆-alkylaminocarbonyl: is a group —C(O)—N(H)C₁-C₆-alkyl, for example methylaminocarbonyl, ethylaminocarbonyl, propylaminocarbonyl, isopropylaminocarbonyl, butylaminocarbonyl and the like;

di-(C₁-C₆-alkyl)-aminocarbonyl: is a group —C(O)—N(C₁-C₆-alkyl)₂, for example dimethylaminocarbonyl, diethylaminocarbonyl, ethylmethylaminocarbonyl, dipropylaminocarbonyl, diisopropylaminocarbonyl, methylpropylaminocarbonyl, methylisopropylaminocarbonyl, ethylpropylaminocarbonyl, ethylisopropylaminocarbonyl, dibutylaminocarbonyl and the like;

C₁-C₆-alkylaminosulfonyl: is a C₁-C₆-alkylamino group as mentioned above, which is bound to the remainder of the molecule by a sulfonyl group (S(O)₂);

di-(C₁-C₆-alkyl)-aminosulfonyl: is a di-(C₁-C₆-alkyl)-amino group as mentioned above, which is bound to the remainder of the molecule by a sulfonyl group;

C₁-C₆-alkoxysulfonyl: is a C₁-C₆-alkoxy group as mentioned above, which is bound to the remainder of the molecule by a sulfonyl group;

phenyl-C₁-C₆-alkyl: C₁-C₆-alkyl, in particular C₁-C₄-alkyl as defined above, such as methyl or ethyl, wherein 1 hydrogen atom is replaced by phenyl, examples including benzyl, 1-phenylethyl, 2-phenylethyl, 1-phenylpropyl, 2-phenylpropyl, 1-phenyl-1-methylethyl etc.;

phenylsulfonyl: is a phenyl group as mentioned above, which is bound to the remainder of the molecule by a sulfonyl group;

phenylcarbonyl: is a phenyl group, which is bound to the reminder molecule by a carbonyl group;

phenoxycarbonyl: is a phenoxy group, which is bound to the reminder molecule by a carbonyl group;

phenylaminosulfonyl: is a group —S(O)₂—NH—R, where R is phenyl;

three- to six-membered heterocyclyl: monocyclic saturated, partially unsaturated or aromatic ring having three to six ring members as mentioned above which, in addition to carbon atoms, contains one or two heteroatoms selected from O, S and N;

for example

saturated heterocycles such as 2-oxiranyl, 2-oxetanyl, 3-oxetanyl, 2-aziridinyl, 3-thietanyl, 1-azetidinyl, 2-azetidinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-piperidinyl, 3-piperidinyl, 4-piperidinyl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,4-dithian-2-yl, 1,3-dithian-5-yl, 2-tetrahydropyranyl, 3-tetrahydropyranyl, 4-tetrahydropyranyl, 2-tetrahydrothiopyranyl, 3-tetrahydrothiopyranyl, 4-tetrahydro-thiopyranyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, tetrahydro-1,3-oxazin-2-yl, tetrahydro-1,3-oxazin-6-yl, 2-morpholinyl, 3-morpholinyl or 4-morpholinyl, for example 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl;

partially unsaturated heterocycles such as 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 4,5-dihydropyrrol-2-yl, 4,5-dihydropyrrol-3-yl, 2,5-dihydropyrrol-2-yl, 2,5-dihydropyrrol-3-yl, 4,5-dihydroisoxazol-3-yl, 2,5-dihydroisoxazol-3-yl, 2,3-dihydroisoxazol-3-yl, 4,5-dihydroisoxazol-4-yl, 2,5-dihydroisoxazol-4-yl, 2,3-dihydroisoxazol-4-yl, 4,5-dihydroisoxazol-5-yl, 2,5-dihydroisoxazol-5-yl, 2,3-dihydroisoxazol-5-yl, 4,5-dihydroisothiazol-3-yl, 2,5-dihydroisothiazol-3-yl, 2,3-dihydroisothiazol-3-yl, 4,5-dihydroisothiazol-4-yl, 2,5-dihydroisothiazol-4-yl, 2,3-dihydroisothiazol-4-yl, 4,5-dihydroisothiazol-5-yl, 2,5-dihydroisothiazol-5-yl, 2,3-dihydroisothiazol-5-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-3-yl, 3,4-dihydropyrazol-4-yl, 3,4-dihydropyrazol-5-yl, 4,5-dihydropyrazol-3-yl, 4,5-dihydropyrazol-4-yl, 4,5-dihydropyrazol-5-yl, 2,3-dihydroimidazol-2-yl, 2,3-dihydroimidazol-3-yl, 2,3-dihydroimidazol-4-yl, 2,3-dihydroimidazol-5-yl, 4,5-dihydroimidazol-2-yl, 4,5-dihydroimidazol-4-yl, 4,5-dihydroimidazol-5-yl, 2,5-dihydroimidazol-2-yl, 2,5-dihydroimidazol-4-yl, 2,5-dihydroimidazol-5-yl, 2,3-dihydrooxazol-3-yl, 2,3-dihydrooxazol-4-yl, 2,3-dihydrooxazol-5-yl, 3,4-dihydrooxazol-3-yl, 3,4-dihydrooxazol-4-yl, 3,4-dihydrooxazol-5-yl, 2,3-dihydrothiazol-3-yl, 2,3-dihydrothiazol-4-yl, 2,3-dihydrothiazol-5-yl, 3,4-dihydrothiazol-3-yl, 3,4-dihydrothiazol-4-yl, 3,4-dihydrothiazol-5-yl, 3,4-dihydrothiazol-2-yl, 3,4-dihydrothiazol-3-yl, 3,4-dihydrothiazol-4-yl, 3,6-dihydro-2H-pyran-2-yl, 3,6-dihydro-2H-pyran-3-yl, 3,6-dihydro-2H-pyran-4-yl, 3,6-dihydro-2H-pyran-5-yl, 3,6-dihydro-2H-pyran-6-yl, 3,4-dihydro-2H-pyran-3-yl, 3,4-dihydro-2H-pyran-4-yl, 3,4-dihydro-2H-pyran-6-yl, 5,6-dihydro-4H-1,3-oxazin-2-yl;

5- or 6-membered aromatic heterocyclic rings, also termed heteroaromatic rings or hetaryl, include: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4 thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

a fused 5- or 6-membered carbocyclic or heterocyclic ring refers to a carbocyclic or heterocyclic ring as defined above fused to a phenyl ring or 6-membered hetaryl ring.

The preferred embodiments of the invention mentioned herein below have to be understood as being preferred either independently from each other or in combination with one another. Particular groups of embodiments of the invention relate to those aminotriazines of formula (I), wherein the variables A, R¹, R², R³, R⁴, R^{b}, and X, either independently of one another or in combination with one another, have the following meanings:

Particular groups of embodiments relate to the aminotriazine compounds of formula (I),

wherein
X is CR^{A4}. In particular
X is CR^{A4};
R^{b} is F;
n 0, 1, 2 or 3, in particular 1, 2 or 3, more particularly 2 or 3;
R^{A} has one of the meanings as defined above, which are
in particular selected from the group consisting of halogen, CN, C₁-C₆-alkyl, C₁-C₆-haloalkyl, C₃-C₆-cycloalkyl, C₂-C₆-alkynyl, C₂-C₆-alkenyl, C₁-C₆-alkoxy, C₃-C₆-alkenyloxy, C₃-C₆-cycloalkoxy, (C₃-C₆-cycloalkyl)methoxy, C₂-C₆-alkynyloxy, C₂-C₆-alkenyloxy or C₁-C₆-haloalkoxy;
more particularly selected from the group consisting of halogen, CN, C₁-C₄-alkyl, C₁-C₄-haloalkyl, C₃-C₆-cycloalkyl, C₂-C₄-alkenyl, C₂-C₄-alkynyl, C₁-C₄-alkoxy, C₃-C₆-alkenyloxy, C₂-C₄-alkenyloxy, C₃-C₆-cycloalkoxy, (C₃-C₆-cycloalkyl)methoxy or C₁-C₄-haloalkoxy; even more particularly selected from the group consisting of F, Cl, Br, CN, CF₃, methyl, vinyl, ethynyl and cyclopropyl, methoxy, ethoxy, isopropyloxy, allyloxy, propargyloxy, cyclopropyloxy, cyclobutyloxy, cyclopentyloxy, (cyclopropyl)methoxy or 2-butyloxy;
even more particularly selected from the group consisting of F, Cl, Br, CN, CF₃, methyl, vinyl, ethynyl and cyclopropyl;
especially selected from the group consisting of F, Cl, Br and CN; and
R^{A4} is H or has one of the meanings from R^{A},
in particular selected from the group consisting of H, halogen, CN, C₁-C₆-alkyl, C₁-C₆-haloalkyl, C₃-C₆-cycloalkyl, C₂-C₆-alkynyl, C₂-C₆-alkenyl, C₁-C₆-alkoxy, C₃-C₆-alkenyloxy, C₃-C₆-cycloalkoxy, (C₃-C₆-cycloalkyl)methoxy, C₂-C₆-alkynyloxy, C₂-C₆-alkenyloxy or C₁-C₆-haloalkoxy;
more particularly selected from the group consisting of H, halogen, CN, C₁-C₄-alkyl, C₁-C₄-haloalkyl, C₃-C₆-cycloalkyl, C₂-C₄-alkenyl, C₂-C₄-alkynyl, C₁-C₄-alkoxy, C₃-C₆-alkenyloxy, C₂-C₄-alkenyloxy, C₃-C₆-cycloalkoxy, (C₃-C₆-cycloalkyl)methoxy or C₁-C₄-haloalkoxy;
even more particularly selected from the group consisting of H, F, Cl, Br, CN, CF₃, methyl, vinyl, ethynyl and cyclopropyl, methoxy, ethoxy, isopropyloxy, allyloxy, propargyloxy, cyclopropyloxy, cyclobutyloxy, cyclopentyloxy, (cyclopropyl)methoxy or 2-butyloxy;
even more particularly selected from the group consisting of H, F, Cl, Br, CN, CF₃, methyl, vinyl, ethynyl and cyclopropyl;
especially selected from the group consisting of H, F, Cl, Br and CN.

More particularly A is a moiety of the following formula,

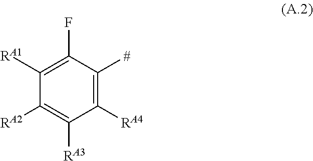

wherein # indicates the point of attachment to the oxygen;
R^{A1} is hydrogen or halogen, in particular hydrogen or fluorine;
R^{A2} is hydrogen or has one of the meanings given for R^{A}, and is in particular selected from the group consisting of hydrogen, halogen, CN, C₁-C₆-alkyl, C₁-C₁-C₆-haloalkyl, C₃-C₆-cycloalkyl, C₂-C₆-alkynyl and C₂-C₆-alkenyl;
   more particularly selected from hydrogen, halogen, CN, C₁-C₄-alkyl, C₂-C₄-alkynyl, C₁-C₄-haloalkyl and C₃-C₆-cycloalkyl;
   even more particularly from the group consisting of H, F, Cl, Br, I, CN, CF₃, methyl, vinyl, ethynyl and cyclopropyl;
   especially selected from the group consisting of H, F, Br, I and CN;
R^{A3} is hydrogen or halogen, in particular hydrogen or fluorine;
R^{A4} is hydrogen or has one of the meanings given for R^{A}, and is in particular selected from the group consisting of hydrogen, halogen, CN, C₁-C₆-alkyl, C₁-C₆-haloalkyl, C₃-C₆-cycloalkyl, C₂-C₆-alkynyl and C₂-C₆-alkenyl;
   more particularly selected from halogen, CN, C₁-C₄-alkyl, C₂-C₄-alkynyl, C₁-C₄-haloalkyl and C₃-C₆-cycloalkyl;
   even more particularly from the group consisting of F, Cl, Br, CN, CF₃, methyl, vinyl, ethynyl and cyclopropyl;
   especially selected from the group consisting of F, Cl, Br and CN.

Especially preferred examples of A include 2,6-difluorophenyl, 2,3-difluorophenyl, 2-chloro-6-fluorophenyl, 2-bromo-6-fluorophenyl, 2-fluoro-6-methylphenyl, 2-fluoro-6-cyanophenyl, 2,3,6-trifluorophenyl, 2,4,6-trifluorophenyl, 2-chloro-4,6-difluorophenyl, 2-chloro-5,6-difluorophenyl, 3-chloro-2,6-difluorophenyl, 2-chloro-3,6-difluorophenyl, 2,3-difluoro-6-cyanophenyl, 2,6-difluoro-3-cyanophenyl, 2,5-difluoro-6-cyanophenyl, 2,4-difluoro-6-cyanophenyl, 2,3,4,6-tetrafluorophenyl, 2,3,4,5-tetrafluorophenyl, 2,3,5,6-tetrafluorophenyl, 2-chloro-3,4,6-trifluorophenyl, 2-chloro-3,5,6-trifluorophenyl, 3-chloro-2,4,6-trifluorophenyl, 3-chloro-2,5,6-trifluorophenyl, 6-cyano-2,4,5-trifluorophenyl, 3-cyano-2,4,6-trifluorophenyl, 6-cyano-2,3,4-trifluorophenyl, 6-cyano-2,3,5-trifluorophenyl, 3-cyano-2,5,6-trifluorophenyl, pentafluorophenyl, 2-chloro-3,4,5,6-tetrafluorophenyl, 3-chloro-2,4,5,6-tetrafluorophenyl, 6-cyano-2,3,4,5-tetrafluorophenyl, 5-cyano-2,3,4,6-tetrafluorophenyl, 4-bromo-2,3,5,6-tetrafluorophenyl, 4-iodo-2,3,5,6-tetrafluorophenyl, 4-ethynyl-2,3,5,6-tetrafluorophenyl, 3-chloro-2-fluoro-6-(trifluoromethyl)phenyl, 2,3,5,6-tetrafluoro-4-iodophenyl, 2,3,5,6-tetrafluoro-4-bromophenyl and 2,3,5,6-tetrafluoro-4-chlorophenyl, preferably 2,3,4,5,6-pentafluorophenyl, 2,3,6-trifluorophenyl, 2,3-difluorophenyl, 2,3,5,6-tetrafluorophenyl, 2,4,5,6-tetrafluorophenyl, 2,3,5,6-tetrafluoro-4-iodophenyl, 2,3,5,6-tetrafluoro-4-bromophenyl and 2,3,5,6-tetrafluoro-4-chlorophenyl, with particular preference given to 2,6-difluorophenyl, 2,3,6-trifluorophenyl, 2,3,5,6-tetrafluorophenyl and 2,3,4,5,6-pentafluorophenyl.

Other particular groups of embodiments relate to the aminotriazine compounds of formula (I), wherein

X is N; R^{A} and R^{b} has one of the meanings as defined above. In particular

X is N;

R^{b} is selected from halogen in particular F;

n is 0, 1, 2 or 3, in particular 1, 2 or 3;

R^{A} has one of the meanings as defined above, which are preferably selected from the group consisting of halogen, CN, C₁-C₆-alkyl, C₁-C₆-alkoxy, C₁-C₆-haloalkyl, C₃-C₆-cycloalkyl, C₃-C₆-cycloalkoxy, (C₃-C₆-cycloalkyl)methoxy, C₂-C₆-alkynyl, C₂-C₆-alkenyl, C₂-C₆-alkynyloxy, C₂-C₆-alkenyloxy or C₁-C₆-haloalkoxy;

particularly selected from the group consisting of halogen, CN, C₁-C₆-alkyl, C₁-C₁-C₆-haloalkyl, C₃-C₆-cycloalkyl, C₂-C₆-alkynyl and C₂-C₆-alkenyl;

more particularly selected from the group consisting of halogen, CN, C₁-C₄-alkyl, C₂-C₄-alkynyl, C₁-C₄-haloalkyl and C₃-C₆-cycloalkyl;

even more particularly selected from the group consisting of F, Cl, Br, CN, CF₃, methyl, vinyl, ethynyl and cyclopropyl;

especially selected from the group consisting of F, Cl, Br and CN.

If X is N, A has especially a moiety of the following formula

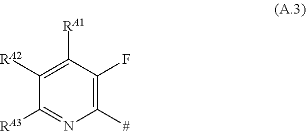

wherein
R^{A1}, R^{A2} and R^{A3} independently of one another are hydrogen, halogen, CN, NO₂, C₁-C₆-alkyl, C₁-C₆-haloalkyl, OH, C₁-C₆-alkoxy, C₁-C₆-alkylthio, (C₁-C₆-alkyl)sulfinyl, (C₁-C₆-alkyl)sulfonyl, amino, (C₁-C₆-alkyl)amino, di(C₁-C₆-alkyl)amino, (C₁-C₆-alkyl)carbonyl, (C₁-C₆-alkoxy)carbonyl;
particularly preferred R^{A1}, R^{A2} and R^{A3} independently of one another are hydrogen, halogen, CN, NO₂, C₁-C₆-alkyl, C₁-C₆-haloalkyl or C₁-C₆-alkoxy;
especially preferred R^{A1}, R^{A2} and R^{A3} independently of one another are hydrogen, halogen, CN, C₁-C₆-alkyl or C₁-C₆-alkoxy;
more preferred R^{A1}, R^{A2} and R^{A3} independently of one another are hydrogen, halogen or CN;
most preferred R^{A1}, R^{A2} and R^{A3} are hydrogen;
also most preferred R^{A1} and R^{A3} are F or Cl; and
R^{A2} is H or F.

Among this group of embodiment A is in particular 4-chloro-3,5,6-trifluoro-2-pyridyl.

Particular groups of embodiments relate to the aminotriazine compounds of formula (I), wherein;
R¹ is H, CN, C₁-C₆-alkyl, (C₁-C₆-alkoxy)-C₁-C₆-alkyl, (C₁-C₆-alkyl)carbonyl or (C₁-C₆-alkyl)sulfonyl, where the aliphatic parts of the 4 aforementioned radicals unsubstituted partly or completely halogenated,
   phenyl and phenyl-C₁-C₆ alkyl,
   wherein phenyl in the last 2 mentioned radical is unsubstituted or substituted by 1, 2, 3, 4, or 5 identical or different substituents selected from the group consisting of halogen, CN, NO₂, C₁-C₆-alkyl, C₁-C₆-haloalkyl, C₁-C₆-alkoxy and C₁-C₆-haloalkoxy;
   in particular H, CN, C₁-C₆-alkyl, C₁-C₆-haloalkyl, (C₁-C₆-alkoxy)-C₁-C₆-alkyl, C₁-C₆-alkoxy, (C₁-C₆-alkyl)carbonyl, (C₁-C₆-haloalkyl)carbonyl, (C₁-C₆-alkyl)sulfonyl or (C₁-C₆-haloalkyl)sulfonyl;
   more particularly H, CN, C₁-C₄-alkyl, (C₁-C₄-alkoxy)-C₁-C₄-alkyl, C₁-C₄-alkoxy, (C₁-C₄-alkyl)carbonyl or (C₁-C₄-alkyl)sulfonyl; even more particularly H, CN, CH₃, CH₂OCH₃, OCH₃, C(O)CH₃ or SO₂CH₃;
   especially hydrogen.

Further particular groups of embodiments relate to the aminotriazine compounds of formula (I), wherein;
R² is selected from the group consisting of H, halogen, CN, C₁-C₆-alkyl, C₁-C₆-haloalkyl, C₁-C₆-alkoxy C₁-C₆-haloalkoxy and phenyl, in particular from the group consisting of H, halogen, CN, C₁-C₆-alkyl, C₁-C₆-haloalkyl, C₁-C₆-alkoxy C₁-C₆-haloalkoxy, more particular from the group consisting of H, fluorine, chlorine, C₁-C₄-alkyl, such as methyl, ethyl, n-propyl, 2-propyl, n-butyl, 2-butyl, isobutyl or tert.-butyl, C₁-C₄-haloalkyl, such as difluoromethyl, trifluoromethyl, 2,2,2-trifluoroethyl, 1,1-difluoroethyl, 1,1,2,2-tetrafluoroethyl or pentafluoroethyl, C₁-C₄-alkoxy, such as methoxy or ethoxy and C₁-C₄-haloalkoxy, such as difluoromethoxy or trifluoromethoxy.

Further particular groups (1) of embodiments relate to the aminotriazine compounds of formula (I), wherein R³ is selected from the group consisting of H, halogen, CN, C₁-C₆-alkyl, C₁-C₆-haloalkyl, C₁-C₆-alkoxy and C₁-C₆-haloalkoxy, in particular from the group consisting of hydrogen, fluorine and C₁-C₄-alkyl, more particularly from hydrogen, fluorine and methyl, especially from hydrogen and fluorine.

In groups (1) of embodiments, R⁴ is as defined above and preferably selected from the group consisting of C₁-C₆-alkyl, C₁-C₆-haloalkyl, C₂-C₆-alkenyl, C₂-C₆-alkynyl, C₃-C₆-cycloalkyl, C₃-C₆-cycloalkenyl, C₁-C₆-alkoxy and C₁-C₆-alkoxy-C₁-C₆-alkyl or from C₁-C₆-alkyl, C₁-C₆-haloalkyl, C₂-C₆-alkenyl, C₃-C₆-alkynyl, C₃-C₆-cycloalkyl, C₃-C₆-cycloalkenyl, C₁-C₆-alkoxy and C₁-C₆-alkoxy-C₁-C₆-alkyl.

In groups (1) of embodiments, R⁴ is in particular selected from the group consisting of C₁-C₄-alkyl, such as ethyl, n-propyl, 2-propyl, n-butyl, 2-butyl, isobutyl or tert.-butyl, C₁-C₄-haloalkyl, such as difluoromethyl, trifluoromethyl, 2,2,2-trifluoroethyl, 1,1-difluoroethyl, 1,1,2,2-tetrafluoroethyl or pentafluoroethyl, C₂-C₄-alkenyl, such as vinyl or allyl, C₃-C₄-alkynyl, such as propargyl, C₃-C₆-cycloalkyl, such as cyclopropyl, cyclobutyl, cyclopentyl or cyclohexyl, and C₁-C₄-alkoxy-C₁-C₄-alkyl, such as methoxymethyl, ethoxymethyl, 2-methoxyethyl or 2-ethoxyethyl.

Further particular groups (2) of embodiments relate to the aminotriazine compounds of formula (I), wherein R³ and R⁴ together with the carbon atom to which they are attached form a moiety selected from the group consisting of carbonyl, C₂-C₆-alkenyl, C₃-C₆-cycloalkyl, C₃-C₆-cycloalkenyl and three- to six-membered heterocyclyl, wherein the C₃-C₆-cycloalkyl, C₃-C₆-cycloalkenyl, or three- to six-membered heterocyclyl is unsubstituted or substituted by one to six substituents selected from halogen, CN, C₁-C₆-alkyl and C₁-C₆-alkoxy and wherein C₂-C₆-alkenyl is a C═CR^{3a}R^{3b} radical, wherein R^{3a} and R^{3b} independently selected from the group of H and C₁-C₄-alkyl, wherein the sum of the carbon atoms of R^{3a} and R^{3b} radicals is an integer from 0 to 4. A skilled person will readily appreciate that the cycloalkyl or cycloalkenyl radical and the heterocyclic radical are ipso-connected, i.e. the radical R² and the triazine ring of formula (I) are bound to the same carbon atom of the carbocyclic radical and the heterocyclic radical formed by R³ and R⁴ together with the carbon atom, to which R³ and R⁴ are attached. Therefore, the carbocyclic radical and the heterocyclic radical are also termed ipso-radicals. The carbocyclic radical and the heterocyclic radical are unsubstituted or substituted by one to six substituents selected from halogen, CN, C₁-C₆-alkyl and C₁-C₆-alkoxy. Further particular groups (2) of embodiments relate to the aminotriazine compounds of formula (I), wherein R² and R³ and R⁴ together with the carbon atom to which they are attached may form a moiety selected from a 5- or 6-membered aromatic heterocyclyl, which is unsubstituted or substituted by one to six substituents selected from halogen, CN, C₁-C₆-alkyl and C₁-C₆-alkoxy.

Suitable ipso-carbocyclic radicals, which are formed by R³ and R⁴ together with the carbon atom to which they are attached, C₃-C₆-cycloalkyl, C₃-C₆-cycloalkan-1,1-diyl, C₃-C₆-cycloalkenyl and ipso-C₃-C₆-cycloalkendiyl as defined above. Suitable ipso-heterocyclic radicals, which are formed by R³ and R⁴ together with the carbon atom to which they are attached, may be saturated partially unsaturated or aromatic, or R² and R³ and R⁴ together with the carbon atom to which they are attached may form a moiety selected from a 5- or 6-membered aromatic heterocyclyl Suitable ipso-heterocyclic radicals are 3- to 6-membered, i.e. they have 3, 4, 5 or 6 ring atoms, wherein at least 1 ring atom, e.g. 1, 2 or 3 ring atoms are a heteroatom, which is preferably selected from O, S and N, while the other ring atoms are carbon atoms. Examples of heterocyclyl radicals and ipso-heterocyclodiyl radicals include oxiran-2,2-diyl, oxiranyl, oxetan-2,2-diyl, oxetan-2-yl, oxetan-3-yl, oxetan-3,3-diyl, oxolan-2,2-diyl, oxolan-2-yl, oxolan-3,3-diyl, oxolan-3-yl, 1,3-dioxolan-2,2-diyl, 1,3-dioxolanyl, oxan-2,2-diyl, oxan-2-yl, oxan-3,3-diyl, oxan-3yl, oxan-4,4-diyl, oxan-4-yl, 1,3-dioxan-2,2-diyl, 1,3-dioxanyl, thiolan-2,2-diyl, thiolan-2-yl, thiolan-3,3-diyl, thiolan-3-yl, pyrrolidin-2,2-diyl, pyrrolidin-2-yl, pyrrolidin-3,3-diyl, pyrrolidin-3-yl, piperidin-2,2,-diyl, piperidin-2-yl, piperidin-3,3-diyl, piperidin-3-yl, piperidin-4,4-diyl, piperidin-4-yl, 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4 thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl where the aforementioned radicals may also be unsubstituted or substituted by one to six substituents selected from halogen, CN, C₁-C₆-alkyl and C₁-C₆-alkoxy.

In groups (2) of embodiments, R³ and R⁴ together with the carbon atom to which they are attached form in particular a moiety selected from the group consisting of C₃-C₆-cycloalkyl, C₃-C₆-cycloalkan-1,1-diyl, C₃-C₆-cycloalkenyl, ipso-C₃-C₆-cycloalkendiyl, three- to six-membered saturated, partially unsaturated or aromatic heterocyclyl and ipso-heterocyclodiyl, where the carbocycle and the heterocycle are unsubstituted or substituted by one to four substituents selected from halogen and C₁-C₆-alkyl groups and where the heterocycle preferably has 1 or 2 oxygen atoms as ring members.

Further in groups (2) of embodiments, R² and R³ and R⁴ together with the carbon atom to which they are attached may form a moiety selected from a 5- or 6-membered aromatic heterocyclyl, which is unsubstituted or substituted by one to four substituents selected from halogen and C₁-C₆-alkyl groups and where the heterocycle preferably has 1 or 2 oxygen atoms as ring members.

In groups (2) of embodiments, R³ and R⁴ together with the carbon atom to which they are attached more particularly form a moiety selected from the group consisting of C₃-C₆-cycloalkyl, C₃-C₆-cycloalkan-1,1-diyl or three- to six-membered saturated, partially unsaturated or aromatic heterocyclyl containing one or two heteroatoms selected from O, S and N, ipso-heterocyclodiyl, where the carbocycle and the heterocycle are unsubstituted or substituted by one to four substituents selected from halogen and C₁-C₆-alkyl groups, and where heterocyclyl preferably has 1 or 2 oxygen or sulfur atoms as ring members.

Further in groups (2) of embodiments, R² and R³ and R⁴ together with the carbon atom to which they are attached may form a moiety selected from a 5- or 6-membered aromatic heterocyclyl, which is unsubstituted or substituted by one to four substituents selected from halogen and C₁-C₆-alkyl groups, and where heterocyclyl preferably has 1 or 2 oxygen or sulfur atoms as ring members.

Preference is given to compounds of formula (I), wherein
R² is selected from the group consisting of H, halogen, C₁-C₆-alkyl, C₁-C₆-haloalkyl, C₁-C₆-alkoxy C₁-C₆-haloalkoxy and phenyl;
   in particular from the group consisting of H, halogen, C₁-C₆-alkyl, C₁-C₆-haloalkyl, C₁-C₆-alkoxy and C₁-C₆-haloalkoxy;
   more particular from the group consisting of H, halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃

Particular groups of embodiments relate to compounds of formula (I), wherein
R³ and R⁴ independently of one another preferably are H, halogen, C₁-C₆-alkyl or C₁-C₆-haloalkyl; or
   together with the carbon atom to which they are attached form a moiety selected from the group consisting of C₃-C₆-cycloalkyl, C₃-C₆-cycloalkenyl and three- to six-membered heterocyclyl, or
   R² and R³ and R⁴ together with the carbon atom to which they are attached may form a moiety selected from a 5- or 6-membered aromatic heterocyclyl,
      wherein the C₃-C₆-cycloalkyl, C₃-C₆-cycloalkenyl or the three- to six-membered heterocyclyl is unsubstituted or substituted by one to three substituents selected from halogen, CN, C₁-C₆-alkyl and C₁-C₆-alkoxy.
   particularly preferred are H, halogen, C₁-C₆-alkyl or C₁-C₆-haloalkyl; or
   together with the carbon atom to which they are attached form a moiety selected from the
   group consisting of C₃-C₆-cycloalkyl and C₃-C₆-cycloalkenyl,
      wherein the C₃-C₆-cycloalkyl or C₃-C₆-cycloalkenyl is unsubstituted or substituted by one to three substituents selected from halogen, CN, C₁-C₆-alkyl and C₁-C₆-alkoxy;
   especially preferred are H, halogen, C₁-C₆-alkyl or C₁-C₆-haloalkyl; or
      together with the carbon atom to which they are attached form a moiety selected from the group consisting of C₃-C₆-cycloalkyl, wherein the C₃-C₆-cycloalkyl is unsubstituted or substituted by one to three substituents selected from halogen, CN, C₁-C₆-alkyl and C₁-C₆-alkoxy;
   more preferred are H, halogen or C₁-C₆-alkyl.

Preference is given to compounds of formula (I), wherein
R² is as defined above and has in particular one of the preferred meanings and is especially selected from the group consisting of fluorine, C₁-C₄-alkyl, such as methyl, C₁-C₄-haloalkyl, such as trifluoromethyl, C₁-C₄-alkoxy, such as methoxy, and C₁-C₆-haloalkoxy such as trifluoromethoxy;
   in particular from the group consisting of H, halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃;
R³ is selected from the group consisting of hydrogen, fluorine, C₁-C₄-alkyl, such as methyl, C₁-C₄-haloalkyl, such as trifluoromethyl, C₁-C₄-alkoxy, such as methoxy, and C₁-C₆-haloalkoxy such as trifluoromethoxy;
R⁴ is selected from the group consisting of C₁-C₆-alkyl, C₁-C₆-haloalkyl, C₂-C₆-alkenyl, C₃-C₆-alkynyl, C₃-C₆-cycloalkyl, C₃-C₆-cycloalkenyl, and C₁-C₆-alkoxy-C₁-C₆-alkyl or
R³ and R⁴ together with the carbon atom to which they are attached form a moiety selected from the group consisting of C₃-C₆-cycloalkyl or five membered heteroaryl wherein the C₃-C₆-cycloalkyl and the five membered heteroaryl is unsubstituted or substituted by one to three substituents selected from halogen, CN, C₁-C₆-alkyl and C₁-C₆-alkoxy, or
R² and R³ and R⁴ together with the carbon atom to which they are attached may form a moiety selected from a 5- or 6-membered aromatic heterocyclyl, which is unsubstituted or substituted by one to three substituents selected from halogen, CN, C₁-C₆-alkyl and C₁-C₆-alkoxy, or
R³ and R⁴ together with the carbon atom to which they are attached form a moiety selected from the group consisting of C₃-C₆-cycloalkyl or five membered saturated or partially saturated heterocyclyl wherein the C₃-C₆-cycloalkyl and the five membered heterocyclyl is unsubstituted or substituted by one to three substituents selected from halogen, CN, C₁-C₆-alkyl and C₁-C₆-alkoxy.

Particular preference is given to compounds of formula (I), wherein
R² is selected from the group consisting of halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃;
R³ is selected from the group consisting of hydrogen, fluorine, C₁-C₄-alkyl, such as methyl, C₁-C₄-haloalkyl, such as trifluoromethyl, C₁-C₄-alkoxy, such as methoxy, and C₁-C₆-haloalkoxy such as trifluoromethoxy;
R⁴ is selected from the group consisting of C₁-C₆-alkyl, C₁-C₆-haloalkyl, C₂-C₆-alkenyl, C₃-C₆-alkynyl, C₃-C₆-cycloalkyl, C₃-C₆-cycloalkenyl, and C₁-C₆-alkoxy-C₁-C₆-alkyl or
R³ and R⁴ together with the carbon atom to which they are attached form a moiety selected from the group consisting of C₃-C₆-cycloalkyl or five membered heteroaryl wherein the C₃-C₆-cycloalkyl or the five membered heteroaryl is unsubstituted or substituted by one to three substituents selected from halogen, CN, C₁-C₆-alkyl and C₁-C₆-alkoxy or
R² and R³ and R⁴ together with the carbon atom to which they are attached may form a moiety selected from a 5- or 6-membered aromatic heterocyclyl, which is unsubstituted or substituted by one to three substituents selected from halogen, CN, C₁-C₆-alkyl and C₁-C₆-alkoxy, or
R³ and R⁴ together with the carbon atom to which they are attached form a moiety selected from the group consisting of C₃-C₆-cycloalkyl or five membered saturated or partially unsaturated heterocyclyl wherein the C₃-C₆-cycloalkyl or the five membered heterocyclyl is unsubstituted or substituted by one to three substituents selected from halogen, CN, C₁-C₆-alkyl and C₁-C₆-alkoxy.

Preference is also given to compounds of formula (I), wherein
R² is as defined above and has in particular one of the preferred meanings and is especially selected from the group consisting of H, fluorine, C₁-C₄-alkyl, such as methyl, C₁-C₄-haloalkyl, such as trifluoromethyl, C₁-C₄-alkoxy, such as methoxy, and C₁-C₆-haloalkoxy such as trifluoromethoxy;
   in particular from the group consisting of H, halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃;
R³ and R⁴ together with the carbon atom to which they are attached, form an ipso carbocyclic radical selected from C₃-C₆-cycloalkyl, C₃-C₆-cycloalkan-1,1-diyl, C₃-C₆-cycloalkenyl, ipso-C₃-C₆-cycloalkendiyl, where the ipso carbocyclic radical is unsubstituted or substituted by one to four substituents selected from halogen, CN and C₁-C₆-alkyl groups.

Preference is also given to compounds of formula (I), wherein
R² is as defined above and has in particular one of the preferred meanings and is especially selected from the group consisting of H, fluorine, C₁-C₄-alkyl, such as methyl, C₁-C₄-haloalkyl, such as trifluoromethyl, C₁-C₄-alkoxy, such as methoxy, and C₁-C₆-haloalkoxy such as trifluoromethoxy;
   in particular from the group consisting of H, halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃;
R³ and R⁴ together with the carbon atom to which they are attached, form three- to six-membered saturated partially unsaturated or aromatic heterocyclyl or ipso-heterocyclodiyl, where heterocyclyl or ipso-heterocyclodiyl is unsubstituted or substituted by one to four substituents selected from halogen, CN and C₁-C₆-alkyl groups and where the heterocyclyl, ipso-heterocyclodiyl preferably has 1 or 2 oxygen or sulfur atoms as ring members;
R² and R³ and R⁴ together with the carbon atom to which they are attached may form a moiety selected from a 5- or 6-membered aromatic heterocyclyl, which is unsubstituted or substituted by one to four substituents selected from halogen, CN and C₁-C₆-alkyl groups and where the where the heterocyclyl preferably has 1 or 2 oxygen or sulfur atoms as ring members.

Particular preference is given to compounds of formula (I), wherein
R² is selected from the group consisting of halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃;
R³ is selected from the group consisting of hydrogen, fluorine, C₁-C₄-alkyl, such as methyl, C₁-C₄-haloalkyl, such as trifluoromethyl, C₁-C₄-alkoxy, such as methoxy, and C₁-C₆-haloalkoxy such as trifluoromethoxy;
R⁴ is selected from the group consisting of C₁-C₆-alkyl, C₁-C₆-haloalkyl, C₂-C₆-alkenyl, C₃-C₆-alkynyl, C₃-C₆-cycloalkyl, C₃-C₆-cycloalkenyl, and C₁-C₆-alkoxy-C₁-C₆-alkyl.

Particular preference is also given to compounds of formula (I), wherein
R² is selected from the group consisting of halogen, C₁-C₄-alkyl, C₁-C₄-alkoxy and C₁-C₄-haloalkyl or from the group consisting of H, halogen, C₁-C₆-alkyl and C₁-C₆-haloalkyl;
   particularly preferred from the group consisting of halogen, C₁-C₄-alkyl, C₁-C₄-alkoxy and C₁-C₄-haloalkyl or from the group consisting of halogen, C₁-C₆-alkyl or C₁-C₆-haloalkyl;
   also particularly preferred is H, F, Cl, CH₃ or CF₃ or OCH₃;
R³ and R⁴ together with the carbon atom to which they are attached more particularly form a moiety selected from the group consisting of C₃-C₆-cycloalkyl, C₃-C₆-cycloalkan-1,1-diyl or three- to six-membered saturated, partially unsaturated or aromatic heterocyclyl or ipso-heterocyclodiyl, where the carbocycle and the heterocycle are unsubstituted or substituted by one to four substituents selected from halogen, CN and C₁-C₆-alkyl groups, and where heterocyclyl preferably has 1 or 2 oxygen or sulfur atoms as ring members;
R² and R³ and R⁴ together with the carbon atom to which they are attached may form a moiety selected from a 5- or 6-membered aromatic heterocyclyl, which is unsubstituted or substituted by one to four substituents selected from halogen, CN and C₁-C₆-alkyl groups and where the where the heterocyclyl preferably has 1 or 2 oxygen or sulfur atoms as ring members.

Also preferred are the azines of formula (I), wherein
R² is H, halogen, C₁-C₆-alkyl; and
R³ and R⁴ are independently of one another H, halogen, C₁-C₆-alkyl, or together with the carbon atom to which they are attached form a C₃-C₆-cycloalkyl;
particularly preferred R² is H, halogen or C₁-C₆-alkyl;
   R³ is C₁-C₆-alkyl;
   R⁴ is H, halogen or C₁-C₆-alkyl;
   R³ and R⁴ together with the carbon atom to which they are attached form a C₃-C₆-cycloalkyl;
   R² and R³ and R⁴ together with the carbon atom to which they are attached may form a moiety selected from a 5-membered aromatic heterocyclyl, containing one or two heteroatoms selected from O, S or N, which is unsubstituted or substituted by one substituent selected from halogen, methyl or ethyl;
especially preferred R² is halogen or C₁-C₆-alkyl;
   R³ is C₁-C₆-alkyl;
   R⁴ is H or C₁-C₆-alkyl;
more preferred R² is halogen; and
   R³ and R⁴ are C₁-C₆-alkyl.

Also preferred are the azines of formula (I), wherein
R² is H, halogen, C₁-C₆-alkyl; and
R³ and R⁴ together with the carbon atom to which they are attached form a saturated or partially unsaturated or aromatic 3, 4, 5- or 6-membered heterocycyl, or
R² and R³ and R⁴ together with the carbon atom to which they are attached may form a moiety selected from a 5- or 6-membered aromatic heterocyclyl, in particular heterocyclyl, which comprises 1 or 2 oxygen or sulfur atoms as ring members, wherein the heretocycles are unsubstituted or substituted by one substituent selected from halogen or methyl, especially an oxiran-2,2-diyl, oxiranyl, oxetan-2,2-diyl, oxetan-2-yl, oxetan-3,3-diyl, oxetan-3-yl, oxolan-2,2-diyl, oxolan-2-yl, oxolan-3-yl, oxan-2,2-diyl, oxan-2-yl, oxan-3,3-diyl, oxan-3-yl, 2-furyl, 3-furyl, 2-thienyl and 3-thienyl, 2-oxazolyl, 4-oxazolyl, 5-oxazolyl, more especially oxolan-2-yl, oxolan-3-yl, 2-furyl, 3-furyl, 2-thienyl and 3-thienyl, 2-oxazolyl, 4-oxazolyl, 5-oxazolyl, wherein the last aforementioned radical are unsubstituted or substituted by one substituent selected from halogen or methyl;
particularly preferred
R² is H, fluorine or C₁-C₄-alkyl;
R³ and R⁴ together with the carbon atom to which they are attached form a saturated, partially unsaturated or aromatic 3, 4, 5- or 6-membered heterocycyl, or
R² and R³ and R⁴ together with the carbon atom to which they are attached may form a moiety selected from a 5- or 6-membered aromatic heterocyclyl, in particular heterocyclyl, which comprises 1 or 2 oxygen or sulfur atoms as ring members wherein the heterocycle are unsubstituted or substituted by one substituent selected from halogen or methyl, especially an oxiran-2,2-diyl, oxiranyl, oxetan-2,2-diyl, oxetan-2-yl, oxolan-2,2-diyl, oxan-2-yl, oxan-2,2-diyl, oxan-3-yl, 2-furyl, 3-furyl, 2-thienyl or 3-thienyl, 2-oxazolyl, 4-oxazolyl, 5-oxazolyl, more especially oxolan-2-yl, oxolan-3-yl, 2-furyl, 3-furyl, 2-thienyl and 3-thienyl, 2-oxazolyl, 4-oxazolyl, 5-oxazolyl, wherein the last aforementioned radical are unsubstituted or substituted by one substituent selected from halogen or methyl.

Also preferred are the azines of formula (I), wherein
R² is H, halogen, C₁-C₆-alkyl or C₁-C₆-alkoxy; and
R³ and R⁴ together with the carbon atom to which they are attached form C₃-C₆-cycloalkan-1,1-diyl.

Also preferred are the azines of formula (I), wherein

R² is H, halogen, C₁-C₆-alkyl or C₁-C₆-alkoxy; and
R³ and R⁴ together with the carbon atom to which they are attached form C₃-C₆-cycloalkyl, in particular cyclopropyl, cyclobutyl, cyclopentyl, cyclohexyl.

Examples of suitable combinations of R², R³ and R⁴ are given in the following table 1:

TABLE 1

| # | R² | R³ | R⁴ |
|---|---|---|---|
| 1. | H | H | H |
| 2. | H | H | CH₃ |
| 3. | H | CH₃ | CH₃ |
| 4. | CH₃ | CH₃ | CH₃ |
| 5. | F | F | F |
| 6. | F | F | CH₃ |
| 7. | F | CH₃ | CH₃ |
| 8. | F | H | CH₃ |
| 9. | F | H | C₂H₅ |
| 10. | H | CH₃ | C₂H₅ |
| 11. | F | CH₃ | C₂H₅ |
| 12. | CN | CN | CN |
| 13. | CN | CN | CH₃ |
| 14. | CN | CH₃ | CH₃ |
| 15. | CN | H | CH₃ |
| 16. | CN | H | C₂H₅ |
| 17. | CN | CH₃ | C₂H₅ |
| 18. | CN | C₂H₅ | C₂H₅ |
| 19. | H | OCH₃ | CH₃ |
| 20. | H | OCH₃ | C₂H₅ |
| 21. | F | C₂H₅ | C₂H₅ |
| 22. | H | OCH₃ | C₂H₅ |
| 23. | H | H | CH(CH₃)₂ |
| 24. | H | F | CH(CH₃)₂ |
| 25. | F | F | CH(CH₃)₂ |
| 26. | H | CH₃ | CH(CH₃)₂ |
| 27. | H | OCH₃ | CH(CH₃)₂ |
| 28. | F | CH₃ | CH(CH₃)₂ |
| 29. | H | H | CH₂CH₂CH₃ |
| 30. | H | F | CH₂CH₂CH₃ |
| 31. | F | F | CH₂CH₂CH₃ |
| 32. | H | CH₃ | CH₂CH₂CH₃ |
| 33. | H | OCH₃ | CH₂CH₂CH₃ |
| 34. | F | CH₃ | CH₂CH₂CH₃ |
| 35. | H | H | C(CH₃)₃ |
| 36. | H | F | C(CH₃)₃ |
| 37. | F | F | C(CH₃)₃ |
| 38. | H | CH₃ | C(CH₃)₃ |
| 39. | H | OCH₃ | C(CH₃)₃ |
| 40. | F | CH₃ | C(CH₃)₃ |
| 41. | H | H | c-C₃H₅ |
| 42. | H | F | c-C₃H₅ |
| 43. | F | F | c-C₃H₅ |
| 44. | H | CH₃ | c-C₃H₅ |
| 45. | H | OCH₃ | c-C₃H₅ |
| 46. | F | CH₃ | c-C₃H₅ |
| 47. | H | CH₃ | CF₃ |
| 48. | F | CH₃ | CF₃ |

| | | |
|---|---|---|
| 49. | H | CH₂—CH₂ |
| 50. | CH₃ | CH₂—CH₂ |
| 51. | OCH₃ | CH₂—CH₂ |
| 52. | F | CH₂—CH₂ |
| 53. | Cl | CH₂—CH₂ |
| 54. | CN | CH₂—CH₂ |
| 55. | H | CH₂—CH₂—CH₂ |
| 56. | CH₃ | CH₂—CH₂—CH₂ |
| 57. | OCH₃ | CH₂—CH₂—CH₂ |
| 58. | F | CH₂—CH₂—CH₂ |
| 59. | Cl | CH₂—CH₂—CH₂ |
| 60. | CN | CH₂—CH₂—CH₂ |
| 61. | H | CH₂—CH₂—CH₂—CH₂ |
| 62. | CH₃ | CH₂—CH₂—CH₂—CH₂ |
| 63. | OCH₃ | CH₂—CH₂—CH₂—CH₂ |
| 64. | F | CH₂—CH₂—CH₂—CH₂ |
| 65. | Cl | CH₂—CH₂—CH₂—CH₂ |
| 66. | CN | CH₂—CH₂—CH₂—CH₂ |
| 67. | H | CH₂—CH₂—CH₂—CH₂—CH₂ |
| 68. | CH₃ | CH₂—CH₂—CH₂—CH₂—CH₂ |
| 69. | OCH₃ | CH₂—CH₂—CH₂—CH₂—CH₂ |
| 70. | F | CH₂—CH₂—CH₂—CH₂—CH₂ |
| 71. | Cl | CH₂—CH₂—CH₂—CH₂—CH₂ |
| 72. | CN | CH₂—CH₂—CH₂—CH₂—CH₂ |
| 73. | H | O—CH₂—CH₂—CH₂ |
| 74. | CH₃ | O—CH₂—CH₂—CH₂ |
| 75. | OCF₃ | O—CH₂—CH₂—CH₂ |
| 76. | H | O—CH₂—CH₂—CH₂—CH₂ |
| 77. | CH₃ | O—CH₂—CH₂—CH₂—CH₂ |
| 78. | OCF₃ | O—CH₂—CH₂—CH₂—CH₂ |
| 79. | H | 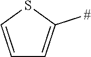 |
| 80. | F | 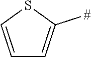 |
| 81. | CH₃ | 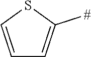 |
| 82. | H | 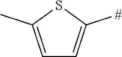 |
| 83. | F | 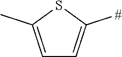 |
| 84. | CH₃ | 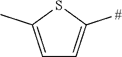 |
| 85. | H | 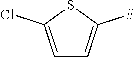 |
| 86. | F | 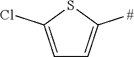 |
| 87. | CH₃ | 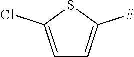 |
| 88. | H | 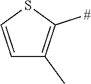 |
| 89. | F | 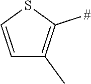 |
| 90. | CH₃ | 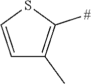 |
| 91. | H | 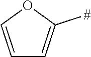 |
| 92. | F | 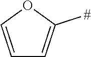 |
| 93. | CH₃ | 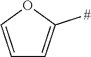 |
| 94. | H | 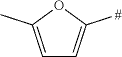 |
| 95. | F | 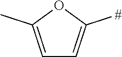 |
| 96. | CH₃ | 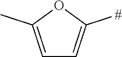 |
| 97. | H | 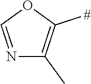 |
| 98. | F | 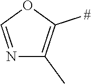 |
| 99. | CH₃ | 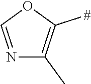 |
| 100. | H | 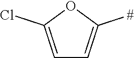 |
| 101. | F | 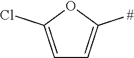 |
| 102. | CH₃ | 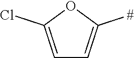 |

The following approvations were used in Table A

c-C₃H₅: Cyclopropyl

Particularly preferred are the aminotriazine compounds of formula (I), wherein X is CR^{A4}, wherein R^{A4} has the meanings as defined above. In particular
X is CR^{A4};
R^{b} is F;
n is 0, 1, 2 or 3, in particular 1, 2 or 3, more particularly 2 or 3;
R^{A} has one of the meanings as defined above, which are
   in particular selected from the group consisting of halogen, CN, C₁-C₆-alkyl, C₁-C₆-haloalkyl, C₃-C₆-cycloalkyl, C₂-C₆-alkynyl, C₂-C₆-alkenyl, C₁-C₆-alkoxy, C₃-C₆-alkenyloxy, C₃-C₆-cycloalkoxy, (C₃-C₆-cycloalkyl)methoxy, C₂-C₆-alkynyloxy, C₂-C₆-alkenyloxy or C₁-C₆-haloalkoxy;
   more particularly selected from the group consisting of halogen, CN, C₁-C₄-alkyl, C₁-C₄-haloalkyl, C₃-C₆-cycloalkyl, C₂-C₄-alkenyl, C₂-C₄-alkynyl, C₁-C₄-alkoxy, C₃-C₆-alkenyloxy, C₂-C₄-alkenyloxy, C₃-C₆-cycloalkoxy, (C₃-C₆-cycloalkyl)methoxy or C₁-C₄-haloalkoxy; even more particularly selected from the group consisting of F, Cl, Br, CN, CF₃, methyl, vinyl, ethynyl and cyclopropyl, methoxy, ethoxy, isopropyloxy, allyloxy, propargyloxy, cyclopropyloxy, cyclobutyloxy, cyclopentyloxy, (cyclopropyl)methoxy or 2-butyloxy;
   even more particularly selected from the group consisting of F, Cl, Br, CN, CF₃, methyl, vinyl, ethynyl and cyclopropyl;
   especially selected from the group consisting of F, Cl, Br and CN; and
R^{A4} is H or has one of the meanings from R^{A},
   in particular selected from the group consisting of H, halogen, CN, C₁-C₆-alkyl, C₁-C₆-haloalkyl, C₃-C₆-cycloalkyl, C₂-C₆-alkynyl, C₂-C₆-alkenyl, C₁-C₆-alkoxy, C₃-C₆-alkenyloxy, C₃-C₆-cycloalkoxy, (C₃-C₆-cycloalkyl)methoxy, C₂-C₆-alkynyloxy, C₂-C₆-alkenyloxy or C₁-C₆-haloalkoxy;
   more particularly selected from the group consisting of H, halogen, CN, C₁-C₄-alkyl, C₁-C₄-haloalkyl, C₃-C₆-cycloalkyl, C₂-C₄-alkenyl, C₂-C₄-alkynyl, C₁-C₄-alkoxy, C₃-C₆-alkenyloxy, C₂-C₄-alkenyloxy, C₃-C₆-cycloalkoxy, (C₃-C₆-cycloalkyl)methoxy or C₁-C₄-haloalkoxy;
   even more particularly selected from the group consisting of H, F, Cl, Br, CN, CF₃, methyl, vinyl, ethynyl and cyclopropyl, methoxy, ethoxy, isopropyloxy, allyloxy, propargyloxy, cyclopropyloxy, cyclobutyloxy, cyclopentyloxy, (cyclopropyl)methoxy or 2-butyloxy;
   even more particularly selected from the group consisting of H, F, Cl, Br, CN, CF₃, methyl, vinyl, ethynyl and cyclopropyl;
   especially selected from the group consisting of H, F, Cl, Br and CN;
   especially selected from the group consisting of H, F, Cl and CN.

Especially preferred A is 2,6-difluorophenyl, 2-chloro-6-fluorophenyl, 2-bromo-6-fluorophenyl, 2-fluoro-6-methylphenyl, 2-fluoro-6-cyanophenyl, 2,3,6-trifluorophenyl, 2,4,6-trifluorophenyl, 2-chloro-4,6-difluorophenyl, 2-chloro-5,6-difluorophenyl, 3-chloro-2,6-difluorophenyl, 2-chloro-3,6-difluorophenyl, 2,3-difluoro-6-cyanophenyl, 2,6-difluoro-3-cyanophenyl, 2,5-difluoro-6-cyanophenyl, 2,4-difluoro-6-cyanophenyl, 2,3,4,6-tetrafluorophenyl, 2,3,4,5-tetrafluorophenyl, 2,3,5,6-tetrafluorophenyl, 2-chloro-3,4,6-trifluorophenyl, 2-chloro-3,5,6-trifluorophenyl, 3-chloro-2,4,6-trifluorophenyl, 3-chloro-2,5,6-trifluorophenyl, 6-cyano-2,4,5-trifluorophenyl, 3-cyano-2,4,6-trifluorophenyl, 6-cyano-2,3,4-trifluorophenyl, 6-cyano-2,3,5-trifluorophenyl, 3-cyano-2,5,6-trifluorophenyl, pentafluorophenyl, 2-chloro-3,4,5,6-tetrafluorophenyl, 3-chloro-2,4,5,6-tetrafluorophenyl, 6-cyano-2,3,4,5-tetrafluorophenyl, 5-cyano-2,3,4,6-tetrafluorophenyl, 4-bromo-2,3,5,6-tetrafluorophenyl, 4-iodo-2,3,5,6-tetrafluorophenyl, 4-ethynyl-2,3,5,6-tetrafluorophenyl and 3-chloro-2-fluoro-6-(trifluoromethyl)phenyl.
R¹ is H, CN, C₁-C₆-alkyl, C₁-C₆-haloalkyl, C₁-C₆-alkoxy-C₁-C₆-alkyl, C₁-C₆-alkoxy, (C₁-C₆-alkyl)carbonyl or (C₁-C₆-alkyl)sulfonyl;
   particularly preferred H, CN, C₁-C₆-alkyl, C₁-C₆-alkoxy-C₁-C₆-alkyl, C₁-C₆-alkoxy, (C₁-C₆-alkyl)carbonyl or (C₁-C₆-alkyl)sulfonyl;
   especially preferred H, CN, CH₃, CH₂OCH₃, OCH₃, COCH₃ or SO₂CH₃;
   more preferred hydrogen;
R² is H, halogen, C₁-C₆-alkyl or C₁-C₆-haloalkyl;
   particularly preferred halogen, C₁-C₆-alkyl or C₁-C₆-haloalkyl;
   also particularly preferred H, F, CH₃ or CF₃;
R³ and R⁴ are independently of one another H, halogen, C₁-C₆-alkyl or C₁-C₆-haloalkyl; or
together with the carbon atom to which they are attached form a moiety selected from the group consisting of C₃-C₆-cycloalkyl, C₃-C₆-cycloalkenyl and three- to six-membered heterocyclyl,
wherein the C₃-C₆-cycloalkyl, C₃-C₆-cycloalkenyl or the three- to six-membered heterocyclyl is unsubstituted or substituted by one to three substituents selected from halogen, CN, C₁-C₆-alkyl and C₁-C₆-alkoxy;
independently of one another particularly preferred H, halogen, C₁-C₆-alkyl or C₁-C₆-haloalkyl; or
together with the carbon atom to which they are attached form a moiety selected from the group consisting of C₃-C₆-cycloalkyl, C₃-C₆-cycloalkenyl or three- to six-membered heterocyclyl containing one or two heteroatoms selected from O, S and N,
wherein the C₃-C₆-cycloalkyl, C₃-C₆-cycloalkenyl or three- to six-membered heterocyclyl is unsubstituted or substituted by one to three substituents selected from halogen, CN, C₁-C₆-alkyl and C₁-C₆-alkoxy;
independently of one another especially preferred H, halogen, C₁-C₆-alkyl or C₁-C₆-haloalkyl;
independently of one another more preferred H, halogen or C₁-C₆-alkyl.

Particular preference is given to aminotriazine compounds of formula (Ia), which correspond to aminotriazines of formula (I) wherein R¹ is hydrogen and where R², R³ and R⁴ are as defined in the rows of the following table A and wherein A is as defined in the following tables A-1 to A-35:

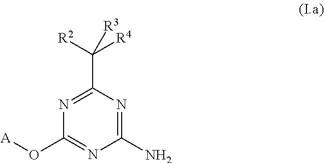

Table A-1: Compounds of the formula I.a, where A is 2,6-difluorophenyl and R², R³ and R⁴ are as defined in the rows of the following table A.

Table A-2: Compounds of the formula I.a, where A is 2-chloro-6-fluorophenyl and R², R³ and R⁴ are as defined in the rows of the following table A.

Table A-3: Compounds of the formula I.a, where A is 2-bromo-6-fluorophenyl and R², R³ and R⁴ are as defined in the rows of the following table A.

Table A-4: Compounds of the formula I.a, where A is 2-fluoro-6-methylphenyl and R², R³ and R⁴ are as defined in the rows of the following table A.

Table A-5: Compounds of the formula I.a, where A is 2-fluoro-6-cyanophenyl and R², R³ and R⁴ are as defined in the rows of the following table A.

Table A-6: Compounds of the formula I.a, where A is 2,3,6-trifluorophenyl and R², R³ and R⁴ are as defined in the rows of the following table A.

Table A-7: Compounds of the formula I.a, where A is 2,4,6-trifluorophenyl and R², R³ and R⁴ are as defined in the rows of the following table A.

Table A-8: Compounds of the formula I.a, where A is 2-chloro-4,6-difluorophenyl and R², R³ and R⁴ are as defined in the rows of the following table A.

Table A-9: Compounds of the formula I.a, where A is 2-chloro-5,6-difluorophenyl and R², R³ and R⁴ are as defined in the rows of the following table A.

Table A-10: Compounds of the formula I.a, where A is 3-chloro-2,6-difluorophenyl and R², R³ and R⁴ are as defined in the rows of the following table A.

Table A-11: Compounds of the formula I.a, where A is 2-chloro-3,6-difluorophenyl and R², R³ and R⁴ are as defined in the rows of the following table A.

Table A-12: Compounds of the formula I.a, where A is 2,3-difluoro-6-cyanophenyl and R², R³ and R⁴ are as defined in the rows of the following table A.

Table A-13: Compounds of the formula I.a, where A is 2,6-difluoro-3-cyanophenyl and R², R³ and R⁴ are as defined in the rows of the following table A.

Table A-14: Compounds of the formula I.a, where A is 2,5-difluoro-6-cyanophenyl and R², R³ and R⁴ are as defined in the rows of the following table A.

Table A-15: Compounds of the formula I.a, where A is 2,3,4,6-tetrafluorophenyl and R², R³ and R⁴ are as defined in the rows of the following table A.

Table A-16: Compounds of the formula I.a, where A is 2,3,4,5-tetrafluorophenyl and R², R³ and R⁴ are as defined in the rows of the following table A.

Table A-17: Compounds of the formula I.a, where A is 2,3,5,6-tetrafluorophenyl and R², R³ and R⁴ are as defined in the rows of the following table A.

Table A-18: Compounds of the formula I.a, where A is 2-chloro-3,4,6-trifluorophenyl and R², R³ and R⁴ are as defined in the rows of the following table A.

Table A-19: Compounds of the formula I.a, where A is 2-chloro-3,5,6-trifluorophenyl and R², R³ and R⁴ are as defined in the rows of the following table A.

Table A-20: Compounds of the formula I.a, where A is 3-chloro-2,4,6-trifluorophenyl and R², R³ and R⁴ are as defined in the rows of the following table A.

Table A-21: Compounds of the formula I.a, where A is 3-chloro-2,5,6-trifluorophenyl and R², R³ and R⁴ are as defined in the rows of the following table A.

Table A-22: Compounds of the formula I.a, where A is 6-cyano-2,4,5-trifluorophenyl and R², R³ and R⁴ are as defined in the rows of the following table A.

Table A-23: Compounds of the formula I.a, where A is 3-cyano-2,4,6-trifluorophenyl and R², R³ and R⁴ are as defined in the rows of the following table A.

Table A-24: Compounds of the formula I.a, where A is 6-cyano-2,3,4-trifluorophenyl and R², R³ and R⁴ are as defined in the rows of the following table A.

Table A-25: Compounds of the formula I.a, where A is 6-cyano-2,3,5-trifluorophenyl and R², R³ and R⁴ are as defined in the rows of the following table A.

Table A-26: Compounds of the formula I.a, where A is 3-cyano-2,5,6-trifluorophenyl and R², R³ and R⁴ are as defined in the rows of the following table A.

Table A-27: Compounds of the formula I.a, where A is pentafluorophenyl and R², R³ and R⁴ are as defined in the rows of the following table A.

Table A-28: Compounds of the formula I.a, where A is 2-chloro-3,4,5,6-tetrafluorophenyl and R², R³ and R⁴ are as defined in the rows of the following table A.

Table A-29: Compounds of the formula I.a, where A is 3-chloro-2,4,5,6-tetrafluorophenyl and R², R³ and R⁴ are as defined in the rows of the following table A.

Table A-30: Compounds of the formula I.a, where A is 6-cyano-2,3,4,5-tetrafluorophenyl and R², R³ and R⁴ are as defined in the rows of the following table A.

Table A-31: Compounds of the formula I.a, where A is 5-cyano-2,3,4,6-tetrafluorophenyl and R², R³ and R⁴ are as defined in the rows of the following table A.

Table A-32: Compounds of the formula I.a, where A is 4-bromo-2,3,5,6-tetrafluorophenyl and R², R³ and R⁴ are as defined in the rows of the following table A.

Table A-33: Compounds of the formula I.a, where A is 4-iodo-2,3,5,6-tetrafluorophenyl and R², R³ and R⁴ are as defined in the rows of the following table A.

Table A-34: Compounds of the formula I.a, where A is 4-ethynyl-2,3,5,6-tetrafluorophenyl and R², R³ and R⁴ are as defined in the rows of the following table A.

Table A-35: Compounds of the formula I.a, where A is and 3-chloro-2-fluoro-6-(trifluoromethyl)phenyl and R², R³ and R⁴ are as defined in the rows of the following table A.

TABLE A

| # | R² | R³ | R⁴ |
|---|---|---|---|
| 1. | H | H | H |
| 2. | H | H | CH₃ |
| 3. | H | CH₃ | CH₃ |
| 4. | CH₃ | CH₃ | CH₃ |
| 5. | F | F | F |
| 6. | F | F | CH₃ |
| 7. | F | CH₃ | CH₃ |
| 8. | F | H | CH₃ |
| 9. | F | H | C₂H₅ |
| 10. | H | CH₃ | C₂H₅ |
| 11. | F | CH₃ | C₂H₅ |
| 12. | CN | CN | CN |
| 13. | CN | CN | CH₃ |
| 14. | CN | CH₃ | CH₃ |
| 15. | CN | H | CH₃ |
| 16. | CN | H | C₂H₅ |
| 17. | CN | CH₃ | C₂H₅ |
| 18. | CN | C₂H₅ | C₂H₅ |
| 19. | H | OCH₃ | CH₃ |
| 20. | H | OCH₃ | C₂H₅ |
| 21. | F | C₂H₅ | C₂H₅ |
| 22. | H | OCH₃ | C₂H₅ |
| 23. | H | H | CH(CH₃)₂ |
| 24. | H | F | CH(CH₃)₂ |
| 25. | F | F | CH(CH₃)₂ |
| 26. | H | CH₃ | CH(CH₃)₂ |
| 27. | H | OCH₃ | CH(CH₃)₂ |
| 28. | F | CH₃ | CH(CH₃)₂ |
| 29. | H | H | CH₂CH₂CH₃ |
| 30. | H | F | CH₂CH₂CH₃ |
| 31. | F | F | CH₂CH₂CH₃ |
| 32. | H | CH₃ | CH₂CH₂CH₃ |
| 33. | H | OCH₃ | CH₂CH₂CH₃ |
| 34. | F | CH₃ | CH₂CH₂CH₃ |
| 35. | H | H | C(CH₃)₃ |
| 36. | H | F | C(CH₃)₃ |
| 37. | F | F | C(CH₃)₃ |
| 38. | H | CH₃ | C(CH₃)₃ |
| 39. | H | OCH₃ | C(CH₃)₃ |
| 40. | F | CH₃ | C(CH₃)₃ |
| 41. | H | H | Cyclopropyl |
| 42. | H | F | Cyclopropyl |
| 43. | F | F | Cyclopropyl |
| 44. | H | CH₃ | Cyclopropyl |
| 45. | H | OCH₃ | Cyclopropyl |
| 46. | F | CH₃ | Cyclopropyl |
| 47. | H | CH₃ | CF₃ |
| 48. | F | CH₃ | CF₃ |

| | | |
|---|---|---|
| 49. | H | CH₂—CH₂ |
| 50. | CH₃ | CH₂—CH₂ |
| 51. | OCH₃ | CH₂—CH₂ |
| 52. | F | CH₂—CH₂ |
| 53. | Cl | CH₂—CH₂ |
| 54. | CN | CH₂—CH₂ |
| 55. | H | CH₂—CH₂—CH₂ |
| 56. | CH₃ | CH₂—CH₂—CH₂ |
| 57. | OCH₃ | CH₂—CH₂—CH₂ |
| 58. | F | CH₂—CH₂—CH₂ |
| 59. | Cl | CH₂—CH₂—CH₂ |
| 60. | CN | CH₂—CH₂—CH₂ |
| 61. | H | CH₂—CH₂—CH₂—CH₂ |
| 62. | CH₃ | CH₂—CH₂—CH₂—CH₂ |
| 63. | OCH₃ | CH₂—CH₂—CH₂—CH₂ |
| 64. | F | CH₂—CH₂—CH₂—CH₂ |
| 65. | Cl | CH₂—CH₂—CH₂—CH₂ |
| 66. | CN | CH₂—CH₂—CH₂—CH₂ |
| 67. | H | CH₂—CH₂—CH₂—CH₂—CH₂ |
| 68. | CH₃ | CH₂—CH₂—CH₂—CH₂—CH₂ |
| 69. | OCH₃ | CH₂—CH₂—CH₂—CH₂—CH₂ |
| 70. | F | CH₂—CH₂—CH₂—CH₂—CH₂ |
| 71. | Cl | CH₂—CH₂—CH₂—CH₂—CH₂ |
| 72. | CN | CH₂—CH₂—CH₂—CH₂—CH₂ |
| 73. | H | O—CH₂—CH₂—CH₂ |
| 74. | CH₃ | O—CH₂—CH₂—CH₂ |
| 75. | OCF₃ | O—CH₂—CH₂—CH₂ |
| 76. | H | O—CH₂—CH₂—CH₂—CH₂ |
| 77. | CH₃ | O—CH₂—CH₂—CH₂—CH₂ |
| 78. | OCF₃ | O—CH₂—CH₂—CH₂—CH₂ |
| 79. | H | 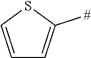 |
| 80. | F | 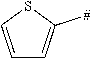 |
| 81. | CH₃ | 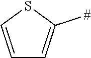 |
| 82. | H | 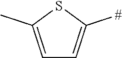 |
| 83. | F | 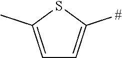 |
| 84. | CH₃ | 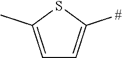 |
| 85. | H | 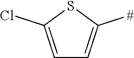 |
| 86. | F | 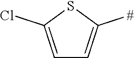 |
| 87. | CH₃ | 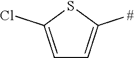 |
| 88. | H | 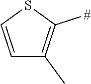 |
| 89. | F | 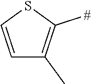 |
| 90. | CH₃ | 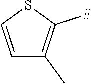 |
| 91. | H | 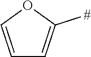 |
| 92. | F | 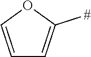 |
| 93. | CH₃ | 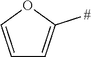 |
| 94. | H | 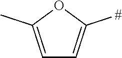 |
| 95. | F | 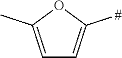 |
| 96. | CH₃ | 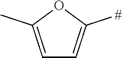 |
| 97. | H | 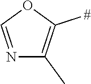 |
| 98. | F | 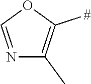 |
| 99. | CH₃ | 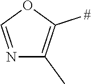 |
| 100. | H | 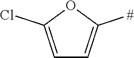 |
| 101. | F | 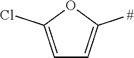 |
| 102. | CH₃ | 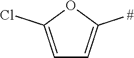 |

Also preferred are the azines of formula (I), wherein X is N, R^{A} and R^{b} have one of the meanings as defined above; in particular wherein
X is N;
R^{b} is selected from halogen in particular F;
n is 0, 1, 2 or 3, in particular 1, 2 or 3;
R^{A} has one of the meanings as defined above, which are in particular selected from
the group consisting of halogen, CN, NO₂, C₁-C₆-alkyl, C₁-C₆-haloalkyl, OH, C₁-C₆-alkoxy, C₁-C₆-alkylthio, (C₁-C₆-alkyl)sulfinyl, (C₁-C₆-alkyl)sulfonyl, amino, (C₁-C₆-alkyl)amino, di(C₁-C₆-alkyl)amino, (C₁-C₆-alkyl)carbonyl, (C₁-C₆-alkoxy)carbonyl;
particularly preferred selected from the group consisting of halogen, CN, C₁-C₆-alkyl, C₁-C₆-alkoxy, C₁-C₆-haloalkyl, C₃-C₆-cycloalkyl, C₃-C₆-cycloalkoxy, (C₃-C₆-cycloalkyl)methoxy, C₂-C₆-alkynyl, C₂-C₆-alkenyl, C₂-C₆-alkynyloxy, C₂-C₆-alkenyloxy or C₁-C₆-haloalkoxy;
more particularly preferred selected from the group consisting of halogen, CN, C₁-C₆-alkyl and C₁-C₆-alkoxy;
especially preferred selected from halogen and CN;
also especially preferred selected from the group consisting of F, Cl, CN and CH₃;
   more preferred selected from the group consisting of F, Cl and CN.
R¹ is H, CN, C₁-C₆-alkyl, C₁-C₆-haloalkyl, C₁-C₆-alkoxy-C₁-C₆-alkyl, C₁-C₆-alkoxy, (C₁-C₆-alkyl)carbonyl or (C₁-C₆-alkyl)sulfonyl;
   particularly preferred H, CN, C₁-C₆-alkyl, C₁-C₆-alkoxy-C₁-C₆-alkyl, C₁-C₆-alkoxy, (C₁-C₆-alkyl)carbonyl or (C₁-C₆-alkyl)sulfonyl;
   especially preferred H, CN, CH₃, CH₂OCH₃, OCH₃, COCH₃ or SO₂CH₃;
   more preferred hydrogen;
R² is H, halogen, C₁-C₆-alkyl or C₁-C₆-haloalkyl;
   particularly preferred halogen, C₁-C₆-alkyl or C₁-C₆-haloalkyl;
   also particularly preferred H, F, CH₃ or CF₃;
R³ and R⁴ are independently of one another H, halogen, C₁-C₆-alkyl or C₁-C₆-haloalkyl; or
together with the carbon atom to which they are attached form a moiety selected from the group consisting of C₃-C₆-cycloalkyl, C₃-C₆-cycloalkenyl and three- to six-membered heterocyclyl containing one or two heteroatoms selected from O, S and N,
wherein the C₃-C₆-cycloalkyl, C₃-C₆-cycloalkenyl or the three- to six-membered heterocyclyl is unsubstituted or substituted by one to three substituents selected from halogen, CN, C₁-C₆-alkyl and C₁-C₆-alkoxy;
independently of one another particularly preferred H, halogen, C₁-C₆-alkyl or C₁-C₆-haloalkyl; or
together with the carbon atom to which they are attached form a moiety selected from the group consisting of C₃-C₆-cycloalkyl, C₃-C₆-cycloalkenyl or three- to six-membered heterocyclyl,
wherein the C₃-C₆-cycloalkyl, C₃-C₆-cycloalkenyl or three- to six-membered heterocyclyl is unsubstituted or substituted by one to three substituents selected from halogen, CN, C₁-C₆-alkyl and C₁-C₆-alkoxy;
independently of one another especially preferred H, halogen, C₁-C₆-alkyl or C₁-C₆-haloalkyl;
independently of one another more preferred H, halogen or C₁-C₆-alkyl.

Particular preference is given to azines of formula (I.b), which correspond to azines of formula (I) wherein A is (A.3) with R^{A2} is H, and R¹ is H:

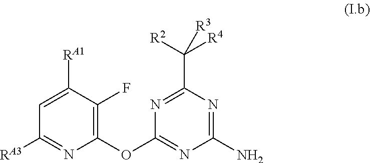

wherein the variables R^{A1}, R^{A3}, R², R³ and R⁴ have the meanings, in particular the preferred meanings, as defined above;
   special preference is given to the azines of the formulae (I.a.1) to (I.a.546) of Table 1, where the definitions of the variables R^{A1}, R^{A3}, R², R³ and R⁴ are of particular importance for the compounds according to the invention not only in combination with one another but in each case also on their own:

TABLE 1

| No. | R^{A1} | R^{A3} | R² | R³ | R⁴ |
|---|---|---|---|---|---|
| I.b.1 | F | H | CH₃ | H | H |
| I.b.2 | F | H | CH₃ | CH₃ | H |
| I.b.3 | F | H | CH₃ | CH₃ | CH₃ |
| I.b.4 | F | H | F | F | F |
| I.b.5 | F | H | F | CF₃ | F |
| I.b.6 | F | H | F | CH₃ | F |
| I.b.7 | F | H | F | CH₃ | H |
| I.b.8 | F | H | F | CH₃ | CH₃ |
| I.b.9 | F | H | Cl | CH₃ | CH₃ |
| I.b.10 | F | H | F | C₂H₅ | CH₃ |
| I.b.11 | F | H | F | C₂H₅ | C₂H₅ |
| I.b.12 | F | H | H | —(CH₂)₂— | |
| I.b.13 | F | H | H | —(CH₂)₃— | |
| I.b.14 | F | H | H | —(CH₂)₄— | |
| I.b.15 | F | H | H | —(CH₂)₅— | |
| I.b.16 | F | H | CH₃ | —(CH₂)₂— | |
| I.b.17 | F | H | CH₃ | —(CH₂)₃— | |
| I.b.18 | F | H | CH₃ | —(CH₂)₄— | |
| I.b.19 | F | H | CH₃ | —(CH₂)₅— | |
| I.b.20 | F | H | F | —(CH₂)₂— | |
| I.b.21 | F | H | F | —(CH₂)₃— | |
| I.b.22 | F | H | F | —(CH₂)₄— | |
| I.b.23 | F | H | F | —(CH₂)₅— | |
| I.b.24 | F | H | Cl | —(CH₂)₂— | |
| I.b.25 | F | H | Cl | —(CH₂)₃— | |
| I.b.26 | F | H | Cl | —(CH₂)₄— | |
| I.b.27 | F | H | Cl | —(CH₂)₅— | |
| I.b.28 | F | F | CH₃ | H | H |
| I.b.29 | F | F | CH₃ | CH₃ | H |
| I.b.30 | F | F | CH₃ | CH₃ | CH₃ |
| I.b.31 | F | F | F | F | F |
| I.b.32 | F | F | F | CF₃ | F |
| I.b.33 | F | F | F | CH₃ | F |
| I.b.34 | F | F | F | CH₃ | H |
| I.b.35 | F | F | F | CH₃ | CH₃ |
| I.b.36 | F | F | Cl | CH₃ | CH₃ |
| I.b.37 | F | F | F | C₂H₅ | CH₃ |
| I.b.38 | F | F | F | C₂H₅ | C₂H₅ |
| I.b.39 | F | F | H | —(CH₂)₂— | |
| I.b.40 | F | F | H | —(CH₂)₃— | |
| I.b.41 | F | F | H | —(CH₂)₄— | |
| I.b.42 | F | F | H | —(CH₂)₅— | |
| I.b.43 | F | F | CH₃ | —(CH₂)₂— | |
| I.b.44 | F | F | CH₃ | —(CH₂)₃— | |
| I.b.45 | F | F | CH₃ | —(CH₂)₄— | |
| I.b.46 | F | F | CH₃ | —(CH₂)₅— | |
| I.b.47 | F | F | F | —(CH₂)₂— | |
| I.b.48 | F | F | F | —(CH₂)₃— | |
| I.b.49 | F | F | F | —(CH₂)₄— | |
| I.b.50 | F | F | F | —(CH₂)₅— | |
| I.b.51 | F | F | Cl | —(CH₂)₂— | |
| I.b.52 | F | F | Cl | —(CH₂)₃— | |
| I.b.53 | F | F | Cl | —(CH₂)₄— | |
| I.b.54 | F | F | Cl | —(CH₂)₅— | |
| I.b.55 | F | Cl | CH₃ | H | H |
| I.b.56 | F | Cl | CH₃ | CH₃ | H |
| I.b.57 | F | Cl | CH₃ | CH₃ | CH₃ |
| I.b.58 | F | Cl | F | F | F |
| I.b.59 | F | Cl | F | CF₃ | F |
| I.b.60 | F | Cl | F | CH₃ | F |
| I.b.61 | F | Cl | F | CH₃ | H |
| I.b.62 | F | Cl | F | CH₃ | CH₃ |
| I.b.63 | F | Cl | Cl | CH₃ | CH₃ |
| I.b.64 | F | Cl | F | C₂H₅ | CH₃ |
| I.b.65 | F | Cl | F | C₂H₅ | C₂H₅ |
| I.b.66 | F | Cl | H | —(CH₂)₂— | |
| I.b.67 | F | Cl | H | —(CH₂)₃— | |
| I.b.68 | F | Cl | H | —(CH₂)₄— | |
| I.b.69 | F | Cl | H | —(CH₂)₅— | |
| I.b.70 | F | Cl | CH₃ | —(CH₂)₂— | |
| I.b.71 | F | Cl | CH₃ | —(CH₂)₃— | |
| I.b.72 | F | Cl | CH₃ | —(CH₂)₄— | |
| I.b.73 | F | Cl | CH₃ | —(CH₂)₅— | |
| I.b.74 | F | Cl | F | —(CH₂)₂— | |
| I.b.75 | F | Cl | F | —(CH₂)₃— | |
| I.b.76 | F | Cl | F | —(CH₂)₄— | |
| I.b.77 | F | Cl | F | —(CH₂)₅— | |
| I.b.78 | F | Cl | Cl | —(CH₂)₂— | |
| I.b.79 | F | Cl | Cl | —(CH₂)₃— | |
| I.b.80 | F | Cl | Cl | —(CH₂)₄— | |
| I.b.81 | F | Cl | Cl | —(CH₂)₅— | |
| I.b.82 | F | CN | CH₃ | H | H |
| I.b.83 | F | CN | CH₃ | CH₃ | H |
| I.b.84 | F | CN | CH₃ | CH₃ | CH₃ |
| I.b.85 | F | CN | F | F | F |
| I.b.86 | F | CN | F | CF₃ | F |
| I.b.87 | F | CN | F | CH₃ | F |
| I.b.88 | F | CN | F | CH₃ | H |
| I.b.89 | F | CN | F | CH₃ | CH₃ |
| I.b.90 | F | CN | Cl | CH₃ | CH₃ |
| I.b.91 | F | CN | F | C₂H₅ | CH₃ |
| I.b.92 | F | CN | F | C₂H₅ | C₂H₅ |
| I.b.93 | F | CN | H | —(CH₂)₂— | |
| I.b.94 | F | CN | H | —(CH₂)₃— | |
| I.b.95 | F | CN | H | —(CH₂)₄— | |
| I.b.96 | F | CN | H | —(CH₂)₅— | |
| I.b.97 | F | CN | CH₃ | —(CH₂)₂— | |
| I.b.98 | F | CN | CH₃ | —(CH₂)₃— | |
| I.b.99 | F | CN | CH₃ | —(CH₂)₄— | |
| I.b.100 | F | CN | CH₃ | —(CH₂)₅— | |
| I.b.101 | F | CN | F | —(CH₂)₂— | |
| I.b.102 | F | CN | F | —(CH₂)₃— | |
| I.b.103 | F | CN | F | —(CH₂)₄— | |
| I.b.104 | F | CN | F | —(CH₂)₅— | |
| I.b.105 | F | CN | Cl | —(CH₂)₂— | |
| I.b.106 | F | CN | Cl | —(CH₂)₃— | |
| I.b.107 | F | CN | Cl | —(CH₂)₄— | |
| I.b.108 | F | CN | Cl | —(CH₂)₅— | |
| I.b.109 | Cl | H | CH₃ | H | H |
| I.b.110 | Cl | H | CH₃ | CH₃ | H |
| I.b.111 | Cl | H | CH₃ | CH₃ | CH₃ |
| I.b.112 | Cl | H | F | F | F |
| I.b.113 | Cl | H | F | CF₃ | F |
| I.b.114 | Cl | H | F | CH₃ | F |
| I.b.115 | Cl | H | F | CH₃ | H |
| I.b.116 | Cl | H | F | CH₃ | CH₃ |
| I.b.117 | Cl | H | Cl | CH₃ | CH₃ |
| I.b.118 | Cl | H | F | C₂H₅ | CH₃ |
| I.b.119 | Cl | H | F | C₂H₅ | C₂H₅ |
| .b.120 | Cl | H | H | —(CH₂)₂— | |
| I.b.121 | Cl | H | H | —(CH₂)₃— | |
| I.b.122 | Cl | H | H | —(CH₂)₄— | |
| I.b.123 | Cl | H | H | —(CH₂)₅— | |
| I.b.124 | Cl | H | CH₃ | —(CH₂)₂— | |
| I.b.125 | Cl | H | CH₃ | —(CH₂)₃— | |
| I.b.126 | Cl | H | CH₃ | —(CH₂)₄— | |
| I.b.127 | Cl | H | CH₃ | —(CH₂)₅— | |
| I.b.128 | Cl | H | F | —(CH₂)₂— | |
| I.b.129 | Cl | H | F | —(CH₂)₃— | |
| I.b.130 | Cl | H | F | —(CH₂)₄— | |
| I.b.131 | Cl | H | F | —(CH₂)₅— | |
| I.b.132 | Cl | H | Cl | —(CH₂)₂— | |
| I.b.133 | Cl | H | Cl | —(CH₂)₃— | |
| I.b.134 | Cl | H | Cl | —(CH₂)₄— | |
| I.b.135 | Cl | H | Cl | —(CH₂)₅— | |
| I.b.136 | Cl | F | CH₃ | H | H |
| I.b.137 | Cl | F | CH₃ | CH₃ | H |
| I.b.138 | Cl | F | CH₃ | CH₃ | CH₃ |
| I.b.139 | Cl | F | F | F | F |
| I.b.140 | Cl | F | F | CF₃ | F |
| I.b.141 | Cl | F | F | CH₃ | F |
| I.b.142 | Cl | F | F | CH₃ | H |
| I.b.143 | Cl | F | F | CH₃ | CH₃ |
| I.b.144 | Cl | F | Cl | CH₃ | CH₃ |
| I.b.145 | Cl | F | F | C₂H₅ | CH₃ |
| I.b.146 | Cl | F | F | C₂H₅ | C₂H₅ |
| I.b.147 | Cl | F | H | —(CH₂)₂— | |
| I.b.148 | Cl | F | H | —(CH₂)₃— | |
| I.b.149 | Cl | F | H | —(CH₂)₄— | |
| I.b.150 | Cl | F | H | —(CH₂)₅— | |
| I.b.151 | Cl | F | CH₃ | —(CH₂)₂— | |
| I.b.152 | Cl | F | CH₃ | —(CH₂)₃— | |
| I.b.153 | Cl | F | CH₃ | —(CH₂)₄— | |
| I.b.154 | Cl | F | CH₃ | —(CH₂)₅— | |
| I.b.155 | Cl | F | F | —(CH₂)₂— | |
| I.b.156 | Cl | F | F | —(CH₂)₃— | |
| I.b.157 | Cl | F | F | —(CH₂)₄— | |
| I.b.158 | Cl | F | F | —(CH₂)₅— | |
| I.b.159 | Cl | F | Cl | —(CH₂)₂— | |
| I.b.160 | Cl | F | Cl | —(CH₂)₃— | |
| I.b.161 | Cl | F | Cl | —(CH₂)₄— | |
| I.b.162 | Cl | F | Cl | —(CH₂)₅— | |
| I.b.163 | CN | H | CH₃ | H | H |
| I.b.164 | CN | H | CH₃ | CH₃ | H |
| I.b.165 | CN | H | CH₃ | CH₃ | CH₃ |
| I.b.166 | CN | H | F | F | F |
| I.b.167 | CN | H | F | CF₃ | F |
| I.b.168 | CN | H | F | CH₃ | F |
| I.b.169 | CN | H | F | CH₃ | H |
| I.b.170 | CN | H | F | CH₃ | CH₃ |
| I.b.171 | CN | H | Cl | CH₃ | CH₃ |
| I.b.172 | CN | H | F | C₂H₅ | CH₃ |
| I.b.173 | CN | H | F | C₂H₅ | C₂H₅ |
| I.b.174 | CN | H | H | —(CH₂)₂— | |
| I.b.175 | CN | H | H | —(CH₂)₃— | |
| I.b.176 | CN | H | H | —(CH₂)₄— | |
| I.b.177 | CN | H | H | —(CH₂)₅— | |
| I.b.178 | CN | H | CH₃ | —(CH₂)₂— | |
| I.b.179 | CN | H | CH₃ | —(CH₂)₃— | |
| I.b.180 | CN | H | CH₃ | —(CH₂)₄— | |
| I.b.181 | CN | H | CH₃ | —(CH₂)₅— | |
| I.b.182 | CN | H | F | —(CH₂)₂— | |
| I.b.183 | CN | H | F | —(CH₂)₃— | |
| I.b.184 | CN | H | F | —(CH₂)₄— | |
| I.b.185 | CN | H | F | —(CH₂)₅— | |
| I.b.186 | CN | H | Cl | —(CH₂)₂— | |
| I.b.187 | CN | H | Cl | —(CH₂)₃— | |
| I.b.188 | CN | H | Cl | —(CH₂)₄— | |
| I.b.189 | CN | H | Cl | —(CH₂)₅— | |
| I.b.190 | CN | F | CH₃ | H | H |
| I.b.191 | CN | F | CH₃ | CH₃ | H |
| I.b.192 | CN | F | CH₃ | CH₃ | CH₃ |
| I.b.193 | CN | F | F | F | F |
| I.b.194 | CN | F | F | CF₃ | F |
| I.b.195 | CN | F | F | CH₃ | F |
| I.b.196 | CN | F | F | CH₃ | H |
| I.b.197 | CN | F | F | CH₃ | CH₃ |
| I.b.198 | CN | F | Cl | CH₃ | CH₃ |
| I.b.199 | CN | F | F | C₂H₅ | CH₃ |
| I.b.200 | CN | F | F | C₂H₅ | C₂H₅ |
| I.b.201 | CN | F | H | —(CH₂)₂— | |
| I.b.202 | CN | F | H | —(CH₂)₃— | |
| I.b.203 | CN | F | H | —(CH₂)₄— | |
| I.b.204 | CN | F | H | —(CH₂)₅— | |
| I.b.205 | CN | F | CH₃ | —(CH₂)₂— | |
| I.b.206 | CN | F | CH₃ | —(CH₂)₃— | |
| I.b.207 | CN | F | CH₃ | —(CH₂)₄— | |
| I.b.208 | CN | F | CH₃ | —(CH₂)₅— | |
| I.b.209 | CN | F | F | —(CH₂)₂— | |
| I.b.210 | CN | F | F | —(CH₂)₃— | |
| I.b.211 | CN | F | F | —(CH₂)₄— | |
| I.b.212 | CN | F | F | —(CH₂)₅— | |
| I.b.213 | CN | F | Cl | —(CH₂)₂— | |
| I.b.214 | CN | F | Cl | —(CH₂)₃— | |
| I.b.215 | CN | F | Cl | —(CH₂)₄— | |
| I.b.216 | CN | F | Cl | —(CH₂)₅— | |
| I.b.217 | F | H | F | H | C₂H₅ |
| I.b.218 | F | H | OCH₃ | H | C₂H₅ |
| I.b.219 | F | H | H | H | C(CH₃)₃ |
| I.b.220 | F | H | F | H | C(CH₃)₃ |
| I.b.221 | F | H | F | F | C(CH₃)₃ |
| I.b.222 | F | H | CH₃ | H | C(CH₃)₃ |
| I.b.223 | F | H | OCH₃ | H | C(CH₃)₃ |
| I.b.224 | F | H | CH₃ | F | C(CH₃)₃ |
| I.b.225 | F | H | H | H | CH(CH₃)₂ |
| I.b.226 | F | H | F | H | CH(CH₃)₂ |
| I.b.227 | F | H | F | F | CH(CH₃)₂ |
| I.b.228 | F | H | CH₃ | H | CH(CH₃)₂ |
| I.b.229 | F | H | OCH₃ | H | CH(CH₃)₂ |
| I.b.230 | F | H | CH₃ | F | CH(CH₃)₂ |
| I.b.231 | F | H | H | H | c-C₃H₅ |
| I.b.232 | F | H | F | H | c-C₃H₅ |
| I.b.233 | F | H | F | F | c-C₃H₅ |
| I.b.234 | F | H | CH₃ | H | c-C₃H₅ |
| I.b.235 | F | H | OCH₃ | H | c-C₃H₅ |
| I.b.236 | F | H | CH₃ | F | c-C₃H₅ |
| I.b.237 | F | H | H | H | CH₂CH₂CH₃ |
| I.b.238 | F | H | F | H | CH₂CH₂CH₃ |
| I.b.239 | F | H | F | F | CH₂CH₂CH₃ |
| I.b.240 | F | H | CH₃ | H | CH₂CH₂CH₃ |
| I.b.241 | F | H | CH₂CH₃ | H | CH₂CH₂CH₃ |
| I.b.242 | F | H | OCH₃ | H | CH₂CH₂CH₃ |
| I.b.243 | F | H | CH₃ | F | CH₂CH₂CH₃ |
| I.b.244 | F | H | CH₃ | H | CF₃ |
| I.b.245 | F | H | CH₃ | F | CF₃ |
| I.b.246 | F | H | H | —(CH₂)₂—CH(CH₃)—(CH₂)₂— | |
| I.b.247 | F | H | H | —O—(CH₂)₂— | |
| I.b.248 | F | H | H | —O—(CH₂)₃— | |
| I.b.249 | F | H | H | —O—(CH₂)₄— | |
| I.b.250 | F | H | CH₃ | —(CH₂)₂—CH(CH₃)—(CH₂)₂— | |
| I.b.251 | F | H | CH₃ | —O—(CH₂)₂— | |
| I.b.252 | F | H | CH₃ | —O—(CH₂)₃— | |
| I.b.253 | F | H | CH₃ | —O—(CH₂)₄— | |
| I.b.254 | F | H | F | —(CH₂)₂—CH(CH₃)—(CH₂)₂— | |
| I.b.255 | F | H | F | —O—(CH₂)₂— | |
| I.b.256 | F | H | F | —O—(CH₂)₃— | |
| I.b.257 | F | H | F | —O—(CH₂)₄— | |
| I.b.258 | F | H | Cl | —(CH₂)₂—CH(CH₃)—(CH₂)₂— | |
| I.b.259 | F | H | Cl | —O—(CH₂)₂— | |
| I.b.260 | F | H | Cl | —O—(CH₂)₃— | |
| I.b.261 | F | H | Cl | —O—(CH₂)₄— | |
| I.b.262 | F | H | CH₃ | —O—(CH₂)₃— | |
| I.b.263 | F | H | CH₃ | —O—(CH₂)₄— | |
| I.b.264 | F | H | OCH₃ | —O—(CH₂)₃— | |
| I.b.265 | F | H | OCH₃ | —O—(CH₂)₄— | |
| I.b.266 | F | H | OCF₃ | —O—(CH₂)₃— | |
| I.b.267 | F | H | OCF₃ | —O—(CH₂)₄— | |
| I.b.268 | F | H | OCH₃ | —(CH₂)₂— | |
| I.b.269 | F | H | OCH₃ | —(CH₂)₃— | |
| I.b.270 | F | H | OCH₃ | —(CH₂)₄— | |
| I.b.271 | F | H | OCH₃ | —(CH₂)₅— | |
| I.b.272 | F | F | F | H | C₂H₅ |
| I.b.273 | F | F | OCH₃ | H | C₂H₅ |
| I.b.274 | F | F | H | H | C(CH₃)₃ |
| I.b.275 | F | F | F | H | C(CH₃)₃ |
| I.b.276 | F | F | F | F | C(CH₃)₃ |
| I.b.277 | F | F | CH₃ | H | C(CH₃)₃ |
| I.b.278 | F | F | OCH₃ | H | C(CH₃)₃ |
| I.b.279 | F | F | CH₃ | F | C(CH₃)₃ |
| I.b.280 | F | F | H | H | CH(CH₃)₂ |
| I.b.281 | F | F | F | H | CH(CH₃)₂ |
| I.b.282 | F | F | F | F | CH(CH₃)₂ |
| I.b.283 | F | F | CH₃ | H | CH(CH₃)₂ |
| I.b.284 | F | F | OCH₃ | H | CH(CH₃)₂ |
| I.b.285 | F | F | CH₃ | F | CH(CH₃)₂ |
| I.b.286 | F | F | H | H | c-C₃H₅ |
| I.b.287 | F | F | F | H | c-C₃H₅ |
| I.b.288 | F | F | F | F | c-C₃H₅ |
| I.b.289 | F | F | CH₃ | H | c-C₃H₅ |
| I.b.290 | F | F | OCH₃ | H | c-C₃H₅ |
| I.b.291 | F | F | CH₃ | F | c-C₃H₅ |
| I.b.292 | F | F | H | H | CH₂CH₂CH₃ |
| I.b.293 | F | F | F | H | CH₂CH₂CH₃ |
| I.b.294 | F | F | F | F | CH₂CH₂CH₃ |
| I.b.295 | F | F | CH₃ | H | CH₂CH₂CH₃ |
| I.b.296 | F | F | CH₂CH₃ | H | CH₂CH₂CH₃ |
| I.b.297 | F | F | OCH₃ | H | CH₂CH₂CH₃ |
| I.b.298 | F | F | CH₃ | F | CH₂CH₂CH₃ |
| I.b.299 | F | F | CH₃ | H | CF₃ |
| I.b.300 | F | F | CH₃ | F | CF₃ |
| I.b.301 | F | F | H | H | OH |
| I.b.302 | F | F | H | H | OCH₃ |
| I.b.303 | F | F | H | H | OCF₃ |
| I.b.304 | F | F | CH₃ | H | OH |
| I.b.305 | F | F | CH₃ | H | OCH₃ |
| I.b.306 | F | F | CH₃ | H | OCF₃ |
| I.b.307 | F | F | CH₃ | CH₃ | OH |
| I.b.308 | F | F | CH₃ | CH₃ | OCH₃ |
| I.b.309 | F | F | CH₃ | CH₃ | OCF₃ |
| I.b.310 | F | F | H | —(CH₂)₂—CH(CH₃)—(CH₂)₂— | |
| I.b.311 | F | F | H | —O—(CH₂)₂— | |
| I.b.312 | F | F | H | —O—(CH₂)₃— | |
| I.b.313 | F | F | H | —O—(CH₂)₄— | |
| I.b.314 | F | F | CH₃ | —(CH₂)₂—CH(CH₃)—(CH₂)₂— | |
| I.b.315 | F | F | CH₃ | —O—(CH₂)₂— | |
| I.b.316 | F | F | CH₃ | —O—(CH₂)₃— | |
| I.b.317 | F | F | CH₃ | —O—(CH₂)₄— | |
| I.b.318 | F | F | F | —(CH₂)₂—CH(CH₃)—(CH₂)₂— | |
| I.b.319 | F | F | F | —O—(CH₂)₂— | |
| I.b.320 | F | F | F | —O—(CH₂)₃— | |
| I.b.321 | F | F | F | —O—(CH₂)₄— | |
| I.b.322 | F | F | Cl | —(CH₂)₂—CH(CH₃)—(CH₂)₂— | |
| I.b.323 | F | F | Cl | —O—(CH₂)₂— | |
| I.b.324 | F | F | Cl | —O—(CH₂)₃— | |
| I.b.325 | F | F | Cl | —O—(CH₂)₄— | |
| I.b.326 | F | F | CH₃ | —O—(CH₂)₃— | |
| I.b.327 | F | F | CH₃ | —O—(CH₂)₄— | |
| I.b.328 | F | F | OCH₃ | —O—(CH₂)₃— | |
| I.b.329 | F | F | OCH₃ | —O—(CH₂)₄— | |
| I.b.330 | F | F | OCF₃ | —O—(CH₂)₃— | |
| I.b.331 | F | F | OCF₃ | —O—(CH₂)₄— | |
| I.b.332 | F | F | OCH₃ | —(CH₂)₂— | |
| I.b.333 | F | F | OCH₃ | —(CH₂)₃— | |
| I.b.334 | F | F | OCH₃ | —(CH₂)₄— | |
| I.b.335 | F | F | OCH₃ | —(CH₂)₅— | |
| I.b.336 | F | F | OH | —(CH₂)₂— | |
| I.b.337 | F | F | OH | —(CH₂)₃— | |
| I.b.338 | F | F | OH | —(CH₂)₄— | |
| I.b.339 | F | F | OH | —(CH₂)₅— | |
| I.b.340 | F | F | OCF₃ | —(CH₂)₂— | |
| I.b.341 | F | F | OCF₃ | —(CH₂)₃— | |
| I.b.342 | F | F | OCF₃ | —(CH₂)₄— | |
| I.b.343 | F | F | OCF₃ | —(CH₂)₅— | |
| I.b.344 | F | H | H | H | OH |
| I.b.345 | F | H | H | H | OCH₃ |
| I.b.346 | F | H | H | H | OCF₃ |
| I.b.347 | F | H | CH₃ | H | OH |
| I.b.348 | F | H | CH₃ | H | OCH₃ |
| I.b.349 | F | H | CH₃ | H | OCF₃ |
| I.b.350 | F | H | CH₃ | CH₃ | OH |
| I.b.351 | F | H | CH₃ | CH₃ | OCH₃ |
| I.b.352 | F | H | CH₃ | CH₃ | OCF₃ |
| I.b.353 | F | H | OH | —(CH₂)₂— | |
| I.b.354 | F | H | OH | —(CH₂)₃— | |
| I.b.355 | F | H | OH | —(CH₂)₄— | |
| I.b.356 | F | H | OH | —(CH₂)₅— | |
| I.b.357 | F | H | OCF₃ | —(CH₂)₂— | |
| I.b.358 | F | H | OCF₃ | —(CH₂)₃— | |
| I.b.359 | F | H | OCF₃ | —(CH₂)₄— | |
| I.b.360 | F | H | OCF₃ | —(CH₂)₅— | |
| I.b.361 | F | Cl | F | H | C₂H₅ |
| I.b.362 | F | Cl | OCH₃ | H | C₂H₅ |
| I.b.363 | F | Cl | H | H | C(CH₃)₃ |
| I.b.364 | F | Cl | F | H | C(CH₃)₃ |
| I.b.365 | F | Cl | F | F | C(CH₃)₃ |
| I.b.366 | F | Cl | CH₃ | H | C(CH₃)₃ |
| I.b.367 | F | Cl | OCH₃ | H | C(CH₃)₃ |
| I.b.368 | F | Cl | CH₃ | F | C(CH₃)₃ |
| I.b.369 | F | Cl | H | H | CH(CH₃)₂ |
| I.b.370 | F | Cl | F | H | CH(CH₃)₂ |
| I.b.371 | F | Cl | F | F | CH(CH₃)₂ |
| I.b.372 | F | Cl | CH₃ | H | CH(CH₃)₂ |
| I.b.373 | F | Cl | OCH₃ | H | CH(CH₃)₂ |
| I.b.374 | F | Cl | CH₃ | F | CH(CH₃)₂ |
| I.b.375 | F | Cl | H | H | c-C₃H₅ |
| I.b.376 | F | Cl | F | H | c-C₃H₅ |
| I.b.377 | F | Cl | F | F | c-C₃H₅ |
| I.b.378 | F | Cl | CH₃ | H | c-C₃H₅ |
| I.b.379 | F | Cl | OCH₃ | H | c-C₃H₅ |
| I.b.380 | F | Cl | CH₃ | F | c-C₃H₅ |
| I.b.381 | F | Cl | H | H | CH₂CH₂CH₃ |
| I.b.382 | F | Cl | F | H | CH₂CH₂CH₃ |
| I.b.383 | F | Cl | F | F | CH₂CH₂CH₃ |
| I.b.384 | F | Cl | CH₃ | H | CH₂CH₂CH₃ |
| I.b.385 | F | Cl | CH₂CH₃ | H | CH₂CH₂CH₃ |
| I.b.386 | F | Cl | OCH₃ | H | CH₂CH₂CH₃ |
| I.b.387 | F | Cl | CH₃ | F | CH₂CH₂CH₃ |
| I.b.388 | F | Cl | CH₃ | H | CF₃ |
| I.b.389 | F | Cl | CH₃ | F | CF₃ |
| I.b.390 | F | Cl | H | —(CH₂)₂—CH(CH₃)—(CH₂)₂— | |
| I.b.391 | F | Cl | H | —O—(CH₂)₂— | |
| I.b.392 | F | Cl | H | —O—(CH₂)₃— | |
| I.b.393 | F | Cl | H | —O—(CH₂)₄— | |
| I.b.394 | F | Cl | CH₃ | —(CH₂)₂—CH(CH₃)—(CH₂)₂— | |
| I.b.395 | F | Cl | CH₃ | —O—(CH₂)₂— | |
| I.b.396 | F | Cl | CH₃ | —O—(CH₂)₃— | |
| I.b.397 | F | Cl | CH₃ | —O—(CH₂)₄— | |
| I.b.398 | F | Cl | F | —(CH₂)₂—CH(CH₃)—(CH₂)₂— | |
| I.b.399 | F | Cl | F | —O—(CH₂)₂— | |
| I.b.400 | F | Cl | F | —O—(CH₂)₃— | |
| I.b.401 | F | Cl | F | —O—(CH₂)₄— | |
| I.b.402 | F | Cl | Cl | —(CH₂)₂—CH(CH₃)—(CH₂)₂— | |
| I.b.403 | F | Cl | Cl | —O—(CH₂)₂— | |
| I.b.404 | F | Cl | Cl | —O—(CH₂)₃— | |
| I.b.405 | F | Cl | Cl | —O—(CH₂)₄— | |
| I.b.406 | F | Cl | CH₃ | —O—(CH₂)₃— | |
| I.b.407 | F | Cl | CH₃ | —O—(CH₂)₄— | |
| I.b.408 | F | Cl | OCH₃ | —O—(CH₂)₃— | |
| I.b.409 | F | Cl | OCH₃ | —O—(CH₂)₄— | |
| I.b.410 | F | Cl | OCF₃ | —O—(CH₂)₃— | |
| I.b.411 | F | Cl | OCF₃ | —O—(CH₂)₄— | |
| I.b.412 | F | Cl | OCH₃ | —(CH₂)₂— | |
| I.b.413 | F | Cl | OCH₃ | —(CH₂)₃— | |
| I.b.414 | F | Cl | OCH₃ | —(CH₂)₄— | |
| I.b.415 | F | Cl | OCH₃ | —(CH₂)₅— | |
| I.b.416 | F | Cl | H | H | OH |
| I.b.417 | F | Cl | H | H | OCH₃ |
| I.b.418 | F | Cl | H | H | OCF₃ |
| I.b.419 | F | Cl | CH₃ | H | OH |
| I.b.420 | F | Cl | CH₃ | H | OCH₃ |
| I.b.421 | F | Cl | CH₃ | H | OCF₃ |
| I.b.422 | F | Cl | CH₃ | CH₃ | OH |
| I.b.423 | F | Cl | CH₃ | CH₃ | OCH₃ |
| I.b.424 | F | Cl | CH₃ | CH₃ | OCF₃ |
| I.b.425 | F | Cl | OH | —(CH₂)₂— | |
| I.b.426 | F | Cl | OH | —(CH₂)₃— | |
| I.b.427 | F | Cl | OH | —(CH₂)₄— | |
| I.b.428 | F | Cl | OH | —(CH₂)₅— | |
| I.b.429 | F | Cl | OCF₃ | —(CH₂)₂— | |
| I.b.430 | F | Cl | OCF₃ | —(CH₂)₃— | |
| I.b.431 | F | Cl | OCF₃ | —(CH₂)₄— | |
| I.b.432 | F | Cl | OCF₃ | —(CH₂)₅— | |
| I.b.433 | Cl | F | F | H | C₂H₅ |
| I.b.434 | Cl | F | OCH₃ | H | C₂H₅ |
| I.b.435 | Cl | F | H | H | C(CH₃)₃ |
| I.b.436 | Cl | F | F | H | C(CH₃)₃ |
| I.b.437 | Cl | F | F | F | C(CH₃)₃ |
| I.b.438 | Cl | F | CH₃ | H | C(CH₃)₃ |
| I.b.439 | Cl | F | OCH₃ | H | C(CH₃)₃ |
| I.b.440 | Cl | F | CH₃ | F | C(CH₃)₃ |
| I.b.441 | Cl | F | H | H | CH(CH₃)₂ |
| I.b.442 | Cl | F | F | H | CH(CH₃)₂ |
| I.b.443 | Cl | F | F | F | CH(CH₃)₂ |
| I.b.444 | Cl | F | CH₃ | H | CH(CH₃)₂ |
| I.b.445 | Cl | F | OCH₃ | H | CH(CH₃)₂ |
| I.b.446 | Cl | F | CH₃ | F | CH(CH₃)₂ |
| I.b.447 | Cl | F | H | H | c-C₃H₅ |
| I.b.448 | Cl | F | F | H | c-C₃H₅ |
| I.b.449 | Cl | F | F | F | c-C₃H₅ |
| I.b.450 | Cl | F | CH₃ | H | c-C₃H₅ |
| I.b.451 | Cl | F | OCH₃ | H | c-C₃H₅ |
| I.b.452 | Cl | F | CH₃ | F | c-C₃H₅ |
| I.b.453 | Cl | F | H | H | CH₂CH₂CH₃ |
| I.b.454 | Cl | F | F | H | CH₂CH₂CH₃ |
| I.b.455 | Cl | F | F | F | CH₂CH₂CH₃ |
| I.b.456 | Cl | F | CH₃ | H | CH₂CH₂CH₃ |
| I.b.457 | Cl | F | CH₂CH₃ | H | CH₂CH₂CH₃ |
| I.b.458 | Cl | F | OCH₃ | H | CH₂CH₂CH₃ |
| I.b.459 | Cl | F | CH₃ | F | CH₂CH₂CH₃ |
| I.b.460 | Cl | F | CH₃ | H | CF₃ |
| I.b.461 | Cl | F | CH₃ | F | CF₃ |
| I.b.462 | Cl | F | H | —(CH₂)₂—CH(CH₃)—(CH₂)₂— | |
| I.b.463 | Cl | F | H | —O—(CH₂)₂— | |
| I.b.464 | Cl | F | H | —O—(CH₂)₃— | |
| I.b.465 | Cl | F | H | —O—(CH₂)₄— | |
| I.b.466 | Cl | F | CH₃ | —(CH₂)₂—CH(CH₃)—(CH₂)₂— | |
| I.b.467 | Cl | F | CH₃ | —O—(CH₂)₂— | |
| I.b.468 | Cl | F | CH₃ | —O—(CH₂)₃— | |
| I.b.469 | Cl | F | CH₃ | —O—(CH₂)₄— | |
| I.b.470 | Cl | F | F | —(CH₂)₂—CH(CH₃)—(CH₂)₂— | |
| I.b.471 | Cl | F | F | —O—(CH₂)₂— | |
| I.b.472 | Cl | F | F | —O—(CH₂)₃— | |
| I.b.473 | Cl | F | F | —O—(CH₂)₄— | |
| I.b.474 | Cl | F | Cl | —(CH₂)₂—CH(CH₃)—(CH₂)₂— | |
| I.b.475 | Cl | F | Cl | —O—(CH₂)₂— | |
| I.b.476 | Cl | F | Cl | —O—(CH₂)₃— | |
| I.b.477 | Cl | F | Cl | —O—(CH₂)₄— | |
| I.b.478 | Cl | F | CH₃ | —O—(CH₂)₃— | |
| I.b.479 | Cl | F | CH₃ | —O—(CH₂)₄— | |
| I.b.480 | Cl | F | OCH₃ | —O—(CH₂)₃— | |
| I.b.481 | Cl | F | OCH₃ | —O—(CH₂)₄— | |
| I.b.482 | Cl | F | OCF₃ | —O—(CH₂)₃— | |
| I.b.483 | Cl | F | OCF₃ | —O—(CH₂)₄— | |
| I.b.484 | Cl | F | OCH₃ | —(CH₂)₂— | |
| I.b.485 | Cl | F | OCH₃ | —(CH₂)₃— | |
| I.b.486 | Cl | F | OCH₃ | —(CH₂)₄— | |
| I.b.487 | Cl | F | OCH₃ | —(CH₂)₅— | |
| I.b.488 | Cl | F | H | H | OH |
| I.b.489 | Cl | F | H | H | OCH₃ |
| I.b.490 | Cl | F | H | H | OCF₃ |
| I.b.491 | Cl | F | CH₃ | H | OH |
| I.b.492 | Cl | F | CH₃ | H | OCH₃ |
| I.b.493 | Cl | F | CH₃ | H | OCF₃ |
| I.b.494 | Cl | F | CH₃ | CH₃ | OH |
| I.b.495 | Cl | F | CH₃ | CH₃ | OCH₃ |
| I.b.496 | Cl | F | CH₃ | CH₃ | OCF₃ |
| I.b.497 | Cl | F | OH | —(CH₂)₂— | |
| I.b.498 | Cl | F | OH | —(CH₂)₃— | |
| I.b.499 | Cl | F | OH | —(CH₂)₄— | |
| I.b.500 | Cl | F | OH | —(CH₂)₅— | |
| I.b.501 | Cl | F | OCF₃ | —(CH₂)₂— | |
| I.b.502 | Cl | F | OCF₃ | —(CH₂)₃— | |
| I.b.503 | Cl | F | OCF₃ | —(CH₂)₄— | |
| I.b.504 | Cl | F | OCF₃ | —(CH₂)₅— | |
| I.b.505 | Cl | H | F | H | C₂H₅ |
| I.b.506 | Cl | H | OCH₃ | H | C₂H₅ |
| I.b.507 | Cl | H | H | H | C(CH₃)₃ |
| I.b.508 | Cl | H | F | H | C(CH₃)₃ |
| I.b.509 | Cl | H | F | F | C(CH₃)₃ |
| I.b.510 | Cl | H | CH₃ | H | C(CH₃)₃ |
| I.b.511 | Cl | H | OCH₃ | H | C(CH₃)₃ |
| I.b.512 | Cl | H | CH₃ | F | C(CH₃)₃ |
| I.b.513 | Cl | H | H | H | CH(CH₃)₂ |
| I.b.514 | Cl | H | F | H | CH(CH₃)₂ |
| I.b.515 | Cl | H | F | F | CH(CH₃)₂ |
| I.b.516 | Cl | H | CH₃ | H | CH(CH₃)₂ |
| I.b.517 | Cl | H | OCH₃ | H | CH(CH₃)₂ |
| I.b.518 | Cl | H | CH₃ | F | CH(CH₃)₂ |
| I.b.519 | Cl | H | H | H | c-C₃H₅ |
| I.b.520 | Cl | H | F | H | c-C₃H₅ |
| I.b.521 | Cl | H | F | F | c-C₃H₅ |
| I.b.522 | Cl | H | CH₃ | H | c-C₃H₅ |
| I.b.523 | Cl | H | OCH₃ | H | c-C₃H₅ |
| I.b.524 | Cl | H | CH₃ | F | c-C₃H₅ |
| I.b.525 | Cl | H | H | H | CH₂CH₂CH₃ |
| I.b.526 | Cl | H | F | H | CH₂CH₂CH₃ |
| I.b.527 | Cl | H | F | F | CH₂CH₂CH₃ |
| I.b.528 | Cl | H | CH₃ | H | CH₂CH₂CH₃ |
| I.b.529 | Cl | H | CH₂CH₃ | H | CH₂CH₂CH₃ |
| I.b.530 | Cl | H | OCH₃ | H | CH₂CH₂CH₃ |
| I.b.531 | Cl | H | CH₃ | F | CH₂CH₂CH₃ |
| I.b.532 | Cl | H | CH₃ | H | CF₃ |
| I.b.533 | Cl | H | CH₃ | F | CF₃ |
| I.b.534 | Cl | H | H | —(CH₂)₂—CH(CH₃)—(CH₂)₂— | |
| I.b.535 | Cl | H | H | —O—(CH₂)₂— | |
| I.b.536 | Cl | H | H | —O—(CH₂)₃— | |
| I.b.537 | Cl | H | H | —O—(CH₂)₄— | |
| I.b.538 | Cl | H | CH₃ | —(CH₂)₂—CH(CH₃)—(CH₂)₂— | |
| I.b.539 | Cl | H | CH₃ | —O—(CH₂)₂— | |
| I.b.540 | Cl | H | CH₃ | —O—(CH₂)₃— | |
| I.b.541 | Cl | H | CH₃ | —O—(CH₂)₄— | |
| I.b.542 | Cl | H | F | —(CH₂)₂—CH(CH₃)—(CH₂)₂— | |
| I.b.543 | Cl | H | F | —O—(CH₂)₂— | |
| I.b.544 | Cl | H | F | —O—(CH₂)₃— | |
| I.b.545 | Cl | H | F | —O—(CH₂)₄— | |
| I.b.546 | Cl | H | Cl | —(CH₂)₂—CH(CH₃)—(CH₂)₂— | |
| I.b.547 | Cl | H | Cl | —O—(CH₂)₂— | |
| I.b.548 | Cl | H | Cl | —O—(CH₂)₃— | |
| I.b.549 | Cl | H | Cl | —O—(CH₂)₄— | |
| I.b.550 | Cl | H | CH₃ | —O—(CH₂)₃— | |
| I.b.551 | Cl | H | CH₃ | —O—(CH₂)₄— | |
| I.b.552 | Cl | H | OCH₃ | —O—(CH₂)₃— | |
| I.b.553 | Cl | H | OCH₃ | —O—(CH₂)₄— | |
| I.b.554 | Cl | H | OCF₃ | —O—(CH₂)₃— | |
| I.b.555 | Cl | H | OCF₃ | —O—(CH₂)₄— | |
| I.b.556 | Cl | H | OCH₃ | —(CH₂)₂— | |
| I.b.557 | Cl | H | OCH₃ | —(CH₂)₃— | |
| I.b.558 | Cl | H | OCH₃ | —(CH₂)₄— | |
| I.b.559 | Cl | H | OCH₃ | —(CH₂)₅— | |
| I.b.560 | Cl | H | H | H | OH |
| I.b.561 | Cl | H | H | H | OCH₃ |
| I.b.562 | Cl | H | H | H | OCF₃ |
| I.b.563 | Cl | H | CH₃ | H | OH |
| I.b.564 | Cl | H | CH₃ | H | OCH₃ |
| I.b.565 | Cl | H | CH₃ | H | OCF₃ |
| I.b.566 | Cl | H | CH₃ | CH₃ | OH |
| I.b.567 | Cl | H | CH₃ | CH₃ | OCH₃ |
| I.b.568 | Cl | H | CH₃ | CH₃ | OCF₃ |
| I.b.569 | Cl | H | OH | —(CH₂)₂— | |
| I.b.570 | Cl | H | OH | —(CH₂)₃— | |
| I.b.571 | Cl | H | OH | —(CH₂)₄— | |
| I.b.572 | Cl | H | OH | —(CH₂)₅— | |
| I.b.573 | Cl | H | OCF₃ | —(CH₂)₂— | |
| I.b.574 | Cl | H | OCF₃ | —(CH₂)₃— | |
| I.b.575 | Cl | H | OCF₃ | —(CH₂)₄— | |
| I.b.576 | Cl | H | OCF₃ | —(CH₂)₅— | |
| I.b.577 | CN | F | F | H | C₂H₅ |
| I.b.578 | CN | F | OCH₃ | H | C₂H₅ |
| I.b.579 | CN | F | H | H | C(CH₃)₃ |
| I.b.580 | CN | F | F | H | C(CH₃)₃ |
| I.b.581 | CN | F | F | F | C(CH₃)₃ |
| I.b.582 | CN | F | CH₃ | H | C(CH₃)₃ |
| I.b.583 | CN | F | OCH₃ | H | C(CH₃)₃ |
| I.b.584 | CN | F | CH₃ | F | C(CH₃)₃ |
| I.b.585 | CN | F | H | H | CH(CH₃)₂ |
| I.b.586 | CN | F | F | H | CH(CH₃)₂ |
| I.b.587 | CN | F | F | F | CH(CH₃)₂ |
| I.b.588 | CN | F | CH₃ | H | CH(CH₃)₂ |
| I.b.589 | CN | F | OCH₃ | H | CH(CH₃)₂ |
| I.b.590 | CN | F | CH₃ | F | CH(CH₃)₂ |
| I.b.591 | CN | F | H | H | c-C₃H₅ |
| I.b.592 | CN | F | F | H | c-C₃H₅ |
| I.b.593 | CN | F | F | F | c-C₃H₅ |
| I.b.594 | CN | F | CH₃ | H | c-C₃H₅ |
| I.b.595 | CN | F | OCH₃ | H | c-C₃H₅ |
| I.b.596 | CN | F | CH₃ | F | c-C₃H₅ |
| I.b.597 | CN | F | H | H | CH₂CH₂CH₃ |
| I.b.598 | CN | F | F | H | CH₂CH₂CH₃ |
| I.b.599 | CN | F | F | F | CH₂CH₂CH₃ |
| I.b.600 | CN | F | CH₃ | H | CH₂CH₂CH₃ |
| I.b.601 | CN | F | CH₂CH₃ | H | CH₂CH₂CH₃ |
| I.b.602 | CN | F | OCH₃ | H | CH₂CH₂CH₃ |
| I.b.603 | CN | F | CH₃ | F | CH₂CH₂CH₃ |
| I.b.604 | CN | F | CH₃ | H | CF₃ |
| I.b.605 | CN | F | CH₃ | F | CF₃ |
| I.b.606 | CN | F | H | —(CH₂)₂—CH(CH₃)—(CH₂)₂— | |
| I.b.607 | CN | F | H | —O—(CH₂)₂— | |
| I.b.608 | CN | F | H | —O—(CH₂)₃— | |
| I.b.609 | CN | F | H | —O—(CH₂)₄— | |
| I.b.610 | CN | F | CH₃ | —(CH₂)₂—CH(CH₃)—(CH₂)₂— | |
| I.b.611 | CN | F | CH₃ | —O—(CH₂)₂— | |
| I.b.612 | CN | F | CH₃ | —O—(CH₂)₃— | |
| I.b.613 | CN | F | CH₃ | —O—(CH₂)₄— | |
| I.b.614 | CN | F | F | —(CH₂)₂—CH(CH₃)—(CH₂)₂— | |
| I.b.615 | CN | F | F | —O—(CH₂)₂— | |
| I.b.616 | CN | F | F | —O—(CH₂)₃— | |
| I.b.617 | CN | F | F | —O—(CH₂)₄— | |
| I.b.618 | CN | F | Cl | —(CH₂)₂—CH(CH₃)—(CH₂)₂— | |
| I.b.619 | CN | F | Cl | —O—(CH₂)₂— | |
| I.b.620 | CN | F | Cl | —O—(CH₂)₃— | |
| I.b.621 | CN | F | Cl | —O—(CH₂)₄— | |
| I.b.622 | CN | F | CH₃ | —O—(CH₂)₃— | |
| I.b.623 | CN | F | CH₃ | —O—(CH₂)₄— | |
| I.b.624 | CN | F | OCH₃ | —O—(CH₂)₃— | |
| I.b.625 | CN | F | OCH₃ | —O—(CH₂)₄— | |
| I.b.626 | CN | F | OCF₃ | —O—(CH₂)₃— | |
| I.b.627 | CN | F | OCF₃ | —O—(CH₂)₄— | |
| I.b.628 | CN | F | OCH₃ | —(CH₂)₂— | |
| I.b.629 | CN | F | OCH₃ | —(CH₂)₃— | |
| I.b.630 | CN | F | OCH₃ | —(CH₂)₄— | |
| I.b.631 | CN | F | OCH₃ | —(CH₂)₅— | |
| I.b.632 | CN | H | F | H | C₂H₅ |
| I.b.633 | CN | H | OCH₃ | H | C₂H₅ |
| I.b.634 | CN | H | H | H | C(CH₃)₃ |
| I.b.635 | CN | H | F | H | C(CH₃)₃ |
| I.b.636 | CN | H | F | F | C(CH₃)₃ |
| I.b.637 | CN | H | CH₃ | H | C(CH₃)₃ |
| I.b.638 | CN | H | OCH₃ | H | C(CH₃)₃ |
| I.b.639 | CN | H | CH₃ | F | C(CH₃)₃ |
| I.b.640 | CN | H | H | H | CH(CH₃)₂ |
| I.b.641 | CN | H | F | H | CH(CH₃)₂ |
| I.b.642 | CN | H | F | F | CH(CH₃)₂ |
| I.b.643 | CN | H | CH₃ | H | CH(CH₃)₂ |
| I.b.644 | CN | H | OCH₃ | H | CH(CH₃)₂ |
| I.b.645 | CN | H | CH₃ | F | CH(CH₃)₂ |
| I.b.646 | CN | H | H | H | c-C₃H₅ |
| I.b.647 | CN | H | F | H | c-C₃H₅ |
| I.b.648 | CN | H | F | F | c-C₃H₅ |
| I.b.649 | CN | H | CH₃ | H | c-C₃H₅ |
| I.b.650 | CN | H | OCH₃ | H | c-C₃H₅ |
| I.b.651 | CN | H | CH₃ | F | c-C₃H₅ |
| I.b.652 | CN | H | H | H | CH₂CH₂CH₃ |
| I.b.653 | CN | H | F | H | CH₂CH₂CH₃ |
| I.b.654 | CN | H | F | F | CH₂CH₂CH₃ |
| I.b.655 | CN | H | CH₃ | H | CH₂CH₂CH₃ |
| I.b.656 | CN | H | CH₂CH₃ | H | CH₂CH₂CH₃ |
| I.b.657 | CN | H | OCH₃ | H | CH₂CH₂CH₃ |
| I.b.658 | CN | H | CH₃ | F | CH₂CH₂CH₃ |
| I.b.659 | CN | H | CH₃ | H | CF₃ |
| I.b.660 | CN | H | CH₃ | F | CF₃ |
| I.b.661 | CN | H | H | —(CH₂)₂—CH(CH₃)—(CH₂)₂— | |
| I.b.662 | CN | H | H | —O—(CH₂)₂— | |
| I.b.663 | CN | H | H | —O—(CH₂)₃— | |
| I.b.664 | CN | H | H | —O—(CH₂)₄— | |
| I.b.665 | CN | H | CH₃ | —(CH₂)₂—CH(CH₃)—(CH₂)₂— | |
| I.b.666 | CN | H | CH₃ | —O—(CH₂)₂— | |
| I.b.667 | CN | H | CH₃ | —O—(CH₂)₃— | |
| I.b.668 | CN | H | CH₃ | —O—(CH₂)₄— | |
| I.b.669 | CN | H | F | —(CH₂)₂—CH(CH₃)—(CH₂)₂— | |
| I.b.670 | CN | H | F | —O—(CH₂)₂— | |
| I.b.671 | CN | H | F | —O—(CH₂)₃— | |
| I.b.672 | CN | H | F | —O—(CH₂)₄— | |
| I.b.673 | CN | H | Cl | —(CH₂)₂—CH(CH₃)—(CH₂)₂— | |
| I.b.674 | CN | H | Cl | —O—(CH₂)₂— | |
| I.b.675 | CN | H | Cl | —O—(CH₂)₃— | |
| I.b.676 | CN | H | Cl | —O—(CH₂)₄— | |
| I.b.677 | CN | H | CH₃ | —O—(CH₂)₃— | |
| I.b.678 | CN | H | CH₃ | —O—(CH₂)₄— | |
| I.b.679 | CN | H | OCH₃ | —O—(CH₂)₃— | |
| I.b.680 | CN | H | OCH₃ | —O—(CH₂)₄— | |
| I.b.681 | CN | H | OCF₃ | —O—(CH₂)₃— | |
| I.b.682 | CN | H | OCF₃ | —O—(CH₂)₄— | |
| I.b.683 | CN | H | OCH₃ | —(CH₂)₂— | |
| I.b.684 | CN | H | OCH₃ | —(CH₂)₃— | |
| I.b.685 | CN | H | OCH₃ | —(CH₂)₄— | |
| I.b.686 | CN | H | OCH₃ | —(CH₂)₅— | |
| I.b.687 | F | CN | F | H | C₂H₅ |
| I.b.688 | F | CN | OCH₃ | H | C₂H₅ |
| I.b.689 | F | CN | H | H | C(CH₃)₃ |
| I.b.690 | F | CN | F | H | C(CH₃)₃ |
| I.b.691 | F | CN | F | F | C(CH₃)₃ |
| I.b.692 | F | CN | CH₃ | H | C(CH₃)₃ |
| I.b.693 | F | CN | OCH₃ | H | C(CH₃)₃ |
| I.b.694 | F | CN | CH₃ | F | C(CH₃)₃ |
| I.b.695 | F | CN | H | H | CH(CH₃)₂ |
| I.b.696 | F | CN | F | H | CH(CH₃)₂ |
| I.b.697 | F | CN | F | F | CH(CH₃)₂ |
| I.b.698 | F | CN | CH₃ | H | CH(CH₃)₂ |
| I.b.699 | F | CN | OCH₃ | H | CH(CH₃)₂ |
| I.b.700 | F | CN | CH₃ | F | CH(CH₃)₂ |
| I.b.701 | F | CN | H | H | c-C₃H₅ |
| I.b.702 | F | CN | F | H | c-C₃H₅ |
| I.b.703 | F | CN | F | F | c-C₃H₅ |
| I.b.704 | F | CN | CH₃ | H | c-C₃H₅ |
| I.b.705 | F | CN | OCH₃ | H | c-C₃H₅ |
| I.b.706 | F | CN | CH₃ | F | c-C₃H₅ |
| I.b.707 | F | CN | H | H | CH₂CH₂CH₃ |
| I.b.708 | F | CN | F | H | CH₂CH₂CH₃ |
| I.b.709 | F | CN | F | F | CH₂CH₂CH₃ |
| I.b.710 | F | CN | CH₃ | H | CH₂CH₂CH₃ |
| I.b.711 | F | CN | CH₂CH₃ | H | CH₂CH₂CH₃ |
| I.b.712 | F | CN | OCH₃ | H | CH₂CH₂CH₃ |
| I.b.713 | F | CN | CH₃ | F | CH₂CH₂CH₃ |
| I.b.714 | F | CN | CH₃ | H | CF₃ |
| I.b.715 | F | CN | CH₃ | F | CF₃ |

| | | | | |
|---|---|---|---|---|
| I.b.716 | F | CN | H | —(CH₂)₂—CH(CH₃)—(CH₂)₂— |
| I.b.717 | F | CN | H | —O—(CH₂)₂— |
| I.b.718 | F | CN | H | —O—(CH₂)₃— |
| I.b.719 | F | CN | H | —O—(CH₂)₄— |
| I.b.720 | F | CN | CH₃ | —(CH₂)₂—CH(CH₃)—(CH₂)₂— |
| I.b.721 | F | CN | CH₃ | —O—(CH₂)₂— |
| I.b.722 | F | CN | CH₃ | —O—(CH₂)₃— |
| I.b.723 | F | CN | CH₃ | —O—(CH₂)₄— |
| I.b.724 | F | CN | F | —(CH₂)₂—CH(CH₃)—(CH₂)₂— |
| I.b.725 | F | CN | F | —O—(CH₂)₂— |
| I.b.726 | F | CN | F | —O—(CH₂)₃— |
| I.b.727 | F | CN | F | —O—(CH₂)₄— |
| I.b.728 | F | CN | Cl | —(CH₂)₂—CH(CH₃)—(CH₂)₂— |
| I.b.729 | F | CN | Cl | —O—(CH₂)₂— |
| I.b.730 | F | CN | Cl | —O—(CH₂)₃— |
| I.b.731 | F | CN | Cl | —O—(CH₂)₄— |
| I.b.732 | F | CN | CH₃ | —O—(CH₂)₃— |
| I.b.733 | F | CN | CH₃ | —O—(CH₂)₄— |
| I.b.734 | F | CN | OCH₃ | —O—(CH₂)₃— |
| I.b.735 | F | CN | OCH₃ | —O—(CH₂)₄— |
| I.b.736 | F | CN | OCF₃ | —O—(CH₂)₃— |
| I.b.737 | F | CN | OCF₃ | —O—(CH₂)₄— |
| I.b.738 | F | CN | OCH₃ | —(CH₂)₂— |
| I.b.739 | F | CN | OCH₃ | —(CH₂)₃— |
| I.b.740 | F | CN | OCH₃ | —(CH₂)₄— |
| I.b.741 | F | CN | OCH₃ | —(CH₂)₅— |

Also preferred are the azines of formula (I.c), particularly preferred the azines of formulae (I.c.1) to (I.c.741) which differ from the corresponding azines of formulae (I.b.1) to (I.b.741) only in that R^{A1} is Cl:

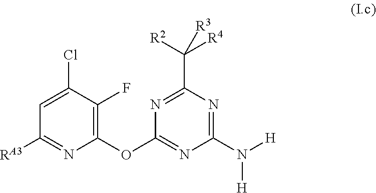

Also preferred are the azines of formula (I.d), particularly preferred the azines of formulae (I.d.1) to (I.d.741) which differ from the corresponding azines of formulae (I.b.1) to (I.b.741) only in that R^{A1} is Br:

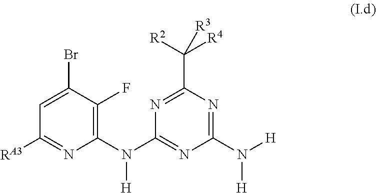

Also preferred are the azines of formula (I.e), particularly preferred the azines of formulae (I.e.1) to (I.e.741) which differ from the corresponding azines of formulae (I.b.1) to (I.b.741) only in that, R^{A1} and R^{A2} are F:

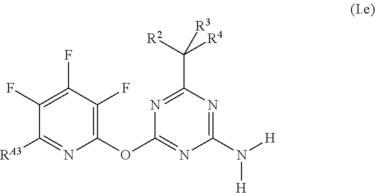

Also preferred are the azines of formula (I.f), particularly preferred the azines of formulae (I.f.1) to (I.f.741) which differ from the corresponding azines of formulae (I.b.1) to (I.b.741) only in that R^{A1} is F and R^{A2} is Cl:

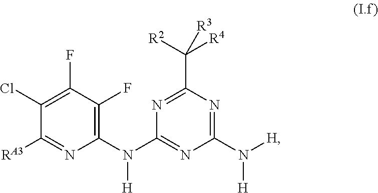

Also preferred are the azines of formula (I.g), particularly preferred the azines of formulae (I.g.1) to (I.g.741) which differ from the corresponding azines of formulae (I.b.1) to (I.b.741) only in that R^{A1} is F and R^{A2} is Br:

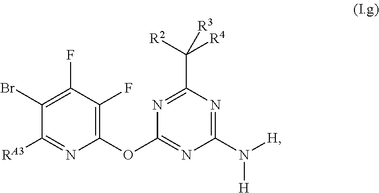

Also preferred are the azines of formula (I.h), particularly preferred the azines of formulae (I.h.1) to (I.h.741) which differ from the corresponding azines of formulae (I.b.1) to (I.b.741) only in that R^{A1} is Cl and R^{A2} is F:

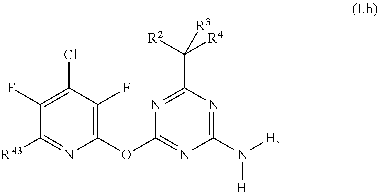

Also preferred are the azines of formula (I.i), particularly preferred the azines of formulae (I.i.1) to (I.i.741) which differ from the corresponding azines of formulae (I.b.1) to (I.b.741) only in that R^{A1} is Br and R^{A2} is F:

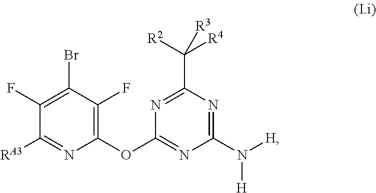

Also preferred are the azines of formula (I.j), particularly preferred the azines of formulae (I.j.1) to (I.j.741) which differ from the corresponding azines of formulae (I.b.1) to (I.b.741) only in that R^{A1} and R^{A2} is Cl:

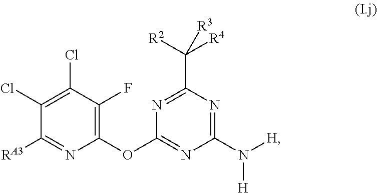

A special group of embodiments relates to compounds of the formula (I′)

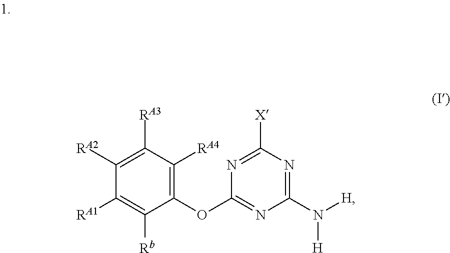

and to their agriculturally acceptable salts, where X′ corresponds to the group CR²R³R⁴, R^{b} and R^{A4} are as defined herein and R^{A1}, R^{A2}, R^{A3} are hydrogen or have one of the meanings given for R^{A}. Particular examples of these groups of embodiments are the compounds of formula (I′), which are selected from the group consisting of compounds E.1 to E.45 and their agriculturally acceptable salts, where R^{A1}, R^{A2}, R^{A3}, R^{A4} R^{b} and X′ are as defined in the following table:

| Com- | | | | | | |
|---|---|---|---|---|---|---|
| pound | R^{b} | R^{A4} | R^{A3} | R^{A2} | R^{A1} | X′ |
| E.1 | F | F | F | F | F | CHFCH₃ |
| E.2 | F | F | F | F | F | CF(CH₃)₂ |
| E.3 | F | H | H | F | H | CHFCH₃ |
| E.4 | CN | H | H | H | H | CHFCH₃ |
| E.5 | F | F | H | H | F | CF(CH₃)₂ |
| E.6 | F | H | H | H | F | CF(CH₃)₂ |
| E.7 | F | H | H | H | F | CHFCH₃ |
| E.8 | F | F | H | H | F | CHFCH₃ |
| E.9 | F | F | H | F | F | CHFCH₃ |
| E.10 | F | F | H | F | F | CF₂CH₃ |
| E.11 | F | F | H | F | F | CF₃ |
| E.12 | F | F | H | F | F | C(CH₃)₃ |
| E.13 | F | F | H | F | F | 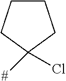 |
| E.14 | F | F | H | H | F | C(CH₃)₃ |
| E.15 | F | F | F | F | F | 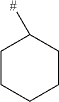 |
| E.16 | F | H | H | H | F | 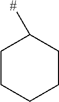 |
| E.17 | F | F | F | F | F | 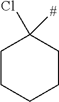 |
| E.18 | F | F | H | H | F | CF₂CH₃ |
| E.19 | F | F | H | F | F | 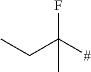 |
| E.20 | F | F | H | H | F | 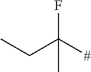 |
| E.21 | F | F | H | F | F | 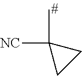 |
| E.22 | F | F | F | F | F | C(CH₃)₂CN |
| E.23 | F | F | H | F | F | 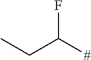 |
| E.24 | F | F | F | F | F | 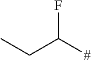 |
| E.25 | F | F | H | F | F | 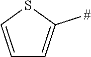 |
| E.26 | F | F | H | F | F | 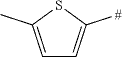 |
| E.27 | F | F | H | F | F | 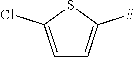 |
| E.28 | F | F | H | F | F | 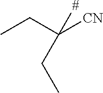 |
| E.29 | F | F | H | F | F | 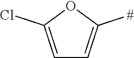 |
| E.30 | F | F | H | F | F | 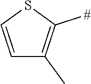 |
| E.31 | F | F | H | F | F | 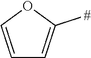 |
| E.32 | F | F | H | F | F | 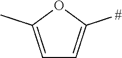 |
| E.33 | F | F | H | F | F | 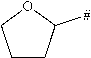 |
| E.34 | F | F | H | F | F | 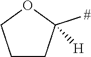 |
| E.35 | F | F | H | F | F | 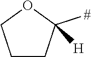 |
| E.36 | F | F | H | F | F | 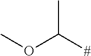 |
| E.37 | F | Cl | H | H | F | CHFCH₃ |
| E.38 | F | F | H | F | F | 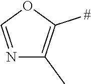 |
| E.39 | F | F | I | F | F | CHFCH₃ |
| E.40 | F | F | Br | F | F | CHFCH₃ |
| E.41 | F | F | Cl | F | F | CHFCH₃ |
| E.42 | F | F | H | F | F | 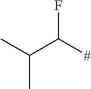 |
| E.43 | F | F | H | F | F | CH₂CH₃ |
| E.44 | F | F | H | F | F | CH(CH₃)₂ |
| E.45 | F | F | H | F | F | 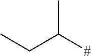 |

The aminotriazine compounds of formula (I) according to the invention can be prepared by standard processes of organic chemistry, for example by the following processes:

### Process A)

The aminotriazine compounds of formula (I), wherein R¹ is as defined above and in particular H, C₁-C₆-alkyl or (C₁-C₆-alkoxy)-C₁-C₆-alkyl, can be prepared by reacting halotriazines of formula (II) with alkohols in the presence of a base or the corresponding alkoholates of formula (III) optionally in the presence of a catalyst as depicted in the following scheme 1:

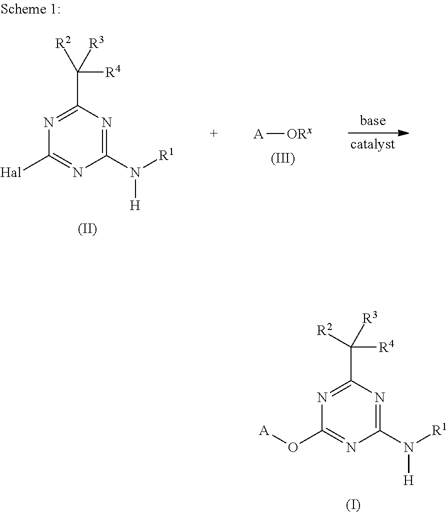

In scheme 1, the variables R¹, R², R³, R⁴ and A have the above meanings, in particular the preferred meanings, as in formula (I) mentioned above; while Hal is halogen, in particular bromine or chlorine and especially chlorine,
R¹ is in particular H, C₁-C₆-alkyl, (C₁-C₆-alkoxy)-C₁-C₆-alkyl;
   more particularly H, C₁-C₄-alkoxy-C₁-C₄-alkyl, such as CH₂OCH₃;
   especially hydrogen;
R^{x} is particularly H, Na, K, Li, NH₄ or positively charged ion equivalent;
   especially hydrogen.

The reaction of the halotriazines of formula (II) with the alkohols in the presence of a base or the corresponding alkoholates of formula (III) is usually carried out at temperatures in the range from 15° C. to the boiling point of the reaction mixture, preferably from 20° C. to 150° C., particularly preferably from 20° C. to 80° C., in an inert organic solvent (e.g.—R. J. Goetz et al. Chem. Comm. 2008, 3384-3386).

The reaction can be carried out at atmospheric pressure or under elevated pressure, if appropriate, under an inert gas, continuously or batchwise.

In one embodiment of the process according to the invention, the halotriazines of formula (II) and the alkohols in the presence of a base or the corresponding alkoholates of formula (III) are used in equimolar amounts.

In another embodiment of the process according to the invention, the alkohols in the presence of a base or the corresponding alkoholates of formula (III) are used in excess with regard to the halotriazines of formula (II).

Preferably the molar ratio of the alkohols in the presence of a base or the corresponding alkoholates of formula (III) to the halotriazines of formula (II) is in the range from 2:1 to 1:1, preferably 1.5:1 to 1:1, especially preferred 1.2:1.

The reaction of the halotriazines of formula (II) with the alkohols in the presence of a base or the corresponding alkoholates of formula (III) is usually carried out in an organic solvent. Suitable in principle are all solvents which are capable of dissolving the halotriazines of formula (II) and the alkohols in the presence of a base or the corresponding alkoholates of formula (III) at least partly and preferably fully under reaction conditions. Examples of suitable solvents are aliphatic hydrocarbons such as pentane, hexane, cyclohexane, nitromethane and mixtures of C₅-C₈-alkanes, aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP). Preferred solvents are ethers as defined above. The term solvent as used herein also includes mixtures of two or more of the above compounds.

The reaction of the halotriazines of formula (II) with the alkohols in the presence of a base or the corresponding alkoholates of formula (III) is usually carried out in the presence of a base. Examples of suitable bases include metal-containing bases and nitrogen-containing bases. Examples of suitable metal-containing bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide; alkali metal and alkaline earth metal oxide, and other metal oxides, such as lithium oxide, sodium oxide, potassium oxide, magnesium oxide, calcium oxide and magnesium oxide, iron oxide, silver oxide; alkali metal and alkaline earth metal hydrides such as lithium hydride, sodium hydride, potassium hydride and calcium hydride, alkali metal and alkaline earth metal formates, acetates and other metal salts of carboxylic acids, such as sodium formate, sodium benzoate, lithium acetate, sodium acetate, potassium acetate, magnesium acetate, and calcium acetate; alkali metal and alkaline earth metal carbonates such as lithium carbonate, sodium carbonate, potassium carbonate, magnesium carbonate, and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as sodium phosphate, potassium phosphate and calcium phosphate; alkali metal and alkaline earth metal alkoxides such as sodium methoxide, sodium ethoxide, potassium ethoxide, potassium tert-butoxide, potassium tert-pentoxide and dimethoxymagnesium; and furthermore organic bases, such as tertiary amines such as tri-C₁-C₆-alkylamines, for example triethylamine, trimethylamine, N-ethyldiisopropylamine, and N-methylpiperidine, pyridine, substituted pyridines such as collidine, lutidine, N-methylmorpholine and also bicyclic amines such as 1,8-diazabicyclo[5.4.0]undec-7-ene (DBU) or 1,5-diazabicyclo[4.3.0]non-5-ene (DBN). Preferred bases are alkali metal and alkaline earth metal alkoxides as defined above. The term base as used herein also includes mixtures of two or more, preferably two of the above compounds. Particular preference is given to the use of one base. The bases can be used in excess, preferably from 1 to 10, especially preferred from 2 to 4 base equivalents based on the halotriazines of formula (II), and they may also be used as the solvent.

The reaction of the halotriazines of formula (II) with the alkohols in the presence of a base or the corresponding alkoholates of formula (III) may be carried out in the presence of a catalyst. Examples of suitable catalysts include for example, palladium based catalysts like, for example, Palladium(II)acetate, tetrakis(triphenylphosphine)palladium(0), bis(triphenylphosphine)palladium(II)chloride or (1,1,-bis(diphenylphosphino)ferrocene)dichloropalladium(II), and optionally suitable additives such as, for example, phosphines like, for example, P(o-tolyl)₃, triphenylphosphine or BINAP (2,2′-Bis(diphenylphosphino)-1,1′-binaphthyl). The amount of catalyst is usually 10 to 20 mol % (0.1 to 0.2 equivalents) based on the halotriazines of formula (II).

The end of the reaction can easily be determined by the skilled worker by means of routine methods.

The reaction mixtures are worked up in a customary manner, for example by mixing with water, separation of the phases and, if appropriate, chromatographic purification of the crude product.

The alkohols in the presence of a base or the corresponding alkoholates of formula (III) used for the preparation of aminotriazine compounds of formula (I), wherein R¹ is H, C₁-C₆-alkyl, (C₁-C₆-alkoxy)-C₁-C₆-alkyl or C₁-C₆-alkoxy, are commercially available and/or can be prepared by analogy to known literature.

The halotriazines of formula (II) required for the preparation of aminotriazine compounds of formula (I), wherein R¹ is H, C₁-C₆-alkyl, (C₁-C₆-alkoxy)-C₁-C₆-alkyl or C₁-C₆-alkoxy, can be prepared by analogy (e.g. J. K. Chakrabarti et al., Tetrahedron 1975, 31, 1879-1882) by reacting thiotriazines of formula (IV) with a halogen, as depicted in scheme 2:

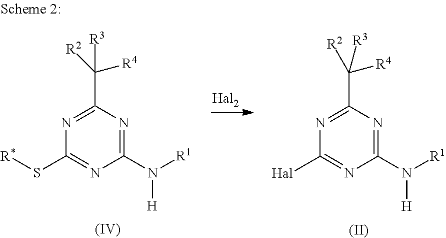

The variables R¹, R², R³, R⁴ in formulae (II) and (IV) have the meanings, in particular the preferred meanings, as defined above in context of formula (I);
Hal is halogen;
   Preferably Cl or Br;
Particular preferred Cl;
R* is C₁-C₆-alkyl, C₂-C₆-haloalkyl or phenyl;
   in particular C₁-C₄-alkyl or C₂-C₄-haloalkyl;
   more particularly C₁-C₄-alkyl;
   especially CH₃.

The reaction of the thiotriazines of formula (IV) with the halogen is usually carried out from 0° C. to the boiling point of the reaction mixture, preferably from 15° C. to the boiling point of the reaction mixture, particularly preferably from 15° C. to 40° C., in an inert organic solvent (e.g. J. K. Chakrabarti et al., Tetrahedron 1975, 31, 1879-1882).

The reaction can be carried out at atmospheric pressure or under elevated pressure, if appropriate under an inert gas, continuously or batchwise.

In the reaction of the thiotriazines of formula (IV) with, halogen is generally used in excess with regard to the thiotriazines of formula (IV).

The reaction of the thiotriazines of formula (IV) with the halogen is usually carried out in an organic solvent.

Suitable in principle are all solvents which are capable of dissolving the thiotriazines of formula (IV) and the halogen at least partly and preferably fully under reaction conditions. Examples of suitable solvents are aliphatic hydrocarbons such as pentane, hexane, cyclohexane and mixtures of C₅-C₈-alkanes, halogenated hydrocarbons such as dichloromethane, 1,2-dichloroethane, chloroform and carbon tetrachloride; ethers such as diethyl ether, diisopropyl ether, tert.-butyl methylether (TBME), dioxane, anisole and tetrahydrofuran (THF), alcohols such as methanol, ethanol, n-propanol, isopropanol, n-butanol and tert.-butanol, as well as organic acids like formic acid, acetic acid, propionic acid, oxalic acid, citric acid, trifluoroacetic acid. Preferred solvents are halogenated hydrocarbons and organic acids as defined above. The term solvent as used herein also includes mixtures of two or more of the above compounds. The end of the reaction can easily be determined by the skilled worker by means of routine methods. The reaction mixtures are worked up in a customary manner, for example by mixing with water, separation of the phases and, if appropriate, chromatographic purification of the crude product.

The halotriazines of formula (II) required for the preparation of aminotriazine compounds of formula (I), wherein R¹ is H, C₁-C₆-alkyl, (C₁-C₆-alkoxy)-C₁-C₆-alkyl, can also be prepared by reacting 2,4-dichlorotriazines of formula (VII) with a an amine H₂N—R¹, in particular with ammonia, as depicted in scheme 3:

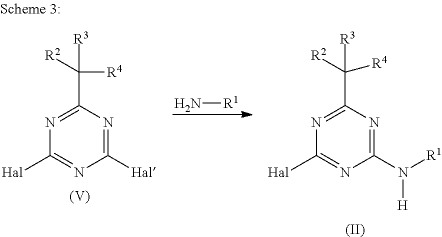

The variables R², R³ and R⁴ in formulae (II) and (V) has the meanings, in particular the preferred meanings, as defined above in context of formula (I).

Hal and Hal′ are each, independently, halogen, in particular bromine or chlorine, especially chlorine.

The reaction depicted in scheme 3 can be performed by simply mixing the required amounts of the compound of formula (V) with the amine H₂N—R¹ or by analogy to the reaction depicted in step 1.

Preferably the molar ratio of the amine to the halotriazines of formula (II) is in the range from 10:1 to 1:1, preferably 5:1 to 1:1.

The reaction depicted in scheme 3 is preferably carried out in an inert solvent. Examples of suitable solvents are nitromethane, aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, tert.-butyl methylether (TBME), dioxane, anisole and tetrahydrofuran (THF), esters such as ethyl acetate and butyl acetate; nitriles such as acetonitrile and propionitrile, as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NM P) and mixtures thereof with water or with aliphatic hydrocarbons such as pentane, hexane, cyclohexane or with mixtures of C₅-C₈-alkane. Preferred solvents are ethers as defined above and mixtures thereof with water. The term solvent as used herein also includes mixtures of two or more of the above compounds.

The reaction depicted in scheme 1 may be performed in the presence of an auxiliary base. Suitable bases are those mentioned in context with the reaction depicted in scheme 1. However, the amine H₂N—R¹ may itself serve as an auxiliary base. In this case, usually an excess of the amine H₂N—R¹ is used.

The thiotriazines of formula (IV) required for the preparation of halotriazines of formula (II) can be prepared in accordance by reacting guanidine-salts of formula (VIII) with carbonyl compounds of formula (IX) in the presence of a base, depicted in scheme 2a:

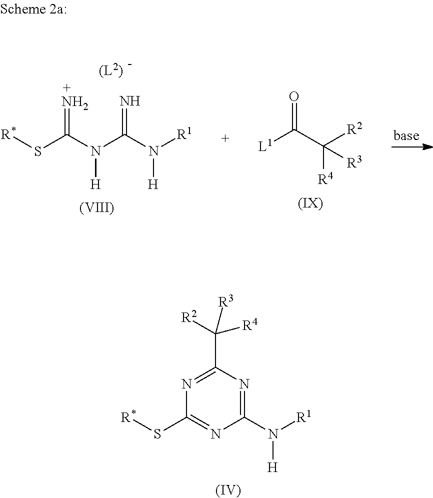

In scheme 2a, the variables R¹, R², R³, R⁴ have the above meanings, in particular the preferred meanings, as in formula (I) mentioned above; while
R* is C₁-C₆-alkyl, C₂-C₆-haloalkyl or phenyl;
   preferably C₁-C₆-alkyl or C₂-C₆-haloalkyl;
   particularly preferred C₁-C₆-alkyl;
   especially preferred CH₃;
L¹ is a nucleophilically displaceable leaving group such as halogen, CN, C₁-C₆-alkoxy, C₁-C₆-alkoxycarbonyl, C₁-C₆-alkylcarbonyloxy or C₁-C₆-alkoxycarbonyloxy;
   preferably halogen or C₁-C₆-alkoxy;
   particularly preferred Cl or C₁-C₆-alkoxy,
   also particularly preferred halogen;
   especially preferred Cl; and
L² is a nucleophilically displaceable leaving group such as halogen, C₁-C₆-alkylsulfonyloxy, C₁-C₆-haloalkylsufonyloxy, C₁-C₆-alkoxysulfonyloxy or phenylsulfonyloxy;
   preferably halogen or C₁-C₆-haloalkylsufonyloxy;
   particularly preferred halogen;
   especially preferred I; and

The reaction of the guanidine-salt of formula (VIII) with the carbonyl compound of formula (IX) is usually carried out at temperatures from 50° C. to the boiling point of the reaction mixture, preferably from 50° C. to 100° C.

The reaction can be carried out at atmospheric pressure or under elevated pressure, if appropriate under an inert gas, continuously or batchwise.

In one embodiment of the process according to the invention, the guanidine-salts of formula (VIII) and the carbonyl compound of formula (IX) are used in equimolar amounts.

In another embodiment of the process according to the invention, the carbonyl compound of formula (IX) is used in excess with regard to the guanidine-salts of formula (VIII).

Preferably the molar ratio of the carbonyl compound of formula (IX) to the guanidine-salt of formula (VIII) is in the range from 1.5:1 to 1:1, preferably 1.2:1 to 1:1, especially preferred 1.2:1, also especially preferred 1:1.

The reaction of the guanidine-salt of formula (VIII) with the carbonyl compound of formula (IX) is usually carried out in an organic solvent.

Suitable in principle are all solvents which are capable of dissolving the guanidine-salt of formula (VIII) and the carbonyl compound of formula (IX) at least partly and preferably fully under reaction conditions.

Examples of suitable solvents are halogenated hydrocarbons, ethers, nitriles, dipolar aprotic solvents and mixtures thereof.

Halogenated hydrocarbons are for example dichloromethane, 1,2-dichloroethane, chloroform, carbon tetrachloride, chlorobenzene and mixtures thereof.

Ethers are for example diethyl ether, diisopropyl ether, tert.-butyl methylether (TBME), dioxane, anisole, tetrahydrofuran (THF) and mixtures thereof.

Nitriles are for example acetonitrile, propionitrile and mixtures thereof.

Dipolar aprotic solvents are for example sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO), 1-methyl-2 pyrrolidinone (NMP) and mixtures thereof.

Preferred solvents are ethers and dipolar aprotic solvents as defined above.

More preferred solvents are ethers as defined above.

The reaction of the guanidine-salts of formula (VIII) with the carbonyl compound of formula (IX) is carried out in the presence of a base.

Examples of suitable bases include metal-containing bases and nitrogen-containing bases.

Examples of suitable metal-containing bases are inorganic compounds, alkali metal hydrides, alkaline earth metal hydrides, alkali metal carbonates, alkaline earth metal carbonates, alkali metal hydrogen carbonates (bicarbonates), of alkali metal phosphates, alkaline earth metal phosphates, organic bases and mixtures thereof.

Examples of inorganic compounds are alkali metal and alkaline earth metal oxide, and other metal oxides, such as lithium oxide, sodium oxide, potassium oxide, magnesium oxide, calcium oxide and magnesium oxide, iron oxide, silver oxide and mixtures thereof.

Examples of alkali metal and alkaline earth metal hydrides are lithium hydride, sodium hydride, potassium hydride and calcium hydride.

Examples of alkali metal and alkaline earth metal carbonates are lithium carbonate, sodium carbonate, potassium carbonate, magnesium carbonate, and calcium carbonate and mixtures thereof.

Examples of alkali metal hydrogen carbonates (bicarbonates) are lithium hydrogen carbonate, sodium hydrogen carbonate, potassium hydrogen carbonate and mixtures thereof.

Examples of alkali metal and alkaline earth metal phosphates are sodium phosphate, potassium phosphate and calcium phosphate and mixtures thereof.

Examples of organic bases, are tertiary amines such as tri-C₁-C₆-alkylamines, for example triethylamine, trimethylamine, N-ethyldiisopropylamine and mixtures thereof, and N-methylpiperidine, pyridine, substituted pyridines such as collidine, lutidine, N-methylmorpholine, and also bicyclic amines such as 1,8-diazabicyclo[5.4.0]undec-7-ene (DBU) or 1,5-diazabicyclo[4.3.0]non-5-ene (DBN) and mixtures thereof.

Preferred bases are tri-C₁-C₆-alkylamines, especially preferred are triethylamine, trimethylamine, N-ethyldiisopropylamine, N-methylpiperidine and mixtures thereof.

The bases are generally employed in excess; however they can also be employed in equimolar amounts, or, if appropriate, can be used as solvent.

Preferably from 1 to 5 equivalents of the base, particularly preferred 3 equivalents of the base are used, based on the guanidine-salts of formula (VIII).

The end of the reaction can easily be determined by the skilled worker by means of routine methods.

The reaction mixtures are worked up in a customary manner, for example by mixing with water, separation of the phases and, if appropriate, chromatographic purification of the crude product.

The carbonyl compounds of formula (IX) required for the preparation of azines of formula (IV) are known from the literature. They can be prepared in accordance and/or are commercially available.

The guanidine-salt of formula (VIII), wherein L² is iodine, required for the preparation of thiotriazines of formula (IV) is known from the literature (e.g. M. Freund et al., Chem. Ber. 1901, 34, 3110-3122; H. Eilingsfeld et al., Chem. Ber. 1967, 100, 1874-1891).

The guanidine-salts of formula (VIII) are commercially available and/or can be prepared in accordance with the literature cited.

### Process B)

The aminotriazine compounds of formula (I), wherein
R¹ is different from hydrogen, e.g. C₁-C₆-alkyl, (C₁-C₆-alkoxy)-C₁-C₆-alkyl, CN, (C₁-C₆-alkyl)carbonyl, (C₁-C₆-alkoxy)carbonyl, (C₁-C₆-alkyl)sulfonyl, phenylsulfonyl, phenyl, phenyl-C₁-C₆ alkyl, phenylcarbonyl or phenoxycarbonyl,
   wherein the phenyl is unsubstituted or substituted as defined above for the respective radicals in formula (I);

can be prepared by reacting azines of formula (I), wherein R¹ is hydrogen with a compound of formula (VI) as depicted in scheme 4:

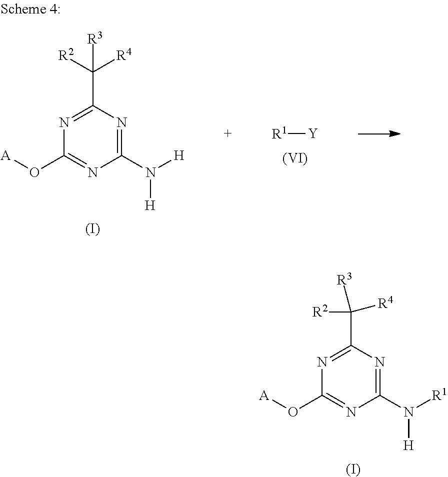

The variables A, R², R³ and R⁴ have the meanings, in particular the preferred meanings, as in formula (I) mentioned above,
R¹ is different from hydrogen, e.g. C₁-C₆-alkyl, (C₁-C₆-alkoxy)-C₁-C₆-alkyl, CN, (C₁-C₆-alkyl)carbonyl, (C₁-C₆-alkoxy)carbonyl, (C₁-C₆-alkyl)sulfonyl, phenylsulfonyl, phenyl, phenyl-C₁-C₆ alkyl, phenylcarbonyl or phenoxycarbonyl, wherein the phenyl is unsubstituted or substituted as defined above for the respective radicals in formula (I);
   in particular C₁-C₄-alkyl, CN, (C₁-C₆-alkyl)carbonyl, (C₁-C₆-alkoxy)carbonyl or (C₁-C₆-alkyl)sulfonyl;
   especially CN, COCH₃, COOCH₃ or SO₂CH₃; and
Y is halogen or oxycarbonyl-C₁-C₆-alkyl;
   in particular halogen;
   especially Cl or Br.

The process B is usually carried out at from 0° C. to the boiling point of the reaction mixture, preferably from 23° C. to 130° C., particularly preferably from 23° C. to 100° C., (e.g. Y. Yuki et al., Polym. J. 1992, 24, 791-799).

The process B can be carried out at atmospheric pressure or under elevated pressure, if appropriate under an inert gas, continuously or batchwise.

In one embodiment of process B according to the invention, the aminotriazine compounds of formula (I), wherein R¹ is hydrogen are used in excess with regard to the compound of formula (VI).

In another embodiment of process B according to the invention the aminotriazine compounds of formula (I), wherein R¹ respectively, is hydrogen and the compound of formula (VI), are used in equimolar amounts.

Preferably the molar ratio of the aminotriazine compounds of formula (I), wherein R¹ is hydrogen to the compound of formula (VI) is in the range from 1:1.5 to 1:1, preferably 1:1.2 to 1:1, especially preferred 1:1.

The process B is usually carried out in an organic solvent. Suitable in principle are all solvents which are capable of dissolving the aminotriazine compounds of formula (I), wherein R¹ is hydrogen and the compound of formula (VI), at least partly and preferably fully under reaction conditions. Examples of suitable solvents are halogenated hydrocarbons such as dichloromethane, 1,2-dichloroethane, chloroform, carbon tetrachloride and chlorobenzene; ethers such as diethyl ether, diisopropyl ether, tert.-butyl methylether (TBME), dioxane, anisole and tetrahydrofuran (THF); nitriles such as acetonitrile and propionitrile; alcohols such as methanol, ethanol, n-propanol, isopropanol, n-butanol and tert.-butanol; organic acids like formic acid, acetic acid, propionic acid, oxalic acid, methylbenzenesulfonic acid, benzenesulfonic acid, camphorsulfonic acid, citric acid, trifluoroacetic acid as well as dipolar aprotic solvents such as sulfolane, dimethylsulfoxide, N,N-dimethylformamide (DMF), N,N-dimethylacetamide (DMAC), 1,3-dimethyl-2-imidazolidinone (DMI), N,N′-dimethylpropylene urea (DMPU), dimethyl sulfoxide (DMSO) and 1-methyl-2 pyrrolidinone (NMP). Preferred solvents are halogenated hydrocarbons, ethers and dipolar aprotic solvents as mentioned above. More preferred solvents are dichloromethane or dioxane. It is also possible to use mixtures of the solvents mentioned. The term solvent as used herein also includes mixtures of two or more of the above compounds.

The process B is optionally carried out in the presence of a base. Examples of suitable bases include metal-containing bases and nitrogen-containing bases. Examples of suitable metal-containing bases are inorganic compounds such as alkali metal and alkaline earth metal hydrides such as lithium hydride, sodium hydride, potassium hydride and calcium hydride, alkali metal and alkaline earth metal carbonates such as lithium carbonate, sodium carbonate, potassium carbonate, magnesium carbonate, and calcium carbonate, as well as alkali metal hydrogen carbonates (bicarbonates) such as lithium hydrogen carbonate, sodium hydrogen carbonate, potassium hydrogen carbonate; alkali metal and alkaline earth metal phosphates such as sodium phosphate, potassium phosphate and calcium phosphate; and furthermore organic bases, such as tertiary amines such as tri-C₁-C₆-alkylamines, for example triethylamine, trimethylamine, N-ethyldiisopropylamine, and N-methylpiperidine, pyridine, substituted pyridines such as collidine, lutidine, N-methylmorpholine and 4-dimethylaminopyridine (DMAP), and also bicyclic amines such as 1,8-diazabicyclo[5.4.0]undec-7-ene (DBU) or 1,5-diazabicyclo[4.3.0]non-5-ene (DBN). Preferred bases are organic bases and alkali metal carbonates as mentioned above. Especially preferred bases are organic bases as mentioned above. The term base as used herein also includes mixtures of two or more, preferably two of the above compounds. The bases are generally employed in excess; however they can also be employed in equimolar amounts, or, if appropriate, can be used as solvent. Preferably from 1 to 5 base equivalents, particularly preferred 3 base equivalents of base are used, based on the aminotriazine compounds of formula (I).

Work-up of the reaction mixture is performed by standard procedures.

The compounds of formula (VI) are known compounds. They are commercially available or can be prepared in analogy to known methods.

The compounds of formula (I) have herbicidal activity. Therefore, they can be used for controlling unwanted or undesired plants or vegetation. They can also be used in a method for controlling unwanted or undesired plants or vegetation, which method comprises allowing at least one compound of formula (I) or a salt thereof to act on plants, their environment or on seed. In order to allow the compound of formula (I) or a salt thereof to act on plants, their environment or on seed the compounds of the invention are applied to the plants, their environment or to the seed of said plants.

To widen the spectrum of action and to achieve synergistic effects, the aminotriazine compounds of formula (I) may be mixed with a large number of representatives of other herbicidal or growth-regulating active ingredient groups and then applied concomitantly.

Suitable components for mixtures are, for example, herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

It may furthermore be beneficial to apply the aminotriazine compounds of formula (I) alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

The invention also relates to agrochemical compositions comprising at least an auxiliary and at least one aminotriazine compound of formula (I) according to the invention.

The invention also relates to combinations of diaminotriazine compounds of formula (I) with at least one further herbicide B and/or at least one safener C).

The further herbicidal compound B (component B) is in particular selected from the herbicides of class b1) to b15):
b1) lipid biosynthesis inhibitors;
b2) acetolactate synthase inhibitors (ALS inhibitors);
b3) photosynthesis inhibitors;
b4) protoporphyrinogen-IX oxidase inhibitors,
b5) bleacher herbicides;
b6) enolpyruvyl shikimate 3-phosphate synthase inhibitors (EPSP inhibitors);
b7) glutamine synthetase inhibitors;
b8) 7,8-dihydropteroate synthase inhibitors (DHP inhibitors);
b9) mitosis inhibitors;
b10) inhibitors of the synthesis of very long chain fatty acids (VLCFA inhibitors);
b11) cellulose biosynthesis inhibitors;
b12) decoupler herbicides;
b13) auxinic herbicides;
b14) auxin transport inhibitors; and b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters;

including their agriculturally acceptable salts or derivatives such as ethers, esters or amides.

In one embodiment of the present invention the compositions according to the present invention comprise at least one diaminotriazine compound of formula (I) and at least one further active compound B (herbicide B).

According to a further embodiment of the invention the compositions contain at least one inhibitor of the lipid biosynthesis (herbicide b1). These are compounds that inhibit lipid biosynthesis. Inhibition of the lipid biosynthesis can be affected either through inhibition of acetylCoA carboxylase (hereinafter termed ACC herbicides) or through a different mode of action (hereinafter termed non-ACC herbicides). The ACC herbicides belong to the group A of the H RAC classification system whereas the non-ACC herbicides belong to the group N of the HRAC classification.

According to a further embodiment of the invention the compositions contain at least one ALS inhibitor (herbicide b2). The herbicidal activity of these compounds is based on the inhibition of acetolactate synthase and thus on the inhibition of the branched chain amino acid biosynthesis. These inhibitors belong to the group B of the HRAC classification system.

According to a further embodiment of the invention the compositions contain at least one inhibitor of photosynthesis (herbicide b3). The herbicidal activity of these compounds is based either on the inhibition of the photosystem II in plants (so-called PSII inhibitors, groups C1, C2 and C3 of H RAC classification) or on diverting the electron transfer in photosystem I in plants (so-called PSI inhibitors, group D of HRAC classification) and thus on an inhibition of photosynthesis. Amongst these, PSII inhibitors are preferred.

According to a further embodiment of the invention the compositions contain at least one inhibitor of protoporphyrinogen-IX-oxidase (herbicide b4). The herbicidal activity of these compounds is based on the inhibition of the protoporphyrinogen-IX-oxidase. These inhibitors belong to the group E of the HRAC classification system.

According to a further embodiment of the invention the compositions contain at least one bleacher-herbicide (herbicide b5). The herbicidal activity of these compounds is based on the inhibition of the carotenoid biosynthesis. These include compounds which inhibit carotenoid biosynthesis by inhibition of phytoene desaturase (so-called PDS inhibitors, group F1 of HRAC classification), compounds that inhibit the 4-hydroxyphenylpyruvate-dioxygenase (HPPD inhibitors, group F2 of HRAC classification), compounds that inhibit DOXsynthase (group F4 of HRAC class) and compounds which inhibit carotenoid biosynthesis by an unknown mode of action (bleacher—unknown target, group F3 of HRAC classification).

According to a further embodiment of the invention the compositions contain at least one EPSP synthase inhibitor (herbicide b6). The herbicidal activity of these compounds is based on the inhibition of enolpyruvyl shikimate 3-phosphate synthase, and thus on the inhibition of the amino acid biosynthesis in plants. These inhibitors belong to the group G of the HRAC classification system.

According to a further embodiment of the invention the compositions contain at least one glutamine synthetase inhibitor (herbicide b7). The herbicidal activity of these compounds is based on the inhibition of glutamine synthetase, and thus on the inhibition of the amino acid biosynthesis in plants. These inhibitors belong to the group H of the HRAC classification system.

According to an further embodiment of the invention the compositions contain at least one DHP synthase inhibitor (herbicide b8). The herbicidal activity of these compounds is based on the inhibition of 7,8-dihydropteroate synthase. These inhibitors belong to the group I of the HRAC classification system.

According to a further embodiment of the invention the compositions contain at least one mitosis inhibitor (herbicide b9). The herbicidal activity of these compounds is based on the disturbance or inhibition of microtubule formation or organization, and thus on the inhibition of mitosis. These inhibitors belong to the groups K1 and K2 of the HRAC classification system. Among these, compounds of the group K1, in particular dinitroanilines, are preferred.

According to a further embodiment of the invention the compositions contain at least one VLCFA inhibitor (herbicide b10). The herbicidal activity of these compounds is based on the inhibition of the synthesis of very long chain fatty acids and thus on the disturbance or inhibition of cell division in plants. These inhibitors belong to the group K3 of the HRAC classification system.

According to an further embodiment of the invention the compositions contain at least one cellulose biosynthesis inhibitor (herbicide b11). The herbicidal activity of these compounds is based on the inhibition of the biosynthesis of cellulose and thus on the inhibition of the synthesis of cell walls in plants. These inhibitors belong to the group L of the HRAC classification system.

According to a further embodiment of the invention the compositions contain at least one decoupler herbicide (herbicide b12). The herbicidal activity of these compounds is based on the disruption of the cell membrane. These inhibitors belong to the group M of the HRAC classification system.

According to a further embodiment of the invention the compositions contain at least one auxinic herbicide (herbicide b13). These include compounds that mimic auxins, i.e. plant hormones, and affect the growth of the plants. These compounds belong to the group O of the HRAC classification system.

According to a further embodiment of the invention the compositions contain at least one auxin transport inhibitor (herbicide b14). The herbicidal activity of these compounds is based on the inhibition of the auxin transport in plants. These compounds belong to the group P of the H RAC classification system.

As to the given mechanisms of action and classification of the active substances, see e.g. “HRAC, Classification of Herbicides According to Mode of Action”, http://www.plantprotection.org/hrac/MOA.html).

Preference is given to those compositions according to the present invention comprising at least one herbicide B selected from herbicides of class b1, b6, b9, b10 and b11.

Preference is also given to those compositions according to the present invention comprising at least one herbicide B selected from herbicides of class b2, b3, b4, b5, b6, b9 and b10.

Specific preference is given to those compositions according to the present invention which comprise at least one herbicide B selected from the herbicides of class b4, b6 b9 and b10.

Particular preference is given to those compositions according to the present invention which comprise at least one herbicide B selected from the herbicides of class b4, b6 and b10.

Examples of herbicides B which can be used in combination with the diaminitriazine compounds of formula (I) according to the present invention are:

b1) from the group of the lipid biosynthesis inhibitors:

ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;

b2) from the group of the ALS inhibitors:

sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl and tritosulfuron,

imidazolinones such as imazamethabenz, imazamethabenz-methyl, imazamox, imazapic, imazapyr, imazaquin and imazethapyr, triazolopyrimidine herbicides and sulfonanilides such as cloransulam, cloransulam-methyl, diclosulam, flumetsulam, florasulam, metosulam, penoxsulam, pyrimisulfan and pyroxsulam,

pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8),

sulfonylaminocarbonyl-triazolinone herbicides such as flucarbazone, flucarbazone-sodium, propoxycarbazone, propoxycarbazone-sodium, thiencarbazone and thiencarbazone-methyl; and triafamone;

among these, a preferred embodiment of the invention relates to those compositions comprising at least one imidazolinone herbicide;

b3) from the group of the photosynthesis inhibitors:

amicarbazone, inhibitors of the photosystem II, e.g. triazine herbicides, including of chlorotriazine, triazinones, triazindiones, methylthiotriazines and pyridazinones such as ametryn, atrazine, chloridazone, cyanazine, desmetryn, dimethametryn, hexazinone, metribuzin, prometon, prometryn, propazine, simazine, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridate, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin (BAS 850 H), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100; Sumitomo; LS 5296489), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7) (LS 4061013), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0) (LS 567 0033=F2-Flumioxazin), 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0) (LS 568 1323=Uracil-F2-PPO), methyl (E)-4-[2-chloro-5-[4-chloro-5-(difluoromethoxy)-1H-methyl-pyrazol-3-yl]-4-fluoro-phenoxy]-3-methoxy-but-2-enoate (CAS 948893-00-3) (Isagro, IR6396), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4) (FMC Trifluoromethyluracil);

b5) from the group of the bleacher herbicides:

PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)pyrimidine (CAS 180608-33-7), HPPD inhibitors: benzobicyclon, benzofenap, bicyclopyrone, clomazone, fenquintrione, isoxaflutole, mesotrione, pyrasulfotole, pyrazolynate, pyrazoxyfen, sulcotrione, tefuryltrione, tembotrione, tolpyralate, topramezone, bleacher, unknown target: aclonifen, amitrole and flumeturon;

b6) from the group of the EPSP synthase inhibitors:

glyphosate, glyphosate-isopropylammonium, glyposate-potassium and glyphosate-trimesium (sulfosate);

b7) from the group of the glutamine synthase inhibitors:

bilanaphos (bialaphos), bilanaphos-sodium, glufosinate, glufosinate-P and glufosinate-ammonium;

b8) from the group of the DHP synthase inhibitors:

asulam;

b9) from the group of the mitosis inhibitors:

compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlorthal-dimethyl, pyridines such as dithiopyr and thiazopyr, benzamides such as propyzamide and tebutam; compounds of group K2: carbetamide, chlorpropham, flamprop, flamprop-isopropyl, flamprop-methyl, flamprop-M-isopropyl, flamprop-M-methyl and propham; among these, compounds of group K1, in particular dinitroanilines are preferred;

b10) from the group of the VLCFA inhibitors:

chloroacetamides such as acetochlor, alachlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, acetanilides such as diphenamid, naproanilide, napropamide and napropamide-M, tetrazolinones such fentrazamide, and other herbicides such as anilofos, cafenstrole, fenoxasulfone, ipfencarbazone, piperophos, pyroxasulfone and isoxazoline compounds of the formulae II.1, II.2, II.3, II.4, II.5, II.6, II.7, II.8 and II.9

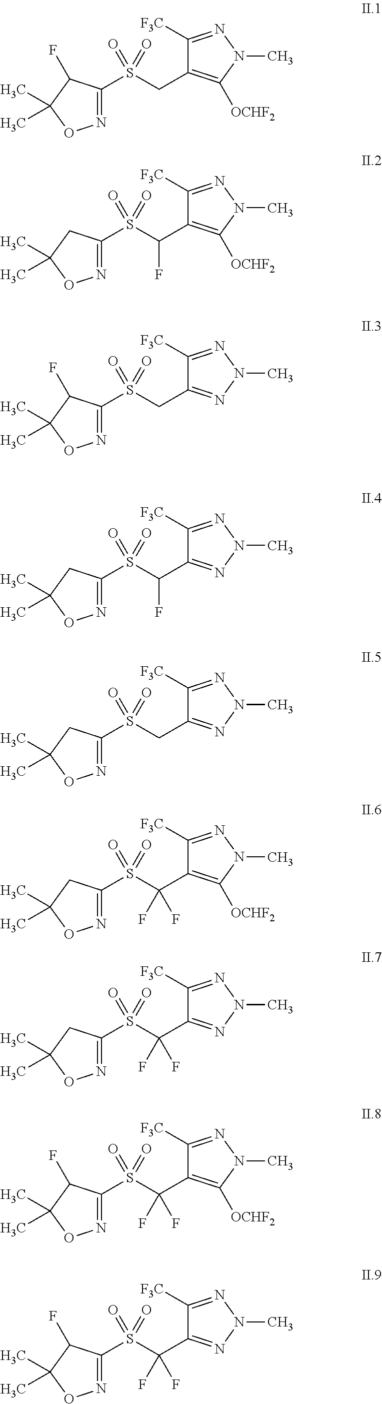

the isoxazoline compounds of the formula (I)I are known in the art, e.g. from WO 2006/024820, WO 2006/037945, WO 2007/071900 and WO 2007/096576;

among the VLCFA inhibitors, preference is given to chloroacetamides and oxyacetamides;

b11) from the group of the cellulose biosynthesis inhibitors: chlorthiamid, dichlobenil, flupoxam, indaziflam, isoxaben, triaziflam and 1-cyclohexyl-5-pentafluorphenyloxy-1⁴-[1,2,4,6]thiatriazin-3-ylamine (CAS 175899-01-1);

b12) from the group of the decoupler herbicides:

dinoseb, dinoterb and DNOC and its salts;

b13) from the group of the auxinic herbicides:

2,4-D and its salts and esters such as clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (CAS 943832-60-8 DOW, LS 566509); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 4-amino-3-chloro-6-(4-chloro-2-fluoro-3-methoxyphenyl)-5-fluoropyridine-2-carboxylic acid (DOW, “Rinskor-acid”) and benzyl 4-amino-3-chloro-6-(4-chloro-2-fluoro-3-methoxyphenyl)-5-fluoropyridine-2-carboxylate (CAS 1390661-72-9) (DOW, “Rinskor”);

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenol-methyl, cinmethylin, cumyluron, cyclopyrimorate (CAS 499223-49-3 Mitsui; SW-065; H-965) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine and tridiphane.

Preferred herbicides B that can be used in combination with the diaminotriazine compounds of the formula (I) according to the present invention are:

b1) from the group of the lipid biosynthesis inhibitors:

clethodim, clodinafop-propargyl, cycloxydim, cyhalofop-butyl, diclofop-methyl, fenoxaprop-P-ethyl, fluazifop-P-butyl, haloxyfop-P-methyl, metamifop, pinoxaden, profoxydim, propaquizafop, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); benfuresate, dimepiperate, EPTC, esprocarb, ethofumesate, molinate, orbencarb, prosulfocarb, thiobencarb and triallate;

b2) from the group of the ALS inhibitors:

amidosulfuron, azimsulfuron, bensulfuron-methyl, bispyribac-sodium, chlorimuron-ethyl, chlorsulfuron, cloransulam-methyl, cyclosulfamuron, diclosulam, ethametsulfuron-methyl, ethoxysulfuron, flazasulfuron, florasulam, flucarbazone-sodium, flucetosulfuron, flumetsulam, flupyrsulfuron-methyl-sodium, foramsulfuron, halosulfuron-methyl, imazamethabenz-methyl, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, metosulam, metsulfuron-methyl, nicosulfuron, orthosulfamuron, oxasulfuron, penoxsulam, primisulfuron-methyl, propoxycarbazon-sodium, propyrisulfuron, prosulfuron, pyrazosulfuron-ethyl, pyribenzoxim, pyrimisulfan, pyriftalid, pyriminobac-methyl, pyrithiobac-sodium, pyroxsulam, rimsulfuron, sulfometuron-methyl, sulfosulfuron, thiencarbazone-methyl, thifensulfuron-methyl, triasulfuron, tribenuron-methyl, trifloxysulfuron, triflusulfuron-methyl, tritosulfuron and triafamone;

b3) from the group of the photosynthesis inhibitors:

ametryn, amicarbazone, atrazine, bentazone, bentazone-sodium, bromoxynil and its salts and esters, chloridazone, chlorotoluron, cyanazine, desmedipham, diquat-dibromide, diuron, fluometuron, hexazinone, ioxynil and its salts and esters, isoproturon, lenacil, linuron, metamitron, methabenzthiazuron, metribuzin, paraquat, paraquat-dichloride, phenmedipham, propanil, pyridate, simazine, terbutryn, terbuthylazine and thidiazuron;

b4) from the group of the protoporphyrinogen-IX oxidase inhibitors:

acifluorfen-sodium, bencarbazone, benzfendizone, butafenacil, carfentrazone-ethyl, cinidon-ethyl, flufenpyr-ethyl, flumiclorac-pentyl, flumioxazin, fluoroglycofen-ethyl, fomesafen, lactofen, oxadiargyl, oxadiazon, oxyfluorfen, pentoxazone, pyraflufen, pyraflufen-ethyl, saflufenacil, sulfentrazone, tiafenacil, trifludimoxazin (BAS 850 H), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100; Sumitomo; LS 5296489), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethyl phenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7) (LS 4061013), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0) (LS 567 0033=F2-Flumioxazin); 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0) (LS 568 1323=Uracil-F2-PPO), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4) (FMC Trifluoromethyluracil);

b5) from the group of the bleacher herbicides:

aclonifen, amitrole, beflubutamid, benzobicyclon, bicyclopyrone, clomazone, diflufenican, fenquintrione, flumeturon, flurochloridone, flurtamone, isoxaflutole, mesotrione, norflurazon, picolinafen, pyrasulfotole, pyrazolynate, sulcotrione, tefuryltrione, tembotrione, tolpyralate, topramezone and 4-(3-trifluoromethylphenoxy)-2-(4-trifluoromethylphenyl)pyrimidine (CAS 180608-33-7);

b6) from the group of the EPSP synthase inhibitors:

glyphosate, glyphosate-isopropylammonium, glyphosate-potassium and glyphosate-trimesium (sulfosate);

b7) from the group of the glutamine synthase inhibitors:

glufosinate, glufosinate-P, glufosinate-ammonium;

b8) from the group of the DHP synthase inhibitors: asulam;

b9) from the group of the mitosis inhibitors:

benfluralin, dithiopyr, ethalfluralin, flamprop, flamprop-isopropyl, flamprop-methyl, flamprop-M-isopropyl, flamprop-M-methyl, oryzalin, pendimethalin, thiazopyr and trifluralin;

b10) from the group of the VLCFA inhibitors:

acetochlor, alachlor, anilofos, butachlor, cafenstrole, dimethenamid, dimethenamid-P, fentrazamide, flufenacet, mefenacet, metazachlor, metolachlor, S-metolachlor, naproanilide, napropamide, napropamide-M, pretilachlor, fenoxasulfone, ipfencarbazone, pyroxasulfone thenylchlor and isoxazoline-compounds of the formulae II.1, II.2, II.3, II.4, II.5, II.6, II.7, II.8 and II.9 as mentioned above;

b11) from the group of the cellulose biosynthesis inhibitors: dichlobenil, flupoxam, indaziflam, isoxaben, triaziflam and 1-cyclohexyl-5-pentafluorphenyloxy-1⁴-[1,2,4,6]thiatriazin-3-ylamine (CAS 175899-01-1);

b13) from the group of the auxinic herbicides:

2,4-D and its salts and esters, aminocyclopyrachlor and its salts and esters, aminopyralid and its salts such as aminopyralid-dimethylammonium, aminopyralid-tris(2-hydroxypropyl)ammonium and its esters, clopyralid and its salts and esters, dicamba and its salts and esters, dichlorprop-P and its salts and esters, fluroxypyr-meptyl, halauxifen and its salts and esters (CAS 943832-60-8 DOW, LS 566509), MCPA and its salts and esters, MCPB and its salts and esters, mecoprop-P and its salts and esters, picloram and its salts and esters, quinclorac, quinmerac, triclopyr and its salts and esters, 4-amino-3-chloro-6-(4-chloro-2-fluoro-3-methoxyphenyl)-5-fluoropyridine-2-carboxylic acid (DOW, “Rinskor-acid”) and benzyl 4-amino-3-chloro-6-(4-chloro-2-fluoro-3-methoxyphenyl)-5-fluoropyridine-2-carboxylate (CAS 1390661-72-9) (DOW, “Rinskor”);

b14) from the group of the auxin transport inhibitors: diflufenzopyr and diflufenzopyr-sodium;

b15) from the group of the other herbicides: bromobutide, cinmethylin, cumyluron, cyclopyrimorate (CAS 499223-49-3 Mitsui; SW-065; H-965) and its salts and esters, dalapon, difenzoquat, difenzoquat-metilsulfate, DSMA, dymron (=daimuron), indanofan, metam, methylbromide, MSMA, oxaziclomefone, pyributicarb and tridiphane.

Particularly preferred herbicides B that can be used in combination with the diaminotriazine compounds of the formula (I) according to the present invention are:

b1) from the group of the lipid biosynthesis inhibitors: clodinafop-propargyl, cycloxydim, cyhalofop-butyl, fenoxaprop-P-ethyl, pinoxaden, profoxydim, tepraloxydim, tralkoxydim, 4-(4′-Chloro-4-cyclopropyl-2′-fluoro[1, 1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); esprocarb, prosulfocarb, thiobencarb and triallate;

b2) from the group of the ALS inhibitors: bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, nicosulfuron, penoxsulam, propoxycarbazon-sodium, propyrisulfuron, pyrazosulfuron-ethyl, pyroxsulam, rimsulfuron, sulfosulfuron, thiencarbazon-methyl, tritosulfuron and triafamone;

b3) from the group of the photosynthesis inhibitors: ametryn, atrazine, diuron, fluometuron, hexazinone, isoproturon, linuron, metribuzin, paraquat, paraquat-dichloride, propanil, terbutryn and terbuthylazine;

b4) from the group of the protoporphyrinogen-IX oxidase inhibitors: flumioxazin, oxyfluorfen, pyraflufen, pyraflufen-ethyl, saflufenacil, sulfentrazone, trifludimoxazin (BAS 850 H), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100; Sumitomo; LS 5296489), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7) (LS 4061013), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0) (LS 567 0033=F2-Flumioxazin), and 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0) (LS 568 1323=Uracil-F2-PPO);

b5) from the group of the bleacher herbicides: amitrole, bicyclopyrone, clomazone, diflufenican, fenquintrione, flumeturon, flurochloridone, isoxaflutole, mesotrione, picolinafen, sulcotrione, tefuryltrione, tembotrione, tolpyralate and topramezone;

b6) from the group of the EPSP synthase inhibitors: glyphosate, glyphosate-isopropylammonium and glyphosate-trimesium (sulfosate);

b7) from the group of the glutamine synthase inhibitors: glufosinate, glufosinate-P and glufosinate-ammonium;

b9) from the group of the mitosis inhibitors: pendimethalin and trifluralin;

b10) from the group of the VLCFA inhibitors: acetochlor, cafenstrole, dimethenamid-P, fentrazamide, flufenacet, mefenacet, metazachlor, metolachlor, S-metolachlor, fenoxasulfone, ipfencarbazone and pyroxasulfone; likewise, preference is given to isoxazoline compounds of the formulae II.1, II.2, II.3, II.4, II.5, II.6, II.7, II.8 and II.9 as mentioned above;

b11) from the group of the cellulose biosynthesis inhibitors: indaziflam, isoxaben and triaziflam;

b13) from the group of the auxinic herbicides: 2,4-D and its salts and esters such as clacyfos, and aminocyclopyrachlor and its salts and esters, aminopyralid and its salts and its esters, clopyralid and its salts and esters, dicamba and its salts and esters, fluroxypyr-meptyl, halauxifen, halauxifen-methyl, quinclorac, quinmerac, 4-amino-3-chloro-6-(4-chloro-2-fluoro-3-methoxyphenyl)-5-fluoropyridine-2-carboxylic acid (DOW, “Rinskor-acid”) and benzyl 4-amino-3-chloro-6-(4-chloro-2-fluoro-3-methoxyphenyl)-5-fluoropyridine-2-carboxylate (CAS 1390661-72-9) (DOW, “Rinskor”);

b14) from the group of the auxin transport inhibitors: diflufenzopyr and diflufenzopyr-sodium, b15) from the group of the other herbicides: dymron (=daimuron), indanofan, oxaziclomefone.

Particularly preferred herbicides B are the herbicides B as defined above; in particular the herbicides B.1-B.196 listed below in table B:

TABLE B

| | Herbicide B |
|---|---|
| B.1 | clethodim |
| B.2 | clodinafop-propargyl |
| B.3 | cycloxydim |
| B.4 | cyhalofop-butyl |
| B.5 | fenoxaprop-ethyl |
| B.6 | fenoxaprop-P-ethyl |
| B.7 | metamifop |
| B.8 | pinoxaden |
| B.9 | profoxydim |
| B.10 | sethoxydim |
| B.11 | tepraloxydim |
| B.12 | tralkoxydim |
| B.13 | esprocarb |
| B.14 | ethofumesate |
| B.15 | molinate |
| B.16 | prosulfocarb |
| B.17 | thiobencarb |
| B.18 | triallate |
| B.19 | bensulfuron-methyl |
| B.20 | bispyribac-sodium |
| B.21 | cloransulam-methyl |
| B.22 | chlorsulfuron |
| B.23 | clorimuron |
| B.24 | cyclosulfamuron |
| B.25 | diclosulam |
| B.26 | florasulam |
| B.27 | flumetsulam |
| B.28 | flupyrsulfuron-methyl- |
| | sodium |
| B.29 | foramsulfuron |
| B.30 | imazamox |
| B.31 | imazamox-ammonium |
| B.32 | imazapic |
| B.33 | imazapic-ammonium |
| B.34 | imazapic- |
| | isopropylammonium |
| B.35 | imazapyr |
| B.36 | imazapyr-ammonium |
| B.37 | imazapyr- |
| | isopropylammonium |
| B.38 | imazaquin |
| B.39 | imazaquin-ammonium |
| B.40 | imazethapyr |
| B.41 | imazethapyr-ammonium |
| B.42 | imazethapyr- |
| | isopropylammonium |
| B.43 | imazosulfuron |
| B.44 | iodosulfuron-methyl- |
| | sodium |
| B.45 | iofensulfuron |
| B.46 | iofensulfuron-sodium |
| B.47 | mesosulfuron-methyl |
| B.48 | metazosulfuron |
| B.49 | metsulfuron-methyl |
| B.50 | metosulam |
| B.51 | nicosulfuron |
| B.52 | penoxsulam |
| B.53 | propoxycarbazon-sodium |
| B.54 | pyrazosulfuron-ethyl |
| B.55 | pyribenzoxim |
| B.56 | pyriftalid |
| B.57 | pyroxsulam |
| B.58 | propyrisulfuron |
| B.59 | rimsulfuron |
| B.60 | sulfosulfuron |
| B.61 | thiencarbazone-methyl |
| B.62 | thifensulfuron-methyl |
| B.63 | tribenuron-methyl |
| B.64 | tritosulfuron |
| B.65 | triafamone |
| B.66 | ametryne |
| B.67 | atrazine |
| B.68 | bentazon |
| B.69 | bromoxynil |
| B.70 | bromoxynil-octanoate |
| B.71 | bromoxynil-heptanoate |
| B.72 | bromoxynil-potassium |
| B.73 | diuron |
| B.74 | fluometuron |
| B.75 | hexazinone |
| B.76 | isoproturon |
| B.77 | linuron |
| B.78 | metamitron |
| B.79 | metribuzin |
| B.80 | propanil |
| B.81 | simazin |
| B.82 | terbuthylazine |
| B.83 | terbutryn |
| B.84 | paraquat-dichloride |
| B.85 | acifluorfen |
| B.86 | butafenacil |
| B.87 | carfentrazone-ethyl |
| B.88 | flumioxazin |
| B.89 | fomesafen |
| B.90 | oxadiargyl |
| B.91 | oxyfluorfen |
| B.92 | pyraflufen |
| B.93 | pyraflufen-ethyl |
| B.94 | saflufenacil |
| B.95 | sulfentrazone |
| B.96 | trifludimoxazin (BAS 850 |
| | H) |
| B.97 | ethyl [3-[2-chloro-4-fluoro- |
| | 5-(1-methyl-6-trifluoromethyl- |
| | 2,4-dioxo-1,2,3,4-tetrahydro- |
| | pyrimidin-3-yl)phenoxy]-2- |
| | pyridyloxy]acetate (CAS |
| | 353292-31-6) Sumitomo; LS |
| | 5296489 |
| B.98 | benzobicyclon |
| B.99 | bicyclopyrone |
| B.100 | clomazone |
| B.101 | diflufenican |
| B.102 | flurochloridone |
| B.103 | isoxaflutole |
| B.104 | mesotrione |
| B.105 | norflurazone |
| B.106 | picolinafen |
| B.107 | sulcotrione |
| B.108 | tefuryltrione |
| B.109 | tembotrione |
| B.110 | tolpyralate |
| B.111 | topramezone |
| B.112 | topramezone-sodium |
| B.113 | amitrole |
| B.114 | fluometuron |
| B.115 | fenquintrione |
| B.116 | glyphosate |
| B.117 | glyphosate-ammonium |
| B.118 | glyphosate- |
| | dimethylammonium |
| B.119 | glyphosate- |
| | isopropylammonium |
| B.120 | glyphosate-trimesium |
| | (sulfosate) |
| B.121 | glyphosate-potassium |
| B.122 | glufosinate |
| B.123 | glufosinate-ammonium |
| B.124 | glufosinate-P |
| B.125 | glufosinate-P-ammonium |
| B.126 | pendimethalin |
| B.127 | trifluralin |
| B.128 | acetochlor |
| B.129 | butachlor |
| B.130 | cafenstrole |
| B.131 | dimethenamid-P |
| B.132 | fentrazamide |
| B.133 | flufenacet |
| B.134 | mefenacet |
| B.135 | metazachlor |
| B.136 | metolachlor |
| B.137 | S-metolachlor |
| B.138 | pretilachlor |
| B.139 | fenoxasulfone |
| B.140 | indaziflam |
| B.141 | isoxaben |
| B.142 | triaziflam |
| B.143 | ipfencarbazone |
| B.144 | pyroxasulfone |
| B.145 | 2,4-D |
| B.146 | 2,4-D-isobutyl |
| B.147 | 2,4-D-dimethylammonium |
| B.148 | 2,4-D-N,N,N- |
| | trimethylethanolammonium |
| B.149 | aminopyralid |
| B.150 | aminopyralid-methyl |
| B.151 | aminopyralid-dimethyl- |
| | ammonium |
| B.152 | aminopyralid-tris(2- |
| | hydroxypropyl)ammonium |
| B.153 | clopyralid |
| B.154 | clopyralid-methyl |
| B.155 | clopyralid-olamine |
| B.156 | dicamba |
| B.157 | dicamba-butotyl |
| B.158 | dicamba-diglycolamine |
| B.159 | dicamba- |
| | dimethylammonium |
| B.160 | dicamba-diolamine |
| B.161 | dicamba- |
| | isopropylammonium |
| B.162 | dicamba-potassium |
| B.163 | dicamba-sodium |
| B.164 | dicamba-trolamine |
| B.165 | dicamba-N,N-bis-(3- |
| | aminopropyl)methylamine |
| B.166 | dicamba- |
| | diethylenetriamine |
| B.167 | fluroxypyr |
| B.168 | fluroxypyr-meptyl |
| B.169 | halauxifen |
| B.170 | halauxifen-methyl |
| B.171 | MCPA |
| B.172 | MCPA-2-ethylhexyl |
| B.173 | MCPA- |
| | dimethylammonium |
| B.174 | quinclorac |
| B.175 | quinclorac- |
| | dimethylammonium |
| B.176 | quinmerac |
| B.177 | quinmerac- |
| | dimethylammonium |
| B.178 | 4-amino-3-chloro-6-(4- |
| | chloro-2-fluoro-3- |
| | methoxyphenyl)-5- |
| | fluoropyridine-2-carboxylic acid |
| | (DOW, “Rinskor-acid”) |
| B.179 | benzyl 4-amino-3-chloro- |
| | 6-(4-chloro-2-fluoro-3-methoxy- |
| | phenyl)-5-fluoropyridine-2- |
| | carboxylate (CAS 1390661-72- |
| | 9) (DOW, “Rinskor”) |
| B.180 | aminocyclopyrachlor |
| B.181 | aminocyclopyrachlor- |
| | potassium |
| B.182 | aminocyclopyrachlor- |
| | methyl |
| B.183 | diflufenzopyr |
| B.184 | diflufenzopyr-sodium |
| B.185 | dymron |
| B.186 | indanofan |
| B.187 | oxaziclomefone |
| B.188 | II.1 |
| B.189 | II.2 |
| B.190 | II.3 |
| B.191 | II.4 |
| B.192 | II.5 |
| B.193 | II.6 |
| B.194 | II.7 |
| B.195 | II.8 |
| B.196 | II.9 |

In another embodiment of the present invention the compositions according to the present invention comprise at least one diaminotriazine compound of formula (I) and at least one safener C.

Safeners are chemical compounds which prevent or reduce damage on useful plants without having a major impact on the herbicidal action of the herbicidal active components of the present compositions towards unwanted plants. They can be applied either before sowings (e.g. on seed treatments, shoots or seedlings) or in the pre-emergence application or post-emergence application of the useful plant. The safeners and the diaminotriazine compound of formula (I) and/or the herbicides B can be applied simultaneously or in succession.

Suitable safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alphaoximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

Examples of preferred safeners C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and N-(2-Methoxybenzoyl)-4-[(methylaminocarbonyl)amino]benzenesulfonamide (CAS 129531-12-0).

Especially preferred safeners C are benoxacor, cloquintocet, cyprosulfamide, dichlormid, fenchlorazole, fenclorim, flurazole, fluxofenim, furilazole, isoxadifen, mefenpyr, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and N-(2-Methoxybenzoyl)-4-[(methylaminocarbonyl)amino]benzenesulfonamide (CAS 129531-12-0).

Particularly preferred safeners C are benoxacor, cloquintocet, cyprosulfamide, dichlormid, fenchlorazole, fenclorim, furilazole, isoxadifen, mefenpyr, naphthalic anhydride, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and N-(2-Methoxybenzoyl)-4-[(methylaminocarbonyl)amino]benzenesulfonamide (CAS 129531-12-0).

Particularly preferred safeners C, which, as component C, are constituent of the composition according to the invention are the safeners C as defined above; in particular the safeners C.1-C.17 listed below in table C:

TABLE C

| | Safener C |
|---|---|
| C.1 | benoxacor |
| C.2 | cloquintocet |
| C.3 | cloquintocet-mexyl |
| C.4 | cyprosulfamide |
| C.5 | dichlormid |
| C.6 | fenchlorazole |
| C.7 | fenchlorazole-ethyl |
| C.8 | fenclorim |
| C.9 | furilazole |
| C.10 | isoxadifen |
| C.11 | isoxadifen-ethyl |
| C.12 | mefenpyr |
| C.13 | mefenpyr-diethyl |
| C.14 | naphtalic acid anhydride |
| C.15 | 4-(dichloroacetyl)-1-oxa-4-azaspiro[4.5]decane |
| | (MON4660, CAS 71526-07-3) |
| C.16 | 2,2,5-trimethyl-3-(dichloroacetyl)-1,3-oxazolidine |
| | (R-29148, CAS 52836-31-4) |
| C.17 | N-(2-Methoxybenzoyl)-4- |
| | [(methylaminocarbonyl)amino]benzenesulfonamide |
| | (CAS 129531-12-0) |

The active compounds B of groups b1) to b15) and the active compounds C are known herbicides and safeners, see, for example, The Compendium of Pesticide Common Names (http://www.alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660.

The assignment of the active compounds to the respective mechanisms of action is based on current knowledge. If several mechanisms of action apply to one active compound, this substance was only assigned to one mechanism of action.

Active compounds B and C having a carboxyl group can be employed in the form of the acid, in the form of an agriculturally suitable salt as mentioned above or else in the form of an agriculturally acceptable derivative in the compositions according to the invention.

In the case of dicamba, suitable salts include those, where the counterion is an agriculturally acceptable cation. For example, suitable salts of dicamba are dicamba-sodium, dicamba-potassium, dicamba-methylammonium, dicamba-dimethylammonium, dicamba-isopropylammonium, dicamba-diglycolamine, dicamba-olamine, dicamba-diolamine, dicamba-trolamine, dicamba-N,N-bis-(3-aminopropyl)methylamine and dicamba-diethylenetriamine. Examples of a suitable ester are dicamba-methyl and dicamba-butotyl.

Suitable salts of 2,4-D are 2,4-D-ammonium, 2,4-D-dimethylammonium, 2,4-D-diethylammonium, 2,4-D-diethanolammonium (2,4-D-diolamine), 2,4-D-triethanolammonium, 2,4-D-isopropylammonium, 2,4-D-triisopropanolammonium, 2,4-D-heptylammonium, 2,4-D-dodecylammonium, 2,4-D-tetradecylammonium, 2,4-D-triethylammonium, 2,4-D-tris(2-hydroxypropyl)ammonium, 2,4-D-tris(isopropyl)ammonium, 2,4-D-trolamine, 2,4-D-lithium, 2,4-D-sodium. Examples of suitable esters of 2,4-D are 2,4-D-butotyl, 2,4-D-2-butoxypropyl, 2,4-D-3-butoxypropyl, 2,4-D-butyl, 2,4-D-ethyl, 2,4-D-ethylhexyl, 2,4-D-isobutyl, 2,4-D-isooctyl, 2,4-Disopropyl, 2,4-D-meptyl, 2,4-D-methyl, 2,4-D-octyl, 2,4-D-pentyl, 2,4-D-propyl, 2,4-D-tefuryl and clacyfos.

Suitable salts of 2,4-DB are for example 2,4-DB-sodium, 2,4-DB-potassium and 2,4-DB-dimethylammonium. Suitable esters of 2,4-DB are for example 2,4-DB-butyl and 2,4-DB-isoctyl.

Suitable salts of dichlorprop are for example dichlorprop-sodium, dichlorprop-potassium and dichlorprop-dimethylammonium. Examples of suitable esters of dichlorprop are dichlorprop-butotyl and dichlorprop-isoctyl.

Suitable salts and esters of MCPA include MCPA-butotyl, MCPA-butyl, MCPA-dimethylammonium, MCPA-diolamine, MCPA-ethyl, MCPA-thioethyl, MCPA-2-ethylhexyl, MCPA-isobutyl, MCPA-isoctyl, MCPA-isopropyl, MCPA-isopropylammonium, MCPA-methyl, MCPA-olamine, MCPA-potassium, MCPA-sodium and MCPA-trolamine.

A suitable salt of MCPB is MCPB sodium. A suitable ester of MCPB is MCPB-ethyl.

Suitable salts of clopyralid are clopyralid-potassium, clopyralid-olamine and clopyralid-tris(2-hydroxypropyl)ammonium. Example of suitable esters of clopyralid is clopyralid-methyl.

Examples of a suitable ester of fluroxypyr are fluroxypyr-meptyl and fluroxypyr-2-butoxy-1-methylethyl, wherein fluroxypyr-meptyl is preferred.

Suitable salts of picloram are picloram-dimethylammonium, picloram-potassium, picloram-triisopropanolammonium, picloram-triisopropylammonium and picloram-trolamine. A suitable ester of picloram is picloram-isoctyl.

A suitable salt of triclopyr is triclopyr-triethylammonium. Suitable esters of triclopyr are for example triclopyr-ethyl and triclopyr-butotyl.

Suitable salts and esters of chloramben include chloramben-ammonium, chloramben-diolamine, chloramben-methyl, chloramben-methylammonium and chloramben-sodium. Suitable salts and esters of 2,3,6-TBA include 2,3,6-TBA-dimethylammonium, 2,3,6-TBA-lithium, 2,3,6-TBA-potassium and 2,3,6-TBA-sodium.

Suitable salts and esters of aminopyralid include aminopyralid-potassium, aminopyralid-dimethylammonium, and aminopyralid-tris(2-hydroxypropyl)ammonium.

Suitable salts of glyphosate are for example glyphosate-ammonium, glyphosate-diammonium, glyphosate-dimethylammonium, glyphosate-isopropylammonium, glyphosate-potassium, glyphosate-sodium, glyphosate-trimesium as well as the ethanolamine and diethanolamine salts, preferably glyphosate-diammonium, glyphosate-isopropylammonium and glyphosate-trimesium (sulfosate).

A suitable salt of glufosinate is for example glufosinate-ammonium.

A suitable salt of glufosinate-P is for example glufosinate-P-ammonium.

Suitable salts and esters of bromoxynil are for example bromoxynil-butyrate, bromoxynil-heptanoate, bromoxynil-octanoate, bromoxynil-potassium and bromoxynil-sodium.

Suitable salts and esters of ioxonil are for example ioxonil-octanoate, ioxonil-potassium and ioxonil-sodium.

Suitable salts and esters of mecoprop include mecoprop-butotyl, mecoprop-dimethylammonium, mecoprop-diolamine, mecoprop-ethadyl, mecoprop-2-ethylhexyl, mecoprop-isoctyl, mecoprop-methyl, mecoprop-potassium, mecoprop-sodium and mecoprop-trolamine.

Suitable salts of mecoprop-P are for example mecoprop-P-butotyl, mecoprop-P-dimethylammonium, mecoprop-P-2-ethylhexyl, mecoprop-P-isobutyl, mecoprop-P-potassium and mecoprop-P-sodium.

A suitable salt of diflufenzopyr is for example diflufenzopyr-sodium.

A suitable salt of naptalam is for example naptalam-sodium.

Suitable salts and esters of aminocyclopyrachlor are for example aminocyclopyrachlor-dimethylammonium, aminocyclopyrachlor-methyl, aminocyclopyrachlor-triisopropanolammonium, aminocyclopyrachlor-sodium and aminocyclopyrachlor-potassium.

A suitable salt of quinclorac is for example quinclorac-dimethylammonium.

A suitable salt of quinmerac is for example quinmerac-dimethylammonium.

A suitable salt of imazamox is for example imazamox-ammonium.

Suitable salts of imazapic are for example imazapic-ammonium and imazapic-isopropylammonium.

Suitable salts of imazapyr are for example imazapyr-ammonium and imazapyr-isopropylammonium.

A suitable salt of imazaquin is for example imazaquin-ammonium.

Suitable salts of imazethapyr are for example imazethapyr-ammonium and imazethapyr-isopropylammonium.

A suitable salt of topramezone is for example topramezone-sodium.

According to a preferred embodiment of the invention, the composition comprises as herbicidal active compound B or component B at least one, preferably exactly one herbicide B.

According to another preferred embodiment of the invention, the composition comprises as herbicidal active compounds B or component B at least two, preferably exactly two herbicides B different from each other.

According to another preferred embodiment of the invention, the composition comprises as herbicidal active compounds B or component B at least three, preferably exactly three herbicides B different from each other.

According to another preferred embodiment of the invention, the composition comprises as safening component C or component C at least one, preferably exactly one safener C.

According to another preferred embodiment of the invention, the composition comprises as component B at least one, preferably exactly one herbicide B, and as component C at least one, preferably exactly one, safener C.

According to another preferred embodiment of the invention, the composition comprises at least two, preferably exactly two, herbicides B different from each other, and as component C at least one, preferably exactly one, safener C.

According to another preferred embodiment of the invention, the composition comprises at least three, preferably exactly three, herbicides B different from each other, and as component C at least one, preferably exactly one, safener C.

According to another preferred embodiment of the invention, the composition comprises at least one, preferably exactly one compound of formula (I) and as component B at least one, preferably exactly one, herbicide B.

According to another preferred embodiment of the invention, the composition comprises at least one, preferably exactly one compound of formula (I) and at least two, preferably exactly two, herbicides B different from each other.

According to another preferred embodiment of the invention, the composition comprises at least one, preferably exactly one compound of formula (I) and at least three, preferably exactly three, herbicides B different from each other.

According to another preferred embodiment of the invention, the composition comprises at least one, preferably exactly one compound of formula (I) and as component C at least one, preferably exactly one, safener C.

According to another preferred embodiment of the invention, the composition comprises at least one, preferably exactly one compound of formula (I) as component B at least one, preferably exactly one, herbicide B, and as component C at least one, preferably exactly one safener C.

According to another preferred embodiment of the invention, the composition comprises at least one, preferably exactly one compound of formula (I), at least two, preferably exactly two herbicides B different from each other, and as component C at least one, preferably exactly one, safener C.

According to another preferred embodiment of the invention, the composition comprises at least one, preferably exactly one compound of formula (I) at least three, preferably exactly three herbicides B different from each other, and as component C at least one, preferably exactly one, safener C.

According to another preferred embodiment of the invention, the composition comprises, in addition to a compounds of formula (I), at least one and especially exactly one herbicidally active compound from group b1), in particular selected from the group consisting of clodinafop-propargyl, cycloxydim, cyhalofop-butyl, fenoxaprop-P-ethyl, pinoxaden, profoxydim, tepraloxydim, tralkoxydim, esprocarb, prosulfocarb, thiobencarb and triallate.

According to another preferred embodiment of the invention, the composition comprises, in addition to a compounds of formula (I), at least one and especially exactly one herbicidally active compound from group b2), in particular selected from the group consisting of bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, mesosulfuron, metazosulfuron, nicosulfuron, penoxsulam, propoxycarbazon-sodium, pyrazosulfuron-ethyl, pyroxsulam, rimsulfuron, sulfosulfuron, thiencarbazon-methyl and tritosulfuron.

According to another preferred embodiment of the invention, the composition comprises, in addition to a compounds of formula (I), at least one and especially exactly one herbicidally active compound from group b3), in particular selected from the group consisting of ametryn, atrazine, diuron, fluometuron, hexazinone, isoproturon, linuron, metribuzin, paraquat, paraquat-dichloride, propanil, terbutryn and terbuthylazine.

According to another preferred embodiment of the invention, the composition comprises, in addition to a compounds of formula (I), at least one and especially exactly one herbicidally active compound from group b4), in particular selected from the group consisting of flumioxazin, oxyfluorfen, pyraflufen, pyraflufen-ethyl, saflufenacil, sulfentrazone, trifludimoxazin (BAS 850 H), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100; Sumitomo; LS 5296489), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7) LS 4061013), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0) (LS 567 0033=F2-Flumioxazin) and 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0) (LS 568 1323=Uracil-F2-PPO).

According to another preferred embodiment of the invention, the composition comprises, in addition to a compounds of formula (I), at least one and especially exactly one herbicidally active compound from group b5), in particular selected from the group consisting of amitrole, bicyclopyrone, clomazone, diflufenican, flumeturon, flurochloridone, isoxaflutole, mesotrione, picolinafen, sulcotrione, tefuryltrione, tembotrione, tolpyralate and topramezone.

According to another preferred embodiment of the invention, the composition comprises, in addition to a compounds of formula (I), at least one and especially exactly one herbicidally active compound from group b6), in particular selected from the group consisting of glyphosate, glyphosate-isopropylammonium and glyphosate-trimesium (sulfosate).

According to another preferred embodiment of the invention, the composition comprises, in addition to a compounds of formula (I), at least one and especially exactly one herbicidally active compound from group b7), in particular selected from the group consisting of glufosinate, glufosinate-P and glufosinate-ammonium.

According to another preferred embodiment of the invention, the composition comprises, in addition to a compounds of formula (I), at least one and especially exactly one herbicidally active compound from group b9), in particular selected from the group consisting of pendimethalin and trifluralin.

According to another preferred embodiment of the invention, the composition comprises, in addition to a compounds of formula (I), at least one and especially exactly one herbicidally active compound from group b10), in particular selected from the group consisting of acetochlor, cafenstrole, dimethenamid-P, fentrazamide, flufenacet, mefenacet, metazachlor, metolachlor, S-metolachlor, fenoxasulfone and pyroxasulfone. Likewise, preference is given to compositions comprising in addition to a compounds of formula (I), at least one and especially exactly one herbicidally active compound from group b10), in particular selected from the group consisting of isoxazoline compounds of the formulae II.1, II.2, II.3, II.4, II.5, II.6, II.7, II.8 and II.9, as defined above.

According to another preferred embodiment of the invention, the composition comprises, in addition to a compounds of formula (I), at least one and especially exactly one herbicidally active compound from group b11), in particular indaziflam, isoxaben and triaziflam.

According to another preferred embodiment of the invention, the composition comprises, in addition to a compounds of formula (I), at least one and especially exactly one herbicidally active compound from group b13), in particular selected from the group consisting of 2,4-D and its salts and esters, aminocyclopyrachlor and its salts and esters, aminopyralid and its salts such as aminopyralid-dimethylammonium, aminopyralid-tris(2-hydroxypropyl)ammonium and its esters, clopyralid and its salts and esters, dicamba and its salts and esters, fluroxypyr-meptyl, halauxifen, halauxifen-methyl, quinclorac, quinmerac, 4-amino-3-chloro-6-(4-chloro-2-fluoro-3-methoxyphenyl)-5-fluoropyridine-2-carboxylic acid (DOW, “Rinskor-acid”) and benzyl 4-amino-3-chloro-6-(4-chloro-2-fluoro-3-methoxyphenyl)-5-fluoropyridine-2-carboxylate (CAS 1390661-72-9) (DOW, “Rinskor”).

According to another preferred embodiment of the invention, the composition comprises, in addition to a compounds of formula (I), at least one and especially exactly one herbicidally active compound from group b14), in particular selected from the group consisting of diflufenzopyr and diflufenzopyr-sodium.

According to another preferred embodiment of the invention, the composition comprises, in addition to a compounds of formula (I), at least one and especially exactly one herbicidally active compound from group b15), in particular selected from the group consisting of dymron (=daimuron), indanofan and oxaziclomefone.

According to another preferred embodiment of the invention, the composition comprises, in addition to a compounds of formula (I), at least one and especially exactly one safener C, in particular selected from the group consisting of benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Further preferred embodiments relate to ternary compositions which correspond to the binary compositions mentioned above and additionally comprise a safener C, in particular selected from the group consisting of benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Here and below, the term “binary compositions” includes compositions comprising one or more, for example 1, 2 or 3, active compounds of the formula (I) and either one or more, for example 1, 2 or 3, herbicides B or one or more safeners C.

Correspondingly, the term “ternary compositions” includes compositions comprising one or more, for example 1, 2 or 3, active compounds of the formula (I), one or more, for example 1, 2 or 3, herbicides B and one or more, for example 1, 2 or 3, safeners C.

In binary compositions comprising at least one compound of the formula (I) as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

In binary compositions comprising at least one compound of the formula (I) as component A and at least one safener C, the weight ratio of the active compounds A:C is generally in the range of from 1:1000 to 1000:1, preferably in the range of from 1:500 to 500:1, in particular in the range of from 1:250 to 250:1 and particularly preferably in the range of from 1:75 to 75:1.

In ternary compositions comprising at least one compound of formula (I) as component A, at least one herbicide B and at least one safener C, the relative proportions by weight of the components A:B are generally in the range of from 1:1000 to 1000:1, preferably in the range of from 1:500 to 500:1, in particular in the range of from 1:250 to 250:1 and particularly preferably in the range of from 1:75 to 75:1, the weight ratio of the components A:C is generally in the range of from 1:1000 to 1000:1, preferably in the range of from 1:500 to 500:1, in particular in the range of from 1:250 to 250:1 and particularly preferably in the range of from 1:75 to 75:1, and the weight ratio of the components B:C is generally in the range of from 1:1000 to 1000:1, preferably in the range of from 1:500 to 500:1, in particular in the range of from 1:250 to 250:1 and particularly preferably in the range of from 1:75 to 75:1. The weight ratio of components A+B to component C is preferably in the range of from 1:500 to 500:1, in particular in the range of from 1:250 to 250:1 and particularly preferably in the range of from 1:75 to 75:1.

The weight ratios of the individual components in the preferred mixtures mentioned below are within the limits given above, in particular within the preferred limits.

Particularly preferred are the compositions mentioned below comprising the compounds of formula (I) as defined and the substance(s) as defined in the respective row of table 1;

especially preferred comprising as only herbicidal active compounds the compounds of formula (I) as defined and the substance(s) as defined in the respective row of table 1;

most preferably comprising as only active compounds the compounds of formula I as defined and the substance(s) as defined in the respective row of table 1.

Particularly preferred are compositions 1.1 to 1.3545, comprising the compounds of formula (I) and the substance(s) as defined in the respective row of table 1:

The following combinations indicated by the code A-X.Y.Z represent particular embodiments of the invention:

### A-1.1.1 to A-1.102.3545,

### A-2.1.1 to A-2.102.3545,

### A-3.1.1 to A-3.102.3545,

### A-4.1.1 to A-4.102.3545,

### A-5.1.1 to A-5.102.3545,

### A-6.1.1 to A-6.102.3545,

### A-7.1.1 to A-7.102.3545,

### A-8.1.1 to A-8.102.3545,

### A-9.1.1 to A-9.102.3545,

### A-10.1.1 to A-10.102.3545,

### A-11.1.1 to A-11.102.3545,

### A-12.1.1 to A-12.102.3545,

### A-13.1.1 to A-13.102.3545,

### A-14.1.1 to A-14.102.3545,

### A-15.1.1 to A-15.102.3545,

### A-16.1.1 to A-16.102.3545,

### A-17.1.1 to A-17.102.3545,

### A-18.1.1 to A-18.102.3545,

### A-19.1.1 to A-19.102.3545,

### A-20.1.1 to A-20.102.3545,

### A-21.1.1 to A-21.102.3545,

### A-22.1.1 to A-22.102.3545,

### A-23.1.1 to A-23.102.3545,

### A-24.1.1 to A-24.102.3545,

### A-25.1.1 to A-25.102.3545,

### A-26.1.1 to A-26.102.3545,

### A-27.1.1 to A-27.102.3545,

### A-28.1.1 to A-28.102.3545,

### A-29.1.1 to A-29.102.3545,

### A-30.1.1 to A-30.102.3545,

### A-31.1.1 to A-31.102.3545,

### A-32.1.1 to A-32.102.3545,

### A-33.1.1 to A-33.102.3545,

### A-34.1.1 to A-34.102.3545,

### A-35.1.1 to A-35.102.3545.

In the above codes A-X refers to the numbers of tables A-1 to A-35. The integer Y refers to the row of table A, while the integer Z refers to the row of table 1 below.

Hence, the code A-1.1.1 refers to the combination of the compound of formula I.a of table A-1, wherein R², R³, R⁴ are as defined in row 1 of table A, with the combination of the herbicide B and the safener C are as defined in combination no. 1.1 of table 1.

The code A-12.2.35 refers to the combination of the compound of formula I.a of table A-12, wherein R², R³, R⁴ are as defined in row 2 of table A, with the combination of the herbicide B and the safener C are as defined in combination no. 1.35 of table 1.

The code A-35.102.3545 refers to the combination of the compound of formula I.a of table A-35, wherein R², R³, R⁴ are as defined in row 102 of table A, with the combination of the herbicide B and the safener C are as defined in combination no. 1.3545 of table 1.

TABLE 1
(compositions 1.1 to 1.3545):

| comp. no. | herbicide B | safener C |
|---|---|---|
| 1.1 | B.1 | — |
| 1.2 | B.2 | — |
| 1.3 | B.3 | — |
| 1.4 | B.4 | — |
| 1.5 | B.5 | — |
| 1.6 | B.6 | — |
| 1.7 | B.7 | — |
| 1.8 | B.8 | — |
| 1.9 | B.9 | — |
| 1.10 | B.10 | — |
| 1.11 | B.11 | — |
| 1.12 | B.12 | — |
| 1.13 | B.13 | — |
| 1.14 | B.14 | — |
| 1.15 | B.15 | — |
| 1.16 | B.16 | — |
| 1.17 | B.17 | — |
| 1.18 | B.18 | — |
| 1.19 | B.19 | — |
| 1.20 | B.20 | — |
| 1.21 | B.21 | — |
| 1.22 | B.22 | — |
| 1.23 | B.23 | — |
| 1.24 | B.24 | — |
| 1.25 | B.25 | — |
| 1.26 | B.26 | — |
| 1.27 | B.27 | — |
| 1.28 | B.28 | — |
| 1.29 | B.29 | — |
| 1.30 | B.30 | — |
| 1.31 | B.31 | — |
| 1.32 | B.32 | — |
| 1.33 | B.33 | — |
| 1.34 | B.34 | — |
| 1.35 | B.35 | — |
| 1.36 | B.36 | — |
| 1.37 | B.37 | — |
| 1.38 | B.38 | — |
| 1.39 | B.39 | — |
| 1.40 | B.40 | — |
| 1.41 | B.41 | — |
| 1.42 | B.42 | — |
| 1.43 | B.43 | — |
| 1.44 | B.44 | — |
| 1.45 | B.45 | — |
| 1.46 | B.46 | — |
| 1.47 | B.47 | — |
| 1.48 | B.48 | — |
| 1.49 | B.49 | — |
| 1.50 | B.50 | — |
| 1.51 | B.51 | — |
| 1.52 | B.52 | — |
| 1.53 | B.53 | — |
| 1.54 | B.54 | — |
| 1.55 | B.55 | — |
| 1.56 | B.56 | — |
| 1.57 | B.57 | — |
| 1.58 | B.58. | — |
| 1.59 | B.59 | — |
| 1.60 | B.60 | — |
| 1.61 | B.61 | — |
| 1.62 | B.62 | — |
| 1.63 | B.63 | — |
| 1.64 | B.64 | — |
| 1.65 | B.65 | — |
| 1.66 | B.66 | — |
| 1.67 | B.67 | — |
| 1.68 | B.68 | — |
| 1.69 | B.69 | — |
| 1.70 | B.70 | — |
| 1.71 | B.71 | — |
| 1.72 | B.72 | — |
| 1.73 | B.73 | — |
| 1.74 | B.74 | — |
| 1.75 | B.75 | — |
| 1.76 | B.76 | — |
| 1.77 | B.77 | — |
| 1.78 | B.78 | — |
| 1.79 | B.79 | — |
| 1.80 | B.80 | — |
| 1.81 | B.81 | — |
| 1.82 | B.82 | — |
| 1.83 | B.83 | — |
| 1.84 | B.84 | — |
| 1.85 | B.85 | — |
| 1.86 | B.86 | — |
| 1.87 | B.87 | — |
| 1.88 | B.88 | — |
| 1.89 | B.89 | — |
| 1.90 | B.90 | — |
| 1.91 | B.91 | — |
| 1.92 | B.92 | — |
| 1.93 | B.93 | — |
| 1.94 | B.94 | — |
| 1.95 | B.95 | — |
| 1.96 | B.96 | — |
| 1.97 | B.97 | — |
| 1.98 | B.98 | — |
| 1.99 | B.99 | — |
| 1.100 | B.100 | — |
| 1.101 | B.101 | — |
| 1.102 | B.102 | — |
| 1.103 | B.103 | — |
| 1.104 | B.104 | — |
| 1.105 | B.105 | — |
| 1.106 | B.106 | — |
| 1.107 | B.107 | — |
| 1.108 | B.108 | — |
| 1.109 | B.109 | — |
| 1.110 | B.110 | — |
| 1.111 | B.111 | — |
| 1.112 | B.112 | — |
| 1.113 | B.113 | — |
| 1.114 | B.114 | — |
| 1.115 | B.115 | — |
| 1.116 | B.116 | — |
| 1.117 | B.117 | — |
| 1.118 | B.118 | — |
| 1.119 | B.119 | — |
| 1.120 | B.120 | — |
| 1.121 | B.121 | — |
| 1.122 | B.122 | — |
| 1.123 | B.123 | — |
| 1.124 | B.124 | — |
| 1.125 | B.125 | — |
| 1.126 | B.126 | — |
| 1.127 | B.127 | — |
| 1.128 | B.128 | — |
| 1.129 | B.129 | — |
| 1.130 | B.130 | — |
| 1.131 | B.131 | — |
| 1.132 | B.132 | — |
| 1.133 | B.133 | — |
| 1.134 | B.134 | — |
| 1.135 | B.135 | — |
| 1.136 | B.136 | — |
| 1.137 | B.137 | — |
| 1.138 | B.138 | — |
| 1.139 | B.139 | — |
| 1.140 | B.140 | — |
| 1.141 | B.141 | — |
| 1.142 | B.142 | — |
| 1.143 | B.143 | — |
| 1.144 | B.144 | — |
| 1.145 | B.145 | — |
| 1.146 | B.146 | — |
| 1.147 | B.147 | — |
| 1.148 | B.148 | — |
| 1.149 | B.149 | — |
| 1.150 | B.150 | — |
| 1.151 | B.151 | — |
| 1.152 | B.152 | — |
| 1.153 | B.153 | — |
| 1.154 | B.154 | — |
| 1.155 | B.155 | — |
| 1.156 | B.156 | — |
| 1.157 | B.157 | — |
| 1.158 | B.158 | — |
| 1.159 | B.159 | — |
| 1.160 | B.160 | — |
| 1.161 | B.161 | — |
| 1.162 | B.162 | — |
| 1.163 | B.163 | — |
| 1.164 | B.164 | — |
| 1.165 | B.165 | — |
| 1.166 | B.166 | — |
| 1.167 | B.167 | — |
| 1.168 | B.168 | — |
| 1.169 | B.169 | — |
| 1.170 | B.170 | — |
| 1.171 | B.171 | — |
| 1.172 | B.172 | — |
| 1.173 | B.173 | — |
| 1.174 | B.174 | — |
| 1.175 | B.175 | — |
| 1.176 | B.176 | — |
| 1.177 | B.177 | — |
| 1.178 | B.178 | — |
| 1.179 | B.179 | — |
| 1.180 | B.180 | — |
| 1.181 | B.181 | — |
| 1.182 | B.182 | — |
| 1.183 | B.183 | — |
| 1.184 | B.184 | — |
| 1.185 | B.185 | — |
| 1.186 | B.186 | — |
| 1.187 | B.187 | — |
| 1.188 | B.188 | — |
| 1.189 | B.189 | — |
| 1.190 | B.190 | |
| 1.191 | B.191 | |
| 1.192 | B.192 | |
| 1.193 | B.193 | |
| 1.194 | B.194 | |
| 1.195 | B.195 | |
| 1.196 | B.196 | |
| 1.197 | B.1 | C.1 |
| 1.198 | B.2 | C.1 |
| 1.199 | B.3 | C.1 |
| 1.200 | B.4 | C.1 |
| 1.201 | B.5 | C.1 |
| 1.202 | B.6 | C.1 |
| 1.203 | B.7 | C.1 |
| 1.204 | B.8 | C.1 |
| 1.205 | B.9 | C.1 |
| 1.206 | B.10 | C.1 |
| 1.207 | B.11 | C.1 |
| 1.208 | B.12 | C.1 |
| 1.209 | B.13 | C.1 |
| 1.210 | B.14 | C.1 |
| 1.211 | B.15 | C.1 |
| 1.212 | B.16 | C.1 |
| 1.213 | B.17 | C.1 |
| 1.214 | B.18 | C.1 |
| 1.215 | B.19 | C.1 |
| 1.216 | B.20 | C.1 |
| 1.217 | B.21 | C.1 |
| 1.218 | B.22 | C.1 |
| 1.219 | B.23 | C.1 |
| 1.220 | B.24 | C.1 |
| 1.221 | B.25 | C.1 |
| 1.222 | B.26 | C.1 |
| 1.223 | B.27 | C.1 |
| 1.224 | B.28 | C.1 |
| 1.225 | B.29 | C.1 |
| 1.226 | B.30 | C.1 |
| 1.227 | B.31 | C.1 |
| 1.228 | B.32 | C.1 |
| 1.229 | B.33 | C.1 |
| 1.230 | B.34 | C.1 |
| 1.231 | B.35 | C.1 |
| 1.232 | B.36 | C.1 |
| 1.233 | B.37 | C.1 |
| 1.234 | B.38 | C.1 |
| 1.235 | B.39 | C.1 |
| 1.236 | B.40 | C.1 |
| 1.237 | B.41 | C.1 |
| 1.238 | B.42 | C.1 |
| 1.239 | B.43 | C.1 |
| 1.240 | B.44 | C.1 |
| 1.241 | B.45 | C.1 |
| 1.242 | B.46 | C.1 |
| 1.243 | B.47 | C.1 |
| 1.244 | B.48 | C.1 |
| 1.245 | B.49 | C.1 |
| 1.246 | B.50 | C.1 |
| 1.247 | B.51 | C.1 |
| 1.248 | B.52 | C.1 |
| 1.249 | B.53 | C.1 |
| 1.250 | B.54 | C.1 |
| 1.251 | B.55 | C.1 |
| 1.252 | B.56 | C.1 |
| 1.253 | B.57 | C.1 |
| 1.254 | B.58. | C.1 |
| 1.255 | B.59 | C.1 |
| 1.256 | B.60 | C.1 |
| 1.257 | B.61 | C.1 |
| 1.258 | B.62 | C.1 |
| 1.259 | B.63 | C.1 |
| 1.260 | B.64 | C.1 |
| 1.261 | B.65 | C.1 |
| 1.262 | B.66 | C.1 |
| 1.263 | B.67 | C.1 |
| 1.264 | B.68 | C.1 |
| 1.265 | B.69 | C.1 |
| 1.266 | B.70 | C.1 |
| 1.267 | B.71 | C.1 |
| 1.268 | B.72 | C.1 |
| 1.269 | B.73 | C.1 |
| 1.270 | B.74 | C.1 |
| 1.271 | B.75 | C.1 |
| 1.272 | B.76 | C.1 |
| 1.273 | B.77 | C.1 |
| 1.274 | B.78 | C.1 |
| 1.275 | B.79 | C.1 |
| 1.276 | B.80 | C.1 |
| 1.277 | B.81 | C.1 |
| 1.278 | B.82 | C.1 |
| 1.279 | B.83 | C.1 |
| 1.280 | B.84 | C.1 |
| 1.281 | B.85 | C.1 |
| 1.282 | B.86 | C.1 |
| 1.283 | B.87 | C.1 |
| 1.284 | B.88 | C.1 |
| 1.285 | B.89 | C.1 |
| 1.286 | B.90 | C.1 |
| 1.287 | B.91 | C.1 |
| 1.288 | B.92 | C.1 |
| 1.289 | B.93 | C.1 |
| 1.290 | B.94 | C.1 |
| 1.291 | B.95 | C.1 |
| 1.292 | B.96 | C.1 |
| 1.293 | B.97 | C.1 |
| 1.294 | B.98 | C.1 |
| 1.295 | B.99 | C.1 |
| 1.296 | B.100 | C.1 |
| 1.297 | B.101 | C.1 |
| 1.298 | B.102 | C.1 |
| 1.299 | B.103 | C.1 |
| 1.300 | B.104 | C.1 |
| 1.301 | B.105 | C.1 |
| 1.302 | B.106 | C.1 |
| 1.303 | B.107 | C.1 |
| 1.304 | B.108 | C.1 |
| 1.305 | B.109 | C.1 |
| 1.306 | B.110 | C.1 |
| 1.307 | B.111 | C.1 |
| 1.308 | B.112 | C.1 |
| 1.309 | B.113 | C.1 |
| 1.310 | B.114 | C.1 |
| 1.311 | B.115 | C.1 |
| 1.312 | B.116 | C.1 |
| 1.313 | B.117 | C.1 |
| 1.314 | B.118 | C.1 |
| 1.315 | B.119 | C.1 |
| 1.316 | B.120 | C.1 |
| 1.317 | B.121 | C.1 |
| 1.318 | B.122 | C.1 |
| 1.319 | B.123 | C.1 |
| 1.320 | B.124 | C.1 |
| 1.321 | B.125 | C.1 |
| 1.322 | B.126 | C.1 |
| 1.323 | B.127 | C.1 |
| 1.324 | B.128 | C.1 |
| 1.325 | B.129 | C.1 |
| 1.326 | B.130 | C.1 |
| 1.327 | B.131 | C.1 |
| 1.328 | B.132 | C.1 |
| 1.329 | B.133 | C.1 |
| 1.330 | B.134 | C.1 |
| 1.331 | B.135 | C.1 |
| 1.332 | B.136 | C.1 |
| 1.333 | B.137 | C.1 |
| 1.334 | B.138 | C.1 |
| 1.335 | B.139 | C.1 |
| 1.336 | B.140 | C.1 |
| 1.337 | B.141 | C.1 |
| 1.338 | B.142 | C.1 |
| 1.339 | B.143 | C.1 |
| 1.340 | B.144 | C.1 |
| 1.341 | B.145 | C.1 |
| 1.342 | B.146 | C.1 |
| 1.343 | B.147 | C.1 |
| 1.344 | B.148 | C.1 |
| 1.345 | B.149 | C.1 |
| 1.346 | B.150 | C.1 |
| 1.347 | B.151 | C.1 |
| 1.348 | B.152 | C.1 |
| 1.349 | B.153 | C.1 |
| 1.350 | B.154 | C.1 |
| 1.351 | B.155 | C.1 |
| 1.352 | B.156 | C.1 |
| 1.353 | B.157 | C.1 |
| 1.354 | B.158 | C.1 |
| 1.355 | B.159 | C.1 |
| 1.356 | B.160 | C.1 |
| 1.357 | B.161 | C.1 |
| 1.358 | B.162 | C.1 |
| 1.359 | B.163 | C.1 |
| 1.360 | B.164 | C.1 |
| 1.361 | B.165 | C.1 |
| 1.362 | B.166 | C.1 |
| 1.363 | B.167 | C.1 |
| 1.364 | B.168 | C.1 |
| 1.365 | B.169 | C.1 |
| 1.366 | B.170 | C.1 |
| 1.367 | B.171 | C.1 |
| 1.368 | B.172 | C.1 |
| 1.369 | B.173 | C.1 |
| 1.370 | B.174 | C.1 |
| 1.371 | B.175 | C.1 |
| 1.372 | B.176 | C.1 |
| 1.373 | B.177 | C.1 |
| 1.374 | B.178 | C.1 |
| 1.375 | B.179 | C.1 |
| 1.376 | B.180 | C.1 |
| 1.377 | B.181 | C.1 |
| 1.378 | B.182 | C.1 |
| 1.379 | B.183 | C.1 |
| 1.380 | B.184 | C.1 |
| 1.381 | B.185 | C.1 |
| 1.382 | B.186 | C.1 |
| 1.383 | B.187 | C.1 |
| 1.384 | B.188 | C.1 |
| 1.385 | B.189 | C.1 |
| 1.386 | B.190 | C.1 |
| 1.387 | B.191 | C.1 |
| 1.388 | B.192 | C.1 |
| 1.389 | B.193 | C.1 |
| 1.390 | B.194 | C.1 |
| 1.391 | B.195 | C.1 |
| 1.392 | B.196 | C.1 |
| 1.393 | B.1 | C.2 |
| 1.394 | B.2 | C.2 |
| 1.395 | B.3 | C.2 |
| 1.396 | B.4 | C.2 |
| 1.397 | B.5 | C.2 |
| 1.398 | B.6 | C.2 |
| 1.399 | B.7 | C.2 |
| 1.400 | B.8 | C.2 |
| 1.401 | B.9 | C.2 |
| 1.402 | B.10 | C.2 |
| 1.403 | B.11 | C.2 |
| 1.404 | B.12 | C.2 |
| 1.405 | B.13 | C.2 |
| 1.406 | B.14 | C.2 |
| 1.407 | B.15 | C.2 |
| 1.408 | B.16 | C.2 |
| 1.409 | B.17 | C.2 |
| 1.410 | B.18 | C.2 |
| 1.411 | B.19 | C.2 |
| 1.412 | B.20 | C.2 |
| 1.413 | B.21 | C.2 |
| 1.414 | B.22 | C.2 |
| 1.415 | B.23 | C.2 |
| 1.416 | B.24 | C.2 |
| 1.417 | B.25 | C.2 |
| 1.418 | B.26 | C.2 |
| 1.419 | B.27 | C.2 |
| 1.420 | B.28 | C.2 |
| 1.421 | B.29 | C.2 |
| 1.422 | B.30 | C.2 |
| 1.423 | B.31 | C.2 |
| 1.424 | B.32 | C.2 |
| 1.425 | B.33 | C.2 |
| 1.426 | B.34 | C.2 |
| 1.427 | B.35 | C.2 |
| 1.428 | B.36 | C.2 |
| 1.429 | B.37 | C.2 |
| 1.430 | B.38 | C.2 |
| 1.431 | B.39 | C.2 |
| 1.432 | B.40 | C.2 |
| 1.433 | B.41 | C.2 |
| 1.434 | B.42 | C.2 |
| 1.435 | B.43 | C.2 |
| 1.436 | B.44 | C.2 |
| 1.437 | B.45 | C.2 |
| 1.438 | B.46 | C.2 |
| 1.439 | B.47 | C.2 |
| 1.440 | B.48 | C.2 |
| 1.441 | B.49 | C.2 |
| 1.442 | B.50 | C.2 |
| 1.443 | B.51 | C.2 |
| 1.444 | B.52 | C.2 |
| 1.445 | B.53 | C.2 |
| 1.446 | B.54 | C.2 |
| 1.447 | B.55 | C.2 |
| 1.448 | B.56 | C.2 |
| 1.449 | B.57 | C.2 |
| 1.450 | B.58. | C.2 |
| 1.451 | B.59 | C.2 |
| 1.452 | B.60 | C.2 |
| 1.453 | B.61 | C.2 |
| 1.454 | B.62 | C.2 |
| 1.455 | B.63 | C.2 |
| 1.456 | B.64 | C.2 |
| 1.457 | B.65 | C.2 |
| 1.458 | B.66 | C.2 |
| 1.459 | B.67 | C.2 |
| 1.460 | B.68 | C.2 |
| 1.461 | B.69 | C.2 |
| 1.462 | B.70 | C.2 |
| 1.463 | B.71 | C.2 |
| 1.464 | B.72 | C.2 |
| 1.465 | B.73 | C.2 |
| 1.466 | B.74 | C.2 |
| 1.467 | B.75 | C.2 |
| 1.468 | B.76 | C.2 |
| 1.469 | B.77 | C.2 |
| 1.470 | B.78 | C.2 |
| 1.471 | B.79 | C.2 |
| 1.472 | B.80 | C.2 |
| 1.473 | B.81 | C.2 |
| 1.474 | B.82 | C.2 |
| 1.475 | B.83 | C.2 |
| 1.476 | B.84 | C.2 |
| 1.477 | B.85 | C.2 |
| 1.478 | B.86 | C.2 |
| 1.479 | B.87 | C.2 |
| 1.480 | B.88 | C.2 |
| 1.481 | B.89 | C.2 |
| 1.482 | B.90 | C.2 |
| 1.483 | B.91 | C.2 |
| 1.484 | B.92 | C.2 |
| 1.485 | B.93 | C.2 |
| 1.486 | B.94 | C.2 |
| 1.487 | B.95 | C.2 |
| 1.488 | B.96 | C.2 |
| 1.489 | B.97 | C.2 |
| 1.490 | B.98 | C.2 |
| 1.491 | B.99 | C.2 |
| 1.492 | B.100 | C.2 |
| 1.493 | B.101 | C.2 |
| 1.494 | B.102 | C.2 |
| 1.495 | B.103 | C.2 |
| 1.496 | B.104 | C.2 |
| 1.497 | B.105 | C.2 |
| 1.498 | B.106 | C.2 |
| 1.499 | B.107 | C.2 |
| 1.500 | B.108 | C.2 |
| 1.501 | B.109 | C.2 |
| 1.502 | B.110 | C.2 |
| 1.503 | B.111 | C.2 |
| 1.504 | B.112 | C.2 |
| 1.505 | B.113 | C.2 |
| 1.506 | B.114 | C.2 |
| 1.507 | B.115 | C.2 |
| 1.508 | B.116 | C.2 |
| 1.509 | B.117 | C.2 |
| 1.510 | B.118 | C.2 |
| 1.511 | B.119 | C.2 |
| 1.512 | B.120 | C.2 |
| 1.513 | B.121 | C.2 |
| 1.514 | B.122 | C.2 |
| 1.515 | B.123 | C.2 |
| 1.516 | B.124 | C.2 |
| 1.517 | B.125 | C.2 |
| 1.518 | B.126 | C.2 |
| 1.519 | B.127 | C.2 |
| 1.520 | B.128 | C.2 |
| 1.521 | B.129 | C.2 |
| 1.522 | B.130 | C.2 |
| 1.523 | B.131 | C.2 |
| 1.524 | B.132 | C.2 |
| 1.525 | B.133 | C.2 |
| 1.526 | B.134 | C.2 |
| 1.527 | B.135 | C.2 |
| 1.528 | B.136 | C.2 |
| 1.529 | B.137 | C.2 |
| 1.530 | B.138 | C.2 |
| 1.531 | B.139 | C.2 |
| 1.532 | B.140 | C.2 |
| 1.533 | B.141 | C.2 |
| 1.534 | B.142 | C.2 |
| 1.535 | B.143 | C.2 |
| 1.536 | B.144 | C.2 |
| 1.537 | B.145 | C.2 |
| 1.538 | B.146 | C.2 |
| 1.539 | B.147 | C.2 |
| 1.540 | B.148 | C.2 |
| 1.541 | B.149 | C.2 |
| 1.542 | B.150 | C.2 |
| 1.543 | B.151 | C.2 |
| 1.544 | B.152 | C.2 |
| 1.545 | B.153 | C.2 |
| 1.546 | B.154 | C.2 |
| 1.547 | B.155 | C.2 |
| 1.548 | B.156 | C.2 |
| 1.549 | B.157 | C.2 |
| 1.550 | B.158 | C.2 |
| 1.551 | B.159 | C.2 |
| 1.552 | B.160 | C.2 |
| 1.553 | B.161 | C.2 |
| 1.554 | B.162 | C.2 |
| 1.555 | B.163 | C.2 |
| 1.556 | B.164 | C.2 |
| 1.557 | B.165 | C.2 |
| 1.558 | B.166 | C.2 |
| 1.559 | B.167 | C.2 |
| 1.560 | B.168 | C.2 |
| 1.561 | B.169 | C.2 |
| 1.562 | B.170 | C.2 |
| 1.563 | B.171 | C.2 |
| 1.564 | B.172 | C.2 |
| 1.565 | B.173 | C.2 |
| 1.566 | B.174 | C.2 |
| 1.567 | B.175 | C.2 |
| 1.568 | B.176 | C.2 |
| 1.569 | B.177 | C.2 |
| 1.570 | B.178 | C.2 |
| 1.571 | B.179 | C.2 |
| 1.572 | B.180 | C.2 |
| 1.573 | B.181 | C.2 |
| 1.574 | B.182 | C.2 |
| 1.575 | B.183 | C.2 |
| 1.576 | B.184 | C.2 |
| 1.577 | B.185 | C.2 |
| 1.578 | B.186 | C.2 |
| 1.579 | B.187 | C.2 |
| 1.580 | B.188 | C.2 |
| 1.581 | B.189 | C.2 |
| 1.582 | B.190 | C.2 |
| 1.583 | B.191 | C.2 |
| 1.584 | B.192 | C.2 |
| 1.585 | B.193 | C.2 |
| 1.586 | B.194 | C.2 |
| 1.587 | B.195 | C.2 |
| 1.588 | B.196 | C.2 |
| 1.589 | B.1 | C.3 |
| 1.590 | B.2 | C.3 |
| 1.591 | B.3 | C.3 |
| 1.592 | B.4 | C.3 |
| 1.593 | B.5 | C.3 |
| 1.594 | B.6 | C.3 |
| 1.595 | B.7 | C.3 |
| 1.596 | B.8 | C.3 |
| 1.597 | B.9 | C.3 |
| 1.598 | B.10 | C.3 |
| 1.599 | B.11 | C.3 |
| 1.600 | B.12 | C.3 |
| 1.601 | B.13 | C.3 |
| 1.602 | B.14 | C.3 |
| 1.603 | B.15 | C.3 |
| 1.604 | B.16 | C.3 |
| 1.605 | B.17 | C.3 |
| 1.606 | B.18 | C.3 |
| 1.607 | B.19 | C.3 |
| 1.608 | B.20 | C.3 |
| 1.609 | B.21 | C.3 |
| 1.610 | B.22 | C.3 |
| 1.611 | B.23 | C.3 |
| 1.612 | B.24 | C.3 |
| 1.613 | B.25 | C.3 |
| 1.614 | B.26 | C.3 |
| 1.615 | B.27 | C.3 |
| 1.616 | B.28 | C.3 |
| 1.617 | B.29 | C.3 |
| 1.618 | B.30 | C.3 |
| 1.619 | B.31 | C.3 |
| 1.620 | B.32 | C.3 |
| 1.621 | B.33 | C.3 |
| 1.622 | B.34 | C.3 |
| 1.623 | B.35 | C.3 |
| 1.624 | B.36 | C.3 |
| 1.625 | B.37 | C.3 |
| 1.626 | B.38 | C.3 |
| 1.627 | B.39 | C.3 |
| 1.628 | B.40 | C.3 |
| 1.629 | B.41 | C.3 |
| 1.630 | B.42 | C.3 |
| 1.631 | B.43 | C.3 |
| 1.632 | B.44 | C.3 |
| 1.633 | B.45 | C.3 |
| 1.634 | B.46 | C.3 |
| 1.635 | B.47 | C.3 |
| 1.636 | B.48 | C.3 |
| 1.637 | B.49 | C.3 |
| 1.638 | B.50 | C.3 |
| 1.639 | B.51 | C.3 |
| 1.640 | B.52 | C.3 |
| 1.641 | B.53 | C.3 |
| 1.642 | B.54 | C.3 |
| 1.643 | B.55 | C.3 |
| 1.644 | B.56 | C.3 |
| 1.645 | B.57 | C.3 |
| 1.646 | B.58. | C.3 |
| 1.647 | B.59 | C.3 |
| 1.648 | B.60 | C.3 |
| 1.649 | B.61 | C.3 |
| 1.650 | B.62 | C.3 |
| 1.651 | B.63 | C.3 |
| 1.652 | B.64 | C.3 |
| 1.653 | B.65 | C.3 |
| 1.654 | B.66 | C.3 |
| 1.655 | B.67 | C.3 |
| 1.656 | B.68 | C.3 |
| 1.657 | B.69 | C.3 |
| 1.658 | B.70 | C.3 |
| 1.659 | B.71 | C.3 |
| 1.660 | B.72 | C.3 |
| 1.661 | B.73 | C.3 |
| 1.662 | B.74 | C.3 |
| 1.663 | B.75 | C.3 |
| 1.664 | B.76 | C.3 |
| 1.665 | B.77 | C.3 |
| 1.666 | B.78 | C.3 |
| 1.667 | B.79 | C.3 |
| 1.668 | B.80 | C.3 |
| 1.669 | B.81 | C.3 |
| 1.670 | B.82 | C.3 |
| 1.671 | B.83 | C.3 |
| 1.672 | B.84 | C.3 |
| 1.673 | B.85 | C.3 |
| 1.674 | B.86 | C.3 |
| 1.675 | B.87 | C.3 |
| 1.676 | B.88 | C.3 |
| 1.677 | B.89 | C.3 |
| 1.678 | B.90 | C.3 |
| 1.679 | B.91 | C.3 |
| 1.680 | B.92 | C.3 |
| 1.681 | B.93 | C.3 |
| 1.682 | B.94 | C.3 |
| 1.683 | B.95 | C.3 |
| 1.684 | B.96 | C.3 |
| 1.685 | B.97 | C.3 |
| 1.686 | B.98 | C.3 |
| 1.687 | B.99 | C.3 |
| 1.688 | B.100 | C.3 |
| 1.689 | B.101 | C.3 |
| 1.690 | B.102 | C.3 |
| 1.691 | B.103 | C.3 |
| 1.692 | B.104 | C.3 |
| 1.693 | B.105 | C.3 |
| 1.694 | B.106 | C.3 |
| 1.695 | B.107 | C.3 |
| 1.696 | B.108 | C.3 |
| 1.697 | B.109 | C.3 |
| 1.698 | B.110 | C.3 |
| 1.699 | B.111 | C.3 |
| 1.700 | B.112 | C.3 |
| 1.701 | B.113 | C.3 |
| 1.702 | B.114 | C.3 |
| 1.703 | B.115 | C.3 |
| 1.704 | B.116 | C.3 |
| 1.705 | B.117 | C.3 |
| 1.706 | B.118 | C.3 |
| 1.707 | B.119 | C.3 |
| 1.708 | B.120 | C.3 |
| 1.709 | B.121 | C.3 |
| 1.710 | B.122 | C.3 |
| 1.711 | B.123 | C.3 |
| 1.712 | B.124 | C.3 |
| 1.713 | B.125 | C.3 |
| 1.714 | B.126 | C.3 |
| 1.715 | B.127 | C.3 |
| 1.716 | B.128 | C.3 |
| 1.717 | B.129 | C.3 |
| 1.718 | B.130 | C.3 |
| 1.719 | B.131 | C.3 |
| 1.720 | B.132 | C.3 |
| 1.721 | B.133 | C.3 |
| 1.722 | B.134 | C.3 |
| 1.723 | B.135 | C.3 |
| 1.724 | B.136 | C.3 |
| 1.725 | B.137 | C.3 |
| 1.726 | B.138 | C.3 |
| 1.727 | B.139 | C.3 |
| 1.728 | B.140 | C.3 |
| 1.729 | B.141 | C.3 |
| 1.730 | B.142 | C.3 |
| 1.731 | B.143 | C.3 |
| 1.732 | B.144 | C.3 |
| 1.733 | B.145 | C.3 |
| 1.734 | B.146 | C.3 |
| 1.735 | B.147 | C.3 |
| 1.736 | B.148 | C.3 |
| 1.737 | B.149 | C.3 |
| 1.738 | B.150 | C.3 |
| 1.739 | B.151 | C.3 |
| 1.740 | B.152 | C.3 |
| 1.741 | B.153 | C.3 |
| 1.742 | B.154 | C.3 |
| 1.743 | B.155 | C.3 |
| 1.744 | B.156 | C.3 |
| 1.745 | B.157 | C.3 |
| 1.746 | B.158 | C.3 |
| 1.747 | B.159 | C.3 |
| 1.748 | B.160 | C.3 |
| 1.749 | B.161 | C.3 |
| 1.750 | B.162 | C.3 |
| 1.751 | B.163 | C.3 |
| 1.752 | B.164 | C.3 |
| 1.753 | B.165 | C.3 |
| 1.754 | B.166 | C.3 |
| 1.755 | B.167 | C.3 |
| 1.756 | B.168 | C.3 |
| 1.757 | B.169 | C.3 |
| 1.758 | B.170 | C.3 |
| 1.759 | B.171 | C.3 |
| 1.760 | B.172 | C.3 |
| 1.761 | B.173 | C.3 |
| 1.762 | B.174 | C.3 |
| 1.763 | B.175 | C.3 |
| 1.764 | B.176 | C.3 |
| 1.765 | B.177 | C.3 |
| 1.766 | B.178 | C.3 |
| 1.767 | B.179 | C.3 |
| 1.768 | B.180 | C.3 |
| 1.769 | B.181 | C.3 |
| 1.770 | B.182 | C.3 |
| 1.771 | B.183 | C.3 |
| 1.772 | B.184 | C.3 |
| 1.773 | B.185 | C.3 |
| 1.774 | B.186 | C.3 |
| 1.775 | B.187 | C.3 |
| 1.776 | B.188 | C.3 |
| 1.777 | B.189 | C.3 |
| 1.778 | B.190 | C.3 |
| 1.779 | B.191 | C.3 |
| 1.780 | B.192 | C.3 |
| 1.781 | B.193 | C.3 |
| 1.782 | B.194 | C.3 |
| 1.783 | B.195 | C.3 |
| 1.784 | B.196 | C.3 |
| 1.785 | B.1 | C.4 |
| 1.786 | B.2 | C.4 |
| 1.787 | B.3 | C.4 |
| 1.788 | B.4 | C.4 |
| 1.789 | B.5 | C.4 |
| 1.790 | B.6 | C.4 |
| 1.791 | B.7 | C.4 |
| 1.792 | B.8 | C.4 |
| 1.793 | B.9 | C.4 |
| 1.794 | B.10 | C.4 |
| 1.795 | B.11 | C.4 |
| 1.796 | B.12 | C.4 |
| 1.797 | B.13 | C.4 |
| 1.798 | B.14 | C.4 |
| 1.799 | B.15 | C.4 |
| 1.800 | B.16 | C.4 |
| 1.801 | B.17 | C.4 |
| 1.802 | B.18 | C.4 |
| 1.803 | B.19 | C.4 |
| 1.804 | B.20 | C.4 |
| 1.805 | B.21 | C.4 |
| 1.806 | B.22 | C.4 |
| 1.807 | B.23 | C.4 |
| 1.808 | B.24 | C.4 |
| 1.809 | B.25 | C.4 |
| 1.810 | B.26 | C.4 |
| 1.811 | B.27 | C.4 |
| 1.812 | B.28 | C.4 |
| 1.813 | B.29 | C.4 |
| 1.814 | B.30 | C.4 |
| 1.815 | B.31 | C.4 |
| 1.816 | B.32 | C.4 |
| 1.817 | B.33 | C.4 |
| 1.818 | B.34 | C.4 |
| 1.819 | B.35 | C.4 |
| 1.820 | B.36 | C.4 |
| 1.821 | B.37 | C.4 |
| 1.822 | B.38 | C.4 |
| 1.823 | B.39 | C.4 |
| 1.824 | B.40 | C.4 |
| 1.825 | B.41 | C.4 |
| 1.826 | B.42 | C.4 |
| 1.827 | B.43 | C.4 |
| 1.828 | B.44 | C.4 |
| 1.829 | B.45 | C.4 |
| 1.830 | B.46 | C.4 |
| 1.831 | B.47 | C.4 |
| 1.832 | B.48 | C.4 |
| 1.833 | B.49 | C.4 |
| 1.834 | B.50 | C.4 |
| 1.835 | B.51 | C.4 |
| 1.836 | B.52 | C.4 |
| 1.837 | B.53 | C.4 |
| 1.838 | B.54 | C.4 |
| 1.839 | B.55 | C.4 |
| 1.840 | B.56 | C.4 |
| 1.841 | B.57 | C.4 |
| 1.842 | B.58. | C.4 |
| 1.843 | B.59 | C.4 |
| 1.844 | B.60 | C.4 |
| 1.845 | B.61 | C.4 |
| 1.846 | B.62 | C.4 |
| 1.847 | B.63 | C.4 |
| 1.848 | B.64 | C.4 |
| 1.849 | B.65 | C.4 |
| 1.850 | B.66 | C.4 |
| 1.851 | B.67 | C.4 |
| 1.852 | B.68 | C.4 |
| 1.853 | B.69 | C.4 |
| 1.854 | B.70 | C.4 |
| 1.855 | B.71 | C.4 |
| 1.856 | B.72 | C.4 |
| 1.857 | B.73 | C.4 |
| 1.858 | B.74 | C.4 |
| 1.859 | B.75 | C.4 |
| 1.860 | B.76 | C.4 |
| 1.861 | B.77 | C.4 |
| 1.862 | B.78 | C.4 |
| 1.863 | B.79 | C.4 |
| 1.864 | B.80 | C.4 |
| 1.865 | B.81 | C.4 |
| 1.866 | B.82 | C.4 |
| 1.867 | B.83 | C.4 |
| 1.868 | B.84 | C.4 |
| 1.869 | B.85 | C.4 |
| 1.870 | B.86 | C.4 |
| 1.871 | B.87 | C.4 |
| 1.872 | B.88 | C.4 |
| 1.873 | B.89 | C.4 |
| 1.874 | B.90 | C.4 |
| 1.875 | B.91 | C.4 |
| 1.876 | B.92 | C.4 |
| 1.877 | B.93 | C.4 |
| 1.878 | B.94 | C.4 |
| 1.879 | B.95 | C.4 |
| 1.880 | B.96 | C.4 |
| 1.881 | B.97 | C.4 |
| 1.882 | B.98 | C.4 |
| 1.883 | B.99 | C.4 |
| 1.884 | B.100 | C.4 |
| 1.885 | B.101 | C.4 |
| 1.886 | B.102 | C.4 |
| 1.887 | B.103 | C.4 |
| 1.888 | B.104 | C.4 |
| 1.889 | B.105 | C.4 |
| 1.890 | B.106 | C.4 |
| 1.891 | B.107 | C.4 |
| 1.892 | B.108 | C.4 |
| 1.893 | B.109 | C.4 |
| 1.894 | B.110 | C.4 |
| 1.895 | B.111 | C.4 |
| 1.896 | B.112 | C.4 |
| 1.897 | B.113 | C.4 |
| 1.898 | B.114 | C.4 |
| 1.899 | B.115 | C.4 |
| 1.900 | B.116 | C.4 |
| 1.901 | B.117 | C.4 |
| 1.902 | B.118 | C.4 |
| 1.903 | B.119 | C.4 |
| 1.904 | B.120 | C.4 |
| 1.905 | B.121 | C.4 |
| 1.906 | B.122 | C.4 |
| 1.907 | B.123 | C.4 |
| 1.908 | B.124 | C.4 |
| 1.909 | B.125 | C.4 |
| 1.910 | B.126 | C.4 |
| 1.911 | B.127 | C.4 |
| 1.912 | B.128 | C.4 |
| 1.913 | B.129 | C.4 |
| 1.914 | B.130 | C.4 |
| 1.915 | B.131 | C.4 |
| 1.916 | B.132 | C.4 |
| 1.917 | B.133 | C.4 |
| 1.918 | B.134 | C.4 |
| 1.919 | B.135 | C.4 |
| 1.920 | B.136 | C.4 |
| 1.921 | B.137 | C.4 |
| 1.922 | B.138 | C.4 |
| 1.923 | B.139 | C.4 |
| 1.924 | B.140 | C.4 |
| 1.925 | B.141 | C.4 |
| 1.926 | B.142 | C.4 |
| 1.927 | B.143 | C.4 |
| 1.928 | B.144 | C.4 |
| 1.929 | B.145 | C.4 |
| 1.930 | B.146 | C.4 |
| 1.931 | B.147 | C.4 |
| 1.932 | B.148 | C.4 |
| 1.933 | B.149 | C.4 |
| 1.934 | B.150 | C.4 |
| 1.935 | B.151 | C.4 |
| 1.936 | B.152 | C.4 |
| 1.937 | B.153 | C.4 |
| 1.938 | B.154 | C.4 |
| 1.939 | B.155 | C.4 |
| 1.940 | B.156 | C.4 |
| 1.941 | B.157 | C.4 |
| 1.942 | B.158 | C.4 |
| 1.943 | B.159 | C.4 |
| 1.944 | B.160 | C.4 |
| 1.945 | B.161 | C.4 |
| 1.946 | B.162 | C.4 |
| 1.947 | B.163 | C.4 |
| 1.948 | B.164 | C.4 |
| 1.949 | B.165 | C.4 |
| 1.950 | B.166 | C.4 |
| 1.951 | B.167 | C.4 |
| 1.952 | B.168 | C.4 |
| 1.953 | B.169 | C.4 |
| 1.954 | B.170 | C.4 |
| 1.955 | B.171 | C.4 |
| 1.956 | B.172 | C.4 |
| 1.957 | B.173 | C.4 |
| 1.958 | B.174 | C.4 |
| 1.959 | B.175 | C.4 |
| 1.960 | B.176 | C.4 |
| 1.961 | B.177 | C.4 |
| 1.962 | B.178 | C.4 |
| 1.963 | B.179 | C.4 |
| 1.964 | B.180 | C.4 |
| 1.965 | B.181 | C.4 |
| 1.966 | B.182 | C.4 |
| 1.967 | B.183 | C.4 |
| 1.968 | B.184 | C.4 |
| 1.969 | B.185 | C.4 |
| 1.970 | B.186 | C.4 |
| 1.971 | B.187 | C.4 |
| 1.972 | B.188 | C.4 |
| 1.973 | B.189 | C.4 |
| 1.974 | B.190 | C.4 |
| 1.975 | B.191 | C.4 |
| 1.976 | B.192 | C.4 |
| 1.977 | B.193 | C.4 |
| 1.978 | B.194 | C.4 |
| 1.979 | B.195 | C.4 |
| 1.980 | B.196 | C.4 |
| 1.981 | B.1 | C.5 |
| 1.982 | B.2 | C.5 |
| 1.983 | B.3 | C.5 |
| 1.984 | B.4 | C.5 |
| 1.985 | B.5 | C.5 |
| 1.986 | B.6 | C.5 |
| 1.987 | B.7 | C.5 |
| 1.988 | B.8 | C.5 |
| 1.989 | B.9 | C.5 |
| 1.990 | B.10 | C.5 |
| 1.991 | B.11 | C.5 |
| 1.992 | B.12 | C.5 |
| 1.993 | B.13 | C.5 |
| 1.994 | B.14 | C.5 |
| 1.995 | B.15 | C.5 |
| 1.996 | B.16 | C.5 |
| 1.997 | B.17 | C.5 |
| 1.998 | B.18 | C.5 |
| 1.999 | B.19 | C.5 |
| 1.1000 | B.20 | C.5 |
| 1.1001 | B.21 | C.5 |
| 1.1002 | B.22 | C.5 |
| 1.1003 | B.23 | C.5 |
| 1.1004 | B.24 | C.5 |
| 1.1005 | B.25 | C.5 |
| 1.1006 | B.26 | C.5 |
| 1.1007 | B.27 | C.5 |
| 1.1008 | B.28 | C.5 |
| 1.1009 | B.29 | C.5 |
| 1.1010 | B.30 | C.5 |
| 1.1011 | B.31 | C.5 |
| 1.1012 | B.32 | C.5 |
| 1.1013 | B.33 | C.5 |
| 1.1014 | B.34 | C.5 |
| 1.1015 | B.35 | C.5 |
| 1.1016 | B.36 | C.5 |
| 1.1017 | B.37 | C.5 |
| 1.1018 | B.38 | C.5 |
| 1.1019 | B.39 | C.5 |
| 1.1020 | B.40 | C.5 |
| 1.1021 | B.41 | C.5 |
| 1.1022 | B.42 | C.5 |
| 1.1023 | B.43 | C.5 |
| 1.1024 | B.44 | C.5 |
| 1.1025 | B.45 | C.5 |
| 1.1026 | B.46 | C.5 |
| 1.1027 | B.47 | C.5 |
| 1.1028 | B.48 | C.5 |
| 1.1029 | B.49 | C.5 |
| 1.1030 | B.50 | C.5 |
| 1.1031 | B.51 | C.5 |
| 1.1032 | B.52 | C.5 |
| 1.1033 | B.53 | C.5 |
| 1.1034 | B.54 | C.5 |
| 1.1035 | B.55 | C.5 |
| 1.1036 | B.56 | C.5 |
| 1.1037 | B.57 | C.5 |
| 1.1038 | B.58. | C.5 |
| 1.1039 | B.59 | C.5 |
| 1.1040 | B.60 | C.5 |
| 1.1041 | B.61 | C.5 |
| 1.1042 | B.62 | C.5 |
| 1.1043 | B.63 | C.5 |
| 1.1044 | B.64 | C.5 |
| 1.1045 | B.65 | C.5 |
| 1.1046 | B.66 | C.5 |
| 1.1047 | B.67 | C.5 |
| 1.1048 | B.68 | C.5 |
| 1.1049 | B.69 | C.5 |
| 1.1050 | B.70 | C.5 |
| 1.1051 | B.71 | C.5 |
| 1.1052 | B.72 | C.5 |
| 1.1053 | B.73 | C.5 |
| 1.1054 | B.74 | C.5 |
| 1.1055 | B.75 | C.5 |
| 1.1056 | B.76 | C.5 |
| 1.1057 | B.77 | C.5 |
| 1.1058 | B.78 | C.5 |
| 1.1059 | B.79 | C.5 |
| 1.1060 | B.80 | C.5 |
| 1.1061 | B.81 | C.5 |
| 1.1062 | B.82 | C.5 |
| 1.1063 | B.83 | C.5 |
| 1.1064 | B.84 | C.5 |
| 1.1065 | B.85 | C.5 |
| 1.1066 | B.86 | C.5 |
| 1.1067 | B.87 | C.5 |
| 1.1068 | B.88 | C.5 |
| 1.1069 | B.89 | C.5 |
| 1.1070 | B.90 | C.5 |
| 1.1071 | B.91 | C.5 |
| 1.1072 | B.92 | C.5 |
| 1.1073 | B.93 | C.5 |
| 1.1074 | B.94 | C.5 |
| 1.1075 | B.95 | C.5 |
| 1.1076 | B.96 | C.5 |
| 1.1077 | B.97 | C.5 |
| 1.1078 | B.98 | C.5 |
| 1.1079 | B.99 | C.5 |
| 1.1080 | B.100 | C.5 |
| 1.1081 | B.101 | C.5 |
| 1.1082 | B.102 | C.5 |
| 1.1083 | B.103 | C.5 |
| 1.1084 | B.104 | C.5 |
| 1.1085 | B.105 | C.5 |
| 1.1086 | B.106 | C.5 |
| 1.1087 | B.107 | C.5 |
| 1.1088 | B.108 | C.5 |
| 1.1089 | B.109 | C.5 |
| 1.1090 | B.110 | C.5 |
| 1.1091 | B.111 | C.5 |
| 1.1092 | B.112 | C.5 |
| 1.1093 | B.113 | C.5 |
| 1.1094 | B.114 | C.5 |
| 1.1095 | B.115 | C.5 |
| 1.1096 | B.116 | C.5 |
| 1.1097 | B.117 | C.5 |
| 1.1098 | B.118 | C.5 |
| 1.1099 | B.119 | C.5 |
| 1.1100 | B.120 | C.5 |
| 1.1101 | B.121 | C.5 |
| 1.1102 | B.122 | C.5 |
| 1.1103 | B.123 | C.5 |
| 1.1104 | B.124 | C.5 |
| 1.1105 | B.125 | C.5 |
| 1.1106 | B.126 | C.5 |
| 1.1107 | B.127 | C.5 |
| 1.1108 | B.128 | C.5 |
| 1.1109 | B.129 | C.5 |
| 1.1110 | B.130 | C.5 |
| 1.1111 | B.131 | C.5 |
| 1.1112 | B.132 | C.5 |
| 1.1113 | B.133 | C.5 |
| 1.1114 | B.134 | C.5 |
| 1.1115 | B.135 | C.5 |
| 1.1116 | B.136 | C.5 |
| 1.1117 | B.137 | C.5 |
| 1.1118 | B.138 | C.5 |
| 1.1119 | B.139 | C.5 |
| 1.1120 | B.140 | C.5 |
| 1.1121 | B.141 | C.5 |
| 1.1122 | B.142 | C.5 |
| 1.1123 | B.143 | C.5 |
| 1.1124 | B.144 | C.5 |
| 1.1125 | B.145 | C.5 |
| 1.1126 | B.146 | C.5 |
| 1.1127 | B.147 | C.5 |
| 1.1128 | B.148 | C.5 |
| 1.1129 | B.149 | C.5 |
| 1.1130 | B.150 | C.5 |
| 1.1131 | B.151 | C.5 |
| 1.1132 | B.152 | C.5 |
| 1.1133 | B.153 | C.5 |
| 1.1134 | B.154 | C.5 |
| 1.1135 | B.155 | C.5 |
| 1.1136 | B.156 | C.5 |
| 1.1137 | B.157 | C.5 |
| 1.1138 | B.158 | C.5 |
| 1.1139 | B.159 | C.5 |
| 1.1140 | B.160 | C.5 |
| 1.1141 | B.161 | C.5 |
| 1.1142 | B.162 | C.5 |
| 1.1143 | B.163 | C.5 |
| 1.1144 | B.164 | C.5 |
| 1.1145 | B.165 | C.5 |
| 1.1146 | B.166 | C.5 |
| 1.1147 | B.167 | C.5 |
| 1.1148 | B.168 | C.5 |
| 1.1149 | B.169 | C.5 |
| 1.1150 | B.170 | C.5 |
| 1.1151 | B.171 | C.5 |
| 1.1152 | B.172 | C.5 |
| 1.1153 | B.173 | C.5 |
| 1.1154 | B.174 | C.5 |
| 1.1155 | B.175 | C.5 |
| 1.1156 | B.176 | C.5 |
| 1.1157 | B.177 | C.5 |
| 1.1158 | B.178 | C.5 |
| 1.1159 | B.179 | C.5 |
| 1.1160 | B.180 | C.5 |
| 1.1161 | B.181 | C.5 |
| 1.1162 | B.182 | C.5 |
| 1.1163 | B.183 | C.5 |
| 1.1164 | B.184 | C.5 |
| 1.1165 | B.185 | C.5 |
| 1.1166 | B.186 | C.5 |
| 1.1167 | B.187 | C.5 |
| 1.1168 | B.188 | C.5 |
| 1.1169 | B.189 | C.5 |
| 1.1170 | B.190 | C.5 |
| 1.1171 | B.191 | C.5 |
| 1.1172 | B.192 | C.5 |
| 1.1173 | B.193 | C.5 |
| 1.1174 | B.194 | C.5 |
| 1.1175 | B.195 | C.5 |
| 1.1176 | B.196 | C.5 |
| 1.1177 | B.1 | C.6 |
| 1.1178 | B.2 | C.6 |
| 1.1179 | B.3 | C.6 |
| 1.1180 | B.4 | C.6 |
| 1.1181 | B.5 | C.6 |
| 1.1182 | B.6 | C.6 |
| 1.1183 | B.7 | C.6 |
| 1.1184 | B.8 | C.6 |
| 1.1185 | B.9 | C.6 |
| 1.1186 | B.10 | C.6 |
| 1.1187 | B.11 | C.6 |
| 1.1188 | B.12 | C.6 |
| 1.1189 | B.13 | C.6 |
| 1.1190 | B.14 | C.6 |
| 1.1191 | B.15 | C.6 |
| 1.1192 | B.16 | C.6 |
| 1.1193 | B.17 | C.6 |
| 1.1194 | B.18 | C.6 |
| 1.1195 | B.19 | C.6 |
| 1.1196 | B.20 | C.6 |
| 1.1197 | B.21 | C.6 |
| 1.1198 | B.22 | C.6 |
| 1.1199 | B.23 | C.6 |
| 1.1200 | B.24 | C.6 |
| 1.1201 | B.25 | C.6 |
| 1.1202 | B.26 | C.6 |
| 1.1203 | B.27 | C.6 |
| 1.1204 | B.28 | C.6 |
| 1.1205 | B.29 | C.6 |
| 1.1206 | B.30 | C.6 |
| 1.1207 | B.31 | C.6 |
| 1.1208 | B.32 | C.6 |
| 1.1209 | B.33 | C.6 |
| 1.1210 | B.34 | C.6 |
| 1.1211 | B.35 | C.6 |
| 1.1212 | B.36 | C.6 |
| 1.1213 | B.37 | C.6 |
| 1.1214 | B.38 | C.6 |
| 1.1215 | B.39 | C.6 |
| 1.1216 | B.40 | C.6 |
| 1.1217 | B.41 | C.6 |
| 1.1218 | B.42 | C.6 |
| 1.1219 | B.43 | C.6 |
| 1.1220 | B.44 | C.6 |
| 1.1221 | B.45 | C.6 |
| 1.1222 | B.46 | C.6 |
| 1.1223 | B.47 | C.6 |
| 1.1224 | B.48 | C.6 |
| 1.1225 | B.49 | C.6 |
| 1.1226 | B.50 | C.6 |
| 1.1227 | B.51 | C.6 |
| 1.1228 | B.52 | C.6 |
| 1.1229 | B.53 | C.6 |
| 1.1230 | B.54 | C.6 |
| 1.1231 | B.55 | C.6 |
| 1.1232 | B.56 | C.6 |
| 1.1233 | B.57 | C.6 |
| 1.1234 | B.58. | C.6 |
| 1.1235 | B.59 | C.6 |
| 1.1236 | B.60 | C.6 |
| 1.1237 | B.61 | C.6 |
| 1.1238 | B.62 | C.6 |
| 1.1239 | B.63 | C.6 |
| 1.1240 | B.64 | C.6 |
| 1.1241 | B.65 | C.6 |
| 1.1242 | B.66 | C.6 |
| 1.1243 | B.67 | C.6 |
| 1.1244 | B.68 | C.6 |
| 1.1245 | B.69 | C.6 |
| 1.1246 | B.70 | C.6 |
| 1.1247 | B.71 | C.6 |
| 1.1248 | B.72 | C.6 |
| 1.1249 | B.73 | C.6 |
| 1.1250 | B.74 | C.6 |
| 1.1251 | B.75 | C.6 |
| 1.1252 | B.76 | C.6 |
| 1.1253 | B.77 | C.6 |
| 1.1254 | B.78 | C.6 |
| 1.1255 | B.79 | C.6 |
| 1.1256 | B.80 | C.6 |
| 1.1257 | B.81 | C.6 |
| 1.1258 | B.82 | C.6 |
| 1.1259 | B.83 | C.6 |
| 1.1260 | B.84 | C.6 |
| 1.1261 | B.85 | C.6 |
| 1.1262 | B.86 | C.6 |
| 1.1263 | B.87 | C.6 |
| 1.1264 | B.88 | C.6 |
| 1.1265 | B.89 | C.6 |
| 1.1266 | B.90 | C.6 |
| 1.1267 | B.91 | C.6 |
| 1.1268 | B.92 | C.6 |
| 1.1269 | B.93 | C.6 |
| 1.1270 | B.94 | C.6 |
| 1.1271 | B.95 | C.6 |
| 1.1272 | B.96 | C.6 |
| 1.1273 | B.97 | C.6 |
| 1.1274 | B.98 | C.6 |
| 1.1275 | B.99 | C.6 |
| 1.1276 | B.100 | C.6 |
| 1.1277 | B.101 | C.6 |
| 1.1278 | B.102 | C.6 |
| 1.1279 | B.103 | C.6 |
| 1.1280 | B.104 | C.6 |
| 1.1281 | B.105 | C.6 |
| 1.1282 | B.106 | C.6 |
| 1.1283 | B.107 | C.6 |
| 1.1284 | B.108 | C.6 |
| 1.1285 | B.109 | C.6 |
| 1.1286 | B.110 | C.6 |
| 1.1287 | B.111 | C.6 |
| 1.1288 | B.112 | C.6 |
| 1.1289 | B.113 | C.6 |
| 1.1290 | B.114 | C.6 |
| 1.1291 | B.115 | C.6 |
| 1.1292 | B.116 | C.6 |
| 1.1293 | B.117 | C.6 |
| 1.1294 | B.118 | C.6 |
| 1.1295 | B.119 | C.6 |
| 1.1296 | B.120 | C.6 |
| 1.1297 | B.121 | C.6 |
| 1.1298 | B.122 | C.6 |
| 1.1299 | B.123 | C.6 |
| 1.1300 | B.124 | C.6 |
| 1.1301 | B.125 | C.6 |
| 1.1302 | B.126 | C.6 |
| 1.1303 | B.127 | C.6 |
| 1.1304 | B.128 | C.6 |
| 1.1305 | B.129 | C.6 |
| 1.1306 | B.130 | C.6 |
| 1.1307 | B.131 | C.6 |
| 1.1308 | B.132 | C.6 |
| 1.1309 | B.133 | C.6 |
| 1.1310 | B.134 | C.6 |
| 1.1311 | B.135 | C.6 |
| 1.1312 | B.136 | C.6 |
| 1.1313 | B.137 | C.6 |
| 1.1314 | B.138 | C.6 |
| 1.1315 | B.139 | C.6 |
| 1.1316 | B.140 | C.6 |
| 1.1317 | B.141 | C.6 |
| 1.1318 | B.142 | C.6 |
| 1.1319 | B.143 | C.6 |
| 1.1320 | B.144 | C.6 |
| 1.1321 | B.145 | C.6 |
| 1.1322 | B.146 | C.6 |
| 1.1323 | B.147 | C.6 |
| 1.1324 | B.148 | C.6 |
| 1.1325 | B.149 | C.6 |
| 1.1326 | B.150 | C.6 |
| 1.1327 | B.151 | C.6 |
| 1.1328 | B.152 | C.6 |
| 1.1329 | B.153 | C.6 |
| 1.1330 | B.154 | C.6 |
| 1.1331 | B.155 | C.6 |
| 1.1332 | B.156 | C.6 |
| 1.1333 | B.157 | C.6 |
| 1.1334 | B.158 | C.6 |
| 1.1335 | B.159 | C.6 |
| 1.1336 | B.160 | C.6 |
| 1.1337 | B.161 | C.6 |
| 1.1338 | B.162 | C.6 |
| 1.1339 | B.163 | C.6 |
| 1.1340 | B.164 | C.6 |
| 1.1341 | B.165 | C.6 |
| 1.1342 | B.166 | C.6 |
| 1.1343 | B.167 | C.6 |
| 1.1344 | B.168 | C.6 |
| 1.1345 | B.169 | C.6 |
| 1.1346 | B.170 | C.6 |
| 1.1347 | B.171 | C.6 |
| 1.1348 | B.172 | C.6 |
| 1.1349 | B.173 | C.6 |
| 1.1350 | B.174 | C.6 |
| 1.1351 | B.175 | C.6 |
| 1.1352 | B.176 | C.6 |
| 1.1353 | B.177 | C.6 |
| 1.1354 | B.178 | C.6 |
| 1.1355 | B.179 | C.6 |
| 1.1356 | B.180 | C.6 |
| 1.1357 | B.181 | C.6 |
| 1.1358 | B.182 | C.6 |
| 1.1359 | B.183 | C.6 |
| 1.1360 | B.184 | C.6 |
| 1.1361 | B.185 | C.6 |
| 1.1362 | B.186 | C.6 |
| 1.1363 | B.187 | C.6 |
| 1.1364 | B.188 | C.6 |
| 1.1365 | B.189 | C.6 |
| 1.1366 | B.190 | C.6 |
| 1.1367 | B.191 | C.6 |
| 1.1368 | B.192 | C.6 |
| 1.1369 | B.193 | C.6 |
| 1.1370 | B.194 | C.6 |
| 1.1371 | B.195 | C.6 |
| 1.1372 | B.196 | C.6 |
| 1.1373 | B.1 | C.7 |
| 1.1374 | B.2 | C.7 |
| 1.1375 | B.3 | C.7 |
| 1.1376 | B.4 | C.7 |
| 1.1377 | B.5 | C.7 |
| 1.1378 | B.6 | C.7 |
| 1.1379 | B.7 | C.7 |
| 1.1380 | B.8 | C.7 |
| 1.1381 | B.9 | C.7 |
| 1.1382 | B.10 | C.7 |
| 1.1383 | B.11 | C.7 |
| 1.1384 | B.12 | C.7 |
| 1.1385 | B.13 | C.7 |
| 1.1386 | B.14 | C.7 |
| 1.1387 | B.15 | C.7 |
| 1.1388 | B.16 | C.7 |
| 1.1389 | B.17 | C.7 |
| 1.1390 | B.18 | C.7 |
| 1.1391 | B.19 | C.7 |
| 1.1392 | B.20 | C.7 |
| 1.1393 | B.21 | C.7 |
| 1.1394 | B.22 | C.7 |
| 1.1395 | B.23 | C.7 |
| 1.1396 | B.24 | C.7 |
| 1.1397 | B.25 | C.7 |
| 1.1398 | B.26 | C.7 |
| 1.1399 | B.27 | C.7 |
| 1.1400 | B.28 | C.7 |
| 1.1401 | B.29 | C.7 |
| 1.1402 | B.30 | C.7 |
| 1.1403 | B.31 | C.7 |
| 1.1404 | B.32 | C.7 |
| 1.1405 | B.33 | C.7 |
| 1.1406 | B.34 | C.7 |
| 1.1407 | B.35 | C.7 |
| 1.1408 | B.36 | C.7 |
| 1.1409 | B.37 | C.7 |
| 1.1410 | B.38 | C.7 |
| 1.1411 | B.39 | C.7 |
| 1.1412 | B.40 | C.7 |
| 1.1413 | B.41 | C.7 |
| 1.1414 | B.42 | C.7 |
| 1.1415 | B.43 | C.7 |
| 1.1416 | B.44 | C.7 |
| 1.1417 | B.45 | C.7 |
| 1.1418 | B.46 | C.7 |
| 1.1419 | B.47 | C.7 |
| 1.1420 | B.48 | C.7 |
| 1.1421 | B.49 | C.7 |
| 1.1422 | B.50 | C.7 |
| 1.1423 | B.51 | C.7 |
| 1.1424 | B.52 | C.7 |
| 1.1425 | B.53 | C.7 |
| 1.1426 | B.54 | C.7 |
| 1.1427 | B.55 | C.7 |
| 1.1428 | B.56 | C.7 |
| 1.1429 | B.57 | C.7 |
| 1.1430 | B.58. | C.7 |
| 1.1431 | B.59 | C.7 |
| 1.1432 | B.60 | C.7 |
| 1.1433 | B.61 | C.7 |
| 1.1434 | B.62 | C.7 |
| 1.1435 | B.63 | C.7 |
| 1.1436 | B.64 | C.7 |
| 1.1437 | B.65 | C.7 |
| 1.1438 | B.66 | C.7 |
| 1.1439 | B.67 | C.7 |
| 1.1440 | B.68 | C.7 |
| 1.1441 | B.69 | C.7 |
| 1.1442 | B.70 | C.7 |
| 1.1443 | B.71 | C.7 |
| 1.1444 | B.72 | C.7 |
| 1.1445 | B.73 | C.7 |
| 1.1446 | B.74 | C.7 |
| 1.1447 | B.75 | C.7 |
| 1.1448 | B.76 | C.7 |
| 1.1449 | B.77 | C.7 |
| 1.1450 | B.78 | C.7 |
| 1.1451 | B.79 | C.7 |
| 1.1452 | B.80 | C.7 |
| 1.1453 | B.81 | C.7 |
| 1.1454 | B.82 | C.7 |
| 1.1455 | B.83 | C.7 |
| 1.1456 | B.84 | C.7 |
| 1.1457 | B.85 | C.7 |
| 1.1458 | B.86 | C.7 |
| 1.1459 | B.87 | C.7 |
| 1.1460 | B.88 | C.7 |
| 1.1461 | B.89 | C.7 |
| 1.1462 | B.90 | C.7 |
| 1.1463 | B.91 | C.7 |
| 1.1464 | B.92 | C.7 |
| 1.1465 | B.93 | C.7 |
| 1.1466 | B.94 | C.7 |
| 1.1467 | B.95 | C.7 |
| 1.1468 | B.96 | C.7 |
| 1.1469 | B.97 | C.7 |
| 1.1470 | B.98 | C.7 |
| 1.1471 | B.99 | C.7 |
| 1.1472 | B.100 | C.7 |
| 1.1473 | B.101 | C.7 |
| 1.1474 | B.102 | C.7 |
| 1.1475 | B.103 | C.7 |
| 1.1476 | B.104 | C.7 |
| 1.1477 | B.105 | C.7 |
| 1.1478 | B.106 | C.7 |
| 1.1479 | B.107 | C.7 |
| 1.1480 | B.108 | C.7 |
| 1.1481 | B.109 | C.7 |
| 1.1482 | B.110 | C.7 |
| 1.1483 | B.111 | C.7 |
| 1.1484 | B.112 | C.7 |
| 1.1485 | B.113 | C.7 |
| 1.1486 | B.114 | C.7 |
| 1.1487 | B.115 | C.7 |
| 1.1488 | B.116 | C.7 |
| 1.1489 | B.117 | C.7 |
| 1.1490 | B.118 | C.7 |
| 1.1491 | B.119 | C.7 |
| 1.1492 | B.120 | C.7 |
| 1.1493 | B.121 | C.7 |
| 1.1494 | B.122 | C.7 |
| 1.1495 | B.123 | C.7 |
| 1.1496 | B.124 | C.7 |
| 1.1497 | B.125 | C.7 |
| 1.1498 | B.126 | C.7 |
| 1.1499 | B.127 | C.7 |
| 1.1500 | B.128 | C.7 |
| 1.1501 | B.129 | C.7 |
| 1.1502 | B.130 | C.7 |
| 1.1503 | B.131 | C.7 |
| 1.1504 | B.132 | C.7 |
| 1.1505 | B.133 | C.7 |
| 1.1506 | B.134 | C.7 |
| 1.1507 | B.135 | C.7 |
| 1.1508 | B.136 | C.7 |
| 1.1509 | B.137 | C.7 |
| 1.1510 | B.138 | C.7 |
| 1.1511 | B.139 | C.7 |
| 1.1512 | B.140 | C.7 |
| 1.1513 | B.141 | C.7 |
| 1.1514 | B.142 | C.7 |
| 1.1515 | B.143 | C.7 |
| 1.1516 | B.144 | C.7 |
| 1.1517 | B.145 | C.7 |
| 1.1518 | B.146 | C.7 |
| 1.1519 | B.147 | C.7 |
| 1.1520 | B.148 | C.7 |
| 1.1521 | B.149 | C.7 |
| 1.1522 | B.150 | C.7 |
| 1.1523 | B.151 | C.7 |
| 1.1524 | B.152 | C.7 |
| 1.1525 | B.153 | C.7 |
| 1.1526 | B.154 | C.7 |
| 1.1527 | B.155 | C.7 |
| 1.1528 | B.156 | C.7 |
| 1.1529 | B.157 | C.7 |
| 1.1530 | B.158 | C.7 |
| 1.1531 | B.159 | C.7 |
| 1.1532 | B.160 | C.7 |
| 1.1533 | B.161 | C.7 |
| 1.1534 | B.162 | C.7 |
| 1.1535 | B.163 | C.7 |
| 1.1536 | B.164 | C.7 |
| 1.1537 | B.165 | C.7 |
| 1.1538 | B.166 | C.7 |
| 1.1539 | B.167 | C.7 |
| 1.1540 | B.168 | C.7 |
| 1.1541 | B.169 | C.7 |
| 1.1542 | B.170 | C.7 |
| 1.1543 | B.171 | C.7 |
| 1.1544 | B.172 | C.7 |
| 1.1545 | B.173 | C.7 |
| 1.1546 | B.174 | C.7 |
| 1.1547 | B.175 | C.7 |
| 1.1548 | B.176 | C.7 |
| 1.1549 | B.177 | C.7 |
| 1.1550 | B.178 | C.7 |
| 1.1551 | B.179 | C.7 |
| 1.1552 | B.180 | C.7 |
| 1.1553 | B.181 | C.7 |
| 1.1554 | B.182 | C.7 |
| 1.1555 | B.183 | C.7 |
| 1.1556 | B.184 | C.7 |
| 1.1557 | B.185 | C.7 |
| 1.1558 | B.186 | C.7 |
| 1.1559 | B.187 | C.7 |
| 1.1560 | B.188 | C.7 |
| 1.1561 | B.189 | C.7 |
| 1.1562 | B.190 | C.7 |
| 1.1563 | B.191 | C.7 |
| 1.1564 | B.192 | C.7 |
| 1.1565 | B.193 | C.7 |
| 1.1566 | B.194 | C.7 |
| 1.1567 | B.195 | C.7 |
| 1.1568 | B.196 | C.7 |
| 1.1569 | B.1 | C.8 |
| 1.1570 | B.2 | C.8 |
| 1.1571 | B.3 | C.8 |
| 1.1572 | B.4 | C.8 |
| 1.1573 | B.5 | C.8 |
| 1.1574 | B.6 | C.8 |
| 1.1575 | B.7 | C.8 |
| 1.1576 | B.8 | C.8 |
| 1.1577 | B.9 | C.8 |
| 1.1578 | B.10 | C.8 |
| 1.1579 | B.11 | C.8 |
| 1.1580 | B.12 | C.8 |
| 1.1581 | B.13 | C.8 |
| 1.1582 | B.14 | C.8 |
| 1.1583 | B.15 | C.8 |
| 1.1584 | B.16 | C.8 |
| 1.1585 | B.17 | C.8 |
| 1.1586 | B.18 | C.8 |
| 1.1587 | B.19 | C.8 |
| 1.1588 | B.20 | C.8 |
| 1.1589 | B.21 | C.8 |
| 1.1590 | B.22 | C.8 |
| 1.1591 | B.23 | C.8 |
| 1.1592 | B.24 | C.8 |
| 1.1593 | B.25 | C.8 |
| 1.1594 | B.26 | C.8 |
| 1.1595 | B.27 | C.8 |
| 1.1596 | B.28 | C.8 |
| 1.1597 | B.29 | C.8 |
| 1.1598 | B.30 | C.8 |
| 1.1599 | B.31 | C.8 |
| 1.1600 | B.32 | C.8 |
| 1.1601 | B.33 | C.8 |
| 1.1602 | B.34 | C.8 |
| 1.1603 | B.35 | C.8 |
| 1.1604 | B.36 | C.8 |
| 1.1605 | B.37 | C.8 |
| 1.1606 | B.38 | C.8 |
| 1.1607 | B.39 | C.8 |
| 1.1608 | B.40 | C.8 |
| 1.1609 | B.41 | C.8 |
| 1.1610 | B.42 | C.8 |
| 1.1611 | B.43 | C.8 |
| 1.1612 | B.44 | C.8 |
| 1.1613 | B.45 | C.8 |
| 1.1614 | B.46 | C.8 |
| 1.1615 | B.47 | C.8 |
| 1.1616 | B.48 | C.8 |
| 1.1617 | B.49 | C.8 |
| 1.1618 | B.50 | C.8 |
| 1.1619 | B.51 | C.8 |
| 1.1620 | B.52 | C.8 |
| 1.1621 | B.53 | C.8 |
| 1.1622 | B.54 | C.8 |
| 1.1623 | B.55 | C.8 |
| 1.1624 | B.56 | C.8 |
| 1.1625 | B.57 | C.8 |
| 1.1626 | B.58. | C.8 |
| 1.1627 | B.59 | C.8 |
| 1.1628 | B.60 | C.8 |
| 1.1629 | B.61 | C.8 |
| 1.1630 | B.62 | C.8 |
| 1.1631 | B.63 | C.8 |
| 1.1632 | B.64 | C.8 |
| 1.1633 | B.65 | C.8 |
| 1.1634 | B.66 | C.8 |
| 1.1635 | B.67 | C.8 |
| 1.1636 | B.68 | C.8 |
| 1.1637 | B.69 | C.8 |
| 1.1638 | B.70 | C.8 |
| 1.1639 | B.71 | C.8 |
| 1.1640 | B.72 | C.8 |
| 1.1641 | B.73 | C.8 |
| 1.1642 | B.74 | C.8 |
| 1.1643 | B.75 | C.8 |
| 1.1644 | B.76 | C.8 |
| 1.1645 | B.77 | C.8 |
| 1.1646 | B.78 | C.8 |
| 1.1647 | B.79 | C.8 |
| 1.1648 | B.80 | C.8 |
| 1.1649 | B.81 | C.8 |
| 1.1650 | B.82 | C.8 |
| 1.1651 | B.83 | C.8 |
| 1.1652 | B.84 | C.8 |
| 1.1653 | B.85 | C.8 |
| 1.1654 | B.86 | C.8 |
| 1.1655 | B.87 | C.8 |
| 1.1656 | B.88 | C.8 |
| 1.1657 | B.89 | C.8 |
| 1.1658 | B.90 | C.8 |
| 1.1659 | B.91 | C.8 |
| 1.1660 | B.92 | C.8 |
| 1.1661 | B.93 | C.8 |
| 1.1662 | B.94 | C.8 |
| 1.1663 | B.95 | C.8 |
| 1.1664 | B.96 | C.8 |
| 1.1665 | B.97 | C.8 |
| 1.1666 | B.98 | C.8 |
| 1.1667 | B.99 | C.8 |
| 1.1668 | B.100 | C.8 |
| 1.1669 | B.101 | C.8 |
| 1.1670 | B.102 | C.8 |
| 1.1671 | B.103 | C.8 |
| 1.1672 | B.104 | C.8 |
| 1.1673 | B.105 | C.8 |
| 1.1674 | B.106 | C.8 |
| 1.1675 | B.107 | C.8 |
| 1.1676 | B.108 | C.8 |
| 1.1677 | B.109 | C.8 |
| 1.1678 | B.110 | C.8 |
| 1.1679 | B.111 | C.8 |
| 1.1680 | B.112 | C.8 |
| 1.1681 | B.113 | C.8 |
| 1.1682 | B.114 | C.8 |
| 1.1683 | B.115 | C.8 |
| 1.1684 | B.116 | C.8 |
| 1.1685 | B.117 | C.8 |
| 1.1686 | B.118 | C.8 |
| 1.1687 | B.119 | C.8 |
| 1.1688 | B.120 | C.8 |
| 1.1689 | B.121 | C.8 |
| 1.1690 | B.122 | C.8 |
| 1.1691 | B.123 | C.8 |
| 1.1692 | B.124 | C.8 |
| 1.1693 | B.125 | C.8 |
| 1.1694 | B.126 | C.8 |
| 1.1695 | B.127 | C.8 |
| 1.1696 | B.128 | C.8 |
| 1.1697 | B.129 | C.8 |
| 1.1698 | B.130 | C.8 |
| 1.1699 | B.131 | C.8 |
| 1.1700 | B.132 | C.8 |
| 1.1701 | B.133 | C.8 |
| 1.1702 | B.134 | C.8 |
| 1.1703 | B.135 | C.8 |
| 1.1704 | B.136 | C.8 |
| 1.1705 | B.137 | C.8 |
| 1.1706 | B.138 | C.8 |
| 1.1707 | B.139 | C.8 |
| 1.1708 | B.140 | C.8 |
| 1.1709 | B.141 | C.8 |
| 1.1710 | B.142 | C.8 |
| 1.1711 | B.143 | C.8 |
| 1.1712 | B.144 | C.8 |
| 1.1713 | B.145 | C.8 |
| 1.1714 | B.146 | C.8 |
| 1.1715 | B.147 | C.8 |
| 1.1716 | B.148 | C.8 |
| 1.1717 | B.149 | C.8 |
| 1.1718 | B.150 | C.8 |
| 1.1719 | B.151 | C.8 |
| 1.1720 | B.152 | C.8 |
| 1.1721 | B.153 | C.8 |
| 1.1722 | B.154 | C.8 |
| 1.1723 | B.155 | C.8 |
| 1.1724 | B.156 | C.8 |
| 1.1725 | B.157 | C.8 |
| 1.1726 | B.158 | C.8 |
| 1.1727 | B.159 | C.8 |
| 1.1728 | B.160 | C.8 |
| 1.1729 | B.161 | C.8 |
| 1.1730 | B.162 | C.8 |
| 1.1731 | B.163 | C.8 |
| 1.1732 | B.164 | C.8 |
| 1.1733 | B.165 | C.8 |
| 1.1734 | B.166 | C.8 |
| 1.1735 | B.167 | C.8 |
| 1.1736 | B.168 | C.8 |
| 1.1737 | B.169 | C.8 |
| 1.1738 | B.170 | C.8 |
| 1.1739 | B.171 | C.8 |
| 1.1740 | B.172 | C.8 |
| 1.1741 | B.173 | C.8 |
| 1.1742 | B.174 | C.8 |
| 1.1743 | B.175 | C.8 |
| 1.1744 | B.176 | C.8 |
| 1.1745 | B.177 | C.8 |
| 1.1746 | B.178 | C.8 |
| 1.1747 | B.179 | C.8 |
| 1.1748 | B.180 | C.8 |
| 1.1749 | B.181 | C.8 |
| 1.1750 | B.182 | C.8 |
| 1.1751 | B.183 | C.8 |
| 1.1752 | B.184 | C.8 |
| 1.1753 | B.185 | C.8 |
| 1.1754 | B.186 | C.8 |
| 1.1755 | B.187 | C.8 |
| 1.1756 | B.188 | C.8 |
| 1.1757 | B.189 | C.8 |
| 1.1758 | B.190 | C.8 |
| 1.1759 | B.191 | C.8 |
| 1.1760 | B.192 | C.8 |
| 1.1761 | B.193 | C.8 |
| 1.1762 | B.194 | C.8 |
| 1.1763 | B.195 | C.8 |
| 1.1764 | B.196 | C.8 |
| 1.1765 | B.1 | C.9 |
| 1.1766 | B.2 | C.9 |
| 1.1767 | B.3 | C.9 |
| 1.1768 | B.4 | C.9 |
| 1.1769 | B.5 | C.9 |
| 1.1770 | B.6 | C.9 |
| 1.1771 | B.7 | C.9 |
| 1.1772 | B.8 | C.9 |
| 1.1773 | B.9 | C.9 |
| 1.1774 | B.10 | C.9 |
| 1.1775 | B.11 | C.9 |
| 1.1776 | B.12 | C.9 |
| 1.1777 | B.13 | C.9 |
| 1.1778 | B.14 | C.9 |
| 1.1779 | B.15 | C.9 |
| 1.1780 | B.16 | C.9 |
| 1.1781 | B.17 | C.9 |
| 1.1782 | B.18 | C.9 |
| 1.1783 | B.19 | C.9 |
| 1.1784 | B.20 | C.9 |
| 1.1785 | B.21 | C.9 |
| 1.1786 | B.22 | C.9 |
| 1.1787 | B.23 | C.9 |
| 1.1788 | B.24 | C.9 |
| 1.1789 | B.25 | C.9 |
| 1.1790 | B.26 | C.9 |
| 1.1791 | B.27 | C.9 |
| 1.1792 | B.28 | C.9 |
| 1.1793 | B.29 | C.9 |
| 1.1794 | B.30 | C.9 |
| 1.1795 | B.31 | C.9 |
| 1.1796 | B.32 | C.9 |
| 1.1797 | B.33 | C.9 |
| 1.1798 | B.34 | C.9 |
| 1.1799 | B.35 | C.9 |
| 1.1800 | B.36 | C.9 |
| 1.1801 | B.37 | C.9 |
| 1.1802 | B.38 | C.9 |
| 1.1803 | B.39 | C.9 |
| 1.1804 | B.40 | C.9 |
| 1.1805 | B.41 | C.9 |
| 1.1806 | B.42 | C.9 |
| 1.1807 | B.43 | C.9 |
| 1.1808 | B.44 | C.9 |
| 1.1809 | B.45 | C.9 |
| 1.1810 | B.46 | C.9 |
| 1.1811 | B.47 | C.9 |
| 1.1812 | B.48 | C.9 |
| 1.1813 | B.49 | C.9 |
| 1.1814 | B.50 | C.9 |
| 1.1815 | B.51 | C.9 |
| 1.1816 | B.52 | C.9 |
| 1.1817 | B.53 | C.9 |
| 1.1818 | B.54 | C.9 |
| 1.1819 | B.55 | C.9 |
| 1.1820 | B.56 | C.9 |
| 1.1821 | B.57 | C.9 |
| 1.1822 | B.58. | C.9 |
| 1.1823 | B.59 | C.9 |
| 1.1824 | B.60 | C.9 |
| 1.1825 | B.61 | C.9 |
| 1.1826 | B.62 | C.9 |
| 1.1827 | B.63 | C.9 |
| 1.1828 | B.64 | C.9 |
| 1.1829 | B.65 | C.9 |
| 1.1830 | B.66 | C.9 |
| 1.1831 | B.67 | C.9 |
| 1.1832 | B.68 | C.9 |
| 1.1833 | B.69 | C.9 |
| 1.1834 | B.70 | C.9 |
| 1.1835 | B.71 | C.9 |
| 1.1836 | B.72 | C.9 |
| 1.1837 | B.73 | C.9 |
| 1.1838 | B.74 | C.9 |
| 1.1839 | B.75 | C.9 |
| 1.1840 | B.76 | C.9 |
| 1.1841 | B.77 | C.9 |
| 1.1842 | B.78 | C.9 |
| 1.1843 | B.79 | C.9 |
| 1.1844 | B.80 | C.9 |
| 1.1845 | B.81 | C.9 |
| 1.1846 | B.82 | C.9 |
| 1.1847 | B.83 | C.9 |
| 1.1848 | B.84 | C.9 |
| 1.1849 | B.85 | C.9 |
| 1.1850 | B.86 | C.9 |
| 1.1851 | B.87 | C.9 |
| 1.1852 | B.88 | C.9 |
| 1.1853 | B.89 | C.9 |
| 1.1854 | B.90 | C.9 |
| 1.1855 | B.91 | C.9 |
| 1.1856 | B.92 | C.9 |
| 1.1857 | B.93 | C.9 |
| 1.1858 | B.94 | C.9 |
| 1.1859 | B.95 | C.9 |
| 1.1860 | B.96 | C.9 |
| 1.1861 | B.97 | C.9 |
| 1.1862 | B.98 | C.9 |
| 1.1863 | B.99 | C.9 |
| 1.1864 | B.100 | C.9 |
| 1.1865 | B.101 | C.9 |
| 1.1866 | B.102 | C.9 |
| 1.1867 | B.103 | C.9 |
| 1.1868 | B.104 | C.9 |
| 1.1869 | B.105 | C.9 |
| 1.1870 | B.106 | C.9 |
| 1.1871 | B.107 | C.9 |
| 1.1872 | B.108 | C.9 |
| 1.1873 | B.109 | C.9 |
| 1.1874 | B.110 | C.9 |
| 1.1875 | B.111 | C.9 |
| 1.1876 | B.112 | C.9 |
| 1.1877 | B.113 | C.9 |
| 1.1878 | B.114 | C.9 |
| 1.1879 | B.115 | C.9 |
| 1.1880 | B.116 | C.9 |
| 1.1881 | B.117 | C.9 |
| 1.1882 | B.118 | C.9 |
| 1.1883 | B.119 | C.9 |
| 1.1884 | B.120 | C.9 |
| 1.1885 | B.121 | C.9 |
| 1.1886 | B.122 | C.9 |
| 1.1887 | B.123 | C.9 |
| 1.1888 | B.124 | C.9 |
| 1.1889 | B.125 | C.9 |
| 1.1890 | B.126 | C.9 |
| 1.1891 | B.127 | C.9 |
| 1.1892 | B.128 | C.9 |
| 1.1893 | B.129 | C.9 |
| 1.1894 | B.130 | C.9 |
| 1.1895 | B.131 | C.9 |
| 1.1896 | B.132 | C.9 |
| 1.1897 | B.133 | C.9 |
| 1.1898 | B.134 | C.9 |
| 1.1899 | B.135 | C.9 |
| 1.1900 | B.136 | C.9 |
| 1.1901 | B.137 | C.9 |
| 1.1902 | B.138 | C.9 |
| 1.1903 | B.139 | C.9 |
| 1.1904 | B.140 | C.9 |
| 1.1905 | B.141 | C.9 |
| 1.1906 | B.142 | C.9 |
| 1.1907 | B.143 | C.9 |
| 1.1908 | B.144 | C.9 |
| 1.1909 | B.145 | C.9 |
| 1.1910 | B.146 | C.9 |
| 1.1911 | B.147 | C.9 |
| 1.1912 | B.148 | C.9 |
| 1.1913 | B.149 | C.9 |
| 1.1914 | B.150 | C.9 |
| 1.1915 | B.151 | C.9 |
| 1.1916 | B.152 | C.9 |
| 1.1917 | B.153 | C.9 |
| 1.1918 | B.154 | C.9 |
| 1.1919 | B.155 | C.9 |
| 1.1920 | B.156 | C.9 |
| 1.1921 | B.157 | C.9 |
| 1.1922 | B.158 | C.9 |
| 1.1923 | B.159 | C.9 |
| 1.1924 | B.160 | C.9 |
| 1.1925 | B.161 | C.9 |
| 1.1926 | B.162 | C.9 |
| 1.1927 | B.163 | C.9 |
| 1.1928 | B.164 | C.9 |
| 1.1929 | B.165 | C.9 |
| 1.1930 | B.166 | C.9 |
| 1.1931 | B.167 | C.9 |
| 1.1932 | B.168 | C.9 |
| 1.1933 | B.169 | C.9 |
| 1.1934 | B.170 | C.9 |
| 1.1935 | B.171 | C.9 |
| 1.1936 | B.172 | C.9 |
| 1.1937 | B.173 | C.9 |
| 1.1938 | B.174 | C.9 |
| 1.1939 | B.175 | C.9 |
| 1.1940 | B.176 | C.9 |
| 1.1941 | B.177 | C.9 |
| 1.1942 | B.178 | C.9 |
| 1.1943 | B.179 | C.9 |
| 1.1944 | B.180 | C.9 |
| 1.1945 | B.181 | C.9 |
| 1.1946 | B.182 | C.9 |
| 1.1947 | B.183 | C.9 |
| 1.1948 | B.184 | C.9 |
| 1.1949 | B.185 | C.9 |
| 1.1950 | B.186 | C.9 |
| 1.1951 | B.187 | C.9 |
| 1.1952 | B.188 | C.9 |
| 1.1953 | B.189 | C.9 |
| 1.1954 | B.190 | C.9 |
| 1.1955 | B.191 | C.9 |
| 1.1956 | B.192 | C.9 |
| 1.1957 | B.193 | C.9 |
| 1.1958 | B.194 | C.9 |
| 1.1959 | B.195 | C.9 |
| 1.1960 | B.196 | C.9 |
| 1.1961 | B.1 | C.10 |
| 1.1962 | B.2 | C.10 |
| 1.1963 | B.3 | C.10 |
| 1.1964 | B.4 | C.10 |
| 1.1965 | B.5 | C.10 |
| 1.1966 | B.6 | C.10 |
| 1.1967 | B.7 | C.10 |
| 1.1968 | B.8 | C.10 |
| 1.1969 | B.9 | C.10 |
| 1.1970 | B.10 | C.10 |
| 1.1971 | B.11 | C.10 |
| 1.1972 | B.12 | C.10 |
| 1.1973 | B.13 | C.10 |
| 1.1974 | B.14 | C.10 |
| 1.1975 | B.15 | C.10 |
| 1.1976 | B.16 | C.10 |
| 1.1977 | B.17 | C.10 |
| 1.1978 | B.18 | C.10 |
| 1.1979 | B.19 | C.10 |
| 1.1980 | B.20 | C.10 |
| 1.1981 | B.21 | C.10 |
| 1.1982 | B.22 | C.10 |
| 1.1983 | B.23 | C.10 |
| 1.1984 | B.24 | C.10 |
| 1.1985 | B.25 | C.10 |
| 1.1986 | B.26 | C.10 |
| 1.1987 | B.27 | C.10 |
| 1.1988 | B.28 | C.10 |
| 1.1989 | B.29 | C.10 |
| 1.1990 | B.30 | C.10 |
| 1.1991 | B.31 | C.10 |
| 1.1992 | B.32 | C.10 |
| 1.1993 | B.33 | C.10 |
| 1.1994 | B.34 | C.10 |
| 1.1995 | B.35 | C.10 |
| 1.1996 | B.36 | C.10 |
| 1.1997 | B.37 | C.10 |
| 1.1998 | B.38 | C.10 |
| 1.1999 | B.39 | C.10 |
| 1.2000 | B.40 | C.10 |
| 1.2001 | B.41 | C.10 |
| 1.2002 | B.42 | C.10 |
| 1.2003 | B.43 | C.10 |
| 1.2004 | B.44 | C.10 |
| 1.2005 | B.45 | C.10 |
| 1.2006 | B.46 | C.10 |
| 1.2007 | B.47 | C.10 |
| 1.2008 | B.48 | C.10 |
| 1.2009 | B.49 | C.10 |
| 1.2010 | B.50 | C.10 |
| 1.2011 | B.51 | C.10 |
| 1.2012 | B.52 | C.10 |
| 1.2013 | B.53 | C.10 |
| 1.2014 | B.54 | C.10 |
| 1.2015 | B.55 | C.10 |
| 1.2016 | B.56 | C.10 |
| 1.2017 | B.57 | C.10 |
| 1.2018 | B.58. | C.10 |
| 1.2019 | B.59 | C.10 |
| 1.2020 | B.60 | C.10 |
| 1.2021 | B.61 | C.10 |
| 1.2022 | B.62 | C.10 |
| 1.2023 | B.63 | C.10 |
| 1.2024 | B.64 | C.10 |
| 1.2025 | B.65 | C.10 |
| 1.2026 | B.66 | C.10 |
| 1.2027 | B.67 | C.10 |
| 1.2028 | B.68 | C.10 |
| 1.2029 | B.69 | C.10 |
| 1.2030 | B.70 | C.10 |
| 1.2031 | B.71 | C.10 |
| 1.2032 | B.72 | C.10 |
| 1.2033 | B.73 | C.10 |
| 1.2034 | B.74 | C.10 |
| 1.2035 | B.75 | C.10 |
| 1.2036 | B.76 | C.10 |
| 1.2037 | B.77 | C.10 |
| 1.2038 | B.78 | C.10 |
| 1.2039 | B.79 | C.10 |
| 1.2040 | B.80 | C.10 |
| 1.2041 | B.81 | C.10 |
| 1.2042 | B.82 | C.10 |
| 1.2043 | B.83 | C.10 |
| 1.2044 | B.84 | C.10 |
| 1.2045 | B.85 | C.10 |
| 1.2046 | B.86 | C.10 |
| 1.2047 | B.87 | C.10 |
| 1.2048 | B.88 | C.10 |
| 1.2049 | B.89 | C.10 |
| 1.2050 | B.90 | C.10 |
| 1.2051 | B.91 | C.10 |
| 1.2052 | B.92 | C.10 |
| 1.2053 | B.93 | C.10 |
| 1.2054 | B.94 | C.10 |
| 1.2055 | B.95 | C.10 |
| 1.2056 | B.96 | C.10 |
| 1.2057 | B.97 | C.10 |
| 1.2058 | B.98 | C.10 |
| 1.2059 | B.99 | C.10 |
| 1.2060 | B.100 | C.10 |
| 1.2061 | B.101 | C.10 |
| 1.2062 | B.102 | C.10 |
| 1.2063 | B.103 | C.10 |
| 1.2064 | B.104 | C.10 |
| 1.2065 | B.105 | C.10 |
| 1.2066 | B.106 | C.10 |
| 1.2067 | B.107 | C.10 |
| 1.2068 | B.108 | C.10 |
| 1.2069 | B.109 | C.10 |
| 1.2070 | B.110 | C.10 |
| 1.2071 | B.111 | C.10 |
| 1.2072 | B.112 | C.10 |
| 1.2073 | B.113 | C.10 |
| 1.2074 | B.114 | C.10 |
| 1.2075 | B.115 | C.10 |
| 1.2076 | B.116 | C.10 |
| 1.2077 | B.117 | C.10 |
| 1.2078 | B.118 | C.10 |
| 1.2079 | B.119 | C.10 |
| 1.2080 | B.120 | C.10 |
| 1.2081 | B.121 | C.10 |
| 1.2082 | B.122 | C.10 |
| 1.2083 | B.123 | C.10 |
| 1.2084 | B.124 | C.10 |
| 1.2085 | B.125 | C.10 |
| 1.2086 | B.126 | C.10 |
| 1.2087 | B.127 | C.10 |
| 1.2088 | B.128 | C.10 |
| 1.2089 | B.129 | C.10 |
| 1.2090 | B.130 | C.10 |
| 1.2091 | B.131 | C.10 |
| 1.2092 | B.132 | C.10 |
| 1.2093 | B.133 | C.10 |
| 1.2094 | B.134 | C.10 |
| 1.2095 | B.135 | C.10 |
| 1.2096 | B.136 | C.10 |
| 1.2097 | B.137 | C.10 |
| 1.2098 | B.138 | C.10 |
| 1.2099 | B.139 | C.10 |
| 1.2100 | B.140 | C.10 |
| 1.2101 | B.141 | C.10 |
| 1.2102 | B.142 | C.10 |
| 1.2103 | B.143 | C.10 |
| 1.2104 | B.144 | C.10 |
| 1.2105 | B.145 | C.10 |
| 1.2106 | B.146 | C.10 |
| 1.2107 | B.147 | C.10 |
| 1.2108 | B.148 | C.10 |
| 1.2109 | B.149 | C.10 |
| 1.2110 | B.150 | C.10 |
| 1.2111 | B.151 | C.10 |
| 1.2112 | B.152 | C.10 |
| 1.2113 | B.153 | C.10 |
| 1.2114 | B.154 | C.10 |
| 1.2115 | B.155 | C.10 |
| 1.2116 | B.156 | C.10 |
| 1.2117 | B.157 | C.10 |
| 1.2118 | B.158 | C.10 |
| 1.2119 | B.159 | C.10 |
| 1.2120 | B.160 | C.10 |
| 1.2121 | B.161 | C.10 |
| 1.2122 | B.162 | C.10 |
| 1.2123 | B.163 | C.10 |
| 1.2124 | B.164 | C.10 |
| 1.2125 | B.165 | C.10 |
| 1.2126 | B.166 | C.10 |
| 1.2127 | B.167 | C.10 |
| 1.2128 | B.168 | C.10 |
| 1.2129 | B.169 | C.10 |
| 1.2130 | B.170 | C.10 |
| 1.2131 | B.171 | C.10 |
| 1.2132 | B.172 | C.10 |
| 1.2133 | B.173 | C.10 |
| 1.2134 | B.174 | C.10 |
| 1.2135 | B.175 | C.10 |
| 1.2136 | B.176 | C.10 |
| 1.2137 | B.177 | C.10 |
| 1.2138 | B.178 | C.10 |
| 1.2139 | B.179 | C.10 |
| 1.2140 | B.180 | C.10 |
| 1.2141 | B.181 | C.10 |
| 1.2142 | B.182 | C.10 |
| 1.2143 | B.183 | C.10 |
| 1.2144 | B.184 | C.10 |
| 1.2145 | B.185 | C.10 |
| 1.2146 | B.186 | C.10 |
| 1.2147 | B.187 | C.10 |
| 1.2148 | B.188 | C.10 |
| 1.2149 | B.189 | C.10 |
| 1.2150 | B.190 | C.10 |
| 1.2151 | B.191 | C.10 |
| 1.2152 | B.192 | C.10 |
| 1.2153 | B.193 | C.10 |
| 1.2154 | B.194 | C.10 |
| 1.2155 | B.195 | C.10 |
| 1.2156 | B.196 | C.10 |
| 1.2157 | B.1 | C.11 |
| 1.2158 | B.2 | C.11 |
| 1.2159 | B.3 | C.11 |
| 1.2160 | B.4 | C.11 |
| 1.2161 | B.5 | C.11 |
| 1.2162 | B.6 | C.11 |
| 1.2163 | B.7 | C.11 |
| 1.2164 | B.8 | C.11 |
| 1.2165 | B.9 | C.11 |
| 1.2166 | B.10 | C.11 |
| 1.2167 | B.11 | C.11 |
| 1.2168 | B.12 | C.11 |
| 1.2169 | B.13 | C.11 |
| 1.2170 | B.14 | C.11 |
| 1.2171 | B.15 | C.11 |
| 1.2172 | B.16 | C.11 |
| 1.2173 | B.17 | C.11 |
| 1.2174 | B.18 | C.11 |
| 1.2175 | B.19 | C.11 |
| 1.2176 | B.20 | C.11 |
| 1.2177 | B.21 | C.11 |
| 1.2178 | B.22 | C.11 |
| 1.2179 | B.23 | C.11 |
| 1.2180 | B.24 | C.11 |
| 1.2181 | B.25 | C.11 |
| 1.2182 | B.26 | C.11 |
| 1.2183 | B.27 | C.11 |
| 1.2184 | B.28 | C.11 |
| 1.2185 | B.29 | C.11 |
| 1.2186 | B.30 | C.11 |
| 1.2187 | B.31 | C.11 |
| 1.2188 | B.32 | C.11 |
| 1.2189 | B.33 | C.11 |
| 1.2190 | B.34 | C.11 |
| 1.2191 | B.35 | C.11 |
| 1.2192 | B.36 | C.11 |
| 1.2193 | B.37 | C.11 |
| 1.2194 | B.38 | C.11 |
| 1.2195 | B.39 | C.11 |
| 1.2196 | B.40 | C.11 |
| 1.2197 | B.41 | C.11 |
| 1.2198 | B.42 | C.11 |
| 1.2199 | B.43 | C.11 |
| 1.2200 | B.44 | C.11 |
| 1.2201 | B.45 | C.11 |
| 1.2202 | B.46 | C.11 |
| 1.2203 | B.47 | C.11 |
| 1.2204 | B.48 | C.11 |
| 1.2205 | B.49 | C.11 |
| 1.2206 | B.50 | C.11 |
| 1.2207 | B.51 | C.11 |
| 1.2208 | B.52 | C.11 |
| 1.2209 | B.53 | C.11 |
| 1.2210 | B.54 | C.11 |
| 1.2211 | B.55 | C.11 |
| 1.2212 | B.56 | C.11 |
| 1.2213 | B.57 | C.11 |
| 1.2214 | B.58. | C.11 |
| 1.2215 | B.59 | C.11 |
| 1.2216 | B.60 | C.11 |
| 1.2217 | B.61 | C.11 |
| 1.2218 | B.62 | C.11 |
| 1.2219 | B.63 | C.11 |
| 1.2220 | B.64 | C.11 |
| 1.2221 | B.65 | C.11 |
| 1.2222 | B.66 | C.11 |
| 1.2223 | B.67 | C.11 |
| 1.2224 | B.68 | C.11 |
| 1.2225 | B.69 | C.11 |
| 1.2226 | B.70 | C.11 |
| 1.2227 | B.71 | C.11 |
| 1.2228 | B.72 | C.11 |
| 1.2229 | B.73 | C.11 |
| 1.2230 | B.74 | C.11 |
| 1.2231 | B.75 | C.11 |
| 1.2232 | B.76 | C.11 |
| 1.2233 | B.77 | C.11 |
| 1.2234 | B.78 | C.11 |
| 1.2235 | B.79 | C.11 |
| 1.2236 | B.80 | C.11 |
| 1.2237 | B.81 | C.11 |
| 1.2238 | B.82 | C.11 |
| 1.2239 | B.83 | C.11 |
| 1.2240 | B.84 | C.11 |
| 1.2241 | B.85 | C.11 |
| 1.2242 | B.86 | C.11 |
| 1.2243 | B.87 | C.11 |
| 1.2244 | B.88 | C.11 |
| 1.2245 | B.89 | C.11 |
| 1.2246 | B.90 | C.11 |
| 1.2247 | B.91 | C.11 |
| 1.2248 | B.92 | C.11 |
| 1.2249 | B.93 | C.11 |
| 1.2250 | B.94 | C.11 |
| 1.2251 | B.95 | C.11 |
| 1.2252 | B.96 | C.11 |
| 1.2253 | B.97 | C.11 |
| 1.2254 | B.98 | C.11 |
| 1.2255 | B.99 | C.11 |
| 1.2256 | B.100 | C.11 |
| 1.2257 | B.101 | C.11 |
| 1.2258 | B.102 | C.11 |
| 1.2259 | B.103 | C.11 |
| 1.2260 | B.104 | C.11 |
| 1.2261 | B.105 | C.11 |
| 1.2262 | B.106 | C.11 |
| 1.2263 | B.107 | C.11 |
| 1.2264 | B.108 | C.11 |
| 1.2265 | B.109 | C.11 |
| 1.2266 | B.110 | C.11 |
| 1.2267 | B.111 | C.11 |
| 1.2268 | B.112 | C.11 |
| 1.2269 | B.113 | C.11 |
| 1.2270 | B.114 | C.11 |
| 1.2271 | B.115 | C.11 |
| 1.2272 | B.116 | C.11 |
| 1.2273 | B.117 | C.11 |
| 1.2274 | B.118 | C.11 |
| 1.2275 | B.119 | C.11 |
| 1.2276 | B.120 | C.11 |
| 1.2277 | B.121 | C.11 |
| 1.2278 | B.122 | C.11 |
| 1.2279 | B.123 | C.11 |
| 1.2280 | B.124 | C.11 |
| 1.2281 | B.125 | C.11 |
| 1.2282 | B.126 | C.11 |
| 1.2283 | B.127 | C.11 |
| 1.2284 | B.128 | C.11 |
| 1.2285 | B.129 | C.11 |
| 1.2286 | B.130 | C.11 |
| 1.2287 | B.131 | C.11 |
| 1.2288 | B.132 | C.11 |
| 1.2289 | B.133 | C.11 |
| 1.2290 | B.134 | C.11 |
| 1.2291 | B.135 | C.11 |
| 1.2292 | B.136 | C.11 |
| 1.2293 | B.137 | C.11 |
| 1.2294 | B.138 | C.11 |
| 1.2295 | B.139 | C.11 |
| 1.2296 | B.140 | C.11 |
| 1.2297 | B.141 | C.11 |
| 1.2298 | B.142 | C.11 |
| 1.2299 | B.143 | C.11 |
| 1.2300 | B.144 | C.11 |
| 1.2301 | B.145 | C.11 |
| 1.2302 | B.146 | C.11 |
| 1.2303 | B.147 | C.11 |
| 1.2304 | B.148 | C.11 |
| 1.2305 | B.149 | C.11 |
| 1.2306 | B.150 | C.11 |
| 1.2307 | B.151 | C.11 |
| 1.2308 | B.152 | C.11 |
| 1.2309 | B.153 | C.11 |
| 1.2310 | B.154 | C.11 |
| 1.2311 | B.155 | C.11 |
| 1.2312 | B.156 | C.11 |
| 1.2313 | B.157 | C.11 |
| 1.2314 | B.158 | C.11 |
| 1.2315 | B.159 | C.11 |
| 1.2316 | B.160 | C.11 |
| 1.2317 | B.161 | C.11 |
| 1.2318 | B.162 | C.11 |
| 1.2319 | B.163 | C.11 |
| 1.2320 | B.164 | C.11 |
| 1.2321 | B.165 | C.11 |
| 1.2322 | B.166 | C.11 |
| 1.2323 | B.167 | C.11 |
| 1.2324 | B.168 | C.11 |
| 1.2325 | B.169 | C.11 |
| 1.2326 | B.170 | C.11 |
| 1.2327 | B.171 | C.11 |
| 1.2328 | B.172 | C.11 |
| 1.2329 | B.173 | C.11 |
| 1.2330 | B.174 | C.11 |
| 1.2331 | B.175 | C.11 |
| 1.2332 | B.176 | C.11 |
| 1.2333 | B.177 | C.11 |
| 1.2334 | B.178 | C.11 |
| 1.2335 | B.179 | C.11 |
| 1.2336 | B.180 | C.11 |
| 1.2337 | B.181 | C.11 |
| 1.2338 | B.182 | C.11 |
| 1.2339 | B.183 | C.11 |
| 1.2340 | B.184 | C.11 |
| 1.2341 | B.185 | C.11 |
| 1.2342 | B.186 | C.11 |
| 1.2343 | B.187 | C.11 |
| 1.2344 | B.188 | C.11 |
| 1.2345 | B.189 | C.11 |
| 1.2346 | B.190 | C.11 |
| 1.2347 | B.191 | C.11 |
| 1.2348 | B.192 | C.11 |
| 1.2349 | B.193 | C.11 |
| 1.2350 | B.194 | C.11 |
| 1.2351 | B.195 | C.11 |
| 1.2352 | B.196 | C.11 |
| 1.2353 | B.1 | C.12 |
| 1.2354 | B.2 | C.12 |
| 1.2355 | B.3 | C.12 |
| 1.2356 | B.4 | C.12 |
| 1.2357 | B.5 | C.12 |
| 1.2358 | B.6 | C.12 |
| 1.2359 | B.7 | C.12 |
| 1.2360 | B.8 | C.12 |
| 1.2361 | B.9 | C.12 |
| 1.2362 | B.10 | C.12 |
| 1.2363 | B.11 | C.12 |
| 1.2364 | B.12 | C.12 |
| 1.2365 | B.13 | C.12 |
| 1.2366 | B.14 | C.12 |
| 1.2367 | B.15 | C.12 |
| 1.2368 | B.16 | C.12 |
| 1.2369 | B.17 | C.12 |
| 1.2370 | B.18 | C.12 |
| 1.2371 | B.19 | C.12 |
| 1.2372 | B.20 | C.12 |
| 1.2373 | B.21 | C.12 |
| 1.2374 | B.22 | C.12 |
| 1.2375 | B.23 | C.12 |
| 1.2376 | B.24 | C.12 |
| 1.2377 | B.25 | C.12 |
| 1.2378 | B.26 | C.12 |
| 1.2379 | B.27 | C.12 |
| 1.2380 | B.28 | C.12 |
| 1.2381 | B.29 | C.12 |
| 1.2382 | B.30 | C.12 |
| 1.2383 | B.31 | C.12 |
| 1.2384 | B.32 | C.12 |
| 1.2385 | B.33 | C.12 |
| 1.2386 | B.34 | C.12 |
| 1.2387 | B.35 | C.12 |
| 1.2388 | B.36 | C.12 |
| 1.2389 | B.37 | C.12 |
| 1.2390 | B.38 | C.12 |
| 1.2391 | B.39 | C.12 |
| 1.2392 | B.40 | C.12 |
| 1.2393 | B.41 | C.12 |
| 1.2394 | B.42 | C.12 |
| 1.2395 | B.43 | C.12 |
| 1.2396 | B.44 | C.12 |
| 1.2397 | B.45 | C.12 |
| 1.2398 | B.46 | C.12 |
| 1.2399 | B.47 | C.12 |
| 1.2400 | B.48 | C.12 |
| 1.2401 | B.49 | C.12 |
| 1.2402 | B.50 | C.12 |
| 1.2403 | B.51 | C.12 |
| 1.2404 | B.52 | C.12 |
| 1.2405 | B.53 | C.12 |
| 1.2406 | B.54 | C.12 |
| 1.2407 | B.55 | C.12 |
| 1.2408 | B.56 | C.12 |
| 1.2409 | B.57 | C.12 |
| 1.2410 | B.58. | C.12 |
| 1.2411 | B.59 | C.12 |
| 1.2412 | B.60 | C.12 |
| 1.2413 | B.61 | C.12 |
| 1.2414 | B.62 | C.12 |
| 1.2415 | B.63 | C.12 |
| 1.2416 | B.64 | C.12 |
| 1.2417 | B.65 | C.12 |
| 1.2418 | B.66 | C.12 |
| 1.2419 | B.67 | C.12 |
| 1.2420 | B.68 | C.12 |
| 1.2421 | B.69 | C.12 |
| 1.2422 | B.70 | C.12 |
| 1.2423 | B.71 | C.12 |
| 1.2424 | B.72 | C.12 |
| 1.2425 | B.73 | C.12 |
| 1.2426 | B.74 | C.12 |
| 1.2427 | B.75 | C.12 |
| 1.2428 | B.76 | C.12 |
| 1.2429 | B.77 | C.12 |
| 1.2430 | B.78 | C.12 |
| 1.2431 | B.79 | C.12 |
| 1.2432 | B.80 | C.12 |
| 1.2433 | B.81 | C.12 |
| 1.2434 | B.82 | C.12 |
| 1.2435 | B.83 | C.12 |
| 1.2436 | B.84 | C.12 |
| 1.2437 | B.85 | C.12 |
| 1.2438 | B.86 | C.12 |
| 1.2439 | B.87 | C.12 |
| 1.2440 | B.88 | C.12 |
| 1.2441 | B.89 | C.12 |
| 1.2442 | B.90 | C.12 |
| 1.2443 | B.91 | C.12 |
| 1.2444 | B.92 | C.12 |
| 1.2445 | B.93 | C.12 |
| 1.2446 | B.94 | C.12 |
| 1.2447 | B.95 | C.12 |
| 1.2448 | B.96 | C.12 |
| 1.2449 | B.97 | C.12 |
| 1.2450 | B.98 | C.12 |
| 1.2451 | B.99 | C.12 |
| 1.2452 | B.100 | C.12 |
| 1.2453 | B.101 | C.12 |
| 1.2454 | B.102 | C.12 |
| 1.2455 | B.103 | C.12 |
| 1.2456 | B.104 | C.12 |
| 1.2457 | B.105 | C.12 |
| 1.2458 | B.106 | C.12 |
| 1.2459 | B.107 | C.12 |
| 1.2460 | B.108 | C.12 |
| 1.2461 | B.109 | C.12 |
| 1.2462 | B.110 | C.12 |
| 1.2463 | B.111 | C.12 |
| 1.2464 | B.112 | C.12 |
| 1.2465 | B.113 | C.12 |
| 1.2466 | B.114 | C.12 |
| 1.2467 | B.115 | C.12 |
| 1.2468 | B.116 | C.12 |
| 1.2469 | B.117 | C.12 |
| 1.2470 | B.118 | C.12 |
| 1.2471 | B.119 | C.12 |
| 1.2472 | B.120 | C.12 |
| 1.2473 | B.121 | C.12 |
| 1.2474 | B.122 | C.12 |
| 1.2475 | B.123 | C.12 |
| 1.2476 | B.124 | C.12 |
| 1.2477 | B.125 | C.12 |
| 1.2478 | B.126 | C.12 |
| 1.2479 | B.127 | C.12 |
| 1.2480 | B.128 | C.12 |
| 1.2481 | B.129 | C.12 |
| 1.2482 | B.130 | C.12 |
| 1.2483 | B.131 | C.12 |
| 1.2484 | B.132 | C.12 |
| 1.2485 | B.133 | C.12 |
| 1.2486 | B.134 | C.12 |
| 1.2487 | B.135 | C.12 |
| 1.2488 | B.136 | C.12 |
| 1.2489 | B.137 | C.12 |
| 1.2490 | B.138 | C.12 |
| 1.2491 | B.139 | C.12 |
| 1.2492 | B.140 | C.12 |
| 1.2493 | B.141 | C.12 |
| 1.2494 | B.142 | C.12 |
| 1.2495 | B.143 | C.12 |
| 1.2496 | B.144 | C.12 |
| 1.2497 | B.145 | C.12 |
| 1.2498 | B.146 | C.12 |
| 1.2499 | B.147 | C.12 |
| 1.2500 | B.148 | C.12 |
| 1.2501 | B.149 | C.12 |
| 1.2502 | B.150 | C.12 |
| 1.2503 | B.151 | C.12 |
| 1.2504 | B.152 | C.12 |
| 1.2505 | B.153 | C.12 |
| 1.2506 | B.154 | C.12 |
| 1.2507 | B.155 | C.12 |
| 1.2508 | B.156 | C.12 |
| 1.2509 | B.157 | C.12 |
| 1.2510 | B.158 | C.12 |
| 1.2511 | B.159 | C.12 |
| 1.2512 | B.160 | C.12 |
| 1.2513 | B.161 | C.12 |
| 1.2514 | B.162 | C.12 |
| 1.2515 | B.163 | C.12 |
| 1.2516 | B.164 | C.12 |
| 1.2517 | B.165 | C.12 |
| 1.2518 | B.166 | C.12 |
| 1.2519 | B.167 | C.12 |
| 1.2520 | B.168 | C.12 |
| 1.2521 | B.169 | C.12 |
| 1.2522 | B.170 | C.12 |
| 1.2523 | B.171 | C.12 |
| 1.2524 | B.172 | C.12 |
| 1.2525 | B.173 | C.12 |
| 1.2526 | B.174 | C.12 |
| 1.2527 | B.175 | C.12 |
| 1.2528 | B.176 | C.12 |
| 1.2529 | B.177 | C.12 |
| 1.2530 | B.178 | C.12 |
| 1.2531 | B.179 | C.12 |
| 1.2532 | B.180 | C.12 |
| 1.2533 | B.181 | C.12 |
| 1.2534 | B.182 | C.12 |
| 1.2535 | B.183 | C.12 |
| 1.2536 | B.184 | C.12 |
| 1.2537 | B.185 | C.12 |
| 1.2538 | B.186 | C.12 |
| 1.2539 | B.187 | C.12 |
| 1.2540 | B.188 | C.12 |
| 1.2541 | B.189 | C.12 |
| 1.2542 | B.190 | C.12 |
| 1.2543 | B.191 | C.12 |
| 1.2544 | B.192 | C.12 |
| 1.2545 | B.193 | C.12 |
| 1.2546 | B.194 | C.12 |
| 1.2547 | B.195 | C.12 |
| 1.2548 | B.196 | C.12 |
| 1.2549 | B.1 | C.13 |
| 1.2550 | B.2 | C.13 |
| 1.2551 | B.3 | C.13 |
| 1.2552 | B.4 | C.13 |
| 1.2553 | B.5 | C.13 |
| 1.2554 | B.6 | C.13 |
| 1.2555 | B.7 | C.13 |
| 1.2556 | B.8 | C.13 |
| 1.2557 | B.9 | C.13 |
| 1.2558 | B.10 | C.13 |
| 1.2559 | B.11 | C.13 |
| 1.2560 | B.12 | C.13 |
| 1.2561 | B.13 | C.13 |
| 1.2562 | B.14 | C.13 |
| 1.2563 | B.15 | C.13 |
| 1.2564 | B.16 | C.13 |
| 1.2565 | B.17 | C.13 |
| 1.2566 | B.18 | C.13 |
| 1.2567 | B.19 | C.13 |
| 1.2568 | B.20 | C.13 |
| 1.2569 | B.21 | C.13 |
| 1.2570 | B.22 | C.13 |
| 1.2571 | B.23 | C.13 |
| 1.2572 | B.24 | C.13 |
| 1.2573 | B.25 | C.13 |
| 1.2574 | B.26 | C.13 |
| 1.2575 | B.27 | C.13 |
| 1.2576 | B.28 | C.13 |
| 1.2577 | B.29 | C.13 |
| 1.2578 | B.30 | C.13 |
| 1.2579 | B.31 | C.13 |
| 1.2580 | B.32 | C.13 |
| 1.2581 | B.33 | C.13 |
| 1.2582 | B.34 | C.13 |
| 1.2583 | B.35 | C.13 |
| 1.2584 | B.36 | C.13 |
| 1.2585 | B.37 | C.13 |
| 1.2586 | B.38 | C.13 |
| 1.2587 | B.39 | C.13 |
| 1.2588 | B.40 | C.13 |
| 1.2589 | B.41 | C.13 |
| 1.2590 | B.42 | C.13 |
| 1.2591 | B.43 | C.13 |
| 1.2592 | B.44 | C.13 |
| 1.2593 | B.45 | C.13 |
| 1.2594 | B.46 | C.13 |
| 1.2595 | B.47 | C.13 |
| 1.2596 | B.48 | C.13 |
| 1.2597 | B.49 | C.13 |
| 1.2598 | B.50 | C.13 |
| 1.2599 | B.51 | C.13 |
| 1.2600 | B.52 | C.13 |
| 1.2601 | B.53 | C.13 |
| 1.2602 | B.54 | C.13 |
| 1.2603 | B.55 | C.13 |
| 1.2604 | B.56 | C.13 |
| 1.2605 | B.57 | C.13 |
| 1.2606 | B.58. | C.13 |
| 1.2607 | B.59 | C.13 |
| 1.2608 | B.60 | C.13 |
| 1.2609 | B.61 | C.13 |
| 1.2610 | B.62 | C.13 |
| 1.2611 | B.63 | C.13 |
| 1.2612 | B.64 | C.13 |
| 1.2613 | B.65 | C.13 |
| 1.2614 | B.66 | C.13 |
| 1.2615 | B.67 | C.13 |
| 1.2616 | B.68 | C.13 |
| 1.2617 | B.69 | C.13 |
| 1.2618 | B.70 | C.13 |
| 1.2619 | B.71 | C.13 |
| 1.2620 | B.72 | C.13 |
| 1.2621 | B.73 | C.13 |
| 1.2622 | B.74 | C.13 |
| 1.2623 | B.75 | C.13 |
| 1.2624 | B.76 | C.13 |
| 1.2625 | B.77 | C.13 |
| 1.2626 | B.78 | C.13 |
| 1.2627 | B.79 | C.13 |
| 1.2628 | B.80 | C.13 |
| 1.2629 | B.81 | C.13 |
| 1.2630 | B.82 | C.13 |
| 1.2631 | B.83 | C.13 |
| 1.2632 | B.84 | C.13 |
| 1.2633 | B.85 | C.13 |
| 1.2634 | B.86 | C.13 |
| 1.2635 | B.87 | C.13 |
| 1.2636 | B.88 | C.13 |
| 1.2637 | B.89 | C.13 |
| 1.2638 | B.90 | C.13 |
| 1.2639 | B.91 | C.13 |
| 1.2640 | B.92 | C.13 |
| 1.2641 | B.93 | C.13 |
| 1.2642 | B.94 | C.13 |
| 1.2643 | B.95 | C.13 |
| 1.2644 | B.96 | C.13 |
| 1.2645 | B.97 | C.13 |
| 1.2646 | B.98 | C.13 |
| 1.2647 | B.99 | C.13 |
| 1.2648 | B.100 | C.13 |
| 1.2649 | B.101 | C.13 |
| 1.2650 | B.102 | C.13 |
| 1.2651 | B.103 | C.13 |
| 1.2652 | B.104 | C.13 |
| 1.2653 | B.105 | C.13 |
| 1.2654 | B.106 | C.13 |
| 1.2655 | B.107 | C.13 |
| 1.2656 | B.108 | C.13 |
| 1.2657 | B.109 | C.13 |
| 1.2658 | B.110 | C.13 |
| 1.2659 | B.111 | C.13 |
| 1.2660 | B.112 | C.13 |
| 1.2661 | B.113 | C.13 |
| 1.2662 | B.114 | C.13 |
| 1.2663 | B.115 | C.13 |
| 1.2664 | B.116 | C.13 |
| 1.2665 | B.117 | C.13 |
| 1.2666 | B.118 | C.13 |
| 1.2667 | B.119 | C.13 |
| 1.2668 | B.120 | C.13 |
| 1.2669 | B.121 | C.13 |
| 1.2670 | B.122 | C.13 |
| 1.2671 | B.123 | C.13 |
| 1.2672 | B.124 | C.13 |
| 1.2673 | B.125 | C.13 |
| 1.2674 | B.126 | C.13 |
| 1.2675 | B.127 | C.13 |
| 1.2676 | B.128 | C.13 |
| 1.2677 | B.129 | C.13 |
| 1.2678 | B.130 | C.13 |
| 1.2679 | B.131 | C.13 |
| 1.2680 | B.132 | C.13 |
| 1.2681 | B.133 | C.13 |
| 1.2682 | B.134 | C.13 |
| 1.2683 | B.135 | C.13 |
| 1.2684 | B.136 | C.13 |
| 1.2685 | B.137 | C.13 |
| 1.2686 | B.138 | C.13 |
| 1.2687 | B.139 | C.13 |
| 1.2688 | B.140 | C.13 |
| 1.2689 | B.141 | C.13 |
| 1.2690 | B.142 | C.13 |
| 1.2691 | B.143 | C.13 |
| 1.2692 | B.144 | C.13 |
| 1.2693 | B.145 | C.13 |
| 1.2694 | B.146 | C.13 |
| 1.2695 | B.147 | C.13 |
| 1.2696 | B.148 | C.13 |
| 1.2697 | B.149 | C.13 |
| 1.2698 | B.150 | C.13 |
| 1.2699 | B.151 | C.13 |
| 1.2700 | B.152 | C.13 |
| 1.2701 | B.153 | C.13 |
| 1.2702 | B.154 | C.13 |
| 1.2703 | B.155 | C.13 |
| 1.2704 | B.156 | C.13 |
| 1.2705 | B.157 | C.13 |
| 1.2706 | B.158 | C.13 |
| 1.2707 | B.159 | C.13 |
| 1.2708 | B.160 | C.13 |
| 1.2709 | B.161 | C.13 |
| 1.2710 | B.162 | C.13 |
| 1.2711 | B.163 | C.13 |
| 1.2712 | B.164 | C.13 |
| 1.2713 | B.165 | C.13 |
| 1.2714 | B.166 | C.13 |
| 1.2715 | B.167 | C.13 |
| 1.2716 | B.168 | C.13 |
| 1.2717 | B.169 | C.13 |
| 1.2718 | B.170 | C.13 |
| 1.2719 | B.171 | C.13 |
| 1.2720 | B.172 | C.13 |
| 1.2721 | B.173 | C.13 |
| 1.2722 | B.174 | C.13 |
| 1.2723 | B.175 | C.13 |
| 1.2724 | B.176 | C.13 |
| 1.2725 | B.177 | C.13 |
| 1.2726 | B.178 | C.13 |
| 1.2727 | B.179 | C.13 |
| 1.2728 | B.180 | C.13 |
| 1.2729 | B.181 | C.13 |
| 1.2730 | B.182 | C.13 |
| 1.2731 | B.183 | C.13 |
| 1.2732 | B.184 | C.13 |
| 1.2733 | B.185 | C.13 |
| 1.2734 | B.186 | C.13 |
| 1.2735 | B.187 | C.13 |
| 1.2736 | B.188 | C.13 |
| 1.2737 | B.189 | C.13 |
| 1.2738 | B.190 | C.13 |
| 1.2739 | B.191 | C.13 |
| 1.2740 | B.192 | C.13 |
| 1.2741 | B.193 | C.13 |
| 1.2742 | B.194 | C.13 |
| 1.2743 | B.195 | C.13 |
| 1.2744 | B.196 | C.13 |
| 1.2745 | B.1 | C.14 |
| 1.2746 | B.2 | C.14 |
| 1.2747 | B.3 | C.14 |
| 1.2748 | B.4 | C.14 |
| 1.2749 | B.5 | C.14 |
| 1.2750 | B.6 | C.14 |
| 1.2751 | B.7 | C.14 |
| 1.2752 | B.8 | C.14 |
| 1.2753 | B.9 | C.14 |
| 1.2754 | B.10 | C.14 |
| 1.2755 | B.11 | C.14 |
| 1.2756 | B.12 | C.14 |
| 1.2757 | B.13 | C.14 |
| 1.2758 | B.14 | C.14 |
| 1.2759 | B.15 | C.14 |
| 1.2760 | B.16 | C.14 |
| 1.2761 | B.17 | C.14 |
| 1.2762 | B.18 | C.14 |
| 1.2763 | B.19 | C.14 |
| 1.2764 | B.20 | C.14 |
| 1.2765 | B.21 | C.14 |
| 1.2766 | B.22 | C.14 |
| 1.2767 | B.23 | C.14 |
| 1.2768 | B.24 | C.14 |
| 1.2769 | B.25 | C.14 |
| 1.2770 | B.26 | C.14 |
| 1.2771 | B.27 | C.14 |
| 1.2772 | B.28 | C.14 |
| 1.2773 | B.29 | C.14 |
| 1.2774 | B.30 | C.14 |
| 1.2775 | B.31 | C.14 |
| 1.2776 | B.32 | C.14 |
| 1.2777 | B.33 | C.14 |
| 1.2778 | B.34 | C.14 |
| 1.2779 | B.35 | C.14 |
| 1.2780 | B.36 | C.14 |
| 1.2781 | B.37 | C.14 |
| 1.2782 | B.38 | C.14 |
| 1.2783 | B.39 | C.14 |
| 1.2784 | B.40 | C.14 |
| 1.2785 | B.41 | C.14 |
| 1.2786 | B.42 | C.14 |
| 1.2787 | B.43 | C.14 |
| 1.2788 | B.44 | C.14 |
| 1.2789 | B.45 | C.14 |
| 1.2790 | B.46 | C.14 |
| 1.2791 | B.47 | C.14 |
| 1.2792 | B.48 | C.14 |
| 1.2793 | B.49 | C.14 |
| 1.2794 | B.50 | C.14 |
| 1.2795 | B.51 | C.14 |
| 1.2796 | B.52 | C.14 |
| 1.2797 | B.53 | C.14 |
| 1.2798 | B.54 | C.14 |
| 1.2799 | B.55 | C.14 |
| 1.2800 | B.56 | C.14 |
| 1.2801 | B.57 | C.14 |
| 1.2802 | B.58. | C.14 |
| 1.2803 | B.59 | C.14 |
| 1.2804 | B.60 | C.14 |
| 1.2805 | B.61 | C.14 |
| 1.2806 | B.62 | C.14 |
| 1.2807 | B.63 | C.14 |
| 1.2808 | B.64 | C.14 |
| 1.2809 | B.65 | C.14 |
| 1.2810 | B.66 | C.14 |
| 1.2811 | B.67 | C.14 |
| 1.2812 | B.68 | C.14 |
| 1.2813 | B.69 | C.14 |
| 1.2814 | B.70 | C.14 |
| 1.2815 | B.71 | C.14 |
| 1.2816 | B.72 | C.14 |
| 1.2817 | B.73 | C.14 |
| 1.2818 | B.74 | C.14 |
| 1.2819 | B.75 | C.14 |
| 1.2820 | B.76 | C.14 |
| 1.2821 | B.77 | C.14 |
| 1.2822 | B.78 | C.14 |
| 1.2823 | B.79 | C.14 |
| 1.2824 | B.80 | C.14 |
| 1.2825 | B.81 | C.14 |
| 1.2826 | B.82 | C.14 |
| 1.2827 | B.83 | C.14 |
| 1.2828 | B.84 | C.14 |
| 1.2829 | B.85 | C.14 |
| 1.2830 | B.86 | C.14 |
| 1.2831 | B.87 | C.14 |
| 1.2832 | B.88 | C.14 |
| 1.2833 | B.89 | C.14 |
| 1.2834 | B.90 | C.14 |
| 1.2835 | B.91 | C.14 |
| 1.2836 | B.92 | C.14 |
| 1.2837 | B.93 | C.14 |
| 1.2838 | B.94 | C.14 |
| 1.2839 | B.95 | C.14 |
| 1.2840 | B.96 | C.14 |
| 1.2841 | B.97 | C.14 |
| 1.2842 | B.98 | C.14 |
| 1.2843 | B.99 | C.14 |
| 1.2844 | B.100 | C.14 |
| 1.2845 | B.101 | C.14 |
| 1.2846 | B.102 | C.14 |
| 1.2847 | B.103 | C.14 |
| 1.2848 | B.104 | C.14 |
| 1.2849 | B.105 | C.14 |
| 1.2850 | B.106 | C.14 |
| 1.2851 | B.107 | C.14 |
| 1.2852 | B.108 | C.14 |
| 1.2853 | B.109 | C.14 |
| 1.2854 | B.110 | C.14 |
| 1.2855 | B.111 | C.14 |
| 1.2856 | B.112 | C.14 |
| 1.2857 | B.113 | C.14 |
| 1.2858 | B.114 | C.14 |
| 1.2859 | B.115 | C.14 |
| 1.2860 | B.116 | C.14 |
| 1.2861 | B.117 | C.14 |
| 1.2862 | B.118 | C.14 |
| 1.2863 | B.119 | C.14 |
| 1.2864 | B.120 | C.14 |
| 1.2865 | B.121 | C.14 |
| 1.2866 | B.122 | C.14 |
| 1.2867 | B.123 | C.14 |
| 1.2868 | B.124 | C.14 |
| 1.2869 | B.125 | C.14 |
| 1.2870 | B.126 | C.14 |
| 1.2871 | B.127 | C.14 |
| 1.2872 | B.128 | C.14 |
| 1.2873 | B.129 | C.14 |
| 1.2874 | B.130 | C.14 |
| 1.2875 | B.131 | C.14 |
| 1.2876 | B.132 | C.14 |
| 1.2877 | B.133 | C.14 |
| 1.2878 | B.134 | C.14 |
| 1.2879 | B.135 | C.14 |
| 1.2880 | B.136 | C.14 |
| 1.2881 | B.137 | C.14 |
| 1.2882 | B.138 | C.14 |
| 1.2883 | B.139 | C.14 |
| 1.2884 | B.140 | C.14 |
| 1.2885 | B.141 | C.14 |
| 1.2886 | B.142 | C.14 |
| 1.2887 | B.143 | C.14 |
| 1.2888 | B.144 | C.14 |
| 1.2889 | B.145 | C.14 |
| 1.2890 | B.146 | C.14 |
| 1.2891 | B.147 | C.14 |
| 1.2892 | B.148 | C.14 |
| 1.2893 | B.149 | C.14 |
| 1.2894 | B.150 | C.14 |
| 1.2895 | B.151 | C.14 |
| 1.2896 | B.152 | C.14 |
| 1.2897 | B.153 | C.14 |
| 1.2898 | B.154 | C.14 |
| 1.2899 | B.155 | C.14 |
| 1.2900 | B.156 | C.14 |
| 1.2901 | B.157 | C.14 |
| 1.2902 | B.158 | C.14 |
| 1.2903 | B.159 | C.14 |
| 1.2904 | B.160 | C.14 |
| 1.2905 | B.161 | C.14 |
| 1.2906 | B.162 | C.14 |
| 1.2907 | B.163 | C.14 |
| 1.2908 | B.164 | C.14 |
| 1.2909 | B.165 | C.14 |
| 1.2910 | B.166 | C.14 |
| 1.2911 | B.167 | C.14 |
| 1.2912 | B.168 | C.14 |
| 1.2913 | B.169 | C.14 |
| 1.2914 | B.170 | C.14 |
| 1.2915 | B.171 | C.14 |
| 1.2916 | B.172 | C.14 |
| 1.2917 | B.173 | C.14 |
| 1.2918 | B.174 | C.14 |
| 1.2919 | B.175 | C.14 |
| 1.2920 | B.176 | C.14 |
| 1.2921 | B.177 | C.14 |
| 1.2922 | B.178 | C.14 |
| 1.2923 | B.179 | C.14 |
| 1.2924 | B.180 | C.14 |
| 1.2925 | B.181 | C.14 |
| 1.2926 | B.182 | C.14 |
| 1.2927 | B.183 | C.14 |
| 1.2928 | B.184 | C.14 |
| 1.2929 | B.185 | C.14 |
| 1.2930 | B.186 | C.14 |
| 1.2931 | B.187 | C.14 |
| 1.2932 | B.188 | C.14 |
| 1.2933 | B.189 | C.14 |
| 1.2934 | B.190 | C.14 |
| 1.2935 | B.191 | C.14 |
| 1.2936 | B.192 | C.14 |
| 1.2937 | B.193 | C.14 |
| 1.2938 | B.194 | C.14 |
| 1.2939 | B.195 | C.14 |
| 1.2940 | B.196 | C.14 |
| 1.2941 | B.1 | C.15 |
| 1.2942 | B.2 | C.15 |
| 1.2943 | B.3 | C.15 |
| 1.2944 | B.4 | C.15 |
| 1.2945 | B.5 | C.15 |
| 1.2946 | B.6 | C.15 |
| 1.2947 | B.7 | C.15 |
| 1.2948 | B.8 | C.15 |
| 1.2949 | B.9 | C.15 |
| 1.2950 | B.10 | C.15 |
| 1.2951 | B.11 | C.15 |
| 1.2952 | B.12 | C.15 |
| 1.2953 | B.13 | C.15 |
| 1.2954 | B.14 | C.15 |
| 1.2955 | B.15 | C.15 |
| 1.2956 | B.16 | C.15 |
| 1.2957 | B.17 | C.15 |
| 1.2958 | B.18 | C.15 |
| 1.2959 | B.19 | C.15 |
| 1.2960 | B.20 | C.15 |
| 1.2961 | B.21 | C.15 |
| 1.2962 | B.22 | C.15 |
| 1.2963 | B.23 | C.15 |
| 1.2964 | B.24 | C.15 |
| 1.2965 | B.25 | C.15 |
| 1.2966 | B.26 | C.15 |
| 1.2967 | B.27 | C.15 |
| 1.2968 | B.28 | C.15 |
| 1.2969 | B.29 | C.15 |
| 1.2970 | B.30 | C.15 |
| 1.2971 | B.31 | C.15 |
| 1.2972 | B.32 | C.15 |
| 1.2973 | B.33 | C.15 |
| 1.2974 | B.34 | C.15 |
| 1.2975 | B.35 | C.15 |
| 1.2976 | B.36 | C.15 |
| 1.2977 | B.37 | C.15 |
| 1.2978 | B.38 | C.15 |
| 1.2979 | B.39 | C.15 |
| 1.2980 | B.40 | C.15 |
| 1.2981 | B.41 | C.15 |
| 1.2982 | B.42 | C.15 |
| 1.2983 | B.43 | C.15 |
| 1.2984 | B.44 | C.15 |
| 1.2985 | B.45 | C.15 |
| 1.2986 | B.46 | C.15 |
| 1.2987 | B.47 | C.15 |
| 1.2988 | B.48 | C.15 |
| 1.2989 | B.49 | C.15 |
| 1.2990 | B.50 | C.15 |
| 1.2991 | B.51 | C.15 |
| 1.2992 | B.52 | C.15 |
| 1.2993 | B.53 | C.15 |
| 1.2994 | B.54 | C.15 |
| 1.2995 | B.55 | C.15 |
| 1.2996 | B.56 | C.15 |
| 1.2997 | B.57 | C.15 |
| 1.2998 | B.58. | C.15 |
| 1.2999 | B.59 | C.15 |
| 1.3000 | B.60 | C.15 |
| 1.3001 | B.61 | C.15 |
| 1.3002 | B.62 | C.15 |
| 1.3003 | B.63 | C.15 |
| 1.3004 | B.64 | C.15 |
| 1.3005 | B.65 | C.15 |
| 1.3006 | B.66 | C.15 |
| 1.3007 | B.67 | C.15 |
| 1.3008 | B.68 | C.15 |
| 1.3009 | B.69 | C.15 |
| 1.3010 | B.70 | C.15 |
| 1.3011 | B.71 | C.15 |
| 1.3012 | B.72 | C.15 |
| 1.3013 | B.73 | C.15 |
| 1.3014 | B.74 | C.15 |
| 1.3015 | B.75 | C.15 |
| 1.3016 | B.76 | C.15 |
| 1.3017 | B.77 | C.15 |
| 1.3018 | B.78 | C.15 |
| 1.3019 | B.79 | C.15 |
| 1.3020 | B.80 | C.15 |
| 1.3021 | B.81 | C.15 |
| 1.3022 | B.82 | C.15 |
| 1.3023 | B.83 | C.15 |
| 1.3024 | B.84 | C.15 |
| 1.3025 | B.85 | C.15 |
| 1.3026 | B.86 | C.15 |
| 1.3027 | B.87 | C.15 |
| 1.3028 | B.88 | C.15 |
| 1.3029 | B.89 | C.15 |
| 1.3030 | B.90 | C.15 |
| 1.3031 | B.91 | C.15 |
| 1.3032 | B.92 | C.15 |
| 1.3033 | B.93 | C.15 |
| 1.3034 | B.94 | C.15 |
| 1.3035 | B.95 | C.15 |
| 1.3036 | B.96 | C.15 |
| 1.3037 | B.97 | C.15 |
| 1.3038 | B.98 | C.15 |
| 1.3039 | B.99 | C.15 |
| 1.3040 | B.100 | C.15 |
| 1.3041 | B.101 | C.15 |
| 1.3042 | B.102 | C.15 |
| 1.3043 | B.103 | C.15 |
| 1.3044 | B.104 | C.15 |
| 1.3045 | B.105 | C.15 |
| 1.3046 | B.106 | C.15 |
| 1.3047 | B.107 | C.15 |
| 1.3048 | B.108 | C.15 |
| 1.3049 | B.109 | C.15 |
| 1.3050 | B.110 | C.15 |
| 1.3051 | B.111 | C.15 |
| 1.3052 | B.112 | C.15 |
| 1.3053 | B.113 | C.15 |
| 1.3054 | B.114 | C.15 |
| 1.3055 | B.115 | C.15 |
| 1.3056 | B.116 | C.15 |
| 1.3057 | B.117 | C.15 |
| 1.3058 | B.118 | C.15 |
| 1.3059 | B.119 | C.15 |
| 1.3060 | B.120 | C.15 |
| 1.3061 | B.121 | C.15 |
| 1.3062 | B.122 | C.15 |
| 1.3063 | B.123 | C.15 |
| 1.3064 | B.124 | C.15 |
| 1.3065 | B.125 | C.15 |
| 1.3066 | B.126 | C.15 |
| 1.3067 | B.127 | C.15 |
| 1.3068 | B.128 | C.15 |
| 1.3069 | B.129 | C.15 |
| 1.3070 | B.130 | C.15 |
| 1.3071 | B.131 | C.15 |
| 1.3072 | B.132 | C.15 |
| 1.3073 | B.133 | C.15 |
| 1.3074 | B.134 | C.15 |
| 1.3075 | B.135 | C.15 |
| 1.3076 | B.136 | C.15 |
| 1.3077 | B.137 | C.15 |
| 1.3078 | B.138 | C.15 |
| 1.3079 | B.139 | C.15 |
| 1.3080 | B.140 | C.15 |
| 1.3081 | B.141 | C.15 |
| 1.3082 | B.142 | C.15 |
| 1.3083 | B.143 | C.15 |
| 1.3084 | B.144 | C.15 |
| 1.3085 | B.145 | C.15 |
| 1.3086 | B.146 | C.15 |
| 1.3087 | B.147 | C.15 |
| 1.3088 | B.148 | C.15 |
| 1.3089 | B.149 | C.15 |
| 1.3090 | B.150 | C.15 |
| 1.3091 | B.151 | C.15 |
| 1.3092 | B.152 | C.15 |
| 1.3093 | B.153 | C.15 |
| 1.3094 | B.154 | C.15 |
| 1.3095 | B.155 | C.15 |
| 1.3096 | B.156 | C.15 |
| 1.3097 | B.157 | C.15 |
| 1.3098 | B.158 | C.15 |
| 1.3099 | B.159 | C.15 |
| 1.3100 | B.160 | C.15 |
| 1.3101 | B.161 | C.15 |
| 1.3102 | B.162 | C.15 |
| 1.3103 | B.163 | C.15 |
| 1.3104 | B.164 | C.15 |
| 1.3105 | B.165 | C.15 |
| 1.3106 | B.166 | C.15 |
| 1.3107 | B.167 | C.15 |
| 1.3108 | B.168 | C.15 |
| 1.3109 | B.169 | C.15 |
| 1.3110 | B.170 | C.15 |
| 1.3111 | B.171 | C.15 |
| 1.3112 | B.172 | C.15 |
| 1.3113 | B.173 | C.15 |
| 1.3114 | B.174 | C.15 |
| 1.3115 | B.175 | C.15 |
| 1.3116 | B.176 | C.15 |
| 1.3117 | B.177 | C.15 |
| 1.3118 | B.178 | C.15 |
| 1.3119 | B.179 | C.15 |
| 1.3120 | B.180 | C.15 |
| 1.3121 | B.181 | C.15 |
| 1.3122 | B.182 | C.15 |
| 1.3123 | B.183 | C.15 |
| 1.3124 | B.184 | C.15 |
| 1.3125 | B.185 | C.15 |
| 1.3126 | B.186 | C.15 |
| 1.3127 | B.187 | C.15 |
| 1.3128 | B.188 | C.15 |
| 1.3129 | B.189 | C.15 |
| 1.3130 | B.190 | C.15 |
| 1.3131 | B.191 | C.15 |
| 1.3132 | B.192 | C.15 |
| 1.3133 | B.193 | C.15 |
| 1.3134 | B.194 | C.15 |
| 1.3135 | B.195 | C.15 |
| 1.3136 | B.196 | C.15 |
| 1.3137 | B.1 | C.16 |
| 1.3138 | B.2 | C.16 |
| 1.3139 | B.3 | C.16 |
| 1.3140 | B.4 | C.16 |
| 1.3141 | B.5 | C.16 |
| 1.3142 | B.6 | C.16 |
| 1.3143 | B.7 | C.16 |
| 1.3144 | B.8 | C.16 |
| 1.3145 | B.9 | C.16 |
| 1.3146 | B.10 | C.16 |
| 1.3147 | B.11 | C.16 |
| 1.3148 | B.12 | C.16 |
| 1.3149 | B.13 | C.16 |
| 1.3150 | B.14 | C.16 |
| 1.3151 | B.15 | C.16 |
| 1.3152 | B.16 | C.16 |
| 1.3153 | B.17 | C.16 |
| 1.3154 | B.18 | C.16 |
| 1.3155 | B.19 | C.16 |
| 1.3156 | B.20 | C.16 |
| 1.3157 | B.21 | C.16 |
| 1.3158 | B.22 | C.16 |
| 1.3159 | B.23 | C.16 |
| 1.3160 | B.24 | C.16 |
| 1.3161 | B.25 | C.16 |
| 1.3162 | B.26 | C.16 |
| 1.3163 | B.27 | C.16 |
| 1.3164 | B.28 | C.16 |
| 1.3165 | B.29 | C.16 |
| 1.3166 | B.30 | C.16 |
| 1.3167 | B.31 | C.16 |
| 1.3168 | B.32 | C.16 |
| 1.3169 | B.33 | C.16 |
| 1.3170 | B.34 | C.16 |
| 1.3171 | B.35 | C.16 |
| 1.3172 | B.36 | C.16 |
| 1.3173 | B.37 | C.16 |
| 1.3174 | B.38 | C.16 |
| 1.3175 | B.39 | C.16 |
| 1.3176 | B.40 | C.16 |
| 1.3177 | B.41 | C.16 |
| 1.3178 | B.42 | C.16 |
| 1.3179 | B.43 | C.16 |
| 1.3180 | B.44 | C.16 |
| 1.3181 | B.45 | C.16 |
| 1.3182 | B.46 | C.16 |
| 1.3183 | B.47 | C.16 |
| 1.3184 | B.48 | C.16 |
| 1.3185 | B.49 | C.16 |
| 1.3186 | B.50 | C.16 |
| 1.3187 | B.51 | C.16 |
| 1.3188 | B.52 | C.16 |
| 1.3189 | B.53 | C.16 |
| 1.3190 | B.54 | C.16 |
| 1.3191 | B.55 | C.16 |
| 1.3192 | B.56 | C.16 |
| 1.3193 | B.57 | C.16 |
| 1.3194 | B.58. | C.16 |
| 1.3195 | B.59 | C.16 |
| 1.3196 | B.60 | C.16 |
| 1.3197 | B.61 | C.16 |
| 1.3198 | B.62 | C.16 |
| 1.3199 | B.63 | C.16 |
| 1.3200 | B.64 | C.16 |
| 1.3201 | B.65 | C.16 |
| 1.3202 | B.66 | C.16 |
| 1.3203 | B.67 | C.16 |
| 1.3204 | B.68 | C.16 |
| 1.3205 | B.69 | C.16 |
| 1.3206 | B.70 | C.16 |
| 1.3207 | B.71 | C.16 |
| 1.3208 | B.72 | C.16 |
| 1.3209 | B.73 | C.16 |
| 1.3210 | B.74 | C.16 |
| 1.3211 | B.75 | C.16 |
| 1.3212 | B.76 | C.16 |
| 1.3213 | B.77 | C.16 |
| 1.3214 | B.78 | C.16 |
| 1.3215 | B.79 | C.16 |
| 1.3216 | B.80 | C.16 |
| 1.3217 | B.81 | C.16 |
| 1.3218 | B.82 | C.16 |
| 1.3219 | B.83 | C.16 |
| 1.3220 | B.84 | C.16 |
| 1.3221 | B.85 | C.16 |
| 1.3222 | B.86 | C.16 |
| 1.3223 | B.87 | C.16 |
| 1.3224 | B.88 | C.16 |
| 1.3225 | B.89 | C.16 |
| 1.3226 | B.90 | C.16 |
| 1.3227 | B.91 | C.16 |
| 1.3228 | B.92 | C.16 |
| 1.3229 | B.93 | C.16 |
| 1.3230 | B.94 | C.16 |
| 1.3231 | B.95 | C.16 |
| 1.3232 | B.96 | C.16 |
| 1.3233 | B.97 | C.16 |
| 1.3234 | B.98 | C.16 |
| 1.3235 | B.99 | C.16 |
| 1.3236 | B.100 | C.16 |
| 1.3237 | B.101 | C.16 |
| 1.3238 | B.102 | C.16 |
| 1.3239 | B.103 | C.16 |
| 1.3240 | B.104 | C.16 |
| 1.3241 | B.105 | C.16 |
| 1.3242 | B.106 | C.16 |
| 1.3243 | B.107 | C.16 |
| 1.3244 | B.108 | C.16 |
| 1.3245 | B.109 | C.16 |
| 1.3246 | B.110 | C.16 |
| 1.3247 | B.111 | C.16 |
| 1.3248 | B.112 | C.16 |
| 1.3249 | B.113 | C.16 |
| 1.3250 | B.114 | C.16 |
| 1.3251 | B.115 | C.16 |
| 1.3252 | B.116 | C.16 |
| 1.3253 | B.117 | C.16 |
| 1.3254 | B.118 | C.16 |
| 1.3255 | B.119 | C.16 |
| 1.3256 | B.120 | C.16 |
| 1.3257 | B.121 | C.16 |
| 1.3258 | B.122 | C.16 |
| 1.3259 | B.123 | C.16 |
| 1.3260 | B.124 | C.16 |
| 1.3261 | B.125 | C.16 |
| 1.3262 | B.126 | C.16 |
| 1.3263 | B.127 | C.16 |
| 1.3264 | B.128 | C.16 |
| 1.3265 | B.129 | C.16 |
| 1.3266 | B.130 | C.16 |
| 1.3267 | B.131 | C.16 |
| 1.3268 | B.132 | C.16 |
| 1.3269 | B.133 | C.16 |
| 1.3270 | B.134 | C.16 |
| 1.3271 | B.135 | C.16 |
| 1.3272 | B.136 | C.16 |
| 1.3273 | B.137 | C.16 |
| 1.3274 | B.138 | C.16 |
| 1.3275 | B.139 | C.16 |
| 1.3276 | B.140 | C.16 |
| 1.3277 | B.141 | C.16 |
| 1.3278 | B.142 | C.16 |
| 1.3279 | B.143 | C.16 |
| 1.3280 | B.144 | C.16 |
| 1.3281 | B.145 | C.16 |
| 1.3282 | B.146 | C.16 |
| 1.3283 | B.147 | C.16 |
| 1.3284 | B.148 | C.16 |
| 1.3285 | B.149 | C.16 |
| 1.3286 | B.150 | C.16 |
| 1.3287 | B.151 | C.16 |
| 1.3288 | B.152 | C.16 |
| 1.3289 | B.153 | C.16 |
| 1.3290 | B.154 | C.16 |
| 1.3291 | B.155 | C.16 |
| 1.3292 | B.156 | C.16 |
| 1.3293 | B.157 | C.16 |
| 1.3294 | B.158 | C.16 |
| 1.3295 | B.159 | C.16 |
| 1.3296 | B.160 | C.16 |
| 1.3297 | B.161 | C.16 |
| 1.3298 | B.162 | C.16 |
| 1.3299 | B.163 | C.16 |
| 1.3300 | B.164 | C.16 |
| 1.3301 | B.165 | C.16 |
| 1.3302 | B.166 | C.16 |
| 1.3303 | B.167 | C.16 |
| 1.3304 | B.168 | C.16 |
| 1.3305 | B.169 | C.16 |
| 1.3306 | B.170 | C.16 |
| 1.3307 | B.171 | C.16 |
| 1.3308 | B.172 | C.16 |
| 1.3309 | B.173 | C.16 |
| 1.3310 | B.174 | C.16 |
| 1.3311 | B.175 | C.16 |
| 1.3312 | B.176 | C.16 |
| 1.3313 | B.177 | C.16 |
| 1.3314 | B.178 | C.16 |
| 1.3315 | B.179 | C.16 |
| 1.3316 | B.180 | C.16 |
| 1.3317 | B.181 | C.16 |
| 1.3318 | B.182 | C.16 |
| 1.3319 | B.183 | C.16 |
| 1.3320 | B.184 | C.16 |
| 1.3321 | B.185 | C.16 |
| 1.3322 | B.186 | C.16 |
| 1.3323 | B.187 | C.16 |
| 1.3324 | B.188 | C.16 |
| 1.3325 | B.189 | C.16 |
| 1.3326 | B.190 | C.16 |
| 1.3327 | B.191 | C.16 |
| 1.3328 | B.192 | C.16 |
| 1.3329 | B.193 | C.16 |
| 1.3330 | B.194 | C.16 |
| 1.3331 | B.195 | C.16 |
| 1.3332 | B.196 | C.16 |
| 1.3333 | B.1 | C.17 |
| 1.3334 | B.2 | C.17 |
| 1.3335 | B.3 | C.17 |
| 1.3336 | B.4 | C.17 |
| 1.3337 | B.5 | C.17 |
| 1.3338 | B.6 | C.17 |
| 1.3339 | B.7 | C.17 |
| 1.3340 | B.8 | C.17 |
| 1.3341 | B.9 | C.17 |
| 1.3342 | B.10 | C.17 |
| 1.3343 | B.11 | C.17 |
| 1.3344 | B.12 | C.17 |
| 1.3345 | B.13 | C.17 |
| 1.3346 | B.14 | C.17 |
| 1.3347 | B.15 | C.17 |
| 1.3348 | B.16 | C.17 |
| 1.3349 | B.17 | C.17 |
| 1.3350 | B.18 | C.17 |
| 1.3351 | B.19 | C.17 |
| 1.3352 | B.20 | C.17 |
| 1.3353 | B.21 | C.17 |
| 1.3354 | B.22 | C.17 |
| 1.3355 | B.23 | C.17 |
| 1.3356 | B.24 | C.17 |
| 1.3357 | B.25 | C.17 |
| 1.3358 | B.26 | C.17 |
| 1.3359 | B.27 | C.17 |
| 1.3360 | B.28 | C.17 |
| 1.3361 | B.29 | C.17 |
| 1.3362 | B.30 | C.17 |
| 1.3363 | B.31 | C.17 |
| 1.3364 | B.32 | C.17 |
| 1.3365 | B.33 | C.17 |
| 1.3366 | B.34 | C.17 |
| 1.3367 | B.35 | C.17 |
| 1.3368 | B.36 | C.17 |
| 1.3369 | B.37 | C.17 |
| 1.3370 | B.38 | C.17 |
| 1.3371 | B.39 | C.17 |
| 1.3372 | B.40 | C.17 |
| 1.3373 | B.41 | C.17 |
| 1.3374 | B.42 | C.17 |
| 1.3375 | B.43 | C.17 |
| 1.3376 | B.44 | C.17 |
| 1.3377 | B.45 | C.17 |
| 1.3378 | B.46 | C.17 |
| 1.3379 | B.47 | C.17 |
| 1.3380 | B.48 | C.17 |
| 1.3381 | B.49 | C.17 |
| 1.3382 | B.50 | C.17 |
| 1.3383 | B.51 | C.17 |
| 1.3384 | B.52 | C.17 |
| 1.3385 | B.53 | C.17 |
| 1.3386 | B.54 | C.17 |
| 1.3387 | B.55 | C.17 |
| 1.3388 | B.56 | C.17 |
| 1.3389 | B.57 | C.17 |
| 1.3390 | B.58. | C.17 |
| 1.3391 | B.59 | C.17 |
| 1.3392 | B.60 | C.17 |
| 1.3393 | B.61 | C.17 |
| 1.3394 | B.62 | C.17 |
| 1.3395 | B.63 | C.17 |
| 1.3396 | B.64 | C.17 |
| 1.3397 | B.65 | C.17 |
| 1.3398 | B.66 | C.17 |
| 1.3399 | B.67 | C.17 |
| 1.3400 | B.68 | C.17 |
| 1.3401 | B.69 | C.17 |
| 1.3402 | B.70 | C.17 |
| 1.3403 | B.71 | C.17 |
| 1.3404 | B.72 | C.17 |
| 1.3405 | B.73 | C.17 |
| 1.3406 | B.74 | C.17 |
| 1.3407 | B.75 | C.17 |
| 1.3408 | B.76 | C.17 |
| 1.3409 | B.77 | C.17 |
| 1.3410 | B.78 | C.17 |
| 1.3411 | B.79 | C.17 |
| 1.3412 | B.80 | C.17 |
| 1.3413 | B.81 | C.17 |
| 1.3414 | B.82 | C.17 |
| 1.3415 | B.83 | C.17 |
| 1.3416 | B.84 | C.17 |
| 1.3417 | B.85 | C.17 |
| 1.3418 | B.86 | C.17 |
| 1.3419 | B.87 | C.17 |
| 1.3420 | B.88 | C.17 |
| 1.3421 | B.89 | C.17 |
| 1.3422 | B.90 | C.17 |
| 1.3423 | B.91 | C.17 |
| 1.3424 | B.92 | C.17 |
| 1.3425 | B.93 | C.17 |
| 1.3426 | B.94 | C.17 |
| 1.3427 | B.95 | C.17 |
| 1.3428 | B.96 | C.17 |
| 1.3429 | B.97 | C.17 |
| 1.3430 | B.98 | C.17 |
| 1.3431 | B.99 | C.17 |
| 1.3432 | B.100 | C.17 |
| 1.3433 | B.101 | C.17 |
| 1.3434 | B.102 | C.17 |
| 1.3435 | B.103 | C.17 |
| 1.3436 | B.104 | C.17 |
| 1.3437 | B.105 | C.17 |
| 1.3438 | B.106 | C.17 |
| 1.3439 | B.107 | C.17 |
| 1.3440 | B.108 | C.17 |
| 1.3441 | B.109 | C.17 |
| 1.3442 | B.110 | C.17 |
| 1.3443 | B.111 | C.17 |
| 1.3444 | B.112 | C.17 |
| 1.3445 | B.113 | C.17 |
| 1.3446 | B.114 | C.17 |
| 1.3447 | B.115 | C.17 |
| 1.3448 | B.116 | C.17 |
| 1.3449 | B.117 | C.17 |
| 1.3450 | B.118 | C.17 |
| 1.3451 | B.119 | C.17 |
| 1.3452 | B.120 | C.17 |
| 1.3453 | B.121 | C.17 |
| 1.3454 | B.122 | C.17 |
| 1.3455 | B.123 | C.17 |
| 1.3456 | B.124 | C.17 |
| 1.3457 | B.125 | C.17 |
| 1.3458 | B.126 | C.17 |
| 1.3459 | B.127 | C.17 |
| 1.3460 | B.128 | C.17 |
| 1.3461 | B.129 | C.17 |
| 1.3462 | B.130 | C.17 |
| 1.3463 | B.131 | C.17 |
| 1.3464 | B.132 | C.17 |
| 1.3465 | B.133 | C.17 |
| 1.3466 | B.134 | C.17 |
| 1.3467 | B.135 | C.17 |
| 1.3468 | B.136 | C.17 |
| 1.3469 | B.137 | C.17 |
| 1.3470 | B.138 | C.17 |
| 1.3471 | B.139 | C.17 |
| 1.3472 | B.140 | C.17 |
| 1.3473 | B.141 | C.17 |
| 1.3474 | B.142 | C.17 |
| 1.3475 | B.143 | C.17 |
| 1.3476 | B.144 | C.17 |
| 1.3477 | B.145 | C.17 |
| 1.3478 | B.146 | C.17 |
| 1.3479 | B.147 | C.17 |
| 1.3480 | B.148 | C.17 |
| 1.3481 | B.149 | C.17 |
| 1.3482 | B.150 | C.17 |
| 1.3483 | B.151 | C.17 |
| 1.3484 | B.152 | C.17 |
| 1.3485 | B.153 | C.17 |
| 1.3486 | B.154 | C.17 |
| 1.3487 | B.155 | C.17 |
| 1.3488 | B.156 | C.17 |
| 1.3489 | B.157 | C.17 |
| 1.3490 | B.158 | C.17 |
| 1.3491 | B.159 | C.17 |
| 1.3492 | B.160 | C.17 |
| 1.3493 | B.161 | C.17 |
| 1.3494 | B.162 | C.17 |
| 1.3495 | B.163 | C.17 |
| 1.3496 | B.164 | C.17 |
| 1.3497 | B.165 | C.17 |
| 1.3498 | B.166 | C.17 |
| 1.3499 | B.167 | C.17 |
| 1.3500 | B.168 | C.17 |
| 1.3501 | B.169 | C.17 |
| 1.3502 | B.170 | C.17 |
| 1.3503 | B.171 | C.17 |
| 1.3504 | B.172 | C.17 |
| 1.3505 | B.173 | C.17 |
| 1.3506 | B.174 | C.17 |
| 1.3507 | B.175 | C.17 |
| 1.3508 | B.176 | C.17 |
| 1.3509 | B.177 | C.17 |
| 1.3510 | B.178 | C.17 |
| 1.3511 | B.179 | C.17 |
| 1.3512 | B.180 | C.17 |
| 1.3513 | B.181 | C.17 |
| 1.3514 | B.182 | C.17 |
| 1.3515 | B.183 | C.17 |
| 1.3516 | B.184 | C.17 |
| 1.3517 | B.185 | C.17 |
| 1.3518 | B.186 | C.17 |
| 1.3519 | B.187 | C.17 |
| 1.3520 | B.188 | C.17 |
| 1.3521 | B.189 | C.17 |
| 1.3522 | B.190 | C.17 |
| 1.3523 | B.191 | C.17 |
| 1.3524 | B.192 | C.17 |
| 1.3525 | B.193 | C.17 |
| 1.3526 | B.194 | C.17 |
| 1.3527 | B.195 | C.17 |
| 1.3528 | B.196 | C.17 |
| 1.3529 | — | C.1 |
| 1.3530 | — | C.2 |
| 1.3531 | — | C.3 |
| 1.3532 | — | C.4 |
| 1.3533 | — | C.5 |
| 1.3534 | — | C.6 |
| 1.3535 | — | C.7 |
| 1.3536 | — | C.8 |
| 1.3537 | — | C.9 |
| 1.3538 | — | C.10 |
| 1.3539 | — | C.11 |
| 1.3540 | — | C.12 |
| 1.3541 | — | C.13 |
| 1.3542 | — | C.14 |
| 1.3543 | — | C.15 |
| 1.3544 | — | C.16 |
| 1.3545 | — | C.17 |

The specific number for each single composition is deductible as follows: Composition 1.200 for example comprises compounds of formula (I) cyhalofop-butyl (B.4) and benoxacor (C.1). Composition 2.200 for example comprises the compounds of formula (I), cyhalofop-butyl (B.4) and benoxacor (C.1).

Composition 7.200 for example comprises compounds of formula (I) imazapyr (B.35), cyhalofop-butyl (B.4) and benoxacor (C.1).

Also especially preferred are compositions 2.1. to 2.3545 which differ from the corresponding compositions 1.1 to 1.3545 only in that they comprise as the active compound A the compounds of formula (Ia).

Also especially preferred are compositions 3.1. to 3.3545 which differ from the corresponding compositions 1.1 to 1.3545 only in that they additionally comprise B.2 clodinafop-propargyl as further herbicide B.

Also especially preferred are compositions 4.1. to 4.3545 which differ from the corresponding compositions 1.1 to 1.3545 only in that they additionally comprise B.8 pinoxaden as further herbicide B.

Also especially preferred are compositions 5.1. to 5.3545 which differ from the corresponding compositions 1.1 to 1.3545 only in that they additionally comprise B.30 imazamox as further herbicide B.

Also especially preferred are compositions 6.1. to 6.3545 which differ from the corresponding compositions 1.1 to 1.3545 only in that they additionally comprise B.32 imazapic as further herbicide B.

Also especially preferred are compositions 7.1. to 7.3545 which differ from the corresponding compositions 1.1 to 1.3545 only in that they additionally comprise B.35 imazapyr as further herbicide B.

Also especially preferred are compositions 8.1. to 8.3545 which differ from the corresponding compositions 1.1 to 1.3545 only in that they additionally comprise B.38 imazaquin as further herbicide B.

Also especially preferred are compositions 9.1. to 9.3545 which differ from the corresponding compositions 1.1 to 1.3545 only in that they additionally comprise B.40 imazethapyr as further herbicide B.

Also especially preferred are compositions 10.1. to 10.3545 which differ from the corresponding compositions 1.1 to 1.3545 only in that they additionally comprise B.51 nicosulfuron as further herbicide B.

Also especially preferred are compositions 11.1. to 11.3545 which differ from the corresponding compositions 1.1 to 1.3545 only in that they additionally comprise B.55 pyribenzoxim as further herbicide B.

Also especially preferred are compositions 12.1. to 12.3545 which differ from the corresponding compositions 1.1 to 1.3545 only in that they additionally comprise B.56 pyriftalid as further herbicide B.

Also especially preferred are compositions 13.1. to 13.3545 which differ from the corresponding compositions 1.1 to 1.3545 only in that they additionally comprise B.64 tritosulfuron as further herbicide B.

Also especially preferred are compositions 14.1. to 14.3545 which differ from the corresponding compositions 1.1 to 1.3545 only in that they additionally comprise B.66 ametryne as further herbicide B.

Also especially preferred are compositions 15.1. to 15.3545 which differ from the corresponding compositions 1.1 to 1.3545 only in that they additionally comprise B.67 atrazine as further herbicide B.

Also especially preferred are compositions 16.1. to 16.3545 which differ from the corresponding compositions 1.1 to 1.3545 only in that they additionally comprise B.68 bentazon as further herbicide B.

Also especially preferred are compositions 17.1. to 17.3545 which differ from the corresponding compositions 1.1 to 1.3545 only in that they additionally comprise B.69 bromoxynil as further herbicide B.

Also especially preferred are compositions 18.1. to 18.3545 which differ from the corresponding compositions 1.1 to 1.3545 only in that they additionally comprise B.73 diuron as further herbicide B.

Also especially preferred are compositions 19.1. to 19.3545 which differ from the corresponding compositions 1.1 to 1.3545 only in that they additionally comprise B.76 isoproturon as further herbicide B.

Also especially preferred are compositions 20.1. to 20.3545 which differ from the corresponding compositions 1.1 to 1.3545 only in that they additionally comprise B.81 simazin as further herbicide B.

Also especially preferred are compositions 21.1. to 21.3545 which differ from the corresponding compositions 1.1 to 1.3545 only in that they additionally comprise B.82 terbuthylazin as further herbicide B.

Also especially preferred are compositions 22.1. to 22.3545 which differ from the corresponding compositions 1.1 to 1.3545 only in that they additionally comprise B.85 acifluorfen as further herbicide B.

Also especially preferred are compositions 23.1. to 23.3545 which differ from the corresponding compositions 1.1 to 1.3545 only in that they additionally comprise B.88 flumioxazin as further herbicide B.

Also especially preferred are compositions 24.1. to 24.3545 which differ from the corresponding compositions 1.1 to 1.3545 only in that they additionally comprise B.89 fomesafen as further herbicide B.

Also especially preferred are compositions 25.1. to 25.3545 which differ from the corresponding compositions 1.1 to 1.3545 only in that they additionally comprise B.94 saflufenacil as further herbicide B.

Also especially preferred are compositions 26.1. to 26.3545 which differ from the corresponding compositions 1.1 to 1.3545 only in that they additionally comprise B.95 sulfentrazone as further herbicide B.

Also especially preferred are compositions 27.1. to 27.3545 which differ from the corresponding compositions 1.1 to 1.3545 only in that they additionally comprise B.98 benzbicyclone as further herbicide B.

Also especially preferred are compositions 28.1. to 28.3545 which differ from the corresponding compositions 1.1 to 1.3545 only in that they additionally comprise B.100 clomazone as further herbicide B.

Also especially preferred are compositions 29.1. to 29.3545 which differ from the corresponding compositions 1.1 to 1.3545 only in that they additionally comprise B.103 isoxaflutole as further herbicide B.

Also especially preferred are compositions 30.1. to 30.3545 which differ from the corresponding compositions 1.1 to 1.3545 only in that they additionally comprise B.103 isoxaflutole and B.67 atrazine as further herbicides B.

Also especially preferred are compositions 31.1. to 31.3545 which differ from the corresponding compositions 1.1 to 1.3545 only in that they additionally comprise B.103 isoxaflutole and B.76 isoproturon as further herbicides B.

Also especially preferred are compositions 32.1. to 32.3545 which differ from the corresponding compositions 1.1 to 1.3545 only in that they additionally comprise B.103 isoxaflutole and B.82 terbutylazin as further herbicides B.

Also especially preferred are compositions 33.1. to 33.3545 which differ from the corresponding compositions 1.1 to 1.3545 only in that they additionally comprise B.104 mesotrione as further herbicide B.

Also especially preferred are compositions 34.1. to 34.3545 which differ from the corresponding compositions 1.1 to 1.3545 only in that they additionally comprise B.104 mesotrione and B.67 atrazine as further herbicides B.

Also especially preferred are compositions 35.1. to 35.3545 which differ from the corresponding compositions 1.1 to 1.3545 only in that they additionally comprise B.104 mesotrione and B.76 isoproturon as further herbicides B.

Also especially preferred are compositions 36.1. to 36.3545 which differ from the corresponding compositions 1.1 to 1.3545 only in that they additionally comprise B.104 mesotrione and B.82 terbutylazin as further herbicides B.

Also especially preferred are compositions 37.1. to 37.3545 which differ from the corresponding compositions 1.1 to 1.3545 only in that they additionally comprise B.106 picolinafen as further herbicide B.

Also especially preferred are compositions 38.1. to 38.3545 which differ from the corresponding compositions 1.1 to 1.3545 only in that they additionally comprise B.107 sulcotrione as further herbicide B.

Also especially preferred are compositions 39.1. to 39.3545 which differ from the corresponding compositions 1.1 to 1.3545 only in that they additionally comprise B. 107 sulcotrione and B.67 atrazine as further herbicides B.

Also especially preferred are compositions 40.1. to 40.3545 which differ from the corresponding compositions 1.1 to 1.3545 only in that they additionally comprise B. 107 sulcotrione and B.76 isoproturon as further herbicides B.

Also especially preferred are compositions 41.1. to 41.3545 which differ from the corresponding compositions 1.1 to 1.3545 only in that they additionally comprise B. 107 sulcotrione and B.82 terbutylazin as further herbicides B.

Also especially preferred are compositions 42.1. to 42.3545 which differ from the corresponding compositions 1.1 to 1.3545 only in that they additionally comprise B.109 tembotrione as further herbicide B.

Also especially preferred are compositions 43.1. to 43.3545 which differ from the corresponding compositions 1.1 to 1.3545 only in that they additionally comprise B.111 topramezone as further herbicide B.

Also especially preferred are compositions 44.1. to 44.3545 which differ from the corresponding compositions 1.1 to 1.3545 only in that they additionally comprise B.111 topramezone and B.67 atrazine as further herbicides B.

Also especially preferred are compositions 45.1. to 45.3545 which differ from the corresponding compositions 1.1 to 1.3545 only in that they additionally comprise B.111 topramezone and B.76 isoproturon as further herbicides B.

Also especially preferred are compositions 46.1. to 46.3545 which differ from the corresponding compositions 1.1 to 1.3545 only in that they additionally comprise B.111 topramezone and B.82 terbutylazin as further herbicides B.

Also especially preferred are compositions 47.1. to 47.3545 which differ from the corresponding compositions 1.1 to 1.3545 only in that they additionally comprise B.116 glyphosate as further herbicide B.

Also especially preferred are compositions 48.1. to 48.3545 which differ from the corresponding compositions 1.1 to 1.3545 only in that they additionally comprise B.116 glyphosate and B.67 atrazine as further herbicides B.

Also especially preferred are compositions 49.1. to 49.3545 which differ from the corresponding compositions 1.1 to 1.3545 only in that they additionally comprise B.116 glyphosate and B.94 saflufenacil as further herbicides B.

Also especially preferred are compositions 50.1. to 50.3545 which differ from the corresponding compositions 1.1 to 1.3545 only in that they additionally comprise B.116 glyphosate and B.103 isoxaflutole as further herbicides B.

Also especially preferred are compositions 51.1. to 51.3545 which differ from the corresponding compositions 1.1 to 1.3545 only in that they additionally comprise B.116 glyphosate and B.128 acetochlor as further herbicides B.

Also especially preferred are compositions 52.1. to 52.3545 which differ from the corresponding compositions 1.1 to 1.3545 only in that they additionally comprise B.116 glyphosate and B.104 mesotrione as further herbicides B.

Also especially preferred are compositions 53.1. to 53.3545 which differ from the corresponding compositions 1.1 to 1.3545 only in that they additionally comprise B.116 glyphosate and B.107 sulcotrione as further herbicides B.

Also especially preferred are compositions 54.1. to 54.3545 which differ from the corresponding compositions 1.1 to 1.3545 only in that they additionally comprise B.116 glyphosate and B.111 topramezone as further herbicides B.

Also especially preferred are compositions 55.1. to 55.3545 which differ from the corresponding compositions 1.1 to 1.3545 only in that they additionally comprise B.122 glufosinate as further herbicide B.

Also especially preferred are compositions 56.1. to 56.3545 which differ from the corresponding compositions 1.1 to 1.3545 only in that they additionally comprise B.126 pendimethalin as further herbicide B.

Also especially preferred are compositions 57.1. to 57.3545 which differ from the corresponding compositions 1.1 to 1.3545 only in that they additionally comprise B.128 acetochlor as further herbicide B.

Also especially preferred are compositions 58.1. to 58.3545 which differ from the corresponding compositions 1.1 to 1.3545 only in that they additionally comprise B.131 dimethenamid-P as further herbicide B.

Also especially preferred are compositions 59.1. to 59.3545 which differ from the corresponding compositions 1.1 to 1.3545 only in that they additionally comprise B.132 fentrazamide as further herbicide B.

Also especially preferred are compositions 60.1. to 60.3545 which differ from the corresponding compositions 1.1 to 1.3545 only in that they additionally comprise B.133 flufenacet as further herbicide B.

Also especially preferred are compositions 61.1. to 61.3545 which differ from the corresponding compositions 1.1 to 1.3545 only in that they additionally comprise B.135 metazachlor as further herbicide B.

Also especially preferred are compositions 62.1. to 62.3545 which differ from the corresponding compositions 1.1 to 1.3545 only in that they additionally comprise B.137 S-metolachlor as further herbicide B.

Also especially preferred are compositions 63.1. to 63.3545 which differ from the corresponding compositions 11.1 to 1.3545 only in that they additionally comprise B.138 pretilachlor as further herbicide B.

Also especially preferred are compositions 64.1. to 64.3545 which differ from the corresponding compositions 1.1 to 1.3545 only in that they additionally comprise B.140 indaziflam as further herbicide B.

Also especially preferred are compositions 65.1. to 65.3545 which differ from the corresponding compositions 1.1 to 1.3545 only in that they additionally comprise B.145 2,4-D as further herbicide B.

Also especially preferred are compositions 66.1. to 66.3545 which differ from the corresponding compositions 1.1 to 1.3545 only in that they additionally comprise B.153 clopyralid as further herbicide B.

Also especially preferred are compositions 67.1. to 67.3545 which differ from the corresponding compositions 1.1 to 1.3545 only in that they additionally comprise B.156 dicamba as further herbicide B.

Also especially preferred are compositions 68.1. to 68.3545 which differ from the corresponding compositions 1.1 to 1.3545 only in that they additionally comprise B.171 MCPA as further herbicide B.

Also especially preferred are compositions 69.1. to 69.3545 which differ from the corresponding compositions 1.1 to 1.3545 only in that they additionally comprise B.174 quinclorac as further herbicide B.

The invention also relates to agrochemical compositions comprising an auxiliary and at least one diaminotriazine compound of formula (I) or a composition according to the invention.

Accordingly, a first embodiment of the invention relates to compositions in the form of a agrochemical composition formulated as a 1-component composition comprising the at least one active compound of formula (I) or the at least one active compound of formula (I) (active compound A) and at least one further active compound selected from the herbicides B and the safeners C and also a solid or liquid carrier and, if appropriate, one or more surfactants.

Accordingly, a second embodiment of the invention relates to compositions in the form of a agrochemical composition formulated as a 2-component composition comprising a first formulation (component) comprising the at least one active compound A, which is a compound of formula (I), a solid or liquid carrier and, if appropriate, one or more surfactants, and a second component comprising at least one further active compound selected from the herbicides B and safeners C, a solid or liquid carrier and, if appropriate, one or more surfactants.

The active compound A, which is a compound of formula (I) and the at least one further active compound B and/or C can be formulated and applied jointly or separately, simultaneously or in succession, before, during or after the emergence of the plants. In case of separate application, the order of the application of the active compounds A, B and/or C is of minor importance. The only thing that is important is that the at least one active compound A and the at least one further active compound B and/or C are present simultaneously at the site of action, i.e. are at the same time in contact with or taken up by the plant to be controlled or to be safened.

An agrochemical composition comprises a pesticidally effective amount of a aminotriazine compound of formula (I). The term “effective amount” denotes an amount of the composition or of the compounds I, which is sufficient for controlling unwanted plants, especially for controlling unwanted plants in cultivated plants and which does not result in a substantial damage to the treated plants. Such an amount can vary in a broad range and is dependent on various factors, such as the plants to be controlled, the treated cultivated plant or material, the climatic conditions and the specific aminotriazine compound of formula (I) used.

The aminotriazine compound of formula (I), their N-oxides or salts can be converted into customary types of agrochemical compositions, e. g. solutions, emulsions, suspensions, dusts, powders, pastes, granules, pressings, capsules, and mixtures thereof. Examples for agrochemical composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further agrochemical compositions types are defined in the “Catalogue of pesticide formulation types and international coding system”, Technical Monograph No. 2, 6^{th} Ed. May 2008, CropLife International.

The agrochemical compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidally activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for agrochemical composition types and their preparation are:

i) Water-Soluble Concentrates (SL, LS)

10-60 wt % of an aminotriazine compound of formula (I) according to the invention or the herbicidal composition according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible Concentrates (DC)

5-25 wt % of an aminotriazine compound of formula (I) according to the invention or the herbicidal composition according to the invention and 1-10 wt % dispersant (e. g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of an aminotriazine compound of formula (I) according to the invention or the herbicidal composition according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

iv) Emulsions (EW, EO, ES)

5-40 wt % of an aminotriazine compound of formula (I) according to the invention or the herbicidal composition according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of an aminotriazine compound of formula (I) according to the invention or the herbicidal composition according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

vi) Water-Dispersible Granules and Water-Soluble Granules (WG, SG)

50-80 wt % of an aminotriazine compound of formula (I) according to the invention or the herbicidal composition according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of an aminotriazine compound of formula (I) according to the invention or the herbicidal composition according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of an aminotriazine compound of formula (I) according to the invention or the herbicidal composition according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

iv) Microemulsion (ME)

5-20 wt % of an aminotriazine compound of formula (I) according to the invention or the herbicidal composition according to the invention are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

iv) Microcapsules (CS)

An oil phase comprising 5-50 wt % of an aminotriazine compound of formula (I) according to the invention or the herbicidal composition according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of an aminotriazine compound of formula (I) according to the invention or the herbicidal composition according to the invention, 0-40 wt % water insoluble organic solvent (e.g. aromatic hydrocarbon), and an isocyanate monomer (e.g. diphenylmethene-4,4′-diisocyanate) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

ix) Dustable Powders (DP, DS)

1-10 wt % of an aminotriazine compound of formula (I) according to the invention or the herbicidal composition according to the invention are ground finely and mixed intimately with solid carrier (e.g. finely divided kaolin) ad 100 wt %.

x) Granules (GR, FG)

0.5-30 wt % of an aminotriazine compound of formula (I) according to the invention or the herbicidal composition according to the invention is ground finely and associated with solid carrier (e.g. silicate) ad 100 wt %. Granulation is achieved by extrusion, spray-drying or the fluidized bed.

xi) Ultra-Low Volume Liquids (UL)

1-50 wt % of an aminotriazine compound of formula (I) according to the invention or the herbicidal composition according to the invention are dissolved in organic solvent (e.g. aromatic hydrocarbon) ad 100 wt %.

The agrochemical compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of the aminotriazine compounds of formula (I). The aminotriazine compounds of formula (I) or the herbicidal composition according to the invention are employed in a purity of from 90% to 100%, preferably from 95% to 100% (according to NMR spectrum).

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The agrochemical compositions in question give, after two-to-tenfold dilution, active substance concentrations of from 0.01 to 60% by weight, preferably from 0.1 to 40% by weight, in the ready-to-use preparations. Application can be carried out before or during sowing.

Methods for applying aminotriazine compounds of formula (I) or agrochemical compositions thereof, on to plant propagation material, especially seeds, include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

Various types of oils, wetters, adjuvants, fertilizer, or micronutrients, and further pesticides (e.g. herbicides, insecticides, fungicides, growth regulators, safeners) may be added to the aminotriazine compounds of formula (I) or the agrochemical compositions comprising them as premix or, if appropriate not until immediately prior to use (tank mix). These agents can be admixed with the agrochemical compositions according to the invention in a weight ratio of 1:100 to 100:1, preferably 1:10 to 10:1.

The user applies the aminotriazine compounds of formula (I) according to the invention or the agrochemical compositions comprising them usually from a pre-dosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, either individual components of the agrochemical composition according to the invention or partially premixed components, e. g. components comprising azines of formula (I) may be mixed by the user in a spray tank and further auxiliaries and additives may be added, if appropriate.

In a further embodiment, individual components of the agrochemical composition according to the invention such as parts of a kit or parts of a binary or ternary mixture may be mixed by the user himself in a spray tank and further auxiliaries may be added, if appropriate.

In a further embodiment, either individual components of the agrochemical composition according to the invention or partially premixed components, e. g components comprising aminotriazine compounds of formula (I), can be applied jointly (e.g. after tank mix) or consecutively.

The aminotriazine compounds of formula (I), are suitable as herbicides. They are suitable as such or as an appropriately formulated composition (agrochemical composition).

The aminotriazine compounds of formula (I), or the agrochemical compositions comprising the azines of formula (I), control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

The aminotriazine compounds of formula (I), or the agrochemical compositions comprising them, are applied to the plants mainly by spraying the leaves or are applied to the soil in which the plant seeds have been sown. Here, the application can be carried out using, for example, water as carrier by customary spraying techniques using spray liquor amounts of from about 100 to 1000 l/ha (for example from 300 to 400 l/ha). The aminotriazine compounds of formula (I), or the agrochemical compositions comprising them, may also be applied by the low-volume or the ultra-low-volume method, or in the form of microgranules.

Application of the aminotriazine compounds of formula (I), or the agrochemical compositions comprising them, can be done before, during and/or after the emergence of the undesirable plants.

The aminotriazine compounds of formula (I), or the agrochemical compositions comprising them, can be applied pre-, post-emergence or pre-plant, or together with the seed of a crop plant. It is also possible to apply the aminotriazine compounds of formula (I), or the agrochemical compositions comprising them, by applying seed, pretreated with the aminotriazine compounds of formula (I), or the agrochemical compositions comprising them, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the aminotriazine compounds of formula (I), or the agrochemical compositions comprising them, can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the aminotriazine compounds of formula (I), or the agrochemical compositions prepared therefrom. Here, the herbicidal compositions can be applied diluted or undiluted.

The term “seed” comprises seed of all types, such as, for example, corns, seeds, fruits, tubers, seedlings and similar forms. Here, preferably, the term seed describes corns and seeds. The seed used can be seed of the useful plants mentioned above, but also the seed of transgenic plants or plants obtained by customary breeding methods.

When employed in plant protection, the amounts of active substances applied, i.e. the aminotriazine compounds of formula (I), without formulation auxiliaries, are, depending on the kind of effect desired, from 0.001 to 2 kg per ha, preferably from 0.005 to 2 kg per ha, more preferably from 0.005 to 0.9 kg per ha and in particular from 0.05 to 0.5 kg per ha.

In another embodiment of the invention, the application rate of the aminotriazine compounds of formula (I) is from 0.001 to 3 kg/ha, preferably from 0.005 to 2.5 kg/ha, of active substance (a.s.).

In another preferred embodiment of the invention, the rates of application of the aminotriazine compounds of formula (I) according to the present invention (total amount of aminotriazine compounds of formula (I)) are from 0.1 g/ha to 3000 g/ha, preferably 10 g/ha to 1000 g/ha, depending on the control target, the season, the target plants and the growth stage.

In another preferred embodiment of the invention, the application rates of the aminotriazine compounds of formula (I) are in the range from 0.1 g/ha to 5000 g/ha and preferably in the range from 1 g/ha to 2500 g/ha or from 5 g/ha to 2000 g/ha.

In another preferred embodiment of the invention, the application rate of the aminotriazine compounds of formula (I) is 0.1 to 1000 g/ha, preferably to 750 g/ha, more preferably 5 to 500 g/ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

In another embodiment of the invention, to treat the seed, the amounts of active substances applied, i.e. the aminotriazine compounds of formula (I) are generally employed in amounts of from 0.001 to 10 kg per 100 kg of seed.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Depending on the application method in question, the aminotriazine compounds of formula (I), or the agrochemical compositions comprising them, can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

Preferred crops are Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera and Zea mays.

Especially preferred crops are crops of cereals, corn, soybeans, rice, oilseed rape, cotton, potatoes, peanuts or permanent crops.

The aminotriazine compounds of formula (I) according to the invention, or the agrochemical compositions comprising them, can also be used in genetically modified plants. The term “genetically modified plants” is to be understood as plants whose genetic material has been modified by the use of recombinant DNA techniques to include an inserted sequence of DNA that is not native to that plant species' genome or to exhibit a deletion of DNA that was native to that species' genome, wherein the modification(s) cannot readily be obtained by cross breeding, mutagenesis or natural recombination alone. Often, a particular genetically modified plant will be one that has obtained its genetic modification(s) by inheritance through a natural breeding or propagation process from an ancestral plant whose genome was the one directly treated by use of a recombinant DNA technique. Typically, one or more genes have been integrated into the genetic material of a genetically modified plant in order to improve certain properties of the plant. Such genetic modifications also include but are not limited to targeted post-translational modification of protein(s), oligo- or polypeptides. e. g., by inclusion therein of amino acid mutation(s) that permit, decrease, or promote glycosylation or polymer additions such as prenylation, acetylation farnesylation, or PEG moiety attachment.

Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxyphenylpyruvate dioxygenase (HPPD) inhibitors or phytoene desaturase (PDS) inhibitors; acetolactate synthase (ALS) inhibitors such as sulfonyl ureas or imidazolinones; enolpyruvyl shikimate 3-phosphate synthase (EPSP) inhibitors such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering; furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are, for example, described in Pest Management Science 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein. Several cultivated plants have been rendered tolerant to herbicides by mutagenesis and conventional methods of breeding, e. g., Clearfield® summer rape (Canola, BASF SE, Germany) being tolerant to imidazolinones, e. g., imazamox, or ExpressSun® sunflowers (DuPont, USA) being tolerant to sulfonyl ureas, e. g., tribenuron. Genetic engineering methods have been used to render cultivated plants such as soybean, cotton, corn, beets and rape, tolerant to herbicides such as glyphosate, imidazolinones and glufosinate, some of which are under development or commercially available under the brands or trade names RoundupReady® (glyphosate tolerant, Monsanto, USA), Cultivance® (imidazolinone tolerant, BASF SE, Germany) and LibertyLink® (glufosinate tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as delta-endotoxins, e. g., CryIA(b), CryIA(c), CryIF, CryIF(a2), CryIIA(b), CryIIIA, CryIIIB(b1) or Cry9c; vegetative insecticidal proteins (VIP), e. g., VIP1, VIP2, VIP3 or VIP3A; insecticidal proteins of bacteria colonizing nematodes, e. g., Photorhabdus spp. or Xenorhabdus spp.; toxins produced by animals, such as scorpion toxins, arachnid toxins, wasp toxins, or other insect-specific neurotoxins; toxins produced by fungi, such as Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMGCoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as including pre-toxins, hybrid proteins, truncated or otherwise modified proteins. Hybrid proteins are characterized by a new combination of protein domains, (see, e. g., WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073.

The methods for producing such genetically modified plants are generally known to the person skilled in the art and are described, e. g., in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g., Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g., EP-A 392 225), plant disease resistance genes (e. g., potato culti-vars, which express resistance genes acting against Phytophthora infestans derived from the Mexican wild potato, Solanum bulbocastanum) or T4-lyso-zym (e.g., potato cultivars capable of synthesizing these proteins with increased resistance against bacteria such as Erwinia amylovora). The methods for producing such genetically modified plants are generally known to the person skilled in the art and are described, e.g., in the publications mentioned above.

Furthermore, plants are also covered that are by the use of recombinant DNA techniques capable to synthesize one or more proteins to increase the productivity (e.g., bio-mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of ingredients or new ingredients, specifically to improve human or animal nutrition, e. g., oil crops that produce health-promoting long-chain omega-3 fatty acids or unsaturated omega-9 fatty acids (e. g., Nexera® rape, Dow AgroSciences, Canada).

Furthermore, plants are also covered that contain by the use of recombinant DNA techniques a modified amount of ingredients or new ingredients, specifically to improve raw material production, e.g., potatoes that produce increased amounts of amylopectin (e.g. Amflora® potato, BASF SE, Germany).

Furthermore, it has been found that the compositions according to the invention are also suitable for the defoliation and/or desiccation of plant parts, for which crop plants such as cotton, potato, oilseed rape, sunflower, soybean or field beans, in particular cotton, are suitable. In this regard compositions have been found for the desiccation and/or defoliation of plants, processes for preparing these compositions, and methods for desiccating and/or defoliating plants using the compositions according to the invention.

As desiccants, the compositions according to the invention are suitable in particular for desiccating the above-ground parts of crop plants such as potato, oilseed rape, sunflower and soybean, but also cereals. This makes possible the fully mechanical harvesting of these important crop plants.

Also of economic interest is the facilitation of harvesting, which is made possible by concentrating within a certain period of time the dehiscence, or reduction of adhesion to the tree, in citrus fruit, olives and other species and varieties of pomaceous fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

Another aspect of the invention is an agrochemical composition comprising a herbicidal active amount of at least one compound of formula (I) as defined above and at least one inert liquid and/or solid carrier and, if appropriate, at least one surface-active substances.

A further aspect of the invention is a method of controlling undesired vegetation, which comprises allowing a herbicidally active amount of at least one compound of formula (I) as defined above to act on plants, their environment or on seed.

The preparation of the aminotriazine compounds of formula (I) is illustrated by examples; however, the subject matter of the present invention is not limited to the examples given.

The products shown below were characterized by the mass ([m/z]) or retention time (RT; [min.]) determined by HPLC-MS spectrometry.

HPLC-MS=high performance liquid chromatography-coupled mass spectrometry; HPLC column:

RP-18 column (Chromolith Speed ROD from Merck KgaA, Germany), 50*4.6 mm; mobile phase: acetonitrile+0.1% trifluoroacetic acid (TFA)/water+0.1% TFA using a gradient from 5:95 to 100:0 over 5 minutes at 40° C., flow rate 1.8 ml/min.

MS: quadrupole electrospray ionization, 80 V (positive mode).

### A PREPARATION EXAMPLES

### Example 1 (E.1): 4-(1-fluoroethyl)-6-(2,3,5,6-tetrafluorophenoxy)-1,3,5-triazin-2-amine

### 4-(1-fluoroethyl)-6-methylsulfanyl-1,3,5-triazin-2-amine

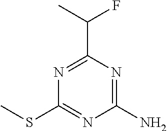

2-Fluoro-propanoyl chloride (24.5 g, 0.22 mol) and triethylamine (112 g, 1.11 mol) were added to a solution of 1-carbamimidoyl-2-methyl-isothiourea hydroiodide (57.7 g, 0.22 mol) in THF via two addition funnels. After the initial weak exothermic reaction was finished, the mixture was stirred for 3 h at 50 C. The reaction mixture was cooled to ambient temperature, diluted with water and ethyl acetate and the phases were separated. The organic phase was dried over anhydrous Na₂SO₄, filtered and concentrated under reduced pressure yielding the title compound as a colorless solid (13.6 g, 41.7% yield).

MS (ESI) m/z 189.1 [M+H⁺]

¹H NMR (400 MHz, CDCl₃): δ=6.18 (brs, 1H), 5.52 (brs, 1H), 5.42-5.29 (m, 1H), 2.50 (s, 3H), 1.69-1.62 (m, 3H) ppm.

### 4-chloro-6-(1-fluoroethyl)-1,3,5-triazin-2-amine

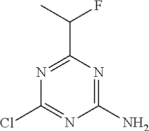

4-(1-fluoroethyl)-6-methylsulfanyl-1,3,5-triazin-2-amine (13.2 g, 70.2 mmol) was dissolved in acetic acid and Cl₂ gas was bubbled through the solution for 30 min. The reaction mixture was stirred for an additional hour at ambient temperature and was then carefully added to a cold solution of NaOH (70 g) in water (1 L). Ethyl acetate was added and the phases were separated. The organic phase was dried over anhydrous Na₂SO₄, filtered and concentrated under reduced pressure yielding the title compound as a colorless solid (5.32 g, 43.0% yield).

MS (ESI) m/z 177.0 [M+H⁺]

¹H NMR (400 MHz, CDCl₃): δ=6.77 (brs, 1H), 6.23 (brs, 1H), 5.42 (dq, J=48.3 Hz, J=6.7 Hz, 1H), 1.68 (dd, J=24.2 Hz, J=6.7 Hz, 3H) ppm.

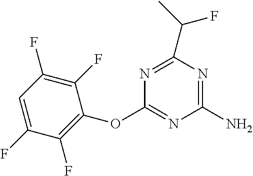 [SMILES: CC(F)c1nc(N)nc(Oc2c(F)c(F)cc(F)c2F)n1]

4-Chloro-6-(1-fluoroethyl)-1,3,5-triazin-2-amine (5.00 g, 28.32 mmol, 1.00 eq.) is added to a mixture of 2,3,5,6-tetrafluorophenol (4.70 g, 28.32 mmol, 1.00 eq.) and K₂CO₃ (7.83 g, 56.63 mmol, 2.00 eq.) in aceton. The reaction mixture is heated to 50° C. stirred for 14 hours before cooled to ambient temperature. Water and EtOAc are added, the phases separated and the organic phase is dried over Na₂SO₄. After filtration of the solids and removal of the volatiles under reduced pressure the crude product is purified via column chromatography (ISCO-CombiFlash Rf, cyclohexane/ethyl acetate) yielding the desired compound as a colorless solid (6.20 g, 71.5%).

MS (ESI) m/z 307.1 [M+H⁺]

¹H NMR (400 MHz, CDCl₃): δ=7.08-7.00 (m, 1H), 6.36 (brs, 1H), 5.58 (brs, 1H), 5.39 (dq, J=48.4 Hz, J=6.7 Hz, 1H), 1.66 (dd, J=24.1 Hz, J=6.7 Hz, 3H) ppm.

The compounds E.2 to E.45 listed below in table C have been prepared by analogy to the example E.1 mentioned above.

TABLE C

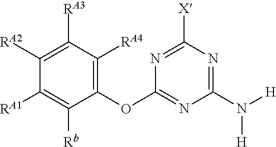

| | | | | | | | MS ^{¹⁾} |
|---|---|---|---|---|---|---|---|
| Example | R^{b} | R^{A4} | R^{A3} | R^{A2} | R^{A1} | X′ | [m/z] |
| E.1 | F | F | F | F | F | CHFCH₃ | 324.9 |
| E.2 | F | F | F | F | F | CF(CH₃)₂ | 339.0 |
| E.3 | F | H | H | F | H | CHFCH₃ | 270.9 |
| E.4 | CN | H | H | H | H | CHFCH₃ | 259.9 |
| E.5 | F | F | H | H | F | CF(CH₃)₂ | 303.1 |
| E.6 | F | H | H | H | F | CF(CH₃)₂ | 285.0 |
| E.7 | F | H | H | H | F | CHFCH₃ | 271.0 |
| E.8 | F | F | H | H | F | CHFCH₃ | 289.0 |
| E.9 | F | F | H | F | F | CHFCH₃ | 307.0 |
| E.10 | F | F | H | F | F | CF₂CH₃ | 325.0 |
| E.11 | F | F | H | F | F | CF₃ | 329.0 |
| E.12 | F | F | H | F | F | C(CH₃)₃ | 317.0 |
| E.13 | F | F | H | F | F | 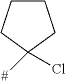 | 363.0 |
| E.14 | F | F | H | H | F | C(CH₃)₃ | 299.0 |
| E.15 | F | F | F | F | F | 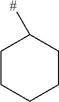 | 361.0 |
| E.16 | F | H | H | H | F | 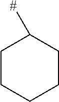 | 307.0 |
| E.17 | F | F | F | F | F | 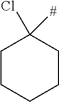 | 395.0 |
| E.18 | F | F | H | H | F | CF₂CH₃ | 307.0 |
| E.19 | F | F | H | F | F | 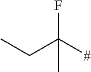 | 335.0 |
| E.20 | F | F | H | H | F | 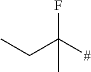 | 317.0 |
| E.21 | F | F | H | F | F | 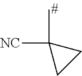 | 326.0 |
| E.22 | F | F | F | F | F | C(CH₃)₂CN | 346.0 |
| E.23 | F | F | H | F | F | 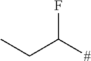 | 321.0 |
| E.24 | F | F | F | F | F | 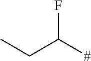 | 339.0 |
| E.25 | F | F | H | F | F | 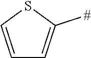 | 343.0 |
| E.26 | F | F | H | F | F | 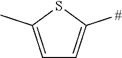 | 357.0 |
| E.27 | F | F | H | F | F | 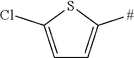 | 377.0 |
| E.28 | F | F | H | F | F | 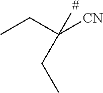 | 356.0 |
| E.29 | F | F | H | F | F | 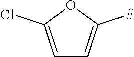 | 361.0 |
| E.30 | F | F | H | F | F | 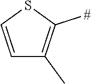 | 357.0 |
| E.31 | F | F | H | F | F | 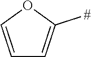 | 327.0 |
| E.32 | F | F | H | F | F | 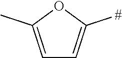 | 341.0 |
| E.33 | F | F | H | F | F | 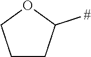 | 331.1 |
| E.34 | F | F | H | F | F | 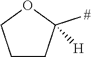 | 331.1 |
| E.35 | F | F | H | F | F | 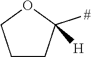 | 331.1 |
| E.36 | F | F | H | F | F | 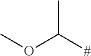 | 319.5 |
| E.37 | F | Cl | H | H | F | CHFCH₃ | 304.7 |
| E.38 | F | F | H | F | F | 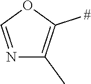 | 342.0 |
| E.39 | F | F | I | F | F | CHFCH₃ | 432.7 |
| E.40 | F | F | Br | F | F | CHFCH₃ | 384.7 |
| E.41 | F | F | Cl | F | F | CHFCH₃ | 340.7 |
| E.42 | F | F | H | F | F | 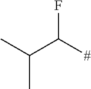 | 335.3 |
| E.43 | F | F | H | F | F | CH₂CH₃ | 288.8 |
| E.44 | F | F | H | F | F | CH(CH₃)₂ | 303.3 |
| E.45 | F | F | H | F | F | 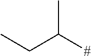 | 317.3 |

¹⁾ Mass Spectrum M⁺ [m/z]

### B USE EXAMPLES

The herbicidal activity of the azines of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this had been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C., respectively.

The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A moderate herbicidal activity is given at values of at least 60, a good herbicidal activity is given at values of at least 70, and a very good herbicidal activity is given at values of at least 85.

The plants used in the greenhouse experiments were of the following species:

| Bayer code | Scientific name |
|---|---|
| ABUTH | Abutilon theophrasti |
| ALOMY | Alopecurus agrestis |
| AMARE | Amaranthus retroflexus |
| APESV | Apera spica-venti |
| ECHCG | Echinocloa crus-galli |
| POLCO | Polygonum convolvulus |
| SETFA | Setaria faberi |
| SETVI | Setaria viridis |
| STEME | Stellaria media |
| VIOAR | Viola arvensis |

Example 1 applied by post-emergence method at an application rate of 3000 g/ha, showed good herbicidal activity against ABUTH and very good herbicidal activity against ECHCG and SETFA.

Example 2 applied by post-emergence method at an application rate of 3000 g/ha, showed very good herbicidal activity against ECHCG and SETFA.

Example 5 applied by post-emergence method at an application rate of 1000 g/ha, showed very good herbicidal activity against AMARE.

Example 7 applied by post-emergence method at an application rate of 1000 g/ha, showed very good herbicidal activity against ECHCG and SETFA.

Example 8 applied by post-emergence method at an application rate of 1000 g/ha, showed very good herbicidal activity against AMARE, SEFTA and VIOR.

Example 9 applied by post-emergence method at an application rate of 1000 g/ha, showed very good herbicidal activity against AMARE, SEFTA and VIOR.

Example 11 applied by post-emergence method at an application rate of 1000 g/ha, showed very good herbicidal activity against SETFA.

Example 19 applied by post-emergence method at an application rate of 500 g/ha, showed very good herbicidal activity against AMARE.

Example 22 applied by post-emergence method at an application rate of 500 g/ha, showed good herbicidal activity against ALOMY.

Example 23 applied by pre-emergence method at an application rate of 1000 g/ha, showed very good herbicidal activity against AMARE, ECHCG and SETFA.

Example 24 applied by pre-emergence method at an application rate of 1000 g/ha, showed very good herbicidal activity against ABUTH, ECHCG and SETFA.

Example 25 applied by post-emergence method at an application rate of 500 g/ha, showed good herbicidal activity against ECHCG.

Example 27 applied by post-emergence method at an application rate of 500 g/ha, showed herbicidal activity against AMARE.

Example 32 applied by post-emergence method at an application rate of 1000 g/ha, showed very herbicidal activity against ABUTH.

Example 33 applied by post-emergence method at an application rate of 125 g/ha, showed very good herbicidal activity against POLCO.

Example 34 applied by pre-emergence method at an application rate of 500 g/ha, showed good herbicidal activity against AMARE and SETFA.

Example 35 applied by post-emergence method at an application rate of 500 g/ha, showed good herbicidal activity against AMARE.

Example 38 applied by post-emergence method at an application rate of 1000 g/ha, showed good herbicidal activity against ECHCG.

Example 39 applied by post-emergence method at an application rate of 500 g/ha, showed good herbicidal activity against ECHCG and very good herbicidal activity against SETFA.

Example 42 applied by pre-emergence method at an application rate of 500 g/ha, showed good herbicidal activity against AMARE and ECHCG and very good herbicidal activity against SETFA.

1.-16. (canceled)

17. A method of controlling undesired vegetation, which comprises allowing a herbicidally active amount of at least one compound of formula (I) 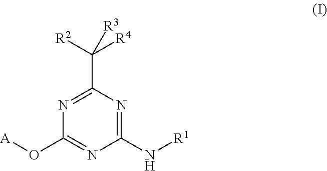
where
A is a radical of formula (A.1) 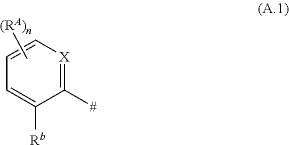
wherein
# indicates the point of attachment to the oxygen; X is CR^{A4} or N;
R^{A} is selected from the group consisting of halogen, OH, CN, amino, NO₂, C₁-C₆-alkyl, C₂-C₆-alkenyl, C₂-C₆-alkynyl, C₁-C₆-alkoxy, C₂-C₆-alkenyloxy, C₂-C₆-alkynyloxy, (C₁-C₆-alkoxy)-C₁-C₆-alkyl, (C₁-C₆-alkoxy)-C₁-C₆-alkoxy, (C₁-C₆-alkoxy)-C₂-C₆-alkenyl, (C₁-C₆-alkoxy)-C₂-C₆-alkynyl, C₁-C₆-alkylthio, (C₁-C₆-alkyl)sulfinyl, (C₁-C₆-alkyl)sulfonyl, (C₁-C₆-alkyl)amino, di(C₁-C₆-alkyl)amino, (C₁-C₆-alkyl)-carbonyl, (C₁-C₆-alkoxy)-carbonyl, (C₁-C₆-alkyl)-carbonyloxy, C₃-C₆-cycloalkyl, C₃-C₆-cycloalkoxy, (C₃-C₆-cycloalkyl)-C₁-C₄-alkyl, and (C₃-C₆-cycloalkyl)-C₁-C₄-alkoxy, where the aliphatic and cycloaliphatic parts of the 22 aforementioned radicals are unsubstituted, partly or completely halogenated and where the cycloaliphatic parts of the last 4 mentioned radicals may carry 1, 2, 3, 4, 5 or 6 methyl groups, it being possible that two radicals R^{A} which are bound to adjacent ring atoms of phenyl or 6-membered hetaryl may form a fused 5- or 6-membered carbocyclic or heterocyclic ring, which itself is substituted or unsubstituted by 1, 2, 3, 4 or 5 identical or different substituents R^{B} selected from the group consisting of halogen, OH, CN, amino, NO₂, C₁-C₆-alkyl, C₂-C₆-alkenyl, C₂-C₆-alkynyl, C₁-C₆-alkoxy, C₂-C₆-alkenyloxy, C₂-C₆-alkynyloxy, (C₁-C₆-alkoxy)-C₁-C₆-alkyl, (C₁-C₆-alkoxy)-C₁-C₆-alkoxy, (C₁-C₆-alkoxy)-C₂-C₆-alkenyl, (C₁-C₆-alkoxy)-C₂-C₆-alkynyl, C₁-C₆-alkylthio, (C₁-C₆-alkyl)sulfinyl, (C₁-C₆-alkyl)sulfonyl, (C₁-C₆-alkyl)amino, di(C₁-C₆-alkyl)amino, (C₁-C₆-alkyl)-carbonyl, (C₁-C₆-alkoxy)-carbonyl, (C₁-C₆-alkyl)-carbonyloxy, C₃-C₆-cycloalkyl, C₃-C₆-cycloalkoxy, (C₃-C₆-cycloalkyl)-C₁-C₄-alkyl, and (C₃-C₆-cycloalkyl)-C₁-C₄-alkoxy, where the aliphatic and cycloaliphatic parts of the 22 aforementioned radicals are unsubstituted, partly or completely halogenated and where the cycloaliphatic parts of the last 4 mentioned radicals may carry 1, 2, 3, 4, 5 or 6 methyl groups, it being possible that two radicals R^{B} which are bound at the same carbon atom may together be ═O or ═NR^{b};
R^{A4} is hydrogen or has one of the meanings given for R^{A};
n is 0, 1, 2 or 3;
R^{b} is selected from the group consisting of halogen and CN;
R¹ is selected from the group consisting of H, OH, S(O)₂NH₂, CN, C₁-C₆-alkyl, C₂-C₆-alkenyl, C₂-C₆-alkynyl, (C₃-C₆-cycloalkyl)-C₁-C₄-alkyl, C₁-C₆-alkoxy, (C₁-C₆-alkoxy)-C₁-C₆-alkyl, (C₁-C₆-alkyl)carbonyl, (C₁-C₆-alkoxy)carbonyl, (C₁-C₆-alkyl)sulfonyl, (C₁-C₆-alkylamino)carbonyl, di(C₁-C₆-alkyl)aminocarbonyl, (C₁-C₆-alkylamino)sulfonyl, di(C₁-C₆-alkyl)aminosulfonyl and (C₁-C₆-alkoxy)sulfonyl, where the aliphatic and cycloaliphatic parts of the 14 aforementioned radicals are unsubstituted, partly or completely halogenated, phenyl, phenylsulfonyl, phenylaminosulfonyl, phenyl-C₁-C₆ alkyl, phenoxy, phenylcarbonyl and phenoxycarbonyl,
wherein phenyl in the last 7 mentioned radicals is unsubstituted or substituted by 1, 2, 3, 4 or 5 identical or different substituents selected from the group consisting of halogen, CN, NO₂, C₁-C₆-alkyl, C₁-C₆-haloalkyl, C₁-C₆-alkoxy and C₁-C₆-haloalkoxy;
R² is H, halogen, OH, CN, C₁-C₆-alkyl, C₂-C₆-alkenyl, C₃-C₆-alkynyl, (C₁-C₆-alkoxy)-C₁-C₆-alkyl, C₃-C₆-cycloalkyl, C₃-C₆-cycloalkenyl, (C₃-C₆-cycloalkyl)-C₁-C₄-alkyl, C₁-C₆-alkoxy, C₂-C₆-alkenyloxy, C₂-C₆-alkynyloxy, C₃-C₆-cycloalkoxy or (C₃-C₆-cycloalkyl)-C₁-C₄-alkoxy, where the aliphatic and cycloaliphatic parts of the 12 aforementioned radicals are unsubstituted, partly or completely halogenated,
phenyl, phenyl-C₁-C₆-alkyl,
wherein phenyl in the last 2 mentioned radicals are unsubstituted or substituted by 1, 2, 3, 4 or 5 identical or different substituents selected from the group consisting of halogen, CN, NO₂, C₁-C₆-alkyl, C₁-C₆-haloalkyl, C₁-C₆-alkoxy and C₁-C₆-haloalkoxy,
R³ is selected from the group consisting of H, halogen, CN, C₁-C₆-alkyl, C₁-C₆-haloalkyl, C₁-C₆-alkoxy and C₁-C₆-haloalkoxy;
R⁴ is selected from the group consisting of H, halogen, CN, C₁-C₆-alkyl, C₂-C₆-alkenyl, C₃-C₆-alkynyl, C₃-C₆-cycloalkyl, (C₃-C₆-cycloalkyl)-C₁-C₄-alkyl, C₃-C₆-cycloalkenyl and C₁-C₆-alkoxy-C₁-C₆-alkyl, where the aliphatic and cycloaliphatic parts of the 7 aforementioned radicals are unsubstituted, partly or completely halogenated; or
R³ and R⁴ together with the carbon atom to which they are attached form a moiety selected from the group consisting of carbonyl, C₂-C₆-alkenyl, C₃-C₆-cycloalkyl, C₃-C₆-cycloalkenyl and three- to six-membered saturated or partially unsaturated heterocyclyl,
wherein the C₃-C₆-cycloalkyl, C₃-C₆-cycloalkenyl, or three- to six-membered heterocyclyl is unsubstituted or substituted by one to six substituents selected from halogen, CN, C₁-C₆-alkyl and C₁-C₆-alkoxy; or
R² and R³ and R⁴ together with the carbon atom to which they are attached may form a moiety selected from a 5- or 6-membered aromatic heterocyclyl, which is unsubstituted or substituted by one to six substituents selected from halogen, CN, C₁-C₆-alkyl and C₁-C₆-alkoxy;
or an agriculturally acceptable salt thereof;
to act on plants, their environment or on seed.

18. The method of claim 17, wherein R^{A} and R^{B}, if present, are selected from the group consisting of halogen, CN, C₁-C₄-alkyl, C₁-C₄-alkoxy and C₁-C₄-haloalkoxy, in particular from fluorine, chlorine or methyl, or two radicals R^{B} which are bound at the same carbon atom may together be ═O or ═NR^{b}, wherein R^{b} is selected from the group consisting of halogen and CN.

19. The method of claim 17, wherein
X is CR^{A4};
R^{b} is F;
n is 0, 1, 2 or 3;
R^{A} is selected from the group consisting of halogen, CN, C₁-C₆-alkyl, C₁-C₆-alkoxy, C₁-C₆-haloalkyl, C₃-C₆-cycloalkyl, C₃-C₆-cycloalkoxy, (C₃-C₆-cycloalkyl)methoxy, C₂-C₆-alkynyl, C₂-C₆-alkenyl, C₂-C₆-alkynyloxy, C₂-C₆-alkenyloxy and C₁-C₆-haloalkoxy; and
R^{A4} is selected from the group consisting of H, halogen, CN, C₁-C₆-alkyl, C₁-C₆-alkoxy, C₁-C₆-haloalkyl, C₃-C₆-cycloalkyl, C₃-C₆-cycloalkoxy, (C₃-C₆-cycloalkyl)methoxy, C₂-C₆-alkynyl, C₂-C₆-alkenyl, C₂-C₆-alkynyloxy, C₂-C₆-alkenyloxy and C₁-C₆-haloalkoxy.

20. The method of claim 19, wherein
X is CR^{A4};
R^{b} is F;
N is 0, 1, 2 or 3;
R^{A} is selected from the group consisting of F, Br, Cl, CN, CF₃, methyl, vinyl, ethynyl, cyclopropyl, methoxy, ethoxy, isopropyloxy, allyloxy, propargyloxy, cyclopropyloxy, cyclobutyloxy, cyclopentyloxy, (cyclopropyl)methoxy and 2-butyloxy; and
R^{A4} is selected from the group consisting of H, F, Cl, CN, CF₃, methyl, vinyl, ethynyl, cyclopropyl, methoxy, ethoxy, isopropyloxy, allyloxy, propargyloxy, cyclopropyloxy, cyclobutyloxy, cyclopentyloxy, (cyclopropyl)methoxy and 2-butyloxy.

21. The method of claim 19, wherein both R^{b} and R^{A4} are F.

22. The method claim 17, wherein A is selected from the group consisting of 2,6-difluorophenyl, 2-chloro-6-fluorophenyl, 2-bromo-6-fluorophenyl, 2-fluoro-6-methylphenyl, 2-fluoro-6-cyanophenyl, 2,3,6-trifluorophenyl, 2,4,6-trifluorophenyl, 2-chloro-4,6-difluorophenyl, 2-chloro-5,6-difluorophenyl, 3-chloro-2,6-difluorophenyl, 2-chloro-3,6-difluorophenyl, 2,3-difluoro-6-cyanophenyl, 2,6-difluoro-3-cyanophenyl, 2,5-difluoro-6-cyanophenyl, 2,4-difluoro-6-cyanophenyl, 2,3,4,6-tetrafluorophenyl, 2,3,4,5-tetrafluorophenyl, 2,3,5,6-tetrafluorophenyl, 2-chloro-3,4,6-trifluorophenyl, 2-chloro-3,5,6-trifluorophenyl, 3-chloro-2,4,6-trifluorophenyl, 3-chloro-2,5,6-trifluorophenyl, 6-cyano-2,4,5-trifluorophenyl, 3-cyano-2,4,6-trifluorophenyl, 6-cyano-2,3,4-trifluorophenyl, 6-cyano-2,3,5-trifluorophenyl, 3-cyano-2,5,6-trifluorophenyl, 2,3,4,5,6-pentafluorophenyl, 2-chloro-3,4,5,6-tetrafluorophenyl, 3-chloro-2,4,5,6-tetrafluorophenyl, 6-cyano-2,3,4,5-tetrafluorophenyl, 5-cyano-2,3,4,6-tetrafluorophenyl, 4-bromo-2,3,5,6-tetrafluorophenyl, 4-iodo-2,3,5,6-tetrafluorophenyl, 4-ethynyl-2,3,5,6-tetrafluorophenyl, 3-chloro-2-fluoro-6-(trifluoromethyl)phenyl, with particular preference given to 2,6-difluorophenyl, 2,3,6-trifluorophenyl, 2,3,5,6-tetrafluorophenyl and 2,3,4,5,6-pentafluorophenyl.

23. The method of claim 17, wherein
X is N;
R^{b} is F;
R^{A} is selected from the group consisting of halogen, CN, C₁-C₆-alkyl, C₁-C₆-alkoxy, C₁-C₆-haloalkyl, C₃-C₆-cycloalkyl, C₃-C₆-cycloalkoxy, (C₃-C₆-cycloalkyl)methoxy, C₂-C₆-alkynyl, C₂-C₆-alkenyl, C₂-C₆-alkynyloxy, C₂-C₆-alkenyloxy and C₁-C₆-haloalkoxy;
n is 0, 1, 2 or 3.

24. The method of claim 17, wherein R¹ is selected from the group consisting of H, CN, C₁-C₆-alkyl, (C₁-C₆-alkoxy)-C₁-C₆-alkyl, (C₁-C₆-alkyl)carbonyl, (C₃-C₆-cycloalkyl)-carbonyl, (C₁-C₆-alkyl)sulfonyl, (C₁-C₆-alkoxy)carbonyl, (C₁-C₆-alkylamino)carbonyl, di(C₁-C₆-alkyl)aminocarbonyl, (C₁-C₆-alkylamino)sulfonyl, di(C₁-C₆-alkyl)aminosulfonyl, where the aliphatic parts of the 10 aforementioned radicals are unsubstituted, partly or completely halogenated,
phenyl, phenylcarbonyl and C₁-C₆ alkylphenyl,
wherein phenyl in the last 3 mentioned radical is unsubstituted or substituted by 1, 2, 3, 4, or 5 identical or different substituents selected from the group consisting of halogen, CN, NO₂, C₁-C₆-alkyl, C₁-C₆-haloalkyl, C₁-C₆-alkoxy and halo-C₁-C₆-alkoxy.

25. The method of claim 17, wherein, R² is selected from the group consisting of H, halogen, C₁-C₆-alkyl, C₁-C₆-haloalkyl, C₁-C₄-alkoxy, and C₁-C₆-haloalkoxy.

26. The method of claim 17, wherein,
R³ is selected from the group consisting of hydrogen, fluorine, C₁-C₄-alkyl, C₁-C₄-haloalkyl, C₁-C₄-alkoxy and C₁-C₆-haloalkoxy;
R⁴ is selected from the group consisting of C₁-C₆-alkyl, C₁-C₆-haloalkyl, C₂-C₆-alkenyl, C₃-C₆-alkynyl, C₃-C₆-cycloalkyl, C₃-C₆-cycloalkenyl, and C₁-C₆-alkoxy-C₁-C₆-alkyl;
or
R³ and R⁴ together with the carbon atom to which they are attached form a moiety selected from the group consisting of C₃-C₆-cycloalkyl, C₃-C₆-cycloalkenyl and three- to six-membered saturated or partially unsaturated heterocyclyl.

27. The method of claim 17, wherein R¹ is H.

28. An agrochemical composition comprising at least one compound as defined in claim 17 and at least one inert liquid and/or solid carrier and, if appropriate, at least one surface-active substances.

29. A process for the preparation of herbicidal active agrochemical compositions, which comprises mixing a herbicidally active amount of at least one compound as defined in claim 17 and at least one inert liquid and/or solid carrier and, if desired, at least one surface-active substance.

30. The method of claim 17, wherein said controlling is desiccation/defoliation of undesired plants.

31. A compound of the formula (I), 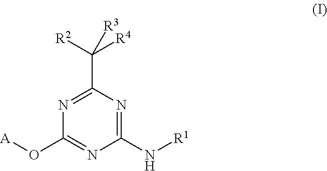
wherein
A is a radical of formula (A.1) 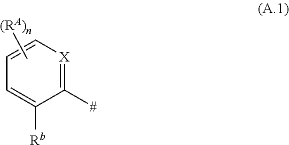
wherein
# indicates the point of attachment to the oxygen; X is CR^{A4} or N;
R^{A} is selected from the group consisting of halogen, OH, CN, amino, NO₂, C₁-C₆-alkyl, C₂-C₆-alkenyl, C₂-C₆-alkynyl, C₁-C₆-alkoxy, C₂-C₆-alkenyloxy, C₂-C₆-alkynyloxy, (C₁-C₆-alkoxy)-C₁-C₆-alkyl, (C₁-C₆-alkoxy)-C₁-C₆-alkoxy, (C₁-C₆-alkoxy)-C₂-C₆-alkenyl, (C₁-C₆-alkoxy)-C₂-C₆-alkynyl, C₁-C₆-alkylthio, (C₁-C₆-alkyl)sulfinyl, (C₁-C₆-alkyl)sulfonyl, (C₁-C₆-alkyl)amino, di(C₁-C₆-alkyl)amino, (C₁-C₆-alkyl)-carbonyl, (C₁-C₆-alkoxy)-carbonyl, (C₁-C₆-alkyl)-carbonyloxy, C₃-C₆-cycloalkyl, C₃-C₆-cycloalkoxy, (C₃-C₆-cycloalkyl)-C₁-C₄-alkyl, and (C₃-C₆-cycloalkyl)-C₁-C₄-alkoxy, where the aliphatic and cycloaliphatic parts of the 22 aforementioned radicals are unsubstituted, partly or completely halogenated and where the cycloaliphatic parts of the last 4 mentioned radicals may carry 1, 2, 3, 4, 5 or 6 methyl groups, it being possible that two radicals R^{A} which are bound to adjacent ring atoms of phenyl or 6-membered hetaryl may form a fused 5- or 6-membered carbocyclic or heterocyclic ring, which itself is substituted or unsubstituted by 1, 2, 3, 4 or 5 identical or different substituents R^{B} selected from the group consisting of halogen, OH, CN, amino, NO₂, C₁-C₆-alkyl, C₂-C₆-alkenyl, C₂-C₆-alkynyl, C₁-C₆-alkoxy, C₂-C₆-alkenyloxy, C₂-C₆-alkynyloxy, (C₁-C₆-alkoxy)-C₁-C₆-alkyl, (C₁-C₆-alkoxy)-C₁-C₆-alkoxy, (C₁-C₆-alkoxy)-C₂-C₆-alkenyl, (C₁-C₆-alkoxy)-C₂-C₆-alkynyl, C₁-C₆-alkylthio, (C₁-C₆-alkyl)sulfinyl, (C₁-C₆-alkyl)sulfonyl, (C₁-C₆-alkyl)amino, di(C₁-C₆-alkyl)amino, (C₁-C₆-alkyl)-carbonyl, (C₁-C₆-alkoxy)-carbonyl, (C₁-C₆-alkyl)-carbonyloxy, C₃-C₆-cycloalkyl, C₃-C₆-cycloalkoxy, (C₃-C₆-cycloalkyl)-C₁-C₄-alkyl, and (C₃-C₆-cycloalkyl)-C₁-C₄-alkoxy, where the aliphatic and cycloaliphatic parts of the 22 aforementioned radicals are unsubstituted, partly or completely halogenated and where the cycloaliphatic parts of the last 4 mentioned radicals may carry 1, 2, 3, 4, 5 or 6 methyl groups, it being possible that two radicals R^{B} which are bound at the same carbon atom may together be ═O or ═NR^{b};
R^{A4} is hydrogen or has one of the meanings given for R^{A};
n is 0, 1, 2 or 3;
R^{b} is selected from the group consisting of halogen and CN;
R¹ is selected from the group consisting of H, OH, S(O)₂NH₂, CN, C₁-C₆-alkyl, C₂-C₆-alkenyl, C₂-C₆-alkynyl, (C₃-C₆-cycloalkyl)-C₁-C₄-alkyl, C₁-C₆-alkoxy, (C₁-C₆-alkoxy)-C₁-C₆-alkyl, (C₁-C₆-alkyl)carbonyl, (C₁-C₆-alkoxy)carbonyl, (C₁-C₆-alkyl)sulfonyl, (C₁-C₆-alkylamino)carbonyl, di(C₁-C₆-alkyl)aminocarbonyl, (C₁-C₆-alkylamino)sulfonyl, di(C₁-C₆-alkyl)aminosulfonyl and (C₁-C₆-alkoxy)sulfonyl, where the aliphatic and cycloaliphatic parts of the 14 aforementioned radicals are unsubstituted, partly or completely halogenated,
phenyl, phenylsulfonyl, phenylaminosulfonyl, phenyl-C₁-C₆ alkyl, phenoxy, phenylcarbonyl and phenoxycarbonyl,
wherein phenyl in the last 7 mentioned radicals is unsubstituted or substituted by 1, 2, 3, 4 or 5 identical or different substituents selected from the group consisting of halogen, CN, NO₂, C₁-C₆-alkyl, C₁-C₆-haloalkyl, C₁-C₆-alkoxy and C₁-C₆-haloalkoxy;
R² is H, halogen, OH, CN, C₁-C₆-alkyl, C₂-C₆-alkenyl, C₃-C₆-alkynyl, (C₁-C₆-alkoxy)-C₁-C₆-alkyl, C₃-C₆-cycloalkyl, C₃-C₆-cycloalkenyl, (C₃-C₆-cycloalkyl)-C₁-C₄-alkyl, C₁-C₆-alkoxy, C₂-C₆-alkenyloxy, C₂-C₆-alkynyloxy, C₃-C₆-cycloalkoxy or (C₃-C₆-cycloalkyl)-C₁-C₄-alkoxy, where the aliphatic and cycloaliphatic parts of the 12 aforementioned radicals are unsubstituted, partly or completely halogenated,
phenyl, phenyl-C₁-C₆-alkyl,
wherein phenyl in the last 2 mentioned radicals are unsubstituted or substituted by 1, 2, 3, 4 or 5 identical or different substituents selected from the group consisting of halogen, CN, NO₂, C₁-C₆-alkyl, C₁-C₆-haloalkyl, C₁-C₆-alkoxy and C₁-C₆-haloalkoxy,
R³ is selected from the group consisting of H, halogen, CN, C₁-C₆-alkyl, C₁-C₆-haloalkyl, C₁-C₆-alkoxy and C₁-C₆-haloalkoxy;
R⁴ is selected from the group consisting of H, halogen, CN, C₁-C₆-alkyl, C₂-C₆-alkenyl, C₃-C₆-alkynyl, C₃-C₆-cycloalkyl, (C₃-C₆-cycloalkyl)-C₁-C₄-alkyl, C₃-C₆-cycloalkenyl and C₁-C₆-alkoxy-C₁-C₆-alkyl, where the aliphatic and cycloaliphatic parts of the 7 aforementioned radicals are unsubstituted, partly or completely halogenated; or
R³ and R⁴ together with the carbon atom to which they are attached form a moiety selected from the group consisting of carbonyl, C₂-C₆-alkenyl, C₃-C₆-cycloalkyl, C₃-C₆-cycloalkenyl and three- to six-membered saturated or partially unsaturated heterocyclyl,
wherein the C₃-C₆-cycloalkyl, C₃-C₆-cycloalkenyl, or three- to six-membered heterocyclyl is unsubstituted or substituted by one to six substituents selected from halogen, CN, C₁-C₆-alkyl and C₁-C₆-alkoxy; or
R² and R³ and R⁴ together with the carbon atom to which they are attached may form a moiety selected from a 5- or 6-membered aromatic heterocyclyl, which is unsubstituted or substituted by one to six substituents selected from halogen, CN, C₁-C₆-alkyl and C₁-C₆-alkoxy;
or an agriculturally acceptable salt thereof;
except for the following compounds:
4-(2-fluorophenoxy)-6-methyl-1,3,5-triazin-2-amine,
4-(2-chlorophenoxy)-6-methyl-1,3,5-triazin-2-amine,
2-[(4-amino-6-methyl-1,3,5-triazin-2-yl)oxy]benzonitrile,
4-methyl-6-(2,4,6-trichlorophenoxy)-1,3,5-triazin-2-amine,
4-[2-chloro-4-(trifluoromethyl)phenoxy]-6-methyl-1,3,5-triazin-2-amine,
4-(2-fluorophenoxy)-6-(trifluoromethyl)-1,3,5-triazin-2-amine,
4-(2-chlorophenoxy)-6-(trifluoromethyl)-1,3,5-triazin-2-amine,
2-[(4-amino-6-(trifluoromethyl)-1,3,5-triazin-2-yl)oxy]benzonitrile,
4-(2,4,6-trichlorophenoxy)-6-(trifluoromethyl)-1,3,5-triazin-2-amine,
4-[2-chloro-4-(trifluoromethyl)phenoxy]-6-(trifluoromethyl)-1,3,5-triazin-2-amine,
4-(2,6-dichlorophenoxy)-6-(trifluoromethyl)-1,3,5-triazin-2-amine,
4-(2-bromo-4-isopropenyl-6-methoxy-phenoxy)-N-[1-(methoxymethyl)propyl]-6-methyl-1,3,5-triazin-2-amine,
4-(2-bromo-4-isopropyl-6-methoxy-phenoxy)-N-[1-(methoxymethyl)propyl]-6-methyl-1,3,5-triazin-2-amine,
4-[2-bromo-4-methoxy-6-(1-methylethenyl)phenoxy]-N-[1-(methoxymethyl)propyl]-6-methyl-1,3,5-triazin-2-amine,
4-[2-bromo-4-methoxy-6-(1-methylethyl)phenoxy]-N-[1-(methoxymethyl)propyl]-6-methyl-1,3,5-triazin-2-amine and
1-[3-bromo-5-methoxy-4-[[4-[[1-(methoxymethyl)propyl]amino]-6-methyl-1,3,5-triazin-2-yl]oxy]phenyl]-ethanone.

32. The compound as claimed in claim 31, selected from the group consisting of compounds E.1 to E.45, which are of the formula (I′), where R^{A1}, R^{A2}, R^{A3}, R^{A4} R^{b} and X′ are as defined in the following table and their agriculturally acceptable salts:
(I′)

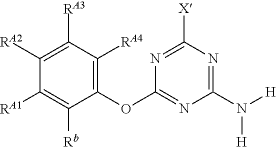

| Com- | | | | | | |
|---|---|---|---|---|---|---|
| pound | R^{b} | R^{A4} | R^{A3} | R^{A2} | R^{A1} | X′ |
| E.1 | F | F | F | F | F | CHFCH₃ |
| E.2 | F | F | F | F | F | CF(CH₃)₂ |
| E.3 | F | H | H | F | H | CHFCH₃ |
| E.4 | CN | H | H | H | H | CHFCH₃ |
| E.5 | F | F | H | H | F | CF(CH₃)₂ |
| E.6 | F | H | H | H | F | CF(CH₃)₂ |
| E.7 | F | H | H | H | F | CHFCH₃ |
| E.8 | F | F | H | H | F | CHFCH₃ |
| E.9 | F | F | H | F | F | CHFCH₃ |
| E.10 | F | F | H | F | F | CF₂CH₃ |
| E.11 | F | F | H | F | F | CF₃ |
| E.12 | F | F | H | F | F | C(CH₃)₃ |
| E.13 | F | F | H | F | F | 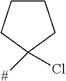 |
| E.14 | F | F | H | H | F | C(CH₃)₃ |
| E.15 | F | F | F | F | F | 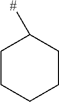 |
| E.16 | F | H | H | H | F | 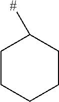 |
| E.17 | F | F | F | F | F | 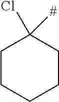 |
| E.18 | F | F | H | H | F | CF₂CH₃ |
| E.19 | F | F | H | F | F | 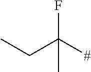 |
| E.20 | F | F | H | H | F | 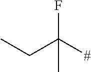 |
| E.21 | F | F | H | F | F | 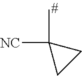 |
| E.22 | F | F | F | F | F | C(CH₃)₂CN |
| E.23 | F | F | H | F | F | 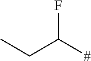 |
| E.24 | F | F | F | F | F | 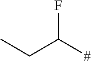 |
| E.25 | F | F | H | F | F | 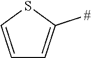 |
| E.26 | F | F | H | F | F | 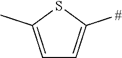 |
| E.27 | F | F | H | F | F | 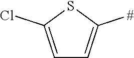 |
| E.28 | F | F | H | F | F | 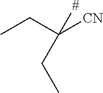 |
| E.29 | F | F | H | F | F | 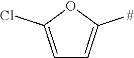 |
| E.30 | F | F | H | F | F | 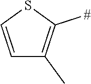 |
| E.31 | F | F | H | F | F | 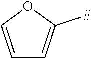 |
| E.32 | F | F | H | F | F | 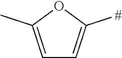 |
| E.33 | F | F | H | F | F | 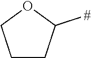 |
| E.34 | F | F | H | F | F | 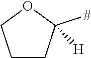 |
| E.35 | F | F | H | F | F | 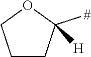 |
| E.36 | F | F | H | F | F | 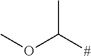 |
| E.37 | F | Cl | H | H | F | CHFCH₃ |
| E.38 | F | F | H | F | F | 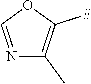 |
| E.39 | F | F | I | F | F | CHFCH₃ |
| E.40 | F | F | Br | F | F | CHFCH₃ |
| E.41 | F | F | Cl | F | F | CHFCH₃ |
| E.42 | F | F | H | F | F | 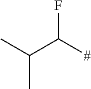 |
| E.43 | F | F | H | F | F | CH₂CH₃ |
| E.44 | F | F | H | F | F | CH(CH₃)₂ |
| E.45 | F | F | H | F | F | 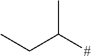 |
